(19) United States
(12) Patent Application Publication
(10) Pub. No.: US 2007/0259879 A1
(43) Pub. Date: Nov. 8, 2007

(54) Piperazine and piperidine biaryl derivatives

(75) Inventors: Rong Jian Lu, Chapel Hill, NC (US); John Alan Tucker, San Diego, CA (US); Jason C. Pickens, San Diego, CA (US); Tatiana V. Zinevitch, Moscow (RU); Sergey I. Sviridov, Moscow (RU); Vitaly E. Konoplev, Toulskaya (RU); Enugurthi Brahmachary, San Diego, CA (US); You-An Ma, Poway, CA (US)

Correspondence Address: MYERS BIGEL SIBLEY & SAJOVEC, PO BOX 37428, RALEIGH, NC, 27627, US

(73) Assignee: Trimeris, Inc.

(21) Appl. No.: 11/714,539

(22) Filed: Mar. 6, 2007

Related U.S. Application Data
(60) Provisional application No. 60/779,462, filed on Mar. 6, 2006.

(51) Int. Cl. A61K 31/505; A61K 31/497; A61P 37/00; C07D 241/36; C07D 401/02; C07D 403/02; C07D 405/02; C07D 413/02; C07D 495/02

(52) U.S. Cl. 514/252.13; 514/253.01; 514/253.06; 514/254.02; 514/254.03; 514/254.05; 514/254.08; 514/255.02; 514/274; 544/278; 544/295; 544/360; 544/363; 544/366; 544/367; 544/369; 544/370; 544/371; 544/373; 544/374; 544/383

(57) ABSTRACT

This invention relates to piperazine and piperidine biaryl compounds of Formula (I): 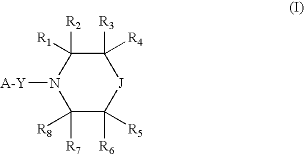 or a pharmaceutically acceptable prodrug, salt, polymorph, solvate, enantiomer, diastereomer, racemate, mixture of stereoisomers thereof, or derivative thereof; and to processes for preparing the compounds or pharmaceutical compositions containing the same. The compounds and pharmaceutical compositions of the present invention are provided for use in the treatment of HIV infection and/or AIDS.

### RELATED APPLICATION DATA

This application claims the benefit of U.S. Provisional Patent Application Ser. No. 60/779,462, filed on Mar. 6, 2006, the contents of which are incorporated herein by reference in its entirety.

### FIELD OF INVENTION

The present invention relates to piperazine derivatives, and to processes of preparation, compositions and methods of using the same. More specifically, the present invention relates piperazine derivatives and compositions, and to methods of using the same in the treatment of Human Immunodeficiency Virus (HIV) infection and Acquired Immunodeficiency Syndrome (AIDS).

### BACKGROUND OF THE INVENTION

Currently, it is estimated that over 42 million individuals, including 2.5 million children, are infected with HIV worldwide. In addition, it is estimated that worldwide over 14,000 people are infected with HIV daily and that 3 million people die each year from HIV-related causes. Advances in antiretroviral therapy, used in the treatment of HIV and AIDS, have resulted in relatively fewer people dying of AIDS. However, the number of HIV-infected people continues to rise. In addition to the personal costs associated with HIV infection, over $10 billion is spent annually on drugs to treat HIV infection and AIDS.

It is estimated that even with antiviral therapy adherence rates of 80 to 90 percent, over time there is up to a 50 percent or greater failure rate for HIV therapy. A number of factors play a role in the development of resistance to antiretroviral therapy, including: the infecting virus subtype; the infected individual's genetics and compliance with therapy; and the therapeutic regimen(s) used. However, the eventual high rates of virologic failure are often ascribed to the ability of the virus to readily mutate to escape the action of a class of drugs, such that a few mutations, or even a single mutation, in the viral sequence may be sufficient to enable resistance broadly across a class of drugs. Currently, there are only a few classes of drugs available for inclusion in a therapeutic regimen of antiretroviral therapy. Thus, there is a need for new drugs to treat HIV infection and AIDS.

It is now well known that cells can be infected by HIV, such as HIV-1, through a process by which fusion occurs between the cellular membrane and the viral membrane. The generally accepted model of this process is that the viral envelope glycoprotein complex (gp120/gp41) interacts with cell surface receptors on the membranes of the target cells. The process involves the interaction of the envelope glycoprotein gp120 with the cell surface receptor CD4. Such interaction may trigger a conformational change in gp120 facilitating its binding to co-receptors (a chemokine receptor such as CCR5 or CXCR4). Following binding of gp120 to cellular receptors, a conformational change may be induced in the gp120/gp41 complex that allows gp41 to insert into the membrane of the target cell and mediate membrane fusion. Thus, gp120 plays an important role in HIV entry and serves as a potential target for the development of HIV-1 entry inhibitors, a new class of anti-HIV drugs that currently includes has one regulatory-approved member, enfuvirtide (T-20, Fuzeon).

Some piperazine and piperidine derivatives have been previously described. For example, WO 2005/004801 and US 2004/0009985 describe piperazine and piperadine deriviatives that incorporate an indole, azaindole, or other fused aromatic ring system linked to a piperazine ring through a ketoamide linker.

### SUMMARY OF THE INVENTION

Embodiments of the present invention provide compounds that may be useful as an active ingredient used in the treatment of HIV infection, in some embodiments, in the treatment of HIV-1 infection.

According to some embodiments of the invention are compounds of Formula (I)

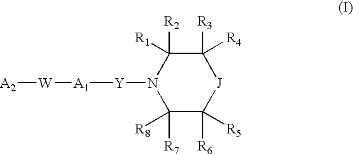

or a pharmaceutically acceptable prodrug, salt, polymorph, solvate, enantiomer, diastereomer, racemate, mixture of stereoisomers thereof, or derivative thereof, wherein:

W is null, oxy, amino, thio, sulfinyl, sulfonyl, carbonyl, amide, alkylene or cycloalkylidene,

wherein at least one carbon atom of the alkylene or cycloalkylidene is optionally substituted with an oxy, amino, thio, sulfinyl, sulfonyl, carbonyl or amide group, and wherein the alkylene or cycloalkylidene is optionally substituted with at least one halogen atom;

A₁ is a monocyclic cycloalkylidene, monocyclic heterocycloalkylidene, monocyclic arylene or monocyclic heteroarylene, each optionally substituted with an alkyl, alkoxy, fluoroalkyl, cycloalkyl, hydroxy, halo, fluoroalkoxy, alkenyl, alkenoxy, phosphoramide, phosphoramidealkyl, phosphonate, phosphonatealkyl or —R₉Q, wherein R₉ is null or alkylene and Q is —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈, or —OR₂₉;

A₂ is null, cycloalkyl, heterocycloalkyl, aryl or heteroaryl, optionally substituted with at least one of an alkyl, alkoxy, fluoroalkyl, cycloalkyl, hydroxy, halo, fluoroalkoxy, alkenyl, alkenoxy and/or —R₉Q, wherein R₉ and Q are as defined above;

R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are each independently hydrogen, alkyl, aryl, heteroaryl, allyl, alkoxy, cycloalkyl, heterocycloalkyl, fluoroalkyl, fluorocycloalkyl, arylalkyl or heteroarylalkyl; or wherein R₁₀ and R₁₁, R₁₆ and R₁₇, R₂₁ and R₂₂ or R₂₃ and R₂₄, taken together with the nitrogen to which they are attached, are part of a heterocycloalkyl or heteroaryl;

Y is —CO—CO—, —SO₂—, —C═NRₓ—CO—, and —CO—C═NRₓ—, —O—CO—, or —NR₃₀CO—; wherein Rₓ is alkyl, fluoroalkyl, alkoxyalkyl, cycloalkyl, heterocycloalkyl, aryl or heteroaryl, optionally substituted with at least one halogen, alkyl, alkoxy, —CF₃, —OCF₃, and/or —CN;

R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge,

wherein the alkyl or alkylene bridge is optionally substituted with at least one halogen, amino, hydroxyl, —CN, —NO₂, alkoxy, —CF₃, —OCF₃, alkyl, allyl, fluoroalkyl, cycloalkyl, fluorocycloalkyl, heterocycloalkyl, aryl, heteroaryl, arylalkyl, heteroarylalkyl, polyether and/or R₃₁-Q′ group, wherein R₃₁ is null or alkylene and Q′ is —SO₂NR₃₂R₃₃, —NR₃₄COR₃₅, —CONR₃₆R₃₇ or —COOR₃₈;

R₃₀, R₃₂, R₃₃, R₃₄, R₃₅, R₃₆, R₃₇ and R₃₈ are each hydrogen, alkyl, allyl, fluoroalkyl, cycloalkyl, heterocycloalkyl, fluorocycloalkyl, alkoxy, aryl, heteroaryl, arylalkyl, or heteroarylalkyl; or R₃₂ and R₃₃ or R₃₆ and R₃₇, taken together with the nitrogen to which they are attached, are part of a heterocycloalkyl or heteroaryl; and

wherein the cycloalkyl, heterocycloalkyl, aryl and heteroaryl are each independently optionally substituted with at least one hydrogen, halo, alkoxy, —CF₃, —OCF₃ and/or —CN;

J is

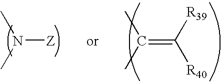

Z is —COR₄₁, —C(═NR₄₃)R₄₁ or R₄₂;

R₄₁ is cycloalkyl, heterocycloalkyl, aryl or heteroaryl; each optionally substituted with at least one alkyl, cycloalkyl, alkoxy, fluoroalkoxy, fluoroalkyl, fluorocycloalkyl, halo, —CN, —CF₃, alkylthio, hydroxy, alkenyl, alkenoxy, acetyl and/or —R₉Q, wherein R₉ and Q are defined above;

R₄₂ is aryl or heteroaryl, optionally substituted with at least one halo, alkoxy, —CF₃, —OCF₃, —CN, alkyl, -cycloalkyl, -fluoroalkoxy, fluoroalkyl, fluorocycloalkyl, alkylthio, hydroxy, acetyl, alkenyl, alkenoxy and/or —R₉Q, wherein R₉ and Q are defined above;

R₄₃ is hydrogen, —CN, alkoxy, fluoroalkoxy, alkyl, fluoroalkyl, cycloalkyl, fluorocycloalkyl, aryl, heteroaryl or heterocycloalkyl;

wherein the cycloalkyl, heterocycloalkyl, aryl and heteroaryl are optionally substituted with at least one halo, alkyl, alkoxy, —CF₃, —OCF₃, —CN, cycloalkyl, fluoroalkoxy, fluoroalkyl, fluorocycloalkyl, alkylthio, hydroxy, alkenyl, alkenoxy and/or —R₉Q, wherein R₉ and Q are defined above;

R₃₉ is cycloalkyl, heterocycloalkyl, aryl or heteroaryl, each optionally substituted with at least one halogen, alkyl, alkoxy, —CF₃, —OCF₃, —CN, cycloalkyl, fluoroalkoxy, fluoroalkyl, fluorocycloalkyl, S-alkyl, hydroxy, alkenyl, alkenoxy, acetyl and/or —R₉Q, wherein R₉ and Q are defined above; and

R₄₀ is hydrogen, —CN, alkyl, halo, —CF₃, cycloalkyl, fluoroalkyl, fluorocycloalkyl, aryl, heteroaryl, arylalkyl, heteroarylalkyl or heterocycloalkyl,

wherein the cycloalkyl, heterocycloalkyl, aryl, heteroaryl, arylalkyl and heteroarylalkyl are optionally substituted with at least one halo, alkyl, alkoxy, —CF₃, —OCF₃, —CN, cycloalkyl, fluoroalkoxy, fluoroalkyl, fluorocycloalkyl, alkylthio, hydroxy, alkenyl, alkenoxy and/or —R₉Q, wherein R₉ and Q are defined above.

In particular embodiments of the invention, in a compound of Formula (I),

W is null, C₀-C₆ alkylene, (C₀-C₃ alkylene)-O—(C₀-C₃ alkylene), (C₀-C₃ alkylene)-NR′—(C₀-C₃ alkylene), (C₀-C₃ alkylene)-S—(C₀-C₃ alkylene), (C₀-C₃ alkylene)-S(═O)—(C₀-C₃ alkylene), (C₀-C₃ alkylene)-SO₂—(C₀ -C₃ alkylene), (C₀-C₃ alkylene)-C(═O)—(C₀-C₃ alkylene), (C₀-C₃ alkylene)-C(═O)NR′—(C₀-C₃ alkylene) or (C₀-C₆ cycloalkylidene), wherein the alkylene and cycloalkylidene groups are optionally substituted with 1 to 3 halogen atoms;

A₁ is phenylene or monocyclic heteroarylene, wherein the phenylene and monocyclic heteroarylene are optionally substituted with 1 to 5 functional groups, wherein each functional group may be a C₁-C₆ alkyl, C₁-C₆ alkoxy, C₁-C₆ fluoroalkyl, C₃-C₇ cycloalkyl, hydroxy, halo, C₁-C₆ fluoroalkoxy, C₂-C₆ alkenyl, C₂-C₆ alkenoxy or —R₉Q, wherein R₉ is null or C₁-C₂ alkylene and Q is —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈, or —OR₂₉;

A₂ is phenyl or heteroaryl, wherein the phenyl and heteroaryl are optionally substituted with 1 to 5 functional groups, wherein each functional group may be a C₁-C₆ alkyl, C₁-C₆ alkoxy, C₁-C₆ fluoroalkyl, C₃-C₇ cycloalkyl, hydroxy, halogen, C₁-C₆ fluoroalkoxy, C₂-C₆ alkenyl, C₂-C₆ alkenoxy or —R₉Q, wherein R₉ is null or C₁-C₂ alkylene and Q is defined above;

R′, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are each independently hydrogen, C₁-C₆ alkyl, allyl, C₁-C₆ fluoroalkyl, C₃-C₇ cycloalkyl, C₃-C₇ fluorocycloalkyl, C₁-C₆ alkoxy, phenyl, phenylmethyl, phenylethyl, heteroaryl, heteroarylmethyl, heteroarylethyl, heterocycloalkyl, heterocycloalkylmethyl or heterocycloalkylethyl; or wherein R₁₀ and R₁₁, R₁₆ and R₁₇, R₂₁ and R₂₂, or R₂₃ and R₂₄, taken together with the nitrogen to which they are attached, are part of a ring selected from the group consisting of azetidine, azetidin-2-one, pyrrolidine, pyrrolidin-2-one, pyrrolidin-3-one, piperidine, piperidin-2-one, piperidin-3-one, piperidin-4-one, morpholine, morpholin-2-one, morpholin-3-one and N-alkylpiperazine;

wherein the heterocycloalkyl includes

0 to 4 nitrogen atoms;

0 to 2 nitrogen atoms and 0 to 1 oxygen atom;

0 to 2 nitrogen atoms and 0 to 1 sulfur atom; or

0 to 2 nitrogen atoms, 0 to 1 oxygen atom and 0 to 1 sulphur atom; and

wherein the heteroaryl imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenzimidazolyl, indazolyl, quinazolinyl, phthalazinyl, benzoxazolyl or quinoxalinyl; and

wherein the phenyl, heteroaryl or heterocycloalkyl is optionally substituted with 1 to 5 functional groups, wherein each functional group may be halo, C₁-C₆ alkoxy, —CF₃, —OCF₃ or —CN;

Rₓ is alkyl, fluoroalkyl, alkoxyalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl; wherein each heteroaryl ring is optionally substituted with 1 to 5 functional groups wherein each functional group may be halo, C₁-C₆ alkyl, C₁-C₆ alkoxy, —CF₃, —OCF₃ or —CN;

R₁, R₂, R₃, R₄, R₅, R₆, R₇, and R₈ are each independently hydrogen or C₁-C₆ alkyl,

wherein the C₁-C₆ alkyl is optionally substituted with 1 to 3 functional groups, wherein each functional group may be halo, amino, hydroxyl, —CN, —NO₂, C₁-C₆ alkoxy, —CF₃, —OCF₃, C₁-C₆ alkyl, allyl, C₁-C₆ fluoroalkyl, C₃-C₇ cycloalkyl, C₃-C₇ fluorocycloalkyl, phenyl, phenylmethyl, phenylethyl, heteroaryl, heteroarylmethyl, heteroarylethyl, heterocycloalkyl, heterocycloalkylmethyl, heterocycloalkylethyl, (CRₐR_{b})_{U}-T-(CR_{c}R_{d})_{U′}Rₑ or R₃₁Q′ wherein R₃₁ is null or C₁-C₂ alkylene and Q′ is —SO₂NR₃₂R₃₃, —NR₃₄COR₃₅, —CONR₃₆R₃₇ or —COOR₃₈;

R₃₀, R₃₂, R₃₃, R₃₄, R₃₅, R₃₆, R₃₇, R₃₈, Rₐ, R_{b}, R_{c}, R_{d} and Rₑ are each independently hydrogen, C₁-C₆ alkyl, allyl, C₁-C₆ fluoroalkyl, C₃-C₇ cycloalkyl, C₃-C₇ fluorocycloalkyl, C₁-C₆ alkoxy, phenyl-(C₀-C₂ alkyl), heteroaryl-(C₀-C₂ alkyl) or heterocycloalkyl-(C₀-C₂ alkyl);

wherein the heterocycloalkyl includes

0 to 4 nitrogen atoms;

0 to 2 nitrogen atoms and 0 to 1 oxygen atom;

0 to 2 nitrogen atoms and 0 to 1 sulfur atom; or

0 to 2 nitrogen atoms, 0 to 1 oxygen atom and 0 to 1 sulphur atom; and

wherein the heteroaryl group is imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenzimidazolyl, indazolyl, quinazolinyl, phthalazinyl, benzoxazolyl, or quinoxalinyl;

wherein the phenyl, heteroaryl or heterocycloalkyl is optionally substituted with 1 to 5 functional groups, wherein each functional group may be halo, C₁-C₆ alkoxy, —CF₃, —OCF₃ or —CN; or wherein R₃₂ and R₃₃ or R₃₆ and R₃₇, taken together with the nitrogen to which they are attached, are part of a heterocycloalkyl selected from the group consisting of aziridine, azetidine, pyrrolidine, pyrrolidin-2-one, piperidine, morpholine and N-alkylpiperazine;

U and U′ are each independently 0, 1 or 2;

T is null or oxy;

R₄₁ is phenyl, pyridinyl, pyrazinyl, pyridazinyl, pyrimidinyl, thienyl, furyl, thiazolyl, isothiazolyl, oxazolyl, isoxazolyl, pyrazoyl, imidazolyl, triazolyl, oxadiazolyl, thiadiazolyl or tetrazolyl; each of which is optionally substituted with at least one C₁-C₃ alkyl, C₃-C₆ cycloalkyl, C₁-C₃ alkoxy, C₁-C₆ fluoroalkoxy, C₁-C₆ fluoroalkyl, C₃-C₇ fluorocycloalkyl, —CN, —F, —Cl, —Br, —CF₃, C₀-C₃ alkylthio, hydroxy, C₂-C₆ alkenyl, C₂-C₆ alkenoxy, acetyl and/or —R₉Q, wherein R₉ is null or C₁-C₂ alkylene and Q is defined above;

R₄₂ is phenyl, heteroaryl, quinolinyl, isoquinolinyl, benzimidazolyl, azabenimidazolyl, benzothienyl, benzofuryl, benzoindazolyl, quinazolinyl, phthalazinyl, benzoxazolyl, quinoxalinyl, thienopyridine, thienopyrimidine, thienopyridazine, thienopyrazine, furopyridine, furoopyrimidine, furopyridazine, furopyrazine, oxazolopyridine, oxazolopyrimidine, oxazolopyridazine,oxazolopyrazine, thiazolopyridine, thiazolopyrimidine, thiazolopyridazine,thiazolopyrazine, napthyridine, pyridopyrimidine, pyridopyridazine or pyridopyrazine;

each optionally substituted with 1 to 5 functional groups, wherein each functional group may be halo, C₁-C₆ alkoxy, —CF₃, —OCF₃ or —CN, C₁-C₃ alkyl, C₃-C₆ cycloalkyl, C₁-C₆ fluoroalkoxy, C₁-C₆ fluoroalkyl, C₃-C₇ fluorocycloalkyl, C₀-C₃ alkylthio, hydroxy, C₂-C₆ alkenyl, C₂-C₆ alkenoxy, acetyl or —R₉Q, wherein R₉ is null or C₁-C₂ alkylene and Q is defined above;

R₄₃ is hydrogen, —CN, C₁-C₆ alkoxy, C₁-C₆ fluoroalkoxy, C₁-C₆ alkyl, C₁-C₆ fluoroalkyl, C₃-C₇ cycloalkyl or C₃-C₇ fluorocycloalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl;

wherein the aryl or heteroaryl are optionally substituted with 1 to 5 functional groups, wherein each functional group may be halo, C₁-C₆ alkyl, C₁-C₆ alkoxy, —CF₃, —OCF₃ or —CN, C₁-C₃ alkyl, C₃-C₆ cycloalkyl, C₁-C₆ fluoroalkoxy, C₁-C₆ fluoroalkyl, C₃-C₇ fluorocycloalkyl, C₀-C₃ alkylthio, hydroxy, C₂-C₆ alkenyl, C₂-C₆ alkenoxy, acetyl or —R₉Q, wherein R₉ is null or C₁-C₂ alkylene and Q is defined above;

R₃₉ is phenyl, imidazolyl, pyrazolyl, triazolyl, tetrazolyl, thienyl, furyl, thiazolyl, isothiazolyl, oxazolyl,isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl;

each optionally substituted with 1 to 5 functional groups, wherein each functional group may be halo, C₁-C₆ alkyl, C₁-C₆ alkoxy, —CF₃, —OCF₃, —CN, C₁-C₃ alkyl, C₃-C₆ cycloalkyl, C₁-C₆ fluoroalkoxy, C₁-C₆ fluoroalkyl, C₃-C₇ fluorocycloalkyl, S—(C₀-C₃ alkyl), hydroxy, C₂-C₆ alkenyl, C₂-C₆ alkenoxy, acetyl or —R₉Q, wherein R₉ is null or C₁-C₂ alkylene and Q is defined above; and

R₄₀ is hydrogen, —CN, C₁-C₆ alkyl, halo, —CF₃, C₃-C₆ cycloalkyl, C₁-C₆ fluoroalkyl, C₃-C₇ fluorocycloalkyl, heterocycloalkyl, heterocycloalkylmethyl, heterocycloalkylethyl, R₄₁, —CH₂R₄₁ and —CH₂CH₂R₄₁;

wherein the heterocycloalkyl includes

0 to 4 nitrogen atoms;

0 to 2 nitrogen atoms and 0 to 1 oxygen atom;

0 to 2 nitrogen atoms and 0 to 1 sulfur atom; or

0 to 2 nitrogen atoms, 0 to 1 oxygen atom and 0 to 1 sulphur atom; and

wherein R₄₁ is phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenimidazolyl, indazolyl, quinazolinyl, phthalazinyl, benzoxazolyl or quinoxalinyl; and is optionally substituted with 1 to 5 functional groups, wherein each functional group may be halogen, C₁-C₆ alkyl, C₁-C₆ alkoxy, —CF₃, —OCF₃, —CN, hydrogen, C₁-C₃ alkyl, C₃-C₆ cycloalkyl, O—(C₁-C₆ fluoroalkyl), C₁-C₆ fluoroalkyl, C₃-C₇ fluorocycloalkyl, S—(C₀-C₃ alkyl), hydroxy, C₂-C₆ alkenyl, C₂-C₆ alkenoxy, acetyl or —R₉Q, wherein R₉ is null or C₁-C₂ alkylene and Q is defined above.

According to some embodiments of the invention, provided are compounds of Formula (II)

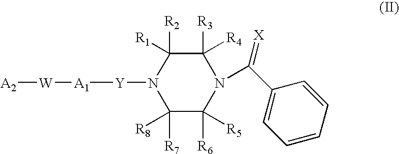

or a pharmaceutically acceptable prodrug, salt, polymorph, solvate, enantiomer, diastereomer, racemate, mixture of stereoisomers thereof, or derivative thereof, wherein:

W is null, oxy, amino, thio, sulfinyl, sulfonyl, carbonyl, amide, alkylene or cycloalkylidene,

wherein at least one carbon atom of the alkylene or cycloalkylidene is

wherein the alkyl or alkylene bridge is optionally substituted with 1 to 3 functional groups, wherein each functional group may be halogen, amino, hydroxyl, —CN, —NO₂, alkoxy, —CF₃, —OCF₃, alkyl, allyl, fluoroalkyl, cycloalkyl, fluorocycloalkyl, heterocycloalkyl, aryl, heteroaryl, arylalkyl, heteroarylalkyl, polyether or R₃₁-Q′ wherein R₃₁ is null or alkylene and Q′ is —SO₂NR₃₂R₃₃, —NR₃₄COR₃₅, —CONR₃₆R₃₇ or —COOR₃₈;

R₃₀, R₃₂, R₃₃, R₃₄, R₃₅, R₃₆, R₃₇ and R₃₈ are each independently hydrogen, alkyl, allyl, fluoroalkyl, cycloalkyl, heterocycloalkyl, fluorocycloalkyl, alkoxy, aryl, heteroaryl, arylalkyl or heteroarylalkyl; or wherein R₃₂ and R₃₃ or R₃₆ and R₃₇, taken together with the nitrogen to which they are attached, are part of a heterocycloalkyl or heteroaryl; and

wherein the cycloalkyl, heterocycloalkyl, aryl and heteroaryl are each independently optionally substituted with 1 to 5 functional groups, wherein each functional group may be halo, alkoxy, —CF₃, —OCF₃ and —CN; and

X is O, S or NR₃₉, wherein R₃₉ is hydrogen, —CN, alkoxy, fluoroalkoxy, alkyl, fluoroalkyl, cycloalkyl, fluorocycloalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl;

optionally substituted with 1 to 5 functional groups, wherein each functional group may be halo, alkyl, alkoxy, —CF₃, —OCF₃ or —CN, cycloalkyl, fluoroalkoxy, fluoroalkyl, fluorocycloalkyl, alkylthio, hydroxy, alkenyl, alkenoxy, acetyl or —R₉Q, wherein R₉ and Q are defined above.

In particular embodiments of the invention, for the compound of Formula (II),

W is —(CH₂)ₓ(CO)_{y}(CH₂)₂—, wherein x, y and z are each independently 0, 1, 2 or 3;

A₁ is a cycloalkylidene, heterocycloalkylidene, arylene or optionally substituted with an oxy, amino, thio, sulfinyl, sulfonyl, carbonyl or amide group, and wherein the alkylene or cycloalkylidene is optionally substituted with 1-3 halogen atoms;

A₁ is a monocyclic cycloalkylidene, monocyclic heterocycloalkylidene, monocyclic arylene and monocyclic heteroarylene, optionally substituted with 1 to 5 functional groups, wherein each functional group may be alkyl, alkoxy, fluoroalkyl, cycloalkyl, hydroxy, halo, fluoroalkoxy, alkenyl, alkenoxy or —R₉Q, wherein Q is —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈ or —OR₂₉;

A₂ is null, cycloalkyl, heterocycloalkyl, aryl pr heteroaryl, wherein the cycloalkyl, heterocycloalkyl, aryl or heteroaryl is optionally substituted with 1 to 5 functional groups, wherein each functional group may be alkyl, alkoxy, fluoroalkyl, cycloalkyl, hydroxy, halo, fluoroalkoxy, alkenyl, alkenoxy and —R₉Q, wherein R₉ and Q are as defined above;

R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are hydrogen, alkyl, aryl, heteroaryl, allyl, alkoxy, cycloalkyl, heterocycloalkyl, fluoroalkyl, fluorocycloalkyl, arylalkyl or heteroarylalkyl; or wherein R₁₀ and R₁₁, R₁₆ and R₁₇, R₂₁ and R₂₂, or R₂₃ or R₂₄, taken together with the nitrogen to which they are attached, are part of a heterocycloalkyl or heteroaryl;

Y is —CO—CO—, —SO₂—, —C═NRₓ—CO—, and —CO—C═NRₓ—, —O—CO—, or —NR₃₀CO—; wherein Rₓ is alkyl, fluoroalkyl, alkoxyalkyl, cycloalkyl, heterocycloalkyl, aryl or heteroaryl, optionally substituted with 1 to 5 functional groups, wherein each functional group may be halogen, alkyl, alkoxy, —CF₃, —OCF₃ and —CN;

R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge, heteroarylene, each optionally substituted with 1 to 3 functional groups, wherein each functional group may be halo, alkyl, alkoxy, fluoroalkyl, fluoroalkoxy, hydroxy, amino, alkylamino, dialkylamino or thiol;

A₂ is a monocyclic or bicyclic cycloalkyl, monocyclic or bicyclic heterocycloalkyl, monocyclic or bicyclic aryl or monocyclic or bicyclic heteroaryl,

each optionally substituted with 1 to 3 functional groups, wherein each functional group may be halo, —CN, alkyl, alkoxy, acetyl, oxo, fluoroalkyl, fluoroalkoxy, hydroxy, amino, methylamino, dimethylamino, —SH, cycloalkyl, heterocycloalkyl, aryl, heteroaryl, arylalkyl or arylcarbonyl;

wherein the cycloalkyl, heterocycloalkyl, aryl or heteroaryl substituted onto the monocyclic or bicyclic ring is optionally substituted with a halo, alkyl, acetyl or alkoxycarbonyl;

Y is —(CH₂)ₘ(C═O)ₙ— or —SO₂—, wherein m and n are each independently 0, 1, 2 or 3;

R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge; and

X is O or N—O-alkyl.

According to some embodiments of the invention, the compounds of Formula (I) and Formula (II) are present as racemic mixtures. However, in some embodiments, compound of Formula (I) and Formula (II) are present substantially as the (R) enantiomer, or in the enantiomerically pure (R) form.

According to other embodiments of the invention, provided are pharmaceutical compositions that include a compound according to an embodiment of the invention, or a pharmaceutically acceptable prodrug, salt, polymorph, solvate, enantiomer, diastereomer, racemate, mixture of stereoisomers thereof, or derivative thereof; and a pharmaceutically acceptable carrier, excipient or diluent.

According to some embodiments, provided are novel intermediates in the synthesis of the compounds of Formula (I) and Formula (II).

Embodiments of the present invention provide uses of the compounds described herein for the preparation of medicaments for carrying out the utilities described herein.

Embodiments of the present invention provide kits including one or more containers having pharmaceutical dosage units including an effective amount of the compounds described herein, wherein the container is packaged with optional instructions for the use thereof

According to other embodiments of the invention, provided are methods for the inhibition of transmission of an HIV virus to a cell, which include contacting the cell with an effective concentration of the compound according to an embodiment of the invention, under conditions sufficient wherein fusion of the virus is inhibited.

According to some embodiments of the invention, provided are methods of treating HIV-1 infection in a subject, which include administering to the subject an effective amount of the compound according to an embodiment of the invention, or a pharmaceutically acceptable prodrug, salt, polymorph, solvate, enantiomer, diastereomer, racemate, mixture of stereoisomers thereof, or derivative thereof.

In particular embodiments, the method further includes administering an effective amount of at least one other therapeutic agent, such as a reverse transcriptase inhibitor, a viral protease inhibitor, a cytokine, a cytokine inhibitor, a glycosylation inhibitor or a viral mRNA processing inhibitor. In some embodiments, a nucleoside analogue, such as azidothymidine (AZT), ddI, ddC, ddA, d4T or 3TC, is the therapeutic agent. In some embodiments, the therapeutic agent is interferon-α, interferon-β or interferon-γ. In some embodiments, the therapeutic agent is a protease inhibitor that is an inhibitor of HIV-1 protease, such as indavir.

According to some embodiments, administration of a compound according to the present invention and another therapeutic agent is sequential, such as with cycling therapy, which may be repeated at least one time in a fixed order. A compound according to an embodiment of the invention may be administered before or after another therapeutic agent. In some embodiments, the cycling therapy includes the administration of a compound according to an embodiment of the invention, or a pharmaceutically acceptable prodrug, salt, polymorph, solvate, enantiomer, diastereomer, racemate, mixture of stereoisomers thereof, or derivative thereof, in alternation with at least one therapeutic agent selected from the group consisting of a reverse transcriptase inhibitor, a viral protease inhibitor, a cytokine, a cytokine inhibitor, a glycosylation inhibitor or a viral mRNA processing inhibitor.

According to some embodiments, administration of a compound according to the present invention and another therapeutic agent is simultaneous.

According to some embodiments, the administration of at least one of the therapeutic agents is oral, and in some embodiments, administration of at least one of the therapeutic agents is parenteral, such as subcutaneous.

According to some embodiments of the invention, methods of treating HIV infection in an individual include administering an effective amount of a compound according to an embodiment of the invention, or a pharmaceutically acceptable prodrug, salt, polymorph, solvate, enantiomer, diastereomer, racemate, mixture of stereoisomers thereof, or derivative thereof. In addition, in some embodiments, the compound according to an embodiment of the invention, or a pharmaceutically acceptable prodrug, salt, polymorph, solvate, enantiomer, diastereomer, racemate, mixture of stereoisomers thereof, or derivative thereof, is administered with at least one other therapeutic agent.

According to some embodiments of the invention, provided are methods of inhibiting HIV replication including administering to a subject an effective amount of the compound according to an embodiment of the invention, or a pharmaceutically acceptable prodrug, salt, polymorph, solvate, enantiomer, diastereomer, racemate, mixture of stereoisomers thereof. In addition, in some embodiments, the compound according to an embodiment of the invention, or a pharmaceutically acceptable prodrug, salt, polymorph, solvate, enantiomer, diastereomer, racemate, mixture of stereoisomers thereof, or derivative thereof, is administered with at least one other therapeutic agent.

According to some embodiments of the invention, provided are methods for the inhibition of transmission of an HIV retrovirus to a cell, including contacting the cell with an effective amount of a compound according to an embodiment of the invention, or a pharmaceutically acceptable prodrug, salt, polymorph, solvate, enantiomer, diastereomer, racemate, mixture of stereoisomers thereof. In addition, in some embodiments, the compound according to an embodiment of the invention, or a pharmaceutically acceptable prodrug, salt, polymorph, solvate, enantiomer, diastereomer, racemate, mixture of stereoisomers thereof, or derivative thereof, is administered with at least one other therapeutic agent.

Embodiments of the present invention provide uses of the compounds described herein for the preparation of medicaments for carrying out the utilities described herein.

Embodiments of the present invention provide kits including one or more containers having pharmaceutical dosage units including an effective amount of the compounds described herein, wherein the container is packaged with optional instructions for the use thereof

### DETAILED DESCRIPTION OF EMBODIMENTS OF THE INVENTION

The invention is described more fully hereinafter. This invention may, however, be embodied in many different forms and should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the term “and/or” includes any and all combinations of one or more of the associated listed items. As used herein, the singular forms “a”, “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein

Definitions

Certain terms and phrases used herein and in the claims have the meanings provided below, unless otherwise stated. A functional group below may be referred to as including both monovalent or divalent radicals. The definition may describe embodiments in terms of the monovalent radicals, but one of skill in the art will understand that the corresponding divalent radicals are also encompassed.

The designation of a group as “Cₓ” refers to such group having x number of carbon atoms. Thus, for example, C₃ alkyl refers to an alkyl group having 3 carbon atoms. Likewise, C₁-C₆ alkyl refers to any alkyl having from one to six carbon atoms.

The term “null” in reference to a functional group means that the group is not present in the structure, and if the null group connects two other groups, it is understood that a bond, a single bond unless otherwise specified, connects the two other functional groups.

The term “alkyl” and “alkylene” refer to a straight or branched monovalent or divalent, respectively, hydrocarbon moiety. Unless specified otherwise, the term alkyl encompasses saturated hydrocarbons (e.g., methyl, ethyl, propyl, isopropyl, butyl, isobutyl, t-butyl, amyl, hexyl, and the like) and unsaturated hydrocarbons, such as alkenyl (including at least one carbon-carbon double bond) and alkynyl (including at least one carbon-carbon triple bond). Thus, the terms alkynyl and alkenyl also encompass both straight and branched chains. In particular embodiments of the invention, the alkyl groups may be C₁-C₂₀, in some embodiments, C₁-C₁₀, in some embodiments C₁-C₆ and, in some embodiments C₁₋₃. The alkyl groups may also be unsubstituted or substituted.

The terms “cycloalkyl” and “cycloalkylidene” refers to a monovalent or divalent, respectively, monocyclic or polycyclic fused ring hydrocarbon moiety. In some embodiments, the cycloalkyl is a C₃-C₁₂ cycloalkyl, and in some embodiments, a C₄-C₆ cycloalkyl. The term cycloalkyl includes both saturated cyclic alkyl groups and unsaturated cycloalkyl groups such as cycloalkenyl and cycloalkynyl groups, provided that a conjugated pi-electron system is not present. Exemplary saturated alkyl include monocyclic cycloalkyl including cyclopropyl, cyclobutyl, cyclopentyl, cyclohexyl and cycloheptyl, as well as other cycloalkyl such as norbonanyl and adamantyl. Exemplary unsaturated cycloalkyl groups include cyclopentenyl, cyclohexadienyl, cycloheptatrienyl and norbornenyl. Furthermore a cycloalkyl may include an alkyl, as defined herein, in combination with a cyclic hydrocarbon moiety. For example, a cycloalkyl group may be a —(CH₂)ₓ-cyclic alkyl-(CH₂)_{y}— wherein x and y are each independently integers such as 0, 1, 2, 3, 4, 5, 6, 7, 8, 9 and 10.

The term “alkoxy” refers to an —OR group, wherein R is an alkyl or a cycloalkyl group, both as defined herein.

The term “alkylthio” refers to an —SR group, wherein R is an alkyl or a cycloalkyl group, both as defined herein.

The term “fluoroalkyl” refers to an alkyl, as defined herein, wherein at least one hydrogen atom of the alkyl is substituted with a fluoro group.

The term “fluoroalkoxy” refers to an alkoxy, as defined herein, wherein at least one hydrogen atom of the alkoxy is substituted with a fluoro group.

The term “fluorocycloalkyl” refers to a cycloalkyl, as defined herein, wherein at least one hydrogen atom of the cycloalkyl is substituted with a fluoro group.

The term “alkenoxy” refers to an alkoxy, as defined herein, wherein the alkyl group is an alkenyl group, as defined herein.

The term “halogen” and “halo” refers to a halogen group, such as a fluoro, chloro, bromo or iodo group.

The term “oxy” refers to an —O— group.

The term “oxo” refers to a ═O group.

The terms “hydroxy” or “hydroxyl” refer to an —OH group.

The term “allyl” refers to a —CH₂—CH═CR₁R₂ group, wherein R₁ and R₂ may each independently be hydrogen, alkyl, cycloalkyl, heterocycloalkyl, aryl, heteroaryl, or another group as otherwise specified.

The term “amino” refers to primary, secondary and tertiary amino groups, such as —NH₂, —NHR, and NR₁R₂, respectively, wherein R₁ and R₂ may each independently be an alkyl, cycloalkyl, heterocycloalkyl, aryl, heteroaryl, or another group as otherwise specified.

The term “thio” refers to a —S— group.

The term “sulfinyl” refers to a —S(═O)— group, which may also be referred to herein as —SO—.

The term “sulfonyl” refers to a —S(═O)₂— group, which may also be referred to herein as —SO₂—.

The term “amide” refers to a —NC(═O)— or an —C(═O)N— group, also referred herein as —NCO—.

The term “phosphonate” refers to a radical —P(═O)(OR₁)(OR₂) or —P(═O)(OH)(OR₂), wherein R₁ and R₂ may each independently be an alkyl, cycloalkyl, heterocycloalkyl, aryl and heteroaryl;

The term “phosphoramide” refers to a radical —P(═O)(NR₂)₃, wherein each R may independently be an alkyl, cycloalkyl, heterocycloalkyl, aryl and heteroaryl;

The term “aryl” refers to a monocyclic or fused-ring polycyclic (e.g., mono-, bi- or tricyclic) carbocyclic aromatic group. In some embodiments the aryl is a C₅-C₁₂ aryl, and in some embodiments a C₅-C₉ aryl. Exemplary aryl include phenyl, naphthyl, anthracenyl, and the like. The aryl may be unsubstituted or substituted.

The term “heterocycloalkyl(idene)” refers to a cycloalkyl, as defined herein, wherein at least one of the atoms comprising the ring(s) is substituted with a heteroatom (O, N or S). For example, the heterocycloalkyl may include 1, 2, 3, 4, 5 or 6 heteroatoms. Exemplary heterocycloalkyl include azetidinyl, piperazinyl, imidazolinyl, thiazolidinyl, 3-pyrrolidin-1-yl, morpholinyl, thiomorpholinyl, oxiranyl, 2H-pyranyl, 4H-pyranyl, parathiazinyl, pyrazolidinyl, imidazolinyl, imidazolidinyl, piperidinyl, piperazinyl, and the like.

The term “heteroaryl(ene)” refers to an aryl(ene), as defined herein, wherein at least one of the ring carbon atoms is substituted with a heteroatom. For example, a heteroaryl group may include 1, 2, 3, 4, 5 or 6 heteroatoms. In some embodiments, the heteroaryl includes 1 to 3 heteroatoms. Exemplary heteroaryl groups are pyridinyl, pyridazinyl, pyrimidyl, pyrazinyl, triazinyl, pyrrolyl, pyrazolyl, imidazolyl, (1,2,3,)- and (1,2,4)-triazolyl, pyrazinyl, pyrimidinyl, tetrazolyl, furyl, thienyl, isothiazolyl, thiazolyl, furyl, isoxazolyl, oxadiazolyl, thiadiazolyl, oxazolyl, pyridonyl, quinolinylene, isoquinolinylene, benzimidazolylene, azabenzimidazol, indazolyl, quinazolinyl, phthalazinyl, benzoxazolyl, and quinoxalinyl. A heteroaryl group can be unsubstituted or substituted.

The terms “arylalkyl”(e.g., phenylmethyl, phenylethyl, and the like), “heteroarylalkyl” “alkoxyalkyl” “phosphonatealkyl” and “phosphoramidealkyl” refer to an alkyl group, as defined herein, wherein at least one hydrogen atom of the alkyl is substituted with an aryl, heteroaryl, alkoxy group, phosphonate or phosphoramide, respectively, each as defined herein.

The term “polyether” refers to an alkyl, as defined herein, that includes at least two ether (R—O—R) linkages. Exemplary polyether are polyethylene oxide [—(CH₂CH₂O)—] and straight or branched polypropylene oxide [e.g., —(CH₂CH₂CH₂O)—] or mixtures thereof.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted). A substitution is made provided that any atom's normal valency is not exceeded and that the substitution results in a stable compound.

The term “pharmaceutically acceptable salt” refers to a salt or salts prepared from at least one pharmaceutically acceptable non-toxic acid or base including inorganic acids and bases, and organic acids and bases. Pharmaceutically acceptable salts of compounds according to embodiments of the invention include the acid addition and base salts thereof, and may be made using techniques known in the art, such as, but not limited to, reacting the compound with the desired base or acid. Suitable pharmaceutically acceptable base addition salts for compounds according to embodiments of the present invention include metallic salts (e.g., alkali metal salts and/or alkaline earth metal salts) made from aluminum, calcium, lithium, magnesium, potassium, sodium, and zinc; or organic salts made from lysine, N,N′-dibenzylethyl-enediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include xinofoate, hydrochloride mesylate, zinc, potassium, or iron salts. In certain embodiments, both water-soluble and water-insoluble salts will be useful based on the mode of administration.

The term “polymorph” refers to one or more forms into which a compound of the present invention may crystallize. For example, depending on changes in temperature, pressure, or both, or other variations during the crystallization process, it is possible that one or more polymorphs of a compound according to the present invention may result. Polymorphs can generally be distinguished from each other by physical characteristics, biophysical properties, and by other techniques well know in the art

The term “solvate” refers to a molecular complex comprising a compound according to an embodiment of the present invention with one or more pharmaceutically acceptable solvent molecules. A solvent may include, but is not limited to, ethanol. Pharmaceutically acceptable solvates in accordance with the present invention include those wherein the solvent of crystallization may be isotopically substituted; e.g., D₂O, d₆-acetone, d₆-DMSO.

The term “prodrug” refers to a derivative of a compound according to an embodiment of the present invention which may have minimal or no pharmacological activity itself, but when administered in vivo, can be converted into a compound of the present invention that has the desired pharmacological activity. For example, the prodrug can hydrolyze (e.g., via it's biohydrolyzable moiety(s) such as a biohydrolyzable amide, a biohydrolyzable ester, a biohydrolyzable carbamate, a biohydrolyzable carbonate, and a biohydrolyzable phosphate), oxidize, or otherwise react in vivo to provide the compound of the present invention. Typically, prodrugs can be prepared using methods well known in the art, such as those described by Burger's Medicinal Chemistry and Drug Discovery, 172-178, 949-982 (Manfred E. Wolff ed., 5th ed. 1995). In some embodiments, a prodrug is a compound that is substantially biologically inactive, but is converted in vivo to a biologically active compound according to an embodiment of the invention.

The term “derivative” when used in reference to a compound according to an embodiment of the present invention means a compound: (a) which otherwise may have structural formula different from those of the active compounds of the present invention, but which is converted in the body to a compound of the present invention upon administration to an individual (e.g., prodrug, or pharmaceutically acceptable bioprecursor); or (b) is a metabolite of a compound of the present invention, formed in the body after, administration of a compound according to the present invention to an individual. It is well known in the pharmaceutical field that an active drug may be modified into a derivative of the active drug, to improve any undesired pharmaceutical property (e.g., related to one or more of stability, solubility, absorbability, and the like) of the active drug. The derivative may have efficacious activity by being converted in the body to the active drug, or may be derived physiologically from a compound of the present invention and exhibit antiviral activity.

The term “an effective amount” refers to that amount of a compound according to the present invention sufficient to result in amelioration of one or more symptoms of HIV infection and/or AIDS. The term “an effective amount” is also meant to include the amount of the compound of the present invention sufficient to result in inhibition of, or interference with, HIV binding events, viral entry, or viral infection. The term also encompasses the inhibition of viral transmission or prevention of viral establishment in its host, as observed by measuring one or more parameters. Such parameters may include, but are not limited to, reduction in viral load (e.g., such as measuring HIV viral RNA in plasma) or viral pathogenesis, or decrease in mortality and/or morbidity associated with HIV infection of an individual treated with a compound according to the present invention, or increase in immune parameters in the treated individual, such as an increase in overall CD4+ cells circulating in the blood, as compared to baseline (before treatment, or at an earlier point in the treatment history of the individual) level of circulating CD4+ cells.

The term “antiviral activity” refers to the ability of a compound according to the present invention to inhibit viral infection of cells, via, for example, inhibiting the ability of HIV-1 to bind to cell receptors and/or co-receptors of human cells which are capable of being infected by HIV-1. In some embodiments, a compound according to the present invention has antiviral activity, against typical strains of HIV-1, as represented by an IC₅₀ of no more than 5 μm (see, for example, Example 1, and Table 3, herein). The term “target cell” is used herein and in the claims to refer to a human cell capable of being infected by HIV, and in some embodiments, HIV-1. A compound of the present invention with antiviral activity can also interfere with or inhibit or prevent viral entry into a host (“viral entry inhibitor”), viral transmission to a host, or viral establishment in its host, as observed by measuring one or more parameters. Such parameters may include, but are not limited to, reduction in viral load (e.g., such as measuring HIV viral RNA in plasma) or viral pathogenesis, or decrease in mortality and/or morbidity associated with HIV infection of an individual treated with a compound according to the present invention, or increase in immune parameters in the treated individual, such as an increase in overall CD4+ cells circulating in the blood, as compared to baseline level of circulating CD4+ cells. When the term “antiviral activity” is used in relation to an individual active ingredient comprising administering a compound according to the present invention by itself, the term refers to the activity of that ingredient alone. When the term “antiviral activity” is used in relation to a combination of active ingredient comprising administering a compound according to the present invention with other therapeutic agents used in the treatment of HIV infection and/or AIDS (antiviral agents, immunomodulators, vaccines, and the like), the term refers to the activity of the combination treatment (e.g., whether administered simultaneously or sequentially, as part of a treatment regimen). As used herein and in the claims, unless otherwise specified, the terms “viral”, “antiviral”, “retroviral”, and “virus”, refer to, or are concerning, HIV, and in some embodiments, HIV-1.

“Subjects” as used herein, also referred to as “individuals”, are generally human subjects. The subjects may be male or female and may be of any race or ethnicity, including, but not limited to, Caucasian, African-American, African, Asian, Hispanic, Indian, etc. The subjects may be of any age, including newborn, neonate, infant, child, adolescent, adult, and geriatric. Subjects may also include animal subjects, particularly mammalian subjects such as dog, cat, horse, mouse, rat, etc., screened for veterinary medicine or pharmaceutical drug development purposes. Subjects further include, but are not limited, to those who may have, possess, have been exposed to, or have been previously diagnosed as afflicted with HIV or AIDS or one or more risk factors for HIV or AIDS.

As used herein and in the claims, the terms “treat”, “treating” and “treatment” means preventing or ameliorating diseases associated with HIV infection. Thus, the terms apply to prophylactic and/or therapeutic applications.

The terms “pharmaceutical composition” and “medicament” are used interchangeably herein to mean a composition comprising a pharmaceutically acceptable carrier and effective amount of a compound according to the present invention. The term “pharmaceutically acceptable carrier” is used herein and for the claims to refer to a carrier medium that does not significantly alter the biological activity of the active ingredient (e.g., the antiviral activity of a compound according to the present invention) to which it is added. The one or more substances of which the pharmaceutically acceptable carrier is comprised, typically depends on factors (or desired features for its intended use) of the pharmaceutical composition such as the intended mode of administration, desired physical state (e.g., solid, liquid, gel, suspension, etc.), desired consistency, desired appearance, desired taste (if any), desired pharmacokinetic properties once administered (e.g., solubility, stability, biological half life), desired release characteristics (e.g., (a) immediate release (e.g., fast-dissolving, fast-disintegrating), or (b) modified release (e.g., delayed release, sustained release, controlled release)), and the like. As known to those skilled in the art, a suitable pharmaceutically acceptable carrier may comprise one or substances, including but not limited to, a diluent, water, buffered water, saline, 0.3% glycine, aqueous alcohol, isotonic aqueous buffer; a water-soluble polymer, glycerol, polyethylene glycol, glycerin, oil, salt (e.g., such as sodium, potassium, magnesium and ammonium), phosphonate, carbonate ester, fatty acid, saccharide, polysaccharide, stabilizing agent (e.g., glycoprotein, and the like for imparting enhanced stability, as necessary and suitable for manufacture and/or distribution of the pharmaceutical composition), excipient, preservative (e.g., to increase shelf-life, as necessary and suitable for manufacture and distribution of the pharmaceutical composition), bulking agent (e.g., microcrystalline cellulose, and the like), suspending agent (e.g., alginic acid, sodium alginate, and the like), viscosity enhancer (e.g., methylcellulose), taste enhancer (e.g., sweetner, flavoring agent, taste-masking agent), binder (generally, to impart cohesive quality to a tablet or solid formulation; e.g., gelatin, natural and/or synthetic gums, polyvinylpyrrolidone, polyethylene glycol, and the like), extender, disintegrant (e.g., sodium starch glycolate, sodium carboxymethyl cellulose, starch, and the like), dispersant, coating (generally to impart a surface active agent to a tablet or solid formulation; e.g., polysorbate, talc, silicon dioxide, and the like), lubricant (e.g., magnesium stearate, calcium stearate, sodium lauryl sulphate, and the like), or colorant. As known to those skilled in the art, an active ingredient may be formulated into a pharmaceutical composition using methods and one or more pharmaceutically acceptable carriers well known in the art, taking the desired features of the pharmaceutical composition, as described above, in mind during formulation. Depending on such desired features, typically a pharmaceutical composition may comprise from about 1% by weight to about 80% by weight of an active ingredient, and from about 10% by weight to about 99% by weight of pharmaceutically acceptable carrier.

Administration of two or more compounds “in combination” means that the two compounds are administered closely enough in time that the presence of one alters the biological effects of the other. The two compounds can be administered simultaneously (i.e., concurrently) or sequentially. Simultaneous administration can be carried out by mixing the compounds prior to administration, or by administering the compounds at the same point in time but at different anatomic sites or using different routes of administration. The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

In terms of specific administration of the compounds and compositions described herein, the most suitable route in any given case will depend on the nature and severity of the condition being treated.

The compounds described herein can be formulated for administration in a pharmaceutical carrier in accordance with known techniques. See, e.g., Remington, The Science And Practice of Pharmacy (9th Ed. 1995). In the manufacture of a pharmaceutical formulation according to the invention, the compounds described herein are typically admixed with, inter alia, an acceptable carrier. The carrier can be a solid or a liquid, or both, and is optionally formulated as a unit-dose formulation, which can be prepared by any of the well-known techniques of pharmacy.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like.

In the case of a solution, it can be lyophilized to a powder and then reconstituted immediately prior to use. For dispersions and suspensions, appropriate carriers and additives include aqueous gums, celluloses, silicates or oils.

For injection, the carrier is typically a liquid, such as sterile pyrogen-free water, pyrogen-free phosphate-buffered saline solution, bacteriostatic water, or Cremophor EL[R] (BASF, Parsippany, N.J.), parenterally acceptable oil including polyethylene glycol, polyvinyl pyrrolidone, lecithin, arachis oil or sesame oil, with other additives for aiding solubility or preservation may also be included. For other methods of administration, the carrier can be either solid or liquid.

For oral administration, the compounds described herein can be administered in solid dosage forms, such as capsules, tablets, and powders, or in liquid dosage forms, such as elixirs, syrups, and suspensions. The compounds described herein can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges including the compounds described herein in a flavored base, usually sucrose and acacia or tragacanth; and pastilles including the compounds described herein in an inert base such as gelatin and glycerin or sucrose and acacia.

Formulations of the present invention suitable for parenteral administration can include sterile aqueous and non-aqueous injection solutions of the compounds described herein, which preparations are generally isotonic with the blood of the intended recipient. These preparations can contain anti-oxidants, buffers, bacteriostats and solutes, which render the formulation isotonic with the blood of the intended recipient. Aqueous and non-aqueous sterile suspensions can include suspending agents and thickening agents. The formulations can be presented in unit\dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets. For example, in one aspect of the present invention, there is provided an injectable, stable, sterile composition including compounds described herein of the invention, in a unit dosage form in a sealed container. Optionally, the composition is provided in the form of a lyophilizate, which is capable of being reconstituted with a suitable pharmaceutically acceptable carrier to form a liquid composition suitable for injection thereof into a subject.

Formulations suitable for rectal or vaginal administration can be presented as suppositories. These can be prepared by admixing the compounds described herein with one or more conventional excipients or carriers, for example, cocoa butter, polyethylene glycol or a suppository wax, which are solid at room temperature, but liquid at body temperature and therefore melt in the rectum or vaginal cavity and release the compounds described herein.

Formulations suitable for topical application to the skin can take the form of an ointment, cream, lotion, paste, gel, spray, aerosol, or oil. Carriers that can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Pharmaceutical Research 3 (6):318 (1986)) and typically take the form of an optionally buffered aqueous solution. Suitable formulations comprise citrate or bistris buffer (pH 6) or ethanol/water.

The compounds described herein can be formulated for nasal administration or otherwise administered to the lungs of a subject by any suitable means, for example, by an aerosol suspension of respirable particles including the compounds described herein, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al. (1992) J. Pharmacol. Toxicol. Methods 27:143-159. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles including the compounds described herein can likewise be produced with any solid particulate medicament aerosol generator, by techniques known in the pharmaceutical art.

Alternatively, one can administer the compounds described herein in a local rather than systemic manner, for example, in a depot or sustained-release formulation.

In particular embodiments of the invention, administration is by subcutaneous or intradermal administration. Subcutaneous and intradermal administration can be by any method known in the art including, but not limited to, injection, gene gun, powderject device, bioject device, microenhancer array, microneedles, and scarification (i.e., abrading the surface and then applying a solution including the compounds described herein).

In other embodiments, the compounds described herein are administered intramuscularly, for example, by intramuscular injection or by local administration.

Novel Compounds and Compositions

Novel compounds according to some embodiments of the invention include the compounds of Formula (I)

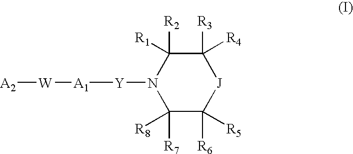

or a pharmaceutically acceptable prodrug, salt, polymorph, solvate, enantiomer, diastereomer, racemate, mixture of stereoisomers thereof, or derivative thereof, wherein:

W is null, oxy, amino, thio, sulfinyl, sulfonyl, carbonyl, amide, alkylene or cycloalkylidene,

wherein at least one carbon atom of the alkylene or cycloalkylidene is optionally substituted with an oxy, amino, thio, sulfinyl, sulfonyl, carbonyl or amide group, and wherein the alkylene or cycloalkylidene is optionally substituted with at least one halogen atom;

A₁ is a monocyclic cycloalkylidene, monocyclic heterocycloalkylidene, monocyclic arylene or monocyclic heteroarylene, each optionally substituted with at least one alkyl, alkoxy, fluoroalkyl, cycloalkyl, hydroxy, halo, fluoroalkoxy, alkenyl, alkenoxy, phosphoramide, phosphoramidealkyl, phosphonate, phosphonatealkyl or —R₉Q, wherein R₉ is null or alkylene and Q is —NR₁₀OR₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈, or —OR₂₉;

A₂ is null, cycloalkyl, heterocycloalkyl, aryl or heteroaryl, optionally substituted with at least one of an alkyl, alkoxy, fluoroalkyl, cycloalkyl, hydroxy, halo, fluoroalkoxy, alkenyl, alkenoxy and/or —R₉Q, wherein R₉ and Q are as defined above;

R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are each independently hydrogen, alkyl, aryl, heteroaryl, allyl, alkoxy, cycloalkyl, heterocycloalkyl, fluoroalkyl, fluorocycloalkyl, arylalkyl or heteroarylalkyl; or wherein R₁₀ and R₁₁, R₁₆ and R₁₇, R₂₁ and R₂₂ or R₂₃ and R₂₄, taken together with the nitrogen to which they are attached, are part of a heterocycloalkyl or heteroaryl;

Y is —CO—CO—, —SO₂—, —C═NRₓ—CO—, and —CO—C═NRₓ—, —O—CO—, or —NR₃₀CO—; wherein Rₓ is alkyl, fluoroalkyl, alkoxyalkyl, cycloalkyl, heterocycloalkyl, aryl or heteroaryl, optionally substituted with at least one halogen, alkyl, alkoxy, —CF₃, —OCF₃, and/or —CN;

R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge,

wherein the alkyl or alkylene bridge is optionally substituted with at least one halogen, amino, hydroxyl, —CN, —NO₂, alkoxy, —CF₃, —OCF₃, alkyl, allyl, fluoroalkyl, cycloalkyl, fluorocycloalkyl, heterocycloalkyl, aryl, heteroaryl, arylalkyl, heteroarylalkyl, polyether and/or R₃₁-Q′ group, wherein R₃₁ is null or alkylene and Q′ is —SO₂NR₃₂R₃₃, —NR₃₄COR₃₅, —CONR₃₆R₃₇ or —COOR₃₈;

R₃₀, R₃₂, R₃₃, R₃₄, R₃₅, R₃₆, R₃₇ and R₃₈ are each hydrogen, alkyl, allyl, fluoroalkyl, cycloalkyl, heterocycloalkyl, fluorocycloalkyl, alkoxy, aryl, heteroaryl, arylalkyl, or heteroarylalkyl; or R₃₂ and R₃₃ or R₃₆ and R₃₇, taken together with the nitrogen to which they are attached, are part of a heterocycloalkyl or heteroaryl; and

wherein the cycloalkyl, heterocycloalkyl, aryl and heteroaryl are each independently optionally substituted with at least one hydrogen, halo, alkoxy, —CF₃, —OCF₃ and/or —CN;

J is

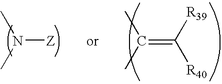

Z is —COR₄₁, —C(═NR₄₃)R₄₁ or R₄₂;

R₄₁ is cycloalkyl, heterocycloalkyl, aryl or heteroaryl; each optionally substituted with at least one alkyl, cycloalkyl, alkoxy, fluoroalkoxy, fluoroalkyl, fluorocycloalkyl, halo, —CN, —CF₃, alkylthio, hydroxy, alkenyl, alkenoxy, acetyl and/or —R₉Q, wherein R₉ and Q are defined above;

R₄₂ is aryl or heteroaryl, optionally substituted with at least one halo, alkoxy, —CF₃, —OCF₃, —CN, alkyl, -cycloalkyl, -fluoroalkoxy, fluoroalkyl, fluorocycloalkyl, alkylthio, hydroxy, acetyl, alkenyl, alkenoxy and/or —R₉Q, wherein R₉ and Q are defined above;

R₄₃ is hydrogen, —CN, alkoxy, fluoroalkoxy, alkyl, fluoroalkyl, cycloalkyl, fluorocycloalkyl, aryl, heteroaryl or heterocycloalkyl;

wherein the cycloalkyl, heterocycloalkyl, aryl and heteroaryl are optionally substituted with at least one halo, alkyl, alkoxy, —CF₃, —OCF₃, —CN, cycloalkyl, fluoroalkoxy, fluoroalkyl, fluorocycloalkyl, alkylthio, hydroxy, alkenyl, alkenoxy and/or —R₉Q, wherein R₉ and Q are defined above;

R₃₉ is cycloalkyl, heterocycloalkyl, aryl or heteroaryl, each optionally substituted with at least one halogen, alkyl, alkoxy, —CF₃, —OCF₃, —CN, cycloalkyl, fluoroalkoxy, fluoroalkyl, fluorocycloalkyl, S-alkyl, hydroxy, alkenyl, alkenoxy, acetyl and/or —R₉Q, wherein R₉ and Q are defined above; and

R₄₀ is hydrogen, —CN, alkyl, halo, —CF₃, cycloalkyl, fluoroalkyl, fluorocycloalkyl, aryl, heteroaryl, arylalkyl, heteroarylalkyl or heterocycloalkyl,

wherein the cycloalkyl, heterocycloalkyl, aryl, heteroaryl, arylalkyl and heteroarylalkyl are optionally substituted with at least one halo, alkyl, alkoxy, —CF₃, —OCF₃, —CN, cycloalkyl, fluoroalkoxy, fluoroalkyl, fluorocycloalkyl, alkylthio, hydroxy, alkenyl, alkenoxy and/or —R₉Q, wherein R₉ and Q are defined above.

In particular embodiments of the invention, in a compound of Formula (I),

W is null, C₀-C₆ alkylene, (C₀-C₃ alkylene)-O—(C₀-C₃ alkylene), (C₀-C₃ alkylene)-NR′—(C₀-C₃ alkylene), (C₀-C₃ alkylene)-S—(C₀-C₃ alkylene), (C₀-C₃ alkylene)-S(═O)—(C₀-C₃ alkylene), (C₀-C₃ alkylene)-SO₂—(C₀-C₃ alkylene), (C₀-C₃ alkylene)-C(═O)—(C₀-C₃ alkylene), (C₀-C₃ alkylene)-C(═O)NR′—(C₀-C₃ alkylene) or (C₀-C₆ cycloalkylidene), wherein the alkylene and cycloalkylidene groups are optionally substituted with 1 to 3 halogen atoms;

A₁ is phenylene or monocyclic heteroarylene, wherein the phenylene and monocyclic heteroarylene are optionally substituted with 1 to 5 functional groups, wherein each functional group may be a C₁-C₆ alkyl, C₁-C₆ alkoxy, C₁-C₆ fluoroalkyl, C₃-C₇ cycloalkyl, hydroxy, halo, C₁-C₆ fluoroalkoxy, C₂-C₆ alkenyl, C₂-C₆ alkenoxy or —R₉Q, wherein R₉ is null or C₁-C₂ alkylene and Q is —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CON R₂₃R₂₄, —NR₂₅SOR₂₆, —R₂₇COR₂₈, or —OR₂₉;

A₂ is phenyl or heteroaryl, wherein the phenyl and heteroaryl are optionally substituted with 1 to 5 functional groups, wherein each functional group may be a C₁-C₆ alkyl, C₁-C₆ alkoxy, C₁-C₆ fluoroalkyl, C₃-C₇ cycloalkyl, hydroxy, halogen, C₁-C₆ fluoroalkoxy, C₂-C₆ alkenyl, C₂-C₆ alkenoxy or —R₉Q, wherein R₉ is null or C₁-C₂ alkylene and Q is defined above;

R′, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are each independently hydrogen, C₁-C₆ alkyl, allyl, C₁-C₆ fluoroalkyl, C₃-C₇ cycloalkyl, C₃-C₇ fluorocycloalkyl, C₁-C₆ alkoxy, phenyl, phenylmethyl, phenylethyl, heteroaryl, heteroarylmethyl, heteroarylethyl, heterocycloalkyl, heterocycloalkylmethyl or heterocycloalkylethyl; or wherein R₁₀and R₁₁, R₁₆ and R₁₇, R₂₁ and R₂₂, or R₂₃ and R₂₄, taken together with the nitrogen to which they are attached, are part of a ring selected from the group consisting of azetidine, azetidin-2-one, pyrrolidine, pyrrolidin-2-one, pyrrolidin-3-one, piperidine, piperidin-2-one, piperidin-3-one, piperidin-4-one, morpholine, morpholin-2-one, morpholin-3-one and N-alkylpiperazine;

wherein the heterocycloalkyl includes

0 to 4 nitrogen atoms;

0 to 2 nitrogen atoms and 0 to 1 oxygen atom;

0 to 2 nitrogen atoms and 0 to 1 sulfur atom; or

0 to 2 nitrogen atoms, 0 to 1 oxygen atom and 0 to 1 sulphur atom; and

wherein the heteroaryl imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenzimidazolyl, indazolyl, quinazolinyl, phthalazinyl, benzoxazolyl or quinoxalinyl; and

wherein the phenyl, heteroaryl or heterocycloalkyl is optionally substituted with 1 to 5 functional groups, wherein each functional group may be halo, C₁-C₆ alkoxy, —CF₃, —OCF₃ or —CN;

Rₓ is alkyl, fluoroalkyl, alkoxyalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl; wherein each heteroaryl ring is optionally substituted with 1 to 5 functional groups wherein each functional group may be halo, C₁-C₆ alkyl, C₁-C₆ alkoxy, —CF₃, —OCF₃ or —CN;

R₁, R₂, R₃, R₄, R₅, R₆, R₇, and R₈ are each independently hydrogen or C₁-C₆ alkyl,

wherein the C₁-C₆ alkyl is optionally substituted with 1 to 3 functional groups, wherein each functional group may be halo, amino, hydroxyl, —CN, —NO₂, C₁-C₆ alkoxy, —CF₃, —OCF₃, C₁-C₆ alkyl, allyl, C₁-C₆ fluoroalkyl, C₃-C₇ cycloalkyl, C₃-C₇ fluorocycloalkyl, phenyl, phenylmethyl, phenylethyl, heteroaryl, heteroarylmethyl, heteroarylethyl, heterocycloalkyl, heterocycloalkylmethyl, heterocycloalkylethyl, (CRₐR_{b})_{U}-T-(CR_{c}R_{d})_{U′}Rₑ or R₃₁Q′ wherein R₃₁ is null or C₁-C₂ alkylene and Q′ is —SO₂NR₃₂R₃₃, —NR₃₄COR₃₅, —CONR₃₆R₃₇ or —COOR₃₈;

R₃₀, R₃₂, R₃₃, R₃₄, R₃₅, R₃₆, R₃₇, R₃₈, Rₐ, R_{b}, R_{c}, R_{d} and Rₑ are each independently hydrogen, C₁-C₆ alkyl, allyl, C₁-C₆ fluoroalkyl, C₃-C₇ cycloalkyl, C₃-C₇ fluorocycloalkyl, C₁-C₆ alkoxy, phenyl-(C₀-C₂ alkyl), heteroaryl-(C₀-C₂ alkyl) or heterocycloalkyl-(C₀-C₂ alkyl);

wherein the heterocycloalkyl includes

0 to 4 nitrogen atoms;

0 to 2 nitrogen atoms and 0 to 1 oxygen atom;

0 to 2 nitrogen atoms and 0 to 1 sulfur atom; or

0 to 2 nitrogen atoms, 0 to 1 oxygen atom and 0 to 1 sulphur atom; and

wherein the heteroaryl group is imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenzimidazolyl, indazolyl, quinazolinyl, phthalazinyl, benzoxazolyl, or quinoxalinyl;

wherein the phenyl, heteroaryl or heterocycloalkyl is optionally substituted with 1 to 5 functional groups, wherein each functional group may be halo, C₁-C₆ alkoxy, —CF₃, —OCF₃ or —CN; or wherein R₃₂ and R₃₃ or R₃₆ and R₃₇, taken together with the nitrogen to which they are attached, are part of a heterocycloalkyl selected from the group consisting of aziridine, azetidine, pyrrolidine, pyrrolidin-2-one, piperidine, morpholine and N-alkylpiperazine;

U and U′ are each independently 0, 1 or 2;

T is null or oxy;

R₄₁ is phenyl, pyridinyl, pyrazinyl, pyridazinyl, pyrimidinyl, thienyl, furyl, thiazolyl, isothiazolyl, oxazolyl, isoxazolyl, pyrazoyl, imidazolyl, triazolyl, oxadiazolyl, thiadiazolyl or tetrazolyl; each of which is optionally substituted with at least one C₁-C₃ alkyl, C₃-C₆ cycloalkyl, C₁-C₃ alkoxy, C₁-C₆ fluoroalkoxy, C₁-C₆ fluoroalkyl, C₃-C₇ fluorocycloalkyl, —CN, —F, —Cl, —Br, —CF₃, C₀-C₃ alkylthio, hydroxy, C₂-C₆ alkenyl, C₂-C₆ alkenoxy, acetyl and/or —R₉Q, wherein R₉ is null or C₁-C₂ alkylene and Q is defined above;

R₄₂ is phenyl, heteroaryl, quinolinyl, isoquinolinyl, benzimidazolyl, azabenimidazolyl, benzothienyl, benzofuryl, benzoindazolyl, quinazolinyl, phthalazinyl, benzoxazolyl, quinoxalinyl, thienopyridine, thienopyrimidine, thienopyridazine, thienopyrazine, furopyridine, furoopyrimidine, furopyridazine, furopyrazine, oxazolopyridine, oxazolopyrimidine, oxazolopyridazine, oxazolopyrazine, thiazolopyridine, thiazolopyrimidine, thiazolopyridazine,thiazolopyrazine, napthyridine, pyridopyrimidine, pyridopyridazine or pyridopyrazine;

each optionally substituted with 1 to 5 functional groups, wherein each functional group may be halo, C₁-C₆ alkoxy, —CF₃, —OCF₃ or —CN, C₁-C₃ alkyl, C₃-C₆ cycloalkyl, C₁-C₆ fluoroalkoxy, C₁-C₆ fluoroalkyl, C₃-C₇ fluorocycloalkyl, C₀-C₃ alkylthio, hydroxy, C₂-C₆ alkenyl, C₂-C₆ alkenoxy, acetyl or —R₉Q, wherein R₉ is null or C₁-C₂ alkylene and Q is defined above;

R₄₃ is hydrogen, —CN, C₁-C₆ alkoxy, C₁-C₆ fluoroalkoxy, C₁-C₆ alkyl, C₁-C₆ fluoroalkyl, C₃-C₇ cycloalkyl or C₃-C₇ fluorocycloalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl;

wherein the aryl or heteroaryl are optionally substituted with 1 to 5 functional groups, wherein each functional group may be halo, C₁-C₆ alkyl, C₁-C₆ alkoxy, —CF₃, —OCF₃ or —CN, C₁-C₃ alkyl, C₃-C₆ cycloalkyl, C₁-C₆ fluoroalkoxy, C₁-C₆ fluoroalkyl, C₃-C₇ fluorocycloalkyl, C₀-C₃ alkylthio, hydroxy, C₂-C₆ alkenyl, C₂-C₆ alkenoxy, acetyl or —R₉Q, wherein R₉ is null or C₁-C₂ alkylene and Q is defined above;

R₃₉ is phenyl, imidazolyl, pyrazolyl, triazolyl, tetrazolyl, thienyl, furyl, thiazolyl, isothiazolyl, oxazolyl,isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl;

each optionally substituted with 1 to 5 functional groups, wherein each functional group may be halo, C₁-C₆ alkyl, C₁-C₆ alkoxy, —CF₃, —OCF₃, —CN, C₁-C₃ alkyl, C₃-C₆ cycloalkyl, C₁-C₆ fluoroalkoxy, C₁-C₆ fluoroalkyl, C₃-C₇ fluorocycloalkyl, S—(C₀-C₃ alkyl), hydroxy, C₂-C₆ alkenyl, C₂-C₆ alkenoxy, acetyl or —R₉Q, wherein R₉ is null or C₁-C₂ alkylene and Q is defined above; and

R₄₀ is hydrogen, —CN, C₁-C₆ alkyl, halo, —CF₃, C₃-C₆ cycloalkyl, C₁-C₆ fluoroalkyl, C₃-C₇ fluorocycloalkyl, heterocycloalkyl, heterocycloalkylmethyl, heterocycloalkylethyl, R₄₁, —CH₂R₄₁ and —CH₂CH₂R₄₁;

wherein the heterocycloalkyl includes

0 to 4 nitrogen atoms;

0 to 2 nitrogen atoms and 0 to 1 oxygen atom;

0 to 2 nitrogen atoms and 0 to 1 sulfur atom; or

0 to 2 nitrogen atoms, 0 to 1 oxygen atom and 0 to 1 sulphur atom; and

wherein R₄₁ is phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenimidazolyl, indazolyl, quinazolinyl, phthalazinyl, benzoxazolyl or quinoxalinyl; and is optionally substituted with 1 to 5 functional groups, wherein each functional group may be halogen, C₁-C₆ alkyl, C₁-C₆ alkoxy, —CF₃, —OCF₃, —CN, hydrogen, C₁-C₃ alkyl, C₃-C₆ cycloalkyl, O—(C₁-C₆ fluoroalkyl), C₁-C₆ fluoroalkyl, C₃-C₇ fluorocycloalkyl, S—(C₀-C₃ alkyl), hydroxy, C₂-C₆ alkenyl, C₂-C₆ alkenoxy, acetyl or —R₉Q, wherein R₉ is null or C₁-C₂ alkylene and Q is defined above.

Novel compounds according to embodiments of the invention also include compounds of Formula (II)

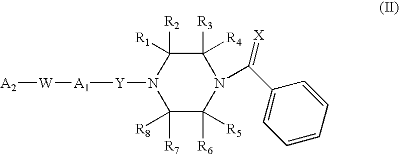

or a pharmaceutically acceptable prodrug, salt, polymorph, solvate, enantiomer, diastereomer, racemate, mixture of stereoisomers thereof, or derivative thereof, wherein:

W is null, oxy, amino, thio, sulfinyl, sulfonyl, carbonyl, amide, alkylene or cycloalkylidene,

wherein at least one carbon atom of the alkylene or cycloalkylidene is optionally substituted with an oxy, amino, thio, sulfinyl, sulfonyl, carbonyl or amide group, and wherein the alkylene or cycloalkylidene is optionally substituted with 1-3 halogen atoms;

A₁ is a monocyclic cycloalkylidene, monocyclic heterocycloalkylidene, monocyclic arylene and monocyclic heteroarylene, optionally substituted with 1 to 5 functional groups, wherein each functional group may be alkyl, alkoxy, fluoroalkyl, cycloalkyl, hydroxy, halo, fluoroalkoxy, alkenyl, alkenoxy or —R₉Q, wherein Q is —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈ or —OR₂₉;

A₂ is null, cycloalkyl, heterocycloalkyl, aryl pr heteroaryl, wherein the cycloalkyl, heterocycloalkyl, aryl or heteroaryl is optionally substituted with 1 to 5 functional groups, wherein each functional group may be alkyl, alkoxy, fluoroalkyl, cycloalkyl, hydroxy, halo, fluoroalkoxy, alkenyl, alkenoxy and —R₉Q, wherein R₉ and Q are as defined above;

R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are hydrogen, alkyl, aryl, heteroaryl, allyl, alkoxy, cycloalkyl, heterocycloalkyl, fluoroalkyl, fluorocycloalkyl, arylalkyl or heteroarylalkyl; or wherein R₁₀ and R₁₁, R₁₆ and R₁₇, R₂₁ and R₂₂, or R₂₃ or R₂₄, taken together with the nitrogen to which they are attached, are part of a heterocycloalkyl or heteroaryl;

Y is —CO—CO—, —SO₂—, —C═NRₓ—CO—, and —CO—C═NRₓ—, —O—CO—, or —NR₃₀CO—; wherein Rₓ is alkyl, fluoroalkyl, alkoxyalkyl, cycloalkyl, heterocycloalkyl, aryl or heteroaryl, optionally substituted with 1 to 5 functional groups, wherein each functional group may be halogen, alkyl, alkoxy, —CF₃, —OCF₃ and —CN;

R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge,

wherein the alkyl or alkylene bridge is optionally substituted with 1 to 3 functional groups, wherein each functional group may be halogen, amino, hydroxyl, —CN, —NO₂, alkoxy, —CF₃, —OCF₃, alkyl, allyl, fluoroalkyl, cycloalkyl, fluorocycloalkyl, heterocycloalkyl, aryl, heteroaryl, arylalkyl, heteroarylalkyl, polyether or R₃₁-Q′ wherein R₃₁ is null or alkylene and Q′ is —SO₂NR₃₂R₃₃, —NR₃₄COR₃₅, —CONR₃₆R₃₇ or —COOR₃₈;

R₃₀, R₃₂, R₃₃, R₃₄, R₃₅, R₃₆, R₃₇ and R₃₈ are each independently hydrogen, alkyl, allyl, fluoroalkyl, cycloalkyl, heterocycloalkyl, fluorocycloalkyl, alkoxy, aryl, heteroaryl, arylalkyl or heteroarylalkyl; or wherein R₃₂ and R₃₃ or R₃₆ and R₃₇, taken together with the nitrogen to which they are attached, are part of a heterocycloalkyl or heteroaryl; and

wherein the cycloalkyl, heterocycloalkyl, aryl and heteroaryl are each independently optionally substituted with 1 to 5 functional groups, wherein each functional group may be halo, alkoxy, —CF₃, —OCF₃ and —CN; and

X is O, S or NR₃₉, wherein R₃₉ is hydrogen, —CN, alkoxy, fluoroalkoxy, alkyl, fluoroalkyl, cycloalkyl, fluorocycloalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl or quinoxalinyl;

optionally substituted with 1 to 5 functional groups, wherein each functional group may be halo, alkyl, alkoxy, —CF₃, —OCF₃ or —CN, cycloalkyl, fluoroalkoxy, fluoroalkyl, fluorocycloalkyl, alkylthio, hydroxy, alkenyl, alkenoxy, acetyl or —R₉Q, wherein R₉ and Q are defined above.

In particular embodiments of the invention, in the compound of Formula (I),

W is —(CH₂)ₓ(CO)_{y}(CH₂)₂—, wherein x, y and z are each independently 0, 1, 2 or 3;

A₁ is a cycloalkylidene, heterocycloalkylidene, arylene or heteroarylene, each optionally substituted with 1 to 3 functional groups, wherein each functional group may be halo, alkyl, alkoxy, fluoroalkyl, fluoroalkoxy, hydroxy, amino, alkylamino, dialkylamino or thiol;

A₂ is a monocyclic or bicyclic cycloalkyl, monocyclic or bicyclic heterocycloalkyl, monocyclic or bicyclic aryl or monocyclic or bicyclic heteroaryl,

each optionally substituted with 1 to 3 functional groups, wherein each functional group may be halo, —CN, alkyl, alkoxy, acetyl, oxo, fluoroalkyl, fluoroalkoxy, hydroxy, amino, methylamino, dimethylamino, —SH, cycloalkyl, heterocycloalkyl, aryl, heteroaryl, arylalkyl or arylcarbonyl;

wherein the cycloalkyl, heterocycloalkyl, aryl or heteroaryl substituted onto the monocyclic or bicyclic ring is optionally substituted with a halo, alkyl, acetyl or alkoxycarbonyl;

Y is —(CH₂)ₘ(C═O)ₙ— or —SO₂—, wherein m and n are each independently 0, 1, 2 or 3;

R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge; and

X is O, —CN or N—O-alkyl.

With regard to the compounds and compositions described herein, according to some embodiments, any of the R groups and/or functional groups represented thereby can be excluded from a particular compound or composition.

According to some embodiments of the invention, the compounds of Formula (I) and Formula (II) are present as racemic mixtures. However, in some embodiments, compound of Formula (I) and Formula (II) are present substantially as one enantiomer or in the enantiomerically pure (R) form. The term “substantially on enantiomer” as used herein, refers to a %(R) enantiomer or %(S) enantiomer of greater than about 60%, in some embodiments about 90%, and in some embodiments greater than 95%.

Pharmaceutical compositions of embodiments of the invention include a compound according to an embodiment of the invention, or a pharmaceutically acceptable prodrug, salt, polymorph, solvate, enantiomer, diastereomer, racemate, mixture of stereoisomers thereof, or derivative thereof; and a pharmaceutically acceptable carrier, excipient or diluent.

Methods

A compound according to the present invention or a pharmaceutically acceptable prodrug, salt, polymorph, solvate, enantiomer, diastereomer, racemate, mixture of stereoisomers thereof, or derivative thereof, or as part of a pharmaceutical composition, may be used in antiviral treatment alone (also known as “monotherapy”), or in combination or in a treatment regimen (e.g., when used simultaneously, or in a cycling on with one drug and cycling off with another) with other therapeutic agents (including antiviral drugs) used for treatment of HIV (e.g., including, but not limited to, other HIV entry inhibitors (e.g., gp41 fusion inhibitors, CCR5 inhibitors, retrocyclin, CD4 inhibitors, gp120 inhibitors, and the like), HIV integrase inhibitors, reverse transcriptase inhibitors (e.g., nucleoside or nonnucleoside), protease inhibitors, viral-specific transcription inhibitors, viral processing inhibitors, HIV maturation inhibitors, inhibitors of uridine phosphorylating enzyme, HIV vaccines, and the like, as well known in the art. One commonly used treatment, involving a combination of antiviral agents, is known as HAART (Highly Active Anti-Retroviral Therapy). HAART typically combines three or more drugs with antiviral activity against HIV, and typically involves more than one class of drug (a “class” referring to the mechanism of action, or viral protein or process targeted by the drug). Thus, a method of treatment, a compound, and a pharmaceutical composition, according to the present invention, may be administered alone (e.g., as monotherapy) or may be administered in a treatment regimen, or co-administered, involving a combination of additional therapeutic agents for the treatment of HIV infection and/or AIDS, as described in more detail herein.

For example, in some embodiments, one or more therapeutic agents may be combined in treatment with a compound (by itself, or in a pharmaceutical composition) according to the present invention. Typically, the combination comprises two or more antiviral agents to increase the efficacy of the treatment by, for example, reducing the ability of the virus to become resistant to the antiviral agents used in the treatment (as compared to monotherapy). Such combinations may be prepared from effective amounts of antiviral agents (useful in treating of HIV infection) currently approved or approved in the future, which include, but are not limited to, one or more additional therapeutic agents selected from the following: reverse transcriptase inhibitor, including but not limited to, abacavir, AZT (zidovudine), ddC (zalcitabine), nevirapine, ddI (didanosine), FTC (emtricitabine), (+) and (−) FTC, reverset, 3TC (lamivudine), GS 840, GW-1592, GW-8248, GW-5634, HBY097, delaviridine, efavirenz, d4T (stavudine), FLT, TMC125, adefovir, tenofovir, and alovudine; protease inhibitor, including but not limited to, amprenivir, CGP-73547, CGP-61755, DMP-450, indinavir, nelfinavir, PNU-140690, ritonavir, saquinavir, telinavir, tipranovir, atazanavir, lopinavir; viral entry inhibitor, including but not limited to, fusion inhibitor (enfuvirtide, T-1249, other fusion inhibitor peptides, and small molecules), chemokine receptor antagonist (e.g., CCR5 antagonist, such as ONO-4128, GW-873140, AMD-887, CMPD-167; CXCR4 antagonist, such as AMD-070), an agent which affects viral binding interactions (e.g., affects gp120 and CD4 receptor interactions, such as BMS806, BMS-488043; and/or PRO 542, PRO140; or lipid and/or cholesterol interactions, such as procaine hydrochloride (SP-01 and SP-01A)); integrase inhibitor, including but not limited to, L-870, and 810; RNAseH inhibitor; inhibitor of rev or REV; inhibitor of vif (e.g., vif-derived proline-enriched peptide, HIV-1 protease N-terminal-derived peptide); viral processing inhibitor, including but not limited to betulin, and dihydrobetulin derivatives (e.g., PA-457); and immunomodulator, including but not limited to, AS-101, granulocyte macrophage colony stimulating factor, IL-2, valproic acid, and thymopentin. As appreciated by one skilled in the art of treatment of HIV infection and/or AIDS, a combination drug treatment may comprise two or more therapeutic agents having the same mechanism of action (viral protein or process as a target), or may comprise two or more therapeutic agents having a different mechanism of action.

Thus, according to some embodiments, administration of a compound according to an embodiment of the invention, or a pharmaceutically acceptable prodrug, salt, polymorph, solvate, enantiomer, diastereomer, racemate, mixture of stereoisomers thereof, or derivative thereof, and another therapeutic agent is sequential, and in other embodiments, administration of a compound according to an embodiment of the invention, or a pharmaceutically acceptable prodrug, salt, polymorph, solvate, enantiomer, diastereomer, racemate, mixture of stereoisomers thereof, or derivative thereof, and another therapeutic agent is simultaneous. According to some embodiments, administration of a compound according to the present invention and another therapeutic agent is simultaneous.

According to some embodiments, the administration of at least one of the therapeutic agents is oral, and in some embodiments, administration of at least one of the therapeutic agents is parenteral, such as subcutaneous.

According to some embodiments of the invention, methods of treating HIV infection in a subject include administering an effective amount of a compound according to an embodiment of the invention, or a pharmaceutically acceptable prodrug, salt, polymorph, solvate, enantiomer, diastereomer, racemate, mixture of stereoisomers thereof, or derivative thereof, and an effective amount of at least one therapeutic agent.

According to some embodiments of the invention, provided are methods of inhibiting HIV replication including administering to a subject an effective amount of the compound according to an embodiment of the invention, or a pharmaceutically acceptable prodrug, salt, polymorph, solvate, enantiomer, diastereomer, racemate, mixture of stereoisomers thereof, and an effective amount of at least one therapeutic agent.

Effective dosages of these illustrative additional therapeutic agents, which may be used in combinations with a compound, or pharmaceutical composition, according to the present invention, are known in the art. Such combinations may include a number of antiviral agents or therapeutic agents that can be administered by one or more routes, sequentially or simultaneously, depending on the route of administration and desired pharmacological effect, as is apparent to one skilled in the art. Effective dosages of a compound or pharmaceutical composition according to the present invention to be administered may be determined through procedures well known to those in the art; e.g., by determining potency, biological half-life, bioavailability, and toxicity. In a preferred embodiment, an effective amount of a compound according to the present invention and its dosage range are determined by one skilled in the art using data from routine in vitro and in vivo studies well know to those skilled in the art. For example, in vitro infectivity assays of antiviral activity, such as described herein, enables one skilled in the art to determine the mean inhibitory concentration (IC) of the compound, as the sole active ingredient or in combination with other active ingredients, necessary to inhibit a predetermined range of viral infectivity (e.g., 50% inhibition, IC₅₀; or 90% inhibition, IC₉₀) or viral replication. Appropriate doses can then be selected by one skilled in the art using pharmacokinetic data from one or more standard models, so that a minimum plasma concentration (C[min]) of the active ingredient is obtained which is equal to or exceeds a predetermined value for inhibition of viral infectivity or viral-replication. While dosage ranges typically depend on the route of administration chosen and the formulation of the dosage, when administered orally, an exemplary dosage range of a compound according to the present invention, as an active ingredient, may be from about 1 mg/kg body weight to about 100 mg/kg body weight; and more preferably no less than 1 mg/kg body weight to no more than 10 mg/kg body weight.

A compound or pharmaceutical composition according to the present invention may be administered to an individual by any means that enables the active ingredient to reach the target cells. Thus, a compound or pharmaceutical composition according to the present invention may be administered by any suitable technique, including oral, parenteral (e.g., intramuscular, intraperitoneal, intravenous, or subcutaneous injection or infusion, intradermal, or implant), nasal (e.g., inhalation spray), pulmonary, vaginal, rectal, sublingual, or other suitable routes of administration; and can be formulated in dosage forms appropriate for each route of administration. The specific route of administration will depend, e.g., on the medical history of the individual, including any perceived or anticipated side effects from such administration, other factors known to medical practitioners, and the formulation of the compound, or a pharmaceutically acceptable prodrug, salt, polymorph, solvate, enantiomer, diastereomer, racemate, mixture of stereoisomers thereof, or derivative thereof (by itself or as part of combination treatment) being administered. In particular embodiments, a compound or pharmaceutical composition according to the present invention is administered to an individual orally.

Thus, in accordance with the present invention, provided are methods for inhibition of transmission of HIV to a cell, comprising administering a compound or a pharmaceutically acceptable prodrug, salt, polymorph, solvate, enantiomer, diastereomer, racemate, mixture of stereoisomers thereof, or derivative thereof, or pharmaceutical composition according to the present invention in an effective amount to inhibit infection of the cell by HIV. The method may further include administering a compound, or a pharmaceutically acceptable prodrug, salt, polymorph, solvate, enantiomer, diastereomer, racemate, mixture of stereoisomers thereof, or derivative thereof, or pharmaceutical composition according to the present invention in combination with other therapeutic agents used to treat HIV infection and/or AIDS to an individual by administering to the individual the combination (simultaneously or sequentially, or a part of a therapeutic regimen) of therapeutic agents which includes an effective amount of the compound, pharmaceutically acceptable prodrug, salt, polymorph, solvate, enantiomer, diastereomer, racemate, mixture of stereoisomers thereof, or derivative thereof, or pharmaceutical composition according to the present invention.

Also provided are methods for inhibiting HIV entry comprising administering to an individual in need of treatment a compound or a pharmaceutically acceptable prodrug, salt, polymorph, solvate, enantiomer, diastereomer, racemate, mixture of stereoisomers thereof, or derivative thereof or pharmaceutical composition according to the present invention in an effective amount to inhibit viral entry of a target cell. The methods may further include administering a compound, a pharmaceutically acceptable prodrug, salt, polymorph, solvate, enantiomer, diastereomer, racemate, mixture of stereoisomers thereof, or derivative thereof, or pharmaceutical composition according to the present invention in combination with one or more additional inhibitors of viral entry useful in treating HIV infection, in an effective amount.

Embodiments of the present invention provide prophylaxis of the diseases and disorders described herein. In some embodiments, the inventive methods eliminate or reduce the incidence or onset of the disease or disorder, as compared to that which would occur in the absence of the measure taken. Alternatively stated, the present methods slow, delay, control, or decrease the likelihood or probability of the disease or disorder in the subject, as compared to that which would occur in the absence of the measure taken.

Embodiments of the invention further provide kits that can include at least one compound according to embodiments of the present invention or a pharmaceutically acceptable prodrug, salt, polymorph, solvate, enantiomer, diastereomer, racemate, mixture of stereoisomers thereof, or derivative thereof, or pharmaceutical composition according to the present invention, and optionally instructions for administering the same. Further, the kits can include additional therapeutic agents useful for the treatment of HIV. In some embodiments, the components of the kits may be packaged together in a common container.

### EXAMPLES

### Example 1

In this example are illustrated experimental procedures for determining biological activity, such as antiviral activity. For determining biological activity, an HIV-1 infection assay was used to determine the antiviral potency for compounds of the present invention. In using an in vitro assay for demonstrating antiviral potency, it is important to note that antiviral effect demonstrated in the in vitro assay has been correlated with, antiviral effect in vivo. For example, one or more antiviral agents known to have an antiviral effect in vivo, were used to demonstrate that such antiviral agents also demonstrated an antiviral effect in this in vitro virus assay.

For determining biological activity, an HIV-1 pseudotyped virus assay was used to determine the respective antiviral potencies of each compound tested in the assay for comparison. The pseudotyped assay scores for a reduction in infection as indicated by decreased signal from the reporter gene encoding a luciferase enzyme (“reporter gene”). The assay employs cell lines expressing CD4 and either of the primary chemokine receptors (CCR5 or CXCR4) that HIV uses as a co-receptor (“target cells”). Pseudotyped virus was prepared by co-transfection of 293T cells with: 1) a plasmid construct carrying the HIV-1 envelope of choice, in combination with 2) a pseudotyped virus backbone construct in which (a) envelope expression has been abrogated due to a frameshift in the envelope sequence, and (b) the reporter gene replaces nef. Expression of HIV-1 envelope on 293T packaging cells results in the production of a pseudotyped virus carrying the reporter gene that is capable of a single cycle of infection.

The compounds of the present invention being tested for antiviral activity were serially diluted and dose responses determined in duplicate in two separate experiments. The compounds to be tested were added directly to the plated, target cells, followed by the addition of pseudotyped virus described above. The cells were cultured for three days prior harvest. Media and compound were removed, the cell monolayer was washed, lysed by detergent, and then frozen at −80 degrees C. for a minimum of 30 minutes. Following thawing and acclimation to room temperature, luciferase production was quatified by injecting 100 □I of a substrate (of the enzyme encoded by the reporter gene) into each well followed by detecting the signal (light emitted from the interaction between the enzyme and substrate) after 5 seconds. In the pseudotyped assay, a 50% reduction in signal is significant, and provides the primary cutoff value for assessing antiviral activity (“IC₅₀” is defined as the dilution resulting in a 50% reduction in enzymatic activity as interpolated from a titration curve). Representative compounds according to the present invention, and their antiviral activity, are illustrated in Table 3.

### Example 2

This example, along with the Schemes herein, illustrate the chemistry and general synthetic procedures to produce compounds according to embodiments of the invention, and intermediates useful for their synthesis. It is understood that reaction conditions, methods, and reactions given in specific examples for specific compounds, are broadly applicable to other compounds of the invention, as described herein. It will be further appreciated by those skilled in the art that it may be necessary or desirable to carry out the synthesis task in the schemes in a different order than described or modify one or more of the transformations, to make the desired compound of Formula (I). It will be still further appreciated by those skilled in the art that, as illustrated in the schemes that follow, it may be necessary or desirable at any stage in the synthesis of compounds of Formula I to protect one or more sensitive groups in the molecules so as to prevent undesirable side reactions. In particular, it may necessary or desirable to protect amino groups, 1-indole or azaindole. The protecting groups that may be used in the preparation of compounds of Formula (I) are well known in the art, and may used in methods well known in the art.

General procedures to prepare biaryl piperazine derivatives are described in Scheme 1. 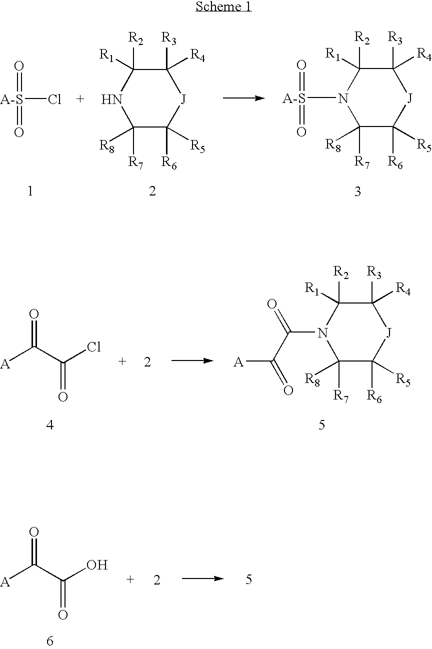
Typically the sulfonyl chloride 1, acid halide 4, or carboxylic acid 6 is coupled with a cyclic amine of general structure 2 using methods well known in the art. In a variation of this procedure, the cyclic amine may be a monoprotected piperazine derivative or a carbonyl-protected analog of 4-piperidinone. In these cases, the protecting group is then cleaved after the reaction steps illustrated in Scheme 2, and additional synthetic transformations are performed on the liberated amino or keto group to provide the final target compounds or the intermediates. The identity of the deprotecting agent will depend on the identity other groups present in the molecule. If cyclic amine 2 is a monoprotected piperazine derivative, a protecting group such as tert-butoxycarconyl (“BOC”) or benzyloxycarbonyl (“CBZ”) may be appropriate. These protecting groups are commonly cleaved with the use of trifluoroacetic acid and hydrogen gas with a palladium catalyst respectively. If 2 is a carbonyl-protected 4-piperidinone, dimethylacetal may be a suitable protecting group, and cleavage may be effected with the use of hydrogen chloride in aqueous methanol. Other useful protecting groups, procedures for the introduction and cleavage are found in the text “Protective Groups in Organic Synthesis” by Theodora W. Greene, Peter G. M. Wuts, (1999), John Wiley and Sons, N.Y., N.Y.)

In a further embodiment, the acid of formula 6 (activated by suitable reagents such as 3-(diethoxyphosphoryloxy)-1,2,3-benzotriazin-4(3H)-one (DEPBT), or HBTU/HATU (HBTU is O-benzotriazol-1-yl-N,N,N′,N′-tetramethyluronium hexafluorophosphate; HATU is O-(7-azabenzotriazol-1-yl)-N,N,N′,N′,-tetramethyl-uronium hexafluorophosphate) and HOBt/HOAt (HOBt is 1-hydroxybenzotriazole hydrate; HOAt is 1-Hydroxy-7-azabenzotriazole), the appropriate cyclic amine of formula 2, and excess amount of an acid acceptor such as triethylamine or N,N-diisopropyl-N-ethylamine, are reacted in a solvent such as haloalkane (e. g. dichloromethane), an ether (e. g. tetrahydrofuran, “THF”), or N,N-dimethylformamide (“DMF”) at room temperature for approximately 4 to 48 hours. The reaction may conveniently be carried out by reacting the relevant piperazine, 1.0 equivalent of the relevant carboxylic acid, 1.2 to about 2 equivalent of HATU, with 1.2 to about 2 equivalent of HOAT, 2.2 to about 10 equivalent of triethylamine in DMF at room temperature for 12 hours.

In yet a further embodiment the acyl chloride of formula 4, the appropriate cyclic amine of formula 2, and excess amount of an acid acceptor such as triethylamine or N,N-diisopropyl-N-ethylamine or N-methyl morpholine, are reacted in a solvent such as haloalkane (e. g. dichloromethane), an ether (e. g. tetrahydrofuran), or DMF at room temperature for about 4 to 48 hours. The reaction may conveniently be carried out by reacting the relevant piperazine, 1.0 equivalent of the relevant acyl chloride, about 2 to 10 equivalent N-methyl morpholine in DCE at room temperature for 12 hours.

In yet a further embodiment the sulfonyl chloride of formula 1, the cyclic amine of formula 2, and excess amount of an acid acceptor such as triethylamine or N,N-diisopropyl-N-ethylamine or N-methyl morpholine or pyridine or the combination of different acid acceptors, are reacted in a solvent such as haloalkane (e. g. dichloromethane), an ether (e. g. tetrahydrofuran), or DMF at room temperature for about 4 to 48 hours. The reaction may conveniently be carried out by reacting the relevant piperazine, 1.0 equivalent of the relevant sulfonyl chloride, about 2 to 10 equivalent N-methyl morpholine in DCE at room temperature for 12 hours.

Piperazines with a single unsubstituted ring nitrogen are available commercially, and can also be produced by a variety of procedures that are illustrated in Scheme 2.

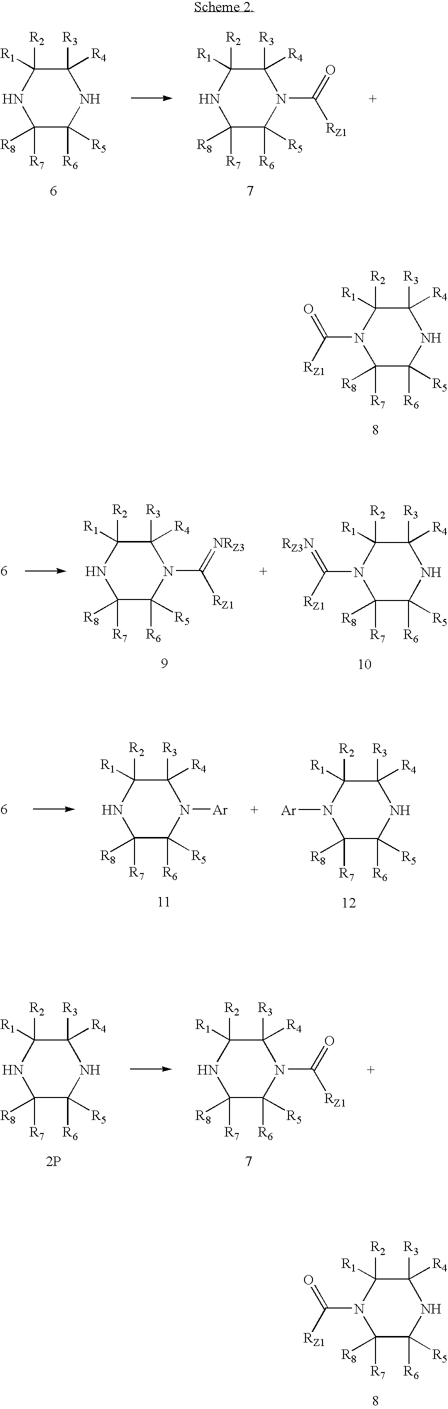

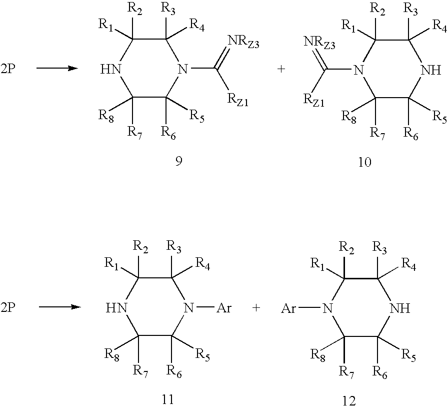

Piperazines with two unsubstituted ring nitrogens typically react with electrophiles such as acid chlorides, activated carboxylic acids, aryl halides, carboxylic esters, imidate esters, etc. to give a mixture of products arising from substitution of one or both nitrogens. In many cases, it is possible to select reaction conditions in which monosubsituted products predominate, and in such cases the major product is usually that arising from substitution of the less-hindered nitrogen atom. For example, treatment of piperazine itself with a butyllithium followed by benzoyl chloride provides primarily the monobenzoyl derivative 13 (Wang, T. et al., J. Organic Chemistry 1999, vol 64, 7661), and treatment of 2-methylpiperazine with methyl benzoate and diethylaluminum chloride gives predominantly the product of monoacylation at the less hindered nitrogen, 14 (Wang, T. et al., J. Organic Chem. 2000, vol 65, 4740).

Certain monoprotected piperazines that are useful for the synthesis of other piperazines with a single unsubstituted nitrogen atom are commercially available. Others can be prepared by selective functionalization of the less hindered piperazine nitrogen with electrophiles such as di-tert-butyl dicarbonate. Commercially available monoprotected piperazine derivatives include the mono-Boc piperazines 15-17.

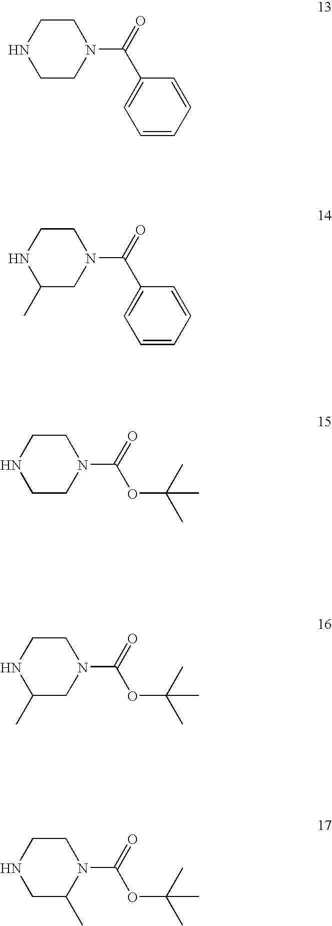

Scheme 3 shows a representative synthesis of a monoacylated piperazine using 15 as starting material. In Scheme 3, the monoprotected piperazine 15 is treated with an acid chloride in the presence of triethylamine as an acid acceptor, and the Boc group is removed from the acylation product by the action of trifluoroacetic acid.

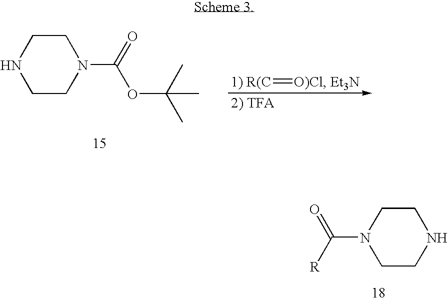

Monoprotected piperazines may be converted into mono amidino derivatives such as 22 in an analogous fashion (Scheme 4). Typically, compounds of formulas 19 are treated with 1 equivalent of an appropriate carboximidoyl chloride in a solvent such as haloalkane (e. g. dichloromethane, “DCM”), or an ether (e. g. tetrahydrofuran) and are treated with excess amount of an acid acceptor such as triethylamine or N,N-diisopropyl-N-ethylamine or N-methyl morpholine at room temperature for approximately 1 to 2 hours. If a carboximidic acid ester or carboximidothioic acid ester is used in place of the caboximidoyl chloride, the addition of a base may not always be necessary. In some cases methanol may be useful as the reaction solvent, depending on the exact nature of the reagent. Published summaries of standard methods of amidine formation include “Chemistry of the Amidines and Imidates (Chemistry of Functional Groups Series)” by S Patai, Saul Patai (Editor), John Wiley and Sons, N.Y., N.Y.; “Amidines and N-Substituted Amidines” Dunn, P. J. Comprehensive Organic Functional Group Transformations II (2005), 5 655-699, Elsevier Ltd., Oxford, UK.

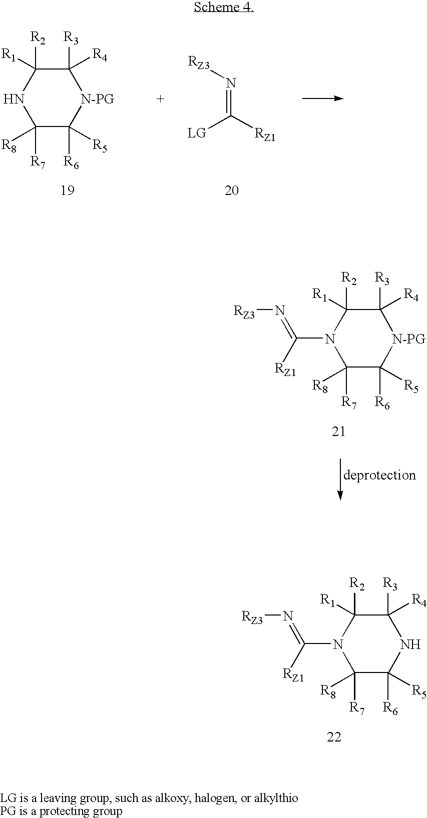

Many N-Aryl and N-Heteroaryl piperazines may be prepared according is to Scheme 5. Electron-deficient heteroaryl halides such as haloquinolines will react with substituted piperazines when heated together in the presence of an acid acceptor such as diisopropylethylamine. In some cases, improved yields will be obtained by the use of a solvent such as dimethylpropylene urea. In some cases, improved yields will be obtained by the use of a catalyst, such as copper powder or a copper salt. A variety of other methods are available for the N-substitution of piperazines with heteroaryl groups, including less electron-deficient aryls and heteroaryls (Antilla, J. C. et al, Organic Letters 2001, vol 3, 2077; Chan, D. M. T. et al., Tetrahedron Lett. 1998, 2933; Kunz, K. et al., Synlett. 2003, 2428; Kwong, F. Y. et al., Organic Letters 2002, vol 4, 581). The method in shown in Scheme 6 is representative.

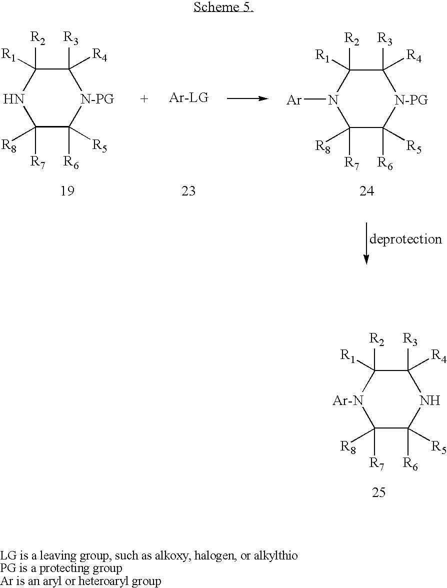

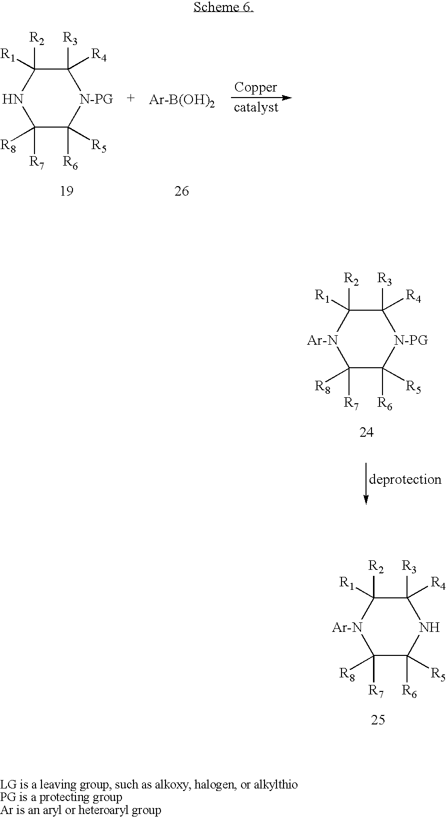

It is apparent to one skilled in the art that in some cases it may be more efficient to change the order of the steps in which the two piperazine nitrogens are functionalized. This approach is outlined in Schemes 7 and 8. In Scheme 7, a monoprotected piperazine is treated with sulfonyl chloride A in the presence of an acid acceptor such as diisopropylethylamine to give sulfonamide 26. Removal of the protecting group with a suitable deprotecting agent (TFA if PG=BOC) gives the intermediate 27. Functionalization of the free NH may be performed using an acid chloride to give a compound of general structure 28. Alternatively, the free NH of the piperazine may be functionalized with an aryl or heteroaryl halide in the presence of an acid acceptor such as diisopropylethylamine. Treating 27 with an appropriate amidation reagent gives an amidine such as 30. It is apparent to one skilled in the art that in certain cases the selective acylation, amidination, and arylation of piperazine derivatives can alternatively be performed without the use of protecting groups.

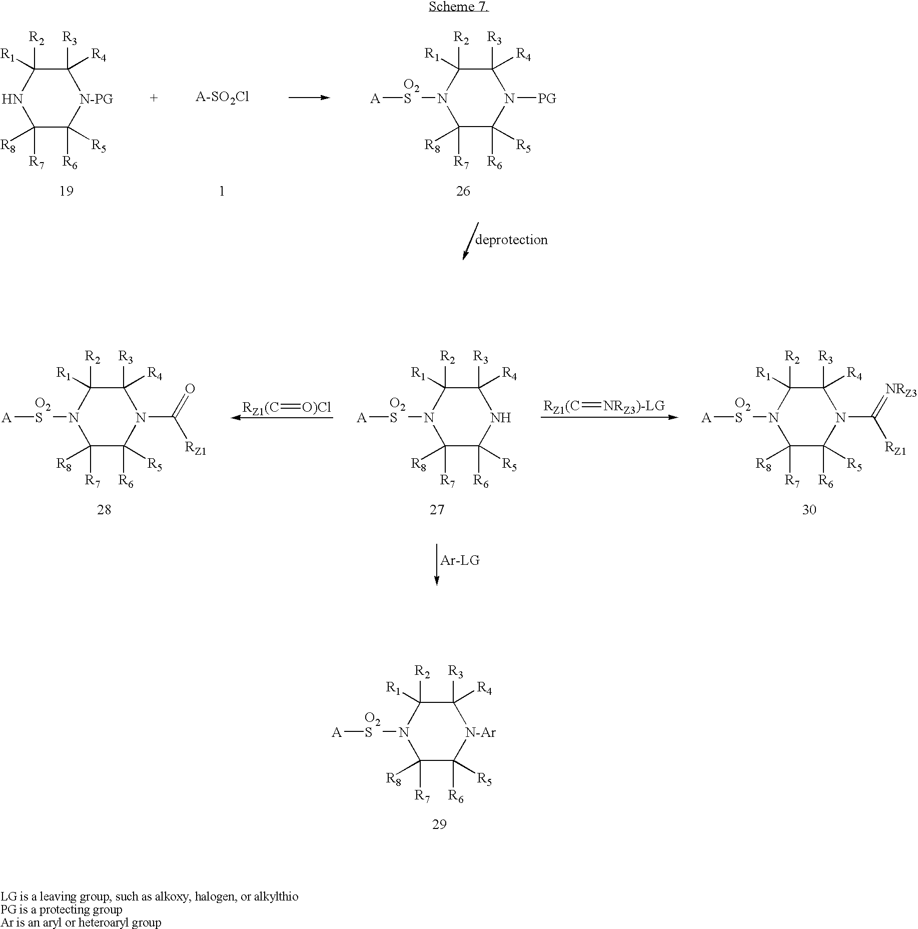

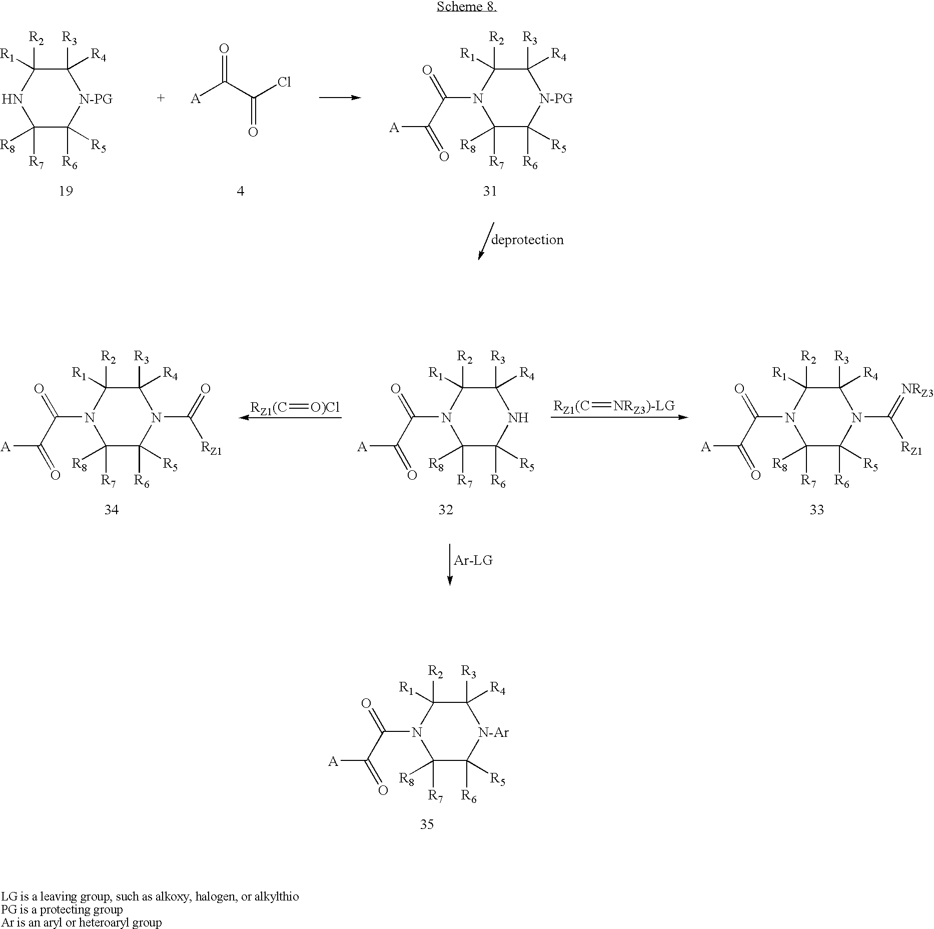

An alternate route for preparing intermediates 31 is described in Scheme 9. In this approach, a cyanomethylpiperazine derivative is prepared by treating a functionalized piperazine with chloroacetonitrile. The resulting aminonitrile is treated with a strong base and an ester to give an adduct that is oxidized with sodium hypochlorite or MCPBA to give a ketoamide (Yang, Z. et al., Organic Letters 2002, vol 4, 1103).

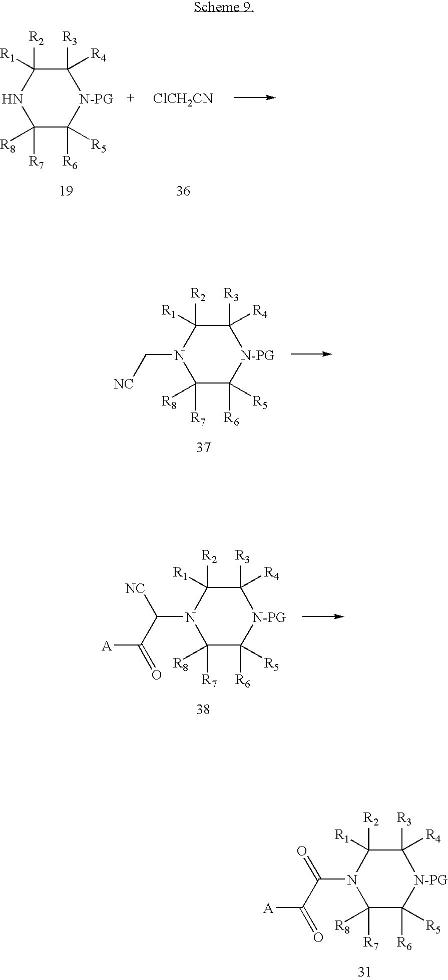

Scheme 10 shows a method for the synthesis of alkylidene piperidines of general structure 42. In this general scheme, an N-protected piperidinone is treated with an active methylene compound in the presence of a suitable base to provide protected alkylidene derivatives of general structure 41. Deprotection of 41 gives the free NH derivative 42, which is an example of generic structure 2.

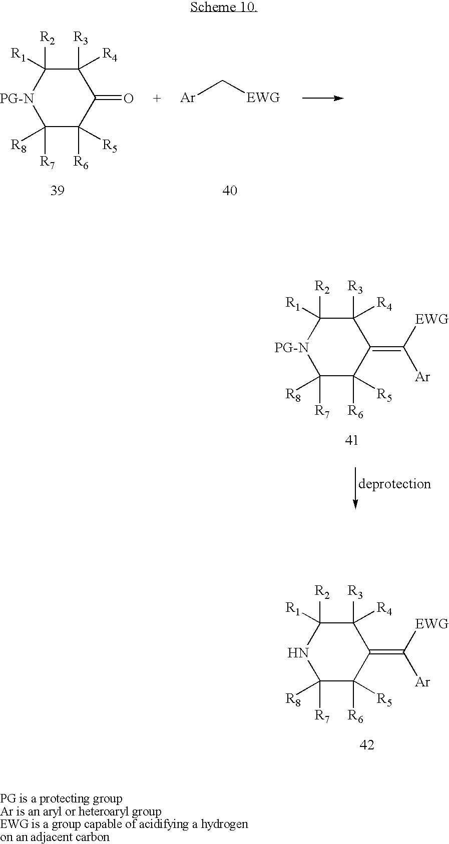

Other alkylidene piperidines may be prepared according to Scheme 11. In this scheme an arylmethylphosphonium salt is treated with strong base and then added to a protected piperidinone according to well-known literature methods. Treating the intermediate 44 with bromine and potassium carbonate in chloroform followed by treatment with sodium hydroxide in aqueous methanol gives the bromide 50. Using methods well-known in the art (Miyura, N et al., Chemical Reviews 1995, vol 95, 2457. Mitchell, T. N., Synthesis 1992, 803. Stille, J. K., Angewandte Chemie Int. Ed. English 1986, 508), the bromide 50 can be coupled with aryltin or arylboron compounds in the presence of a palladium catalyst to give the protected alkylidene derivatives 51. Deprotection gives the free NH intermediate 52. Alternatively, the bromide can be subjected to metal halogen exchange with butyllithium, typically in a solvent such as tetrahydrofuran at −78° C., and treated with carbon dioxide to give the carboxylic acid 46. Carboxylic acids are well-known precursors for a variety of heterocyles, and Scheme 10 illustrates the conversion of a carboxylic acid group into oxadiazole substituents by coupling with a carboxylic acid hydrazide followed by dehydration. Deprotection gives the free NH compound 49.

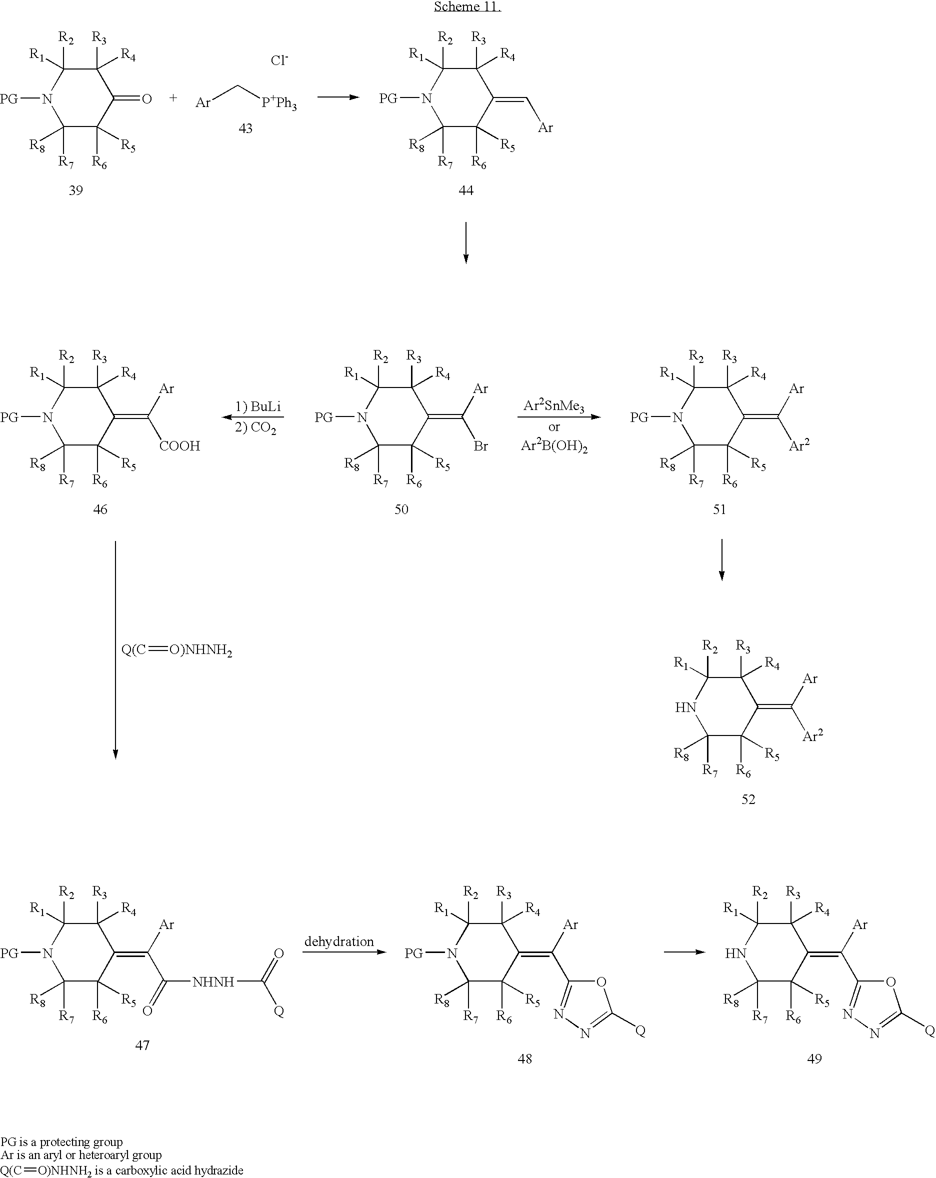

A variety of literature methods are available for the synthesis of sulfonyl is chlorides of general structure 53. These are outlined in Scheme 12. Treating an aromatic compound with chlorosulfonic acid provides sulfonyl chlorides 53. The temperature of the reaction may range between −40° C. to 120° C., and a solvent such as dichloromethane may optionally be used. Best results will usually be obtained when at least a five-fold excess of chlorosulfonic acid is used. Treating an aryl thiol, aryl thiocyanate, or certain aryl thioalkyl derivatives with chlorine gas in acetic acid provides sulfonyl chlorides 53. The reaction is normally-performed at temperatures of 5° C. to 15° C. Treating an aryllithium or aryl Grignard reagent with sulfur dioxide gives an aryl sulfinate salt, which upon treatment with N-chlorosuccinimide provides sulfonyl chlorides of general structure 53. Compounds 53 may be examples of generic structure 1, or the aryl group may be modified by further synthetic operations at a later point in the synthesis to give the compounds of the invention as shown in Scheme 13. 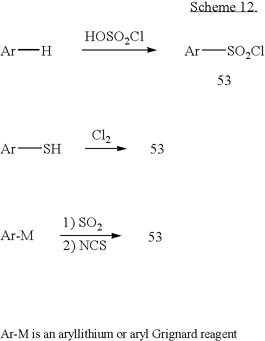 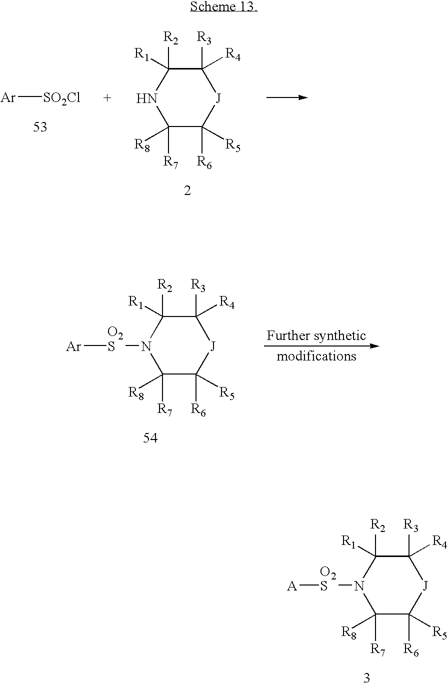
A variety of methods well known in the art are available for the synthesis of aryl ketoacids of generic structure 57. Treating an aryl bromide 55 with magnesium gives an arylmagnesium bromide, which is treated directly with by methyl chlorooxalate and a copper catalyst to give an aryl ketoester 56 (Babudri, F. et al., Tetrahedron 1996, vol 52, 13513). Hydrolysis of the ketoester with sodium hydroxide in a mixture of methanol and water gives a ketoacid of general structure 57. Alternatively, a methyl-substituted arene of general structure 58 is treated with NBS in the presence of light or a free radical initiator to give a bromide of general structure 59. Displacement of the bromine with cyanide gives a nitrile of general structure 60. This reaction may be performed in a variety of solvents, most commonly a polar solvent such as DMSO or DMF. Partial hydrolysis of the nitrile to an ester 61 may be conducted by treating the nitrile with hydrochloric acid in methanol. Oxidation of the resulting ester to a ketoester 56 may be performed in a variety of literature methods, commonly by the use of selenium dioxide. Alternatively, treating an aryl aldehyde of general structure 62 with sodium cyanide in the presence of a buffer acid such as acetic acid gives a cyanohydrin 63 which can be partially hydrolysed to a hydroxyester 64 using hydrochloric acid in methanol. Hydroxyesters 64 can be oxidized to ketoesters 56 using a variety of methods known in the art. Generic structure 57 may be an example of generic structure 6, or it may be a synthetic intermediate that is converted to 6 by further transformations.

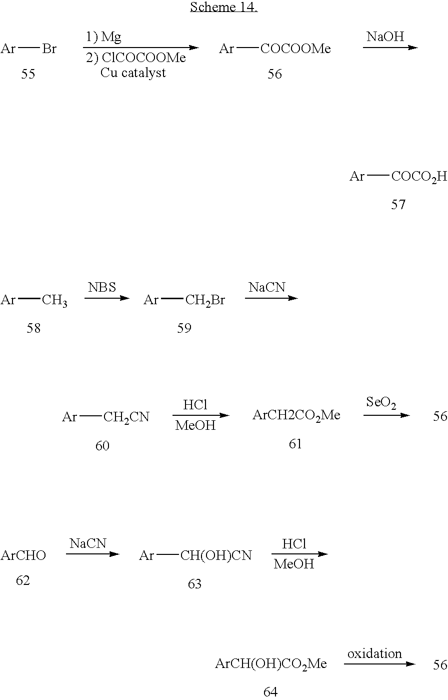

A variety of methods known in the art are available for the synthesis of compounds having an aryl-aryl bond. Most commonly, such compounds are prepared by one of two methods. In the first, a substituted aryl compound is subjected to synthetic transformations in which the substituent is converted into an aryl ring. This approach is most commonly used when the aryl ring being formed is a heteroaryl ring (See, Heterocyclic Chemistry, Gilchrist, T. L., Prentice Hall; 3rd edition (1997), Comprehensive Heterocyclic Chemistry on CD ROM, Katritzky, A. R.; Rees, C. W. (Ed.), Elsevier Science (1997)). The examples shown below should be considered as representative, but not limiting. For the synthesis of a thiazole of type 67, a methyl ketone is brominated with bromine, commonly in acetic acid as solvent at room temperature to reflux. Treating this bromoketone with a thioamide in a polar solvent such as DMF at temperatures of 25° C. to reflux provides 67. Treating an aldehyde with hydroxylamine generated in situ from hydroxylamine hydrochloride and base gives an oxime, which can be chlorinated with NCS in warm DMF to give 69. Adding this chloride slowly to a solution of an acetylene and base at room temperature gives an isoxazole of structure 70. Alternatively, adding the chloride 69 slowly to excess methanolic ammonia gives an amideoxime 71, which is cyclized to 72 by adding an acid chloride, optionally in the presence of a tertiary amine base, and heating to temperatures of 70° C. to 120° C. Alternatively, treating 68 with toluenesulfonyl isocyanate (TOSMIC) gives an oxazole 73. Further examples of this approach are well known in the art of heterocyclic chemistry. Treating a nitrile of structure 74 with hydrogen sulfide in a mixture of pyridine and triethyl amine at temperatures of 25° C. to 50° C. gives the thioamide 75, which can be cyclized to a thiazole 76 upon treatment with a bromoketone in DMF at temperatures of 25° C. to 100° C. Alternatively, treating the nitrile 74 with methanol and hydrogen chloride in ether at temperatures of −10° C. to 10° C. gives an imidate hydrochloride of general structure 77. This reaction typically gives best results when only 1.0 to 1.2 equivalents of methanol are used. In some cases, it may be desirable to use diethyl ether as a dilutant for the reaction. Treating the imidate hydrochloride with a carboxylic acid hydrazide in a solvent such as methanol gives an adduct which can be cyclized by heating in toluene or another high boiling solvent at temperatures of 100° C. to 180° C.; thus, giving the triazole 78. Treating aryl bromide 79 with trimethylsilylacetylene and a palladium catalyst in an amine solvent followed by treatment with methanolic base gives the aryl acetylene 80. The acetylene 80 upon treatment with an alkyl azide, optionally in the presence of a copper catalyst, gives the triazole 81. Details for the performance of these transformations are well known in the art.

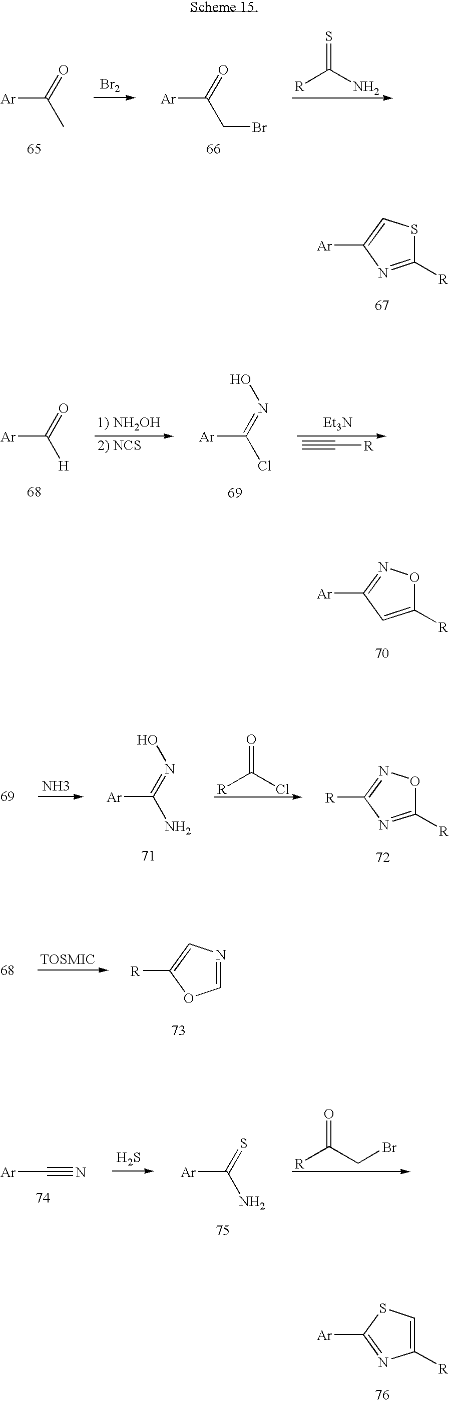

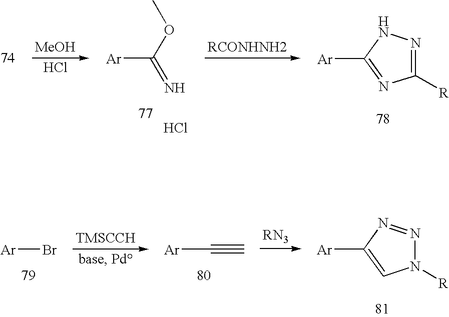

A second commonly used approach to the synthesis of compounds 83 containing an aryl-aryl bond is to use a palladium catalyst to couple an aryl halide with an arylzinc, arylboronate or aryltin compound (See, Miyura, N. et al., Chemical Reviews 1995, vol 95, 2457; Mitchell, T. N. Synthesis 1992, 803; Stille, J. K. Angewandte Chemie Int. Ed. English 1986, 508; Negishi, E-I. et al., J. Organic Chemistry 1977, 1821; Erdik, E., Tetrahedron 1992, 9577), This approach is exemplified in Scheme 16. The coupling reaction between an aryl halide 82 and an aryltin compound is commonly performed using PhCH₂PdCl(Ph₃P)₂ as catalyst in refluxing chloroform. Other solvents and catalysts are occasionally useful for this transformation, and in some cases additives such as lithium chloride or copper salts facilitate the reaction. The coupling between an aryl halide and an aryl boron compound is commonly performed using a two phase mixture of benzene, aqueous sodium carbonate, and ethanol as solvent, and tetrakis(triphenylphosphine)palladium(0) as catalyst. In certain cases, the use of other solvents, catalysts, and bases gives superior results as well known in the art. Coupling between an aryl halide and an arylzinc reagent is usually performed in tetrahydrofuran, dimethylformamide, or a mixture of these two solvents using tetrakis(triphenylphosphine)palladium(0) as catalyst. In certain cases it may be preferable to use the chloride, bromide or iodide as the aryl halide coupling partner, depending on the nature of the two aryl groups being coupled and whether the coupling partner is a boron reagent, tin reagent, or zinc reagent.

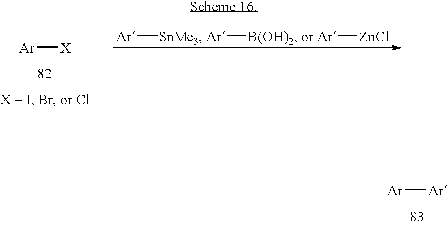

Many aryltin, arylzinc, and arylboronate compounds are commercially available. Others can be prepared by the routes shown in the scheme below. Treating an aryl bromide with butyllithium in tetrahydrofuran at −78° C. gives an aryllithium species that is treated in situ with trimethylborate. Hydrolysis of the resulting borate salt with hydrochloric acid gives the boronic acid 85. Alternatively, treating the aryllithium species with trimethylstannyl chloride gives an isolate aryltin compound 86. Treating an aryllithium with zinc chloride gives the arylzinc species 87 which is usually used directly in a palladium catalyzed coupling step without isolation. Aryltin compounds may also be formed by treating an aryl bromide or iodide with hexamethylditin and a catalytic PhCH₂PdCl(Ph₃P)₂ in dioxane at temperatures of 50° C. to 120° C. (See, Stille, J. K. Angewandte Chemie Int. Ed. English 1986, 508). Arylboronic esters can be formed by treating an aryl bromide or iodide with a palladium catalyst and bis(pinacolborane) in the presence of sodium acetate (See, Baudoin, O. et al., J. Organic Chem. 2000, vol 65, 9268). In palladium-catalyzed coupling reactions, the boronate esters 89 often give results that are equivalent to those obtained with the boronic acids 85.

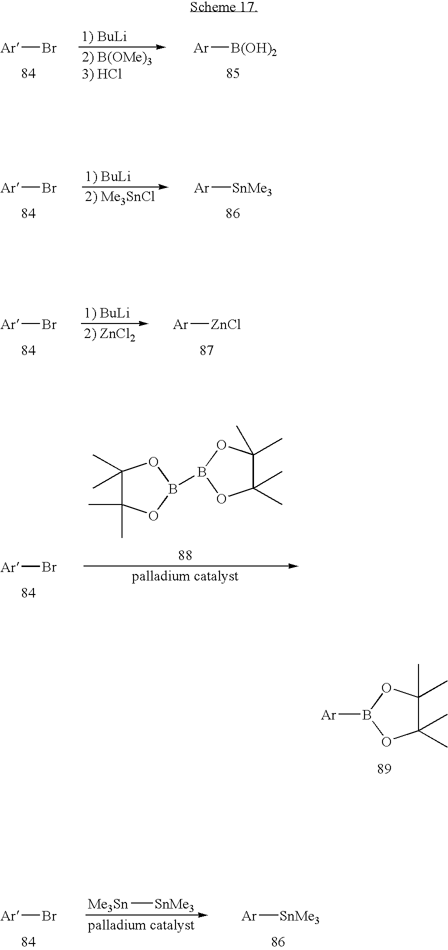

Diaryl ketones may be prepared by treating an aryl aldehyde with a Gringard or organolithium reagent to give a carbinol 88. Oxidation of the carbinol with a suitable oxidizing agent such as manganese dioxide or pyridinium dichromate gives the ketone 89.

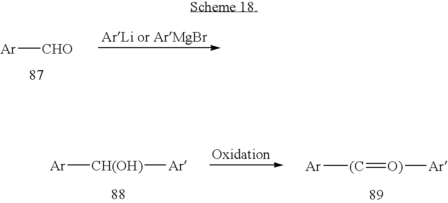

Alternatively, diaryl ketones 91 may be prepared from acid chlorides (Dieter, K. R. Tetrahedron 1999, vol 55, 4177). One method involves treating an acid chloride 90 with an arylzinc or aryltin compound in the presence of a palladium catalyst. In the case of the arylzinc reagents, tetrakis(triphenylphosphine)palladium(o) is commonly a useful catalyst and the reaction is performed at 25° C. to 65° C. in THF. When an aryltin compound is used, the catalyst is commonly PhCH₂PdCl(Ph₃P)₂ and the reaction is performed in refluxing chloroform. Alternatively, the acid chloride can be treated with an aryl Gringard reagent and a copper salt as catalyst.

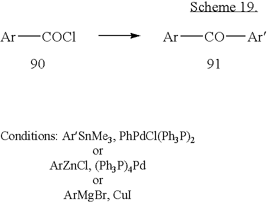

Certain compounds of this invention are prepared by nucleophilic aromatic substitution reactions. Compounds 94 containing a direct aryl-aryl bond, in which one of the aryl rings is bonded through a ring nitrogen, may be formed by treating an aryl halide 92 with a heterocylic amine in the presence of a base such as potassium hydroxide and a copper catalyst such as copper iodide or copper powder. Temperatures for this reaction may vary between 80° C. to 180° C., and in some cases the use of a solvent such as DMPU may facilitate the reaction. Likewise, compounds 97 containing two aryl rings linked through a sulfur atom may be prepared by treating a aryl halide 95 with an aromatic thiol 96 in the presence of a base such as potassium carbonate and a copper catalyst such as copper oxide.

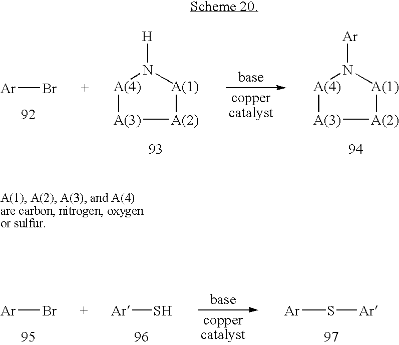

Certain intermediates described above are novel compounds, and it is to be understood that all novel intermediates herein are further aspects of the present invention. Examples of novel intermediates are the following:
((R)-4-{Methoxyimino]-phenyl-methyl}-2-methyl-piperazin-1-yl)-acetonitrile;
((R)-3-Methyl-piperazin-1-yl)-phenyl-methanone O-methyl-oxime;
(3R)-3-methyl-1-(phenylcarbonothioyl)piperazine;
5-(4-Benzoyl-2-methyl-piperazine-1-sulfonyl)-thiophene-2-carboxylic acid ethyl ester;
[4-(5-Bromo-thiophene-2-sulfonyl)-3-methyl-piperazin-1-yl]-phenyl-methanone;
[4-(5-Bromo-thiophene-2-sulfonyl)-3-methyl-piperazin-1-yl]-phenyl-methanone;
tert-butyl 3-methyl-4-(thiophen-2-ylsulfonyl)piperazine-1-carboxylate;
[4-(4-Bromo-benzenesulfonyl)-3-methyl-piperazin-1-yl]-phenyl-methanone;
(R)-(4-(4-ethynylphenylsulfonyl)-3-methylpiperazin-1-yl)(phenyl)methanone;
4-(1-Methyl-1H-pyrazol-3-yl)-benzoic acid methyl ester;
1-(4-Benzoyl-2-methyl-piperazin-1-yl)-2-(4-iodo-phenyl)-ethane-1,2-dione;
1-((R)-4-Benzoyl-2-methyl-piperazin-1-yl)-2-(4-bromo-phenyl)-ethane-1,2-dione;
1-((R)-4-Benzoyl-2-methyl-piperazin-1-yl)-2-(4-bromo-2-fluoro-phenyl)-ethane-1,2-dione;
1-((R)-4-Benzoyl-2-methyl-piperazin-1-yl)-2-(4-bromo-3-methyl-phenyl)-ethane-1,2-dione;
1-((R)-4-Benzoyl-2-methyl-piperazin-1-yl)-2-(4-bromo-2-methyl-phenyl)-ethane-1,2-dione;
1-((R)-4-Benzoyl-2-methyl-piperazin-1-yl)-2-(6-chloro-pyridin-3-yl)-ethane-1,2-dione;
4-[2-((R)-4-Benzoyl-2-methyl-piperazin-1-yl)-2-oxo-acetyl]-boronic acid;
1-((R)-4-Benzoyl-2-methyl-piperazin-1-yl)-2-(4-bromo-2-dimethylamino-phenyl)-ethane-1,2-dione; and
1-(2-Amino-4-bromo-phenyl)-2-((R)-4-benzoyl-2-methyl-piperazin-1-yl)-ethane-1,2-dione

### Example 3

This example, along with the Schemes herein, illustrate typical procedures and characterization of selected examples with respect to representative compounds according to embodiments of the invention, and their synthesis.

### Purification and Analytical Procedures

Purification:

Compounds requiring HPLC purification were purified on a system using a Sedex 75 ELSD as the fraction-determining detector, the Gilson 215 as autosampler and fraction collector, and Gilson 321 pumps. Mobile phases used were one of the following:
1. Water/Acetonitrile/0.05% Trifluoroacetic Acid
2. 20 mM ammonium formate at pH 4-6 and Acetonitrile
3. 0.1% ammonium hydroxide and acetonitrile at pH 9.0.

Columns used were either Phenomenex Gemini, 5 μm, 21.2×50 mm or Peeke Scientific Ultro 60 C18 5 μm, 20 mm×50 mm.

Analytical:

Compounds were analyzed on an Applied Biosystems/Sciex 150EX single quad mass spectrometer in positive ion mode using either an ESI (Electrospray Ionization) source or an APCI (Atmospheric Pressure Chemical Ionization) source. Scan range is 100-1000 amu. Mobile phase used was one of the above as described in the purification section. Sedex 75 ELSD and Agilent PDA (photodiode array) UV detection was used. The column most commonly used was the Phenomenex Gemini, 5 μm, 4.6×50 mm. When necessary to achieve greater separation of close-eluting impurities, other columns were used including the Phenomenex Gemini, 5 μm, 4.6×100 or 250 mm, the Kromasil 100, C18, 5 μm, 4.6×100 or 250, and the DuraGel HS, 5 μm, phenyl 4.6×250 mm from Peeke Scientific. Mass (“MS”) calculations were made using the monoisotopic mass for the compound.

### Example 101

### 1-((R)-4-Benzoyl-2-methyl-piperazin-1-yl)-2-(4-oxazol-5-yl-phenyl)-ethane-1,2-dione

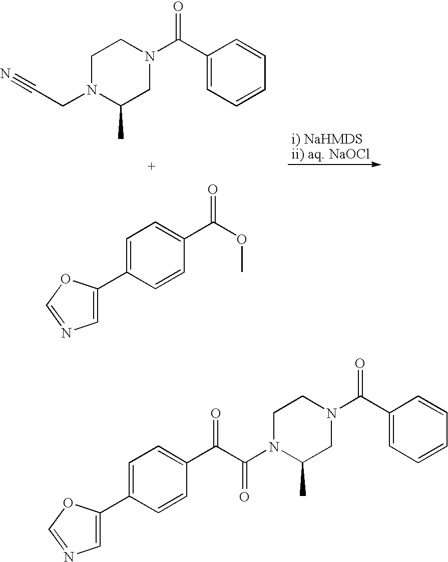

((R)-4-Benzoyl-2-methyl-piperazin-1-yl)-acetonitrile (182 mg, 0.896 mmol) and 4-oxazol-5-yl-benzoic acid methyl ester (245 mg, 1 mmol) were dissolved in 10 mL of anhydrous THF. N-Sodium hexamethyldisilazane (NaHMDS, 500 mg, 2.7 mmol) was then added directly to the solution in portions while stirring at ambient temperature. The resulting mixture was allowed to stir for 2 to 5 hours. Bleach (10 mL) was then added in one portion and the resulting biphasic emhulsion stirred or shaken rapidly for 5 to 10 minutes. 30 mL of EtOAc was added and the resulting organic layer washed once with an equal volume of saturated aqueous sodium sulfite. The organic fraction was then washed with two portions of saturated ammonium chloride and then brine. After drying over MgSO₄, the organic fraction was concentrated in-vacuo to give 265 mg of a brown residue that was subjected to purification by reverse-phase HPLC ultimately affording 49 mg of pure 1-((R)-4-benzoyl-2-methyl-piperazin-1-yl)-2-(4-oxazol-5-yl-phenyl)-ethane-1,2-dione as light yellow syrup (13%, isolated yield). MS: Calculated for C₂₃H₂₁N₃O₄.H⁺: 403.15. Found: 404.5. ¹HNMR (CDCl₃): 8.07-8.00 (m, 3H), 7.82 (d, J=5.7 Hz, 2H), 7.57 (s, 1H), 7.5-7.35 (br. m, 5H), 5.2-2.9 (br. m, 7H), 1.3 (br. s, 3H).

### ((R)-4-Benzoyl-2-methyl-piperazin-1-yl)-acetonitrile

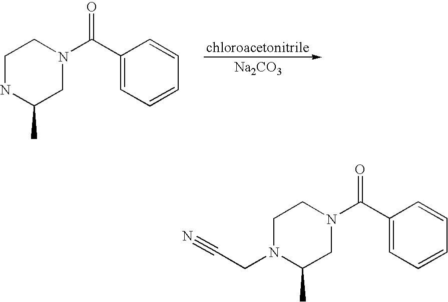

((R)-3-Methyl-piperazin-1-yl)-phenyl-methanone (2 g, 10 mmol) was dissolved in 30 mL of acetonitrile. Chloroacetonitrile (800 microliters, 12.6 mmol) was then added along with 2 g of anhydrous sodium carbonate. The mixture was refluxed for 3 hours and the insoluble salts removed via vacuum filtration. The solvent was removed under vacuum and the resulting residue partitioned between EtOAc and water. The organic fraction was washed one time with brine, dried over MgSO₄, concentrated, and then dried under high vacuum affording 1.25 g of ((R)-4-benzoyl-2-methyl-piperazin-1-yl)-acetonitrile as a colorless syrup (51%). MS: Calculated for C₁₄H₁₇N₃O.H⁺: 243.31. Found: 244.3. This material was used without further purification for subsequent reactions.

### 4-Oxazol-5-yl-benzoic acid methyl ester

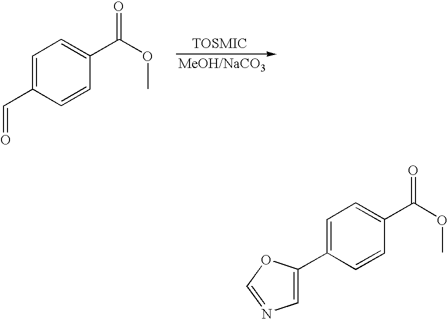

4-Formyl-benzoic acid methyl ester (2 g, 11.2 mmol), toluenesulphonylmethyl isocyanide (TOSMIC, 2 g, 10.2 mmol) and anhydrous sodium carbonate (2 g, 18.8 mmol) were brought up in 50 mL of anhydrous methanol. The mixture was refluxed for 2 hours, allowed to cool to room temperature, then concentrated in-vacuo. The resulting residue was partitioned between EtOAc and saturated aqueous ammonium chloride. The organic fraction was washed one additional time with ammonium chloride and then once with brine. After drying over MgSO₄, the organic fraction was concentrated under vacuum to dryness affording 1.48 g of 4-oxazol-5-yl-benzoic acid methyl ester as a yellow solid.

¹HNMR (CDCl₃): 8.22 (d, J=8.1 Hz, 2H), 7.97 (s, 1H), 7.74 (d, J=6 Hz, 2H), 7.48 (s, 1H), 3.94 (s, 3H).

### Example 102

### 1-((R)-4-Benzoyl-2-methyl-piperazin-1-yl)-2-[4-(3-methyl-isoxazol-5-yl)-phenyl]-ethane-1,2-dione

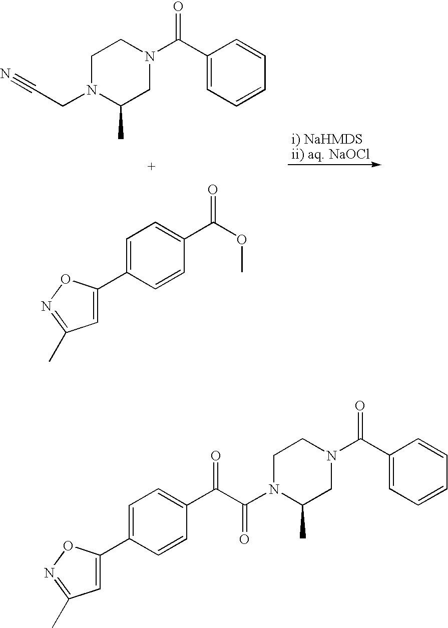

((R)-4-Benzoyl-2-methyl-piperazin-1-yl)-acetonitrile (315 mg, 1.3 mmol) and 4-(3-Methyl-isoxazol-5-yl)-benzoic acid methyl ester (280 mg, 1.3 mmol) were dissolved in 10 mL of anhydrous THF. N-Sodium hexamethyidisilazane (NaHMDS, 600 mg, 3 mmol) was then added directly to the solution in portions while stirring at ambient temperature. The resulting mixture was allowed to stir for 2 to 5 hours. Bleach (10 mL) was then added in one portion and the resulting biphasic emulsion stirred or shaken rapidly for 5 to 10 minutes. 30 mL of EtOAc was added and the resulting organic layer washed once with an equal volume of saturated aqueous sodium sulfite. The organic fraction was then washed with two portions of saturated ammonium chloride and then brine. After drying over MgSO₄, the organic fraction was concentrated in-vacuo to give 200 mg of a brown residue that was subjected to purification by reverse-phase HPLC ultimately affording 407 mg of 1-((R)-4-benzoyl-2-methyl-piperazin-1-yl)-2-[4-(3-methyl-isoxazol-5-yl)-phenyl]-ethane-1,2-dione as light yellow foam (75%). MS: Calculated for C₂₄H₂₃N₃O₄.H⁺: 417.5. Found: 418.4. ¹HNMR (CDCl₃): 8.03 (m, 2H) 7.91 (d, J=8.1 Hz, 2H), 7.53-7.32 (m, 5H), 6.54 (s, 1H), 5.24-2.92 (m, 7H), 2.69 (s, 3H), 1.33 (br. s, 3H).

### 4-(3-Methyl-isoxazol-5-yl)-benzoic acid methyl ester

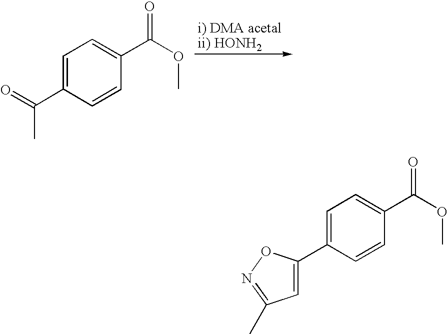

4-Acetyl-benzoic acid methyl ester (0.5 g, 2.8 mmol) was dissolved in 4 mL of DMA dimethyl acetal and heated to 100° C. for 2 hours. Upon cooling to room temperature, 30 mL of EtOAc was added. The sample was concentrated until the crude intermediate eneamine precipitated. The precipitate was isolated by vacuum filtration yielding 412 mg of a brown solid that was immediately dissolved in 10 mL of anhydrous EtOH and combined with 150 mg (2.1 mmol) of hydroxylamine hydrochloride. The resulting mixture was refluxed for three hours and then slowly cooled to 0° C. The sample was then thawed and the insoluble product collected while the mother liquor was still cool affording 280 mg of tan crystals (78%). ¹HNMR (CDCl₃): 8.105 (d, J=8.4 Hz, 2H), 8.00 (d, J=8.1 Hz, 2H), 7.07 (s, 1H), 3.89 (s, 3H), 2.31 (s, 3H).

### Example 103

### 1-((R)-4-Benzoyl-2-methyl-piperazin-1-yl)-2-(4-thiazol-2-yl-phenyl)-ethane-1,2-dione

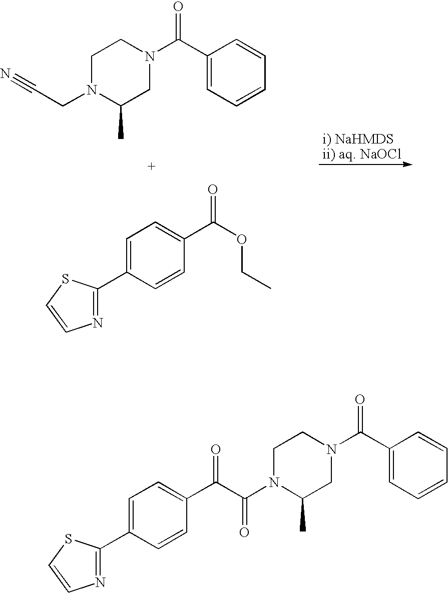

((R)-4-Benzoyl-2-methyl-piperazin-1-yl)-acetonitrile (300 mg, 1.2 mmol) and 4-thiazol-2-yl-benzoic acid ethyl ester (287 mg, 1.2 mmol) were dissolved in 10 mL of anhydrous THF. N-Sodium hexamethyldisilazane (NaHMDS, 600 mg, 3 mmol) was then added directly to the solution in portions while stirring at ambient temperature. The resulting mixture was allowed to stir for 2 to 5 hours. Bleach (10 mL) was then added in one portion and the resulting biphasic emulsion stirred or shaken rapidly for 5 to 10 minutes. 30 mL of EtOAc was added and the resulting organic layer washed once with an equal volume of saturated aqueous sodium sulfite. The organic fraction was then washed with two portions of saturated ammonium chloride and then brine. After drying over MgSO₄, the organic fraction was concentrated in-vacuo to give several hundred milligrams of a yellow residue that was subjected to initial purification by preparative TLC (eluting solvent: 95% DCM, 5% MeOH, 1% TEA) giving 100 mg of off-white semisolid consisting of 60% 1-((R)-4-benzoyl-2-methyl-piperazin-1-yl)-2-(4-thiazol-2-yl-phenyl)-ethane-1,2-dione and 40% ((R)-4-benzoyl-2-methyl-piperazin-1-yl)-acetonitrile. This material was further purified by reverse-phase HPLC ultimately giving the product as 11 mg of colorless syrup 2.2%). MS: Calculated for C₂₃H₂₁N₃O₃S.H⁺: 419.13. Found: 420.4. ¹HNMR (CDCl₃): 8.14 (d, J=8.1 Hz, 2H), 8.05-8.00 (m, 3H), 7.48 (d, J=3.3 Hz, 1H), 7.5-7.3 (m, 5H), 5.24-2.84 (br. m, 7H), 1.3 (br. s, 3H).

### 4-Thiazol-2-yl-benzoic acid ethyl ester

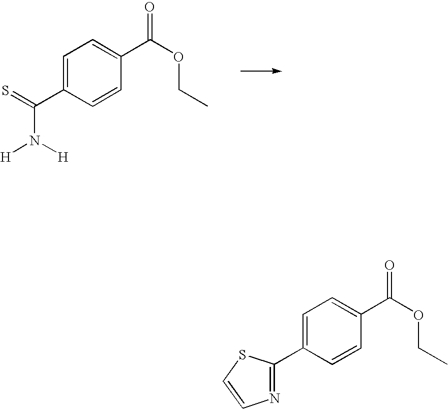

A mixture of 1.0 grams of ethyl 4-carbamothioylbenzoate, 0.76 mL of bromoacetaldehyde diethyl acetal, and 10 mL of DMF was heated to 95° C. over 3 hours and then maintained at this temperature an additional 2 hours. The mixture was cooled to 25° C. and partitioned between ethyl acetate and water. The organic phase was washed with brine, dried over magnesium sulfate, and then the solvent was evaporated at reduced pressure. The residue was chromatographed on silica gel eluting with dichloromethane. The partially purified product thus obtained was crystallized from a mixture of ether and hexanes. The title compound was obtained as 0.48 grams of a tan solid. ¹HNMR (CDCl₃): 8.15 (d, J=6 Hz, 2H), 8.04 (d, J=6 Hz, 2H), 7.93 (d, J=3 Hz, 1H), 7.41 (d, J=3 Hz, 1H), 4.41 (q, J=7 Hz, 2H), 1.42 (q, J=7 Hz, 3H).

### Example 104

### 1-((R)-4-Benzoyl-2-methyl-piperazin-1-yl)-2-[4-(2-methyl-thiazol-4-yl)-phenyl]-ethane-1,2-dione

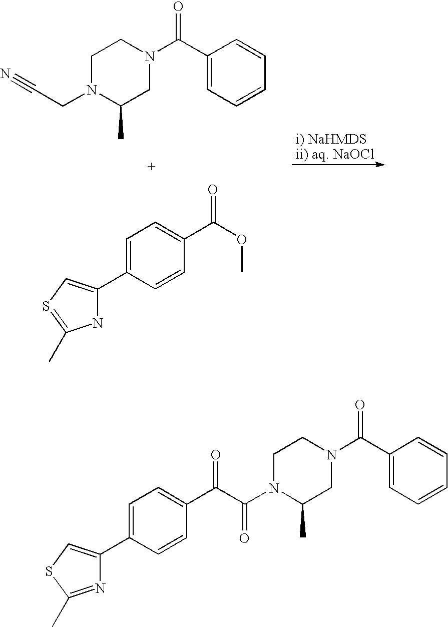

((R)-4-Benzoyl-2-methyl-piperazin-1-yl)-acetonitrile (146 mg, 0.6 mmol) and 4-(2-methyl-thiazol-4-yl)-benzoic acid methyl ester (140 mg, 0.6 mmol) were dissolved in 10 mL of anhydrous THF. N-Sodium hexamethyldisilazane (NaHMDS, 330 mg, 1.8 mmol) was then added directly to the solution in portions while stirring at ambient temperature. The resulting mixture was allowed to stir for 2 to 5 hours. Bleach (10 mL) was then added in one portion and the resulting biphasic emulsion stirred or shaken rapidly for 5 to 10 minutes. 15 mL of EtOAc was added and the resulting organic layer washed once with an equal volume of saturated aqueous sodium sulfite. The organic fraction was then washed with two portions of saturated ammonium chloride and then brine. After drying over MgSO₄, the organic fraction was concentrated in-vacuo to give ca. 150 mg of a yellow residue that was purified by preparative TLC (eluting solvent: 94% DCM, 5%, MeOH, 1% TEA) affording 95 mg of 90% pure 1-((R)-4-benzoyl-2methylpiperazin-1-yl)-2-[4-(2-methyl-thiazol-4-yl)-phenyl]-ethane-1,2-dione (36%) MS: Calculated for C₂₄H₂₃N₃O₃S.H⁺: 433.15. Found: 433.9. ¹HNMR (CDCl₃): 8.07-7.94 (m, 4H), 7.52 (s, 1H), 7.48-7.36 (m, 7.5-7.3, 5H), 5.24-2.84 (br. m, 10H), 1.3 (br. s, 3H).

### 4-(2-Methyl-thiazol-4-yl)-benzoic acid methyl ester

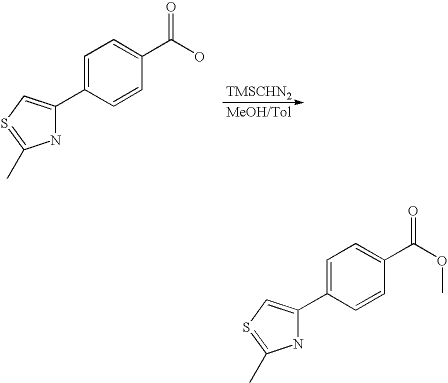

4-(2-Methyl-thiazol-4-yl)-benzoic acid (250 mg, 1.1 mmol) was suspended in 2:1 toluene:MeOH. TMS diazomethane (1.5 mL of 2M solution in diethyl ether) was added dropwise at room temperature. Reaction completion was confirmed by TLC (single spot, 1:1 hexanes:EtOAc). The solvent was removed and remaining yellow solid used without further characterization.

### Example 105

### 1-((R)-4-Benzoyl-2-methyl-piperazin-1-yl)-2-[4-(2-methyl-2H-tetrazol-5-yl)-phenyl]-ethane-1,2-dione

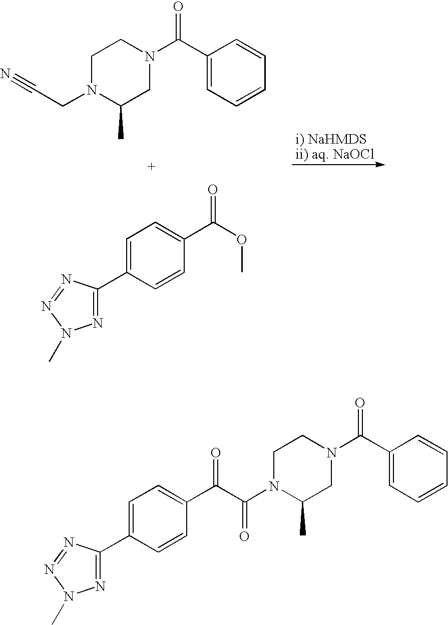

((R)-4-Benzoyl-2-methyl-piperazin-1-yl)-acetonitrile (178 mg, 0.73 mmol) and 4-(2-methyl-2H-tetrazol-5-yl)-benzoic acid methyl ester (150 mg, 0.73 mmol) were dissolved in 5 mL of anhydrous THF. N-Sodium hexamethyldisilazane (NaHMDS, 300 mg, 1.5 mmol) was then added directly to the solution in portions while stirring at ambient temperature. The resulting mixture was allowed to stir for 2 to 5 hours. Bleach (5 mL) was then added in one portion and the resulting biphasic emulsion stirred or shaken rapidly for 5 to 10 minutes. 10 mL of EtOAc was added and the resulting organic layer washed once with an equal volume of saturated aqueous sodium sulfite. The organic fraction was then washed with two portions of saturated ammonium chloride and then brine. After drying over MgSO₄, the organic fraction was concentrated in-vacuo to give 72 mg of a tan residue that was subjected to purification by reverse-phase HPLC ultimately affording 21 mg of 1-((R)-4-benzoyl-2-methyl-piperazin-1-yl)-2-[4-(2-methyl-2H-tetrazol-5-yl)-phenyl]-ethane-1,2-dione as colorless syrup (6.5%). ELSD purity>95%. MS: Calculated for C₂₂H₂₂N₆O₃.H⁺: 418.18. Found: 419.5.

### 4-(2-Methyl-2H-tetrazol-5-yl)-benzoic acid methyl ester

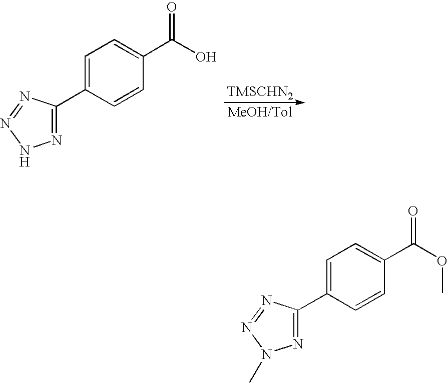

4-(2H-Tetrazol-5-yl)-benzoic acid (500 mg, 2.6 mmol) was suspended in 2:1 toluene:MeOH. TMS diazomethane (4 mL of 2M solution in diethyl ether) was added dropwise at room temperature. Reaction completion was confirmed by TLC (1:1 hexanes:EtOAc). The solvent was removed and remaining yellow solid used without further characterization assuming quantitative conversion.

### Example 106

### 1-((R)-4-{[Methoxyimino]-phenyl-methyl}-2-methyl-piperazin-1-yl)-2-(4-oxazol-5-yl-phenyl)-ethane-1,2-dione

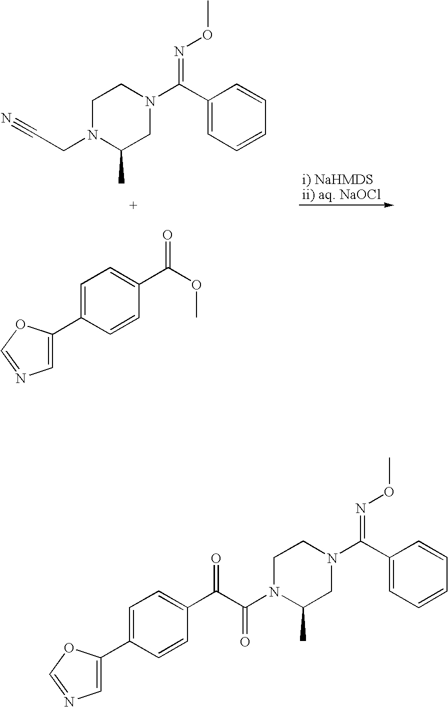

((R)-4-Benzoyl-2-methyl-piperazin-1-yl)-acetonitrile (245 mg, 0.9 mmol) and 4-oxazol-5-yl-benzoic acid methyl ester (182 mg, 0.9 mmol) were dissolved in 10 mL of anhydrous THF. N-Sodium hexamethyidisilazane (NaHMDS, 500 mg, 2.7 mmol) was then added directly to the solution in portions while stirring at ambient temperature. The resulting mixture was allowed to stir for 2 to 5 hours. Bleach (10 mL) was then added in one portion and the resulting biphasic emulsion stirred or shaken rapidly for 5 to 10 minutes. 30 mL of EtOAc was added and the resulting organic layer washed once with an equal volume of saturated aqueous sodium sulfite. The organic fraction was then washed with two portions of saturated ammonium chloride and then brine. After drying over MgSO₄, the organic fraction was concentrated in-vacuo to give 200 mg of a brown residue that was subjected to purification by reverse-phase HPLC ultimately affording 49 mg of 1-((R)-4-{[(E)-methoxyimino]-phenyl-methyl}-2-methyl-piperazin-1-yl)-2-(4-oxazol-5-yl-phenyl)-ethane-1,2-dione. MS: Calculated for C₂₄H₂₄N₄O₄.H⁺: 432.18. Found: 433.4. ¹HNMR (CDCl₃): 8.09-7.99 (m, 3H), 7.82-7.65 (m, 2H), 7.57 (s, 1H), 7.5-7.3 (m, 5H), 5.3-2.8 (m, 10H), 1.42-1.37 (m, 3H).

### ((R)-4-{Methoxyimino]-phenyl-methyl}-2-methyl-piperazin-1-yl)-acetonitrile

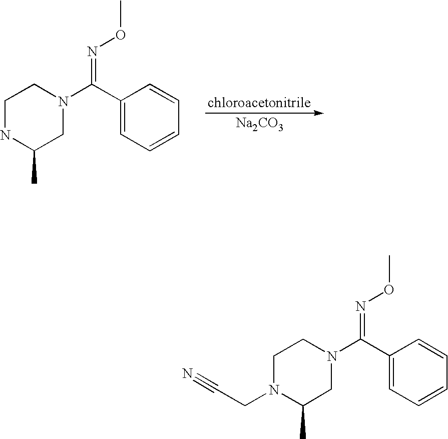

((R)-3-Methyl-piperazin-1-yl)-phenyl-methanone O-methyl-oxime (223 mg, 0.957 mmol), chloroacetonitrile (3 mL, solvent), and anhydrous sodium carbonate (500 mg) were combined in an 8 mL vial. The vial was sealed and the sample heated to 100° C. and held for 1 hour (TLC monitoring, solvent=94% DCM, 5% MeOH, 1% TEA). Upon satisfactory completion, the insoluble salts were removed by vacuum filtration and the filtrate concentrated and then dried under high vacuum. LC-MS analysis showed the material to be >99% pure by ELSD. MS: Calculated for C₁₅H₂₀N₄O.H⁺: 273.1. Found: 273.3. The material was used without further purificiation.

### ((R)-3-Methyl-piperazin-1-yl)-phenyl-methanone O-methyl-oxime

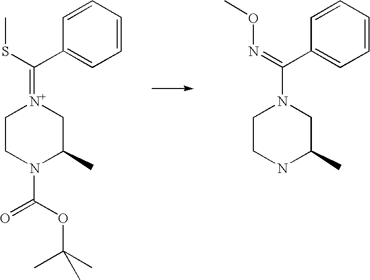

A solution of 0.7 grams (1.5 mmol) of tert-butyl (2R)-2-methyl-4-(phenylcarbonothioyl)-piperazine-1-carboxylate methiodide salt in 2 mL of methanol was treated with a solution of 0.13 grams (1.6 mmol) of methoxylamine hydrochloride and 0.36 mL (2 mmol) of Hunig's base in 2 mL of methanol. After 15 minutes, the mixture was partitioned between ethyl acetate and water. The organic phase was washed with brine, dried over MgSO₄ and stripped. The oily yellow residue was dissolved in 5 mL of TFA and stirred one hour. The solution was partitioned between ethyl acetate and excess aqueous potassium carbonate. The organic phase was washed with brine and dried over MgSO₄. The solvent was evaporated and the residue was crystallized from hexanes. This procedure yielded the product as 170 mg of white solid (49%). MS: calculated for C₁₃H₁₉N₃O H⁺: 234.1. Found: 234.1.

### tert-Butyl (2R)-2-methyl-4-(phenylcarbonothioyl)piperazine-1-carboxylate methiodide salt

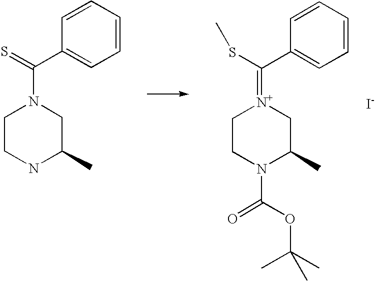

(3R)-3-Methyl-1-(phenylcarbonothioyl)piperazine (0.85 grams, 3.9 mmol) was dissolved in 10 mL of dichloromethane and treated with 0.9 grams (4.1 mmol) of di(tert-butyl)dicarbonate. The mixture was stirred for 18 hours and then the solvent was evaporated. The residue was dissolved in 20 mL of acetone and treated with 1.4 mL of iodomethane. The mixture was refluxed for four hours. The solvent was evaporated at reduced pressure and the residue was triturated with ethyl acetate. The yellow solid was collected by filtration to give 1.5 grams of product (100%). MS: Calculated for C₁₈H₂₇N₂O₂S⁺: 335.2. Found: 335.5.

### (3R)-3-methyl-1-(phenylcarbonothioyl)piperazine

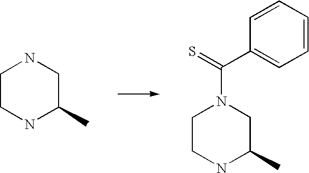

A mixture of 2.8 grams (28 mmol) of (R)-2-methylpiperazine and 6.0 grams (28.0 mmol) of S-(thiobenzoyl)thioglycolic acid was suspended in a solution of 1.6 g (40 mmol) NaOH in 50 mL of distilled water. The mixture was stirred for 5 minutes at 25° C., and then for 5 minutes at 60° C. The mixture was cooled to 25° C., and the ivory precipitate was collected by filtration. It was washed with distilled water, and dried in a stream of air. After further drying under high vacuum overnight there was obtained 5.1 grams of a white solid (80%). ¹HNMR (CDCl₃): 7.35-7.20 (m, 5H), 6.50-5.45 (m. 1H), 4.80-4.65 (m, 1H), 3.90-3.70 (m, 1H), 3.30-2.70 (m, 5H), 1.20 (d, J=7 Hz, 1.5H), 0.95 (d, J=7 Hz, 1.5H).

### Example 107

### {3-Methyl-4-[5-(3-methyl-[1,2,4]oxadiazol-5-yl)-thiophene-2-sulfonyl]-piperazin-1-yl}-phenyl-methanone

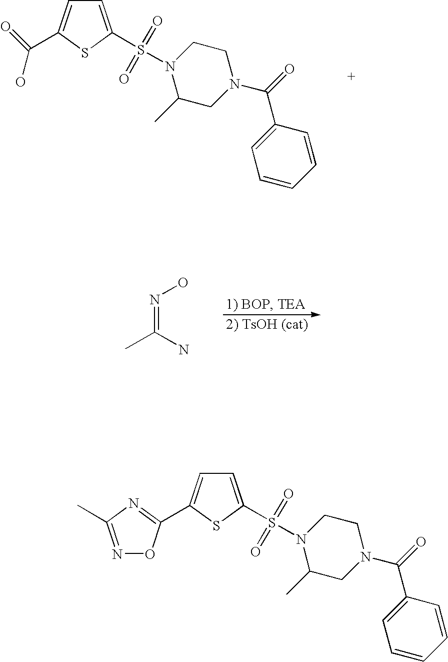

A solution of 5-(4-benzoyl-2-methyl-piperazine-1-sulfonyl)-thiophene-2-carboxylic acid (0.215 g, 0.5 mmol), acetamide oxime (0.04 g, 0.5 mmol), BOP-reagent (0.22 g, 0.5 mmol) and DIPEA (0.17 mL, 0.1 mmol) in DCM (20 mL) was kept at room temperature under stirring overnight. The solvent was then evaporated at reduced pressure. The residue was chromatographed on silica gel eluting with 9:1 ethyl acetate/n-hexane. The solvent was evaporated and the residue was dissolved in xylene (5 mL). p-Toluenesulphonic acid (as catalyst, 0.01 g) was added and the reaction mixture was refluxed under stirring for 12 hours. It was cooled to room temperature, and chromatographed on silica gel eluting with 4:1 ethyl acetate/n-hexane. The solvent was evaporated to afford {3-methyl-4-[5-(3-methyl-[1,2,4]oxadiazol-5-yl)-thiophene-2-sulfonyl]-piperazin-1-yl}-phenyl-methanone (0.015 g, 11%). LCMS: Calc'd for C₁₉H₂₀N₄O₄S₂.H⁺: m/z=433.5. Found: m/z=433, 434. ¹H NMR (DMSO-d₆): 8.05 (d, J=4.2 Hz, 1H); 7.86 (d, J=4.2 Hz, 1H); 7.45-7.37 (m, 5H); 4.44-3.36 (br signal, 4H); 3.20-2.76 (br signal, 3H); 2.42 (s, 3H); 1.04 (br signal, 3H).

### 5-(4-Benzoyl-2-methyl-piperazine-1-sulfonyl)-thiophene-2-carboxylic acid ethyl ester

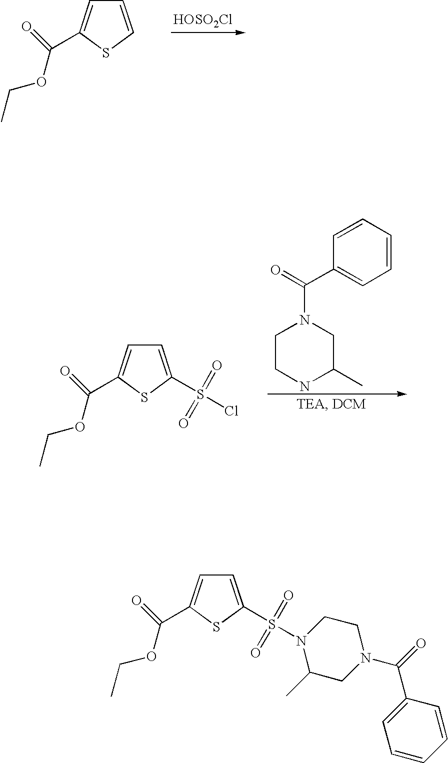

Chlorosulphonic acid (34 mL, 0.512 mol) was added to a solution of thiophene-2-carboxylic acid ethyl ester (20.0 g, 0.128 mol) in DCM (100 mL) at −50° C. under stirring. The reaction mixture was allowed to warm to room temperature and kept for 12 hours. It was then poured into ice water, extracted with DCM, and the organic phase was dried with sodium sulfate. The solvent was evaporated to give a mixture (5.75 g) of 5-chlorosulfonyl-thiophene-2-carboxylic acid ethyl ester and an isomeric side product which was used in the next step without separation. The crude 5-chlorosulfonyl-thiophene-2-carboxylic acid ethyl ester (5.75 g, 0.023 mol), triethylamine (3.98 mL, 0.028 mol) and (3-methyl-piperazin-1-yl)-phenyl-methanone (5.5 g, 0.027 mol) in DCM (150 mL) was kept at room temperature for 12 hours under stirring (TLC monitoring, MeOH/CHCl3 5:95). It was then washed with water, dried with sodium sulfate and the solvent was evaporated. The residue was purified by reverse phase preparative HPLC to afford 5-(4-benzoyl-2-methyl-piperazine-1-sulfonyl)-thiophene-2-carboxylic acid ethyl ester (1 g, 10%). LCMS: Calc'd for C₁₉H₂₂N₂O₅S₂. H⁺: m/z=423.5. Found: m/z=423, 424.

### 5-(4-Benzoyl-2-methyl-piperazine-1-sulfonyl)-thiophene-2-carboxylic acid

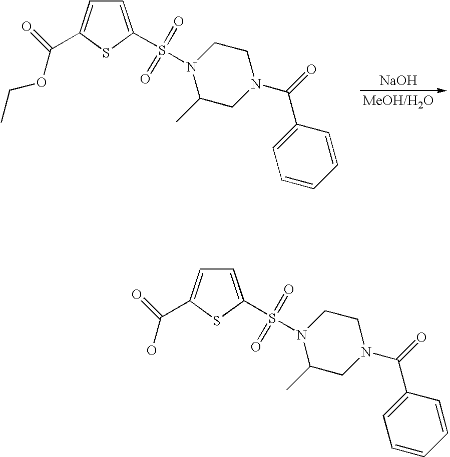

A solution of 5-(4-benzoyl-2-methyl-piperazine-1-sulfonyl)-thiophene-2-carboxylic acid ethyl ester (0.58 g, 1.4 mmol) and NaOH (0.28 g, 7 mmol) in a methanol-water mixture (20 mL: 3 mL) was kept at room temperature under stirring until starting material disappeared (TLC monitoring, 10% MeOH/CHCl₃). The solution was then acidified with HCl to pH3, diluted with water (15 mL), and extracted with chloroform. The organic phase was dried with sodium sulphate, and the solvent was evaporated to afford 5-(4-benzoyl-2-methyl-piperazine-1-sulfonyl)-thiophene-2-carboxylic acid (0.45 g, 84%). ¹H NMR (DMSO-d₆): 13.78 (br s, 1H); 7.74 (d, J=3.9 Hz, 1H); 7.70 (d, J=3.9 Hz, 1H); 7.45-7.36 (m, 5H); 4.47-3.88 (br signal, 2H); 3.80-3.44 (br signal, 2H); 3.22-2.72 (br signal, 3H); 0.99 (br signal, 3H).

### Example 108

### 5-(4-Benzoyl-2-methyl-piperazine-1-sulfonyl)-thiophene-2-carboxylic acid methoxy-methyl-amide

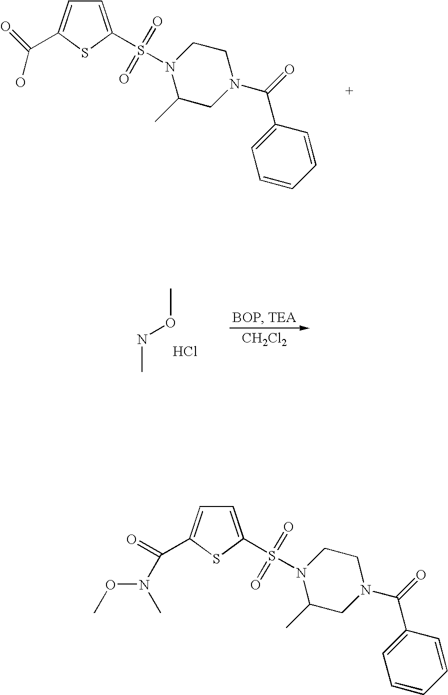

A solution of 5-(4-benzoyl-2-methyl-piperazine-1-sulfonyl)-thiophene-2-carboxylic acid (0.25 g, 0.6 mmol), O,N-dimethyl-hydroxylamine hydrochloride (0.058 g, 0.6 mmol), BOP-reagent (0.26 g, 0.6 mmol) and DIPEA (0.20 mL, 0.12 mmol) in DCM (20 mL) was kept at room temperature under stirring overnight. The solvent was evaporated to ¼ the original volume and then the mixture was chromatographed on silica gel eluting with 2:1 ethyl acetate/n-hexane. The solvent was evaporated to afford 5-(4-benzoyl-2-methyl-piperazine-1-sulfonyl)-thiophene-2-carboxylic acid methoxy-methyl-amide (0.08 g, 36%). LCMS: Calc'd for C₁₉H₂₃N₃O₅S₂.H⁺: m/z=438.5. Found: m/z=438, 439. ¹H NMR (DMSO-d₆): 7.85 (d, J=4.2 Hz, 1H); 7.70 (d, J=4.2 Hz, 1H); 7.44-7.37 (m, 5H); 4.52-3.91 (br signal, 2H); 3.80 (s, 3H); 3.78-3.46 (br signal, 2H); 3.32 (s, 3H); 3.27-2.70 (br signal, 3H); 0.99 (br signal, 3H).

### Example 109

### [3-Methyl-4-(5-[1,2,4]triazol-1-yl-thiophene-2-sulfonyl)-piperazin-1-yl]-phenyl-methanone

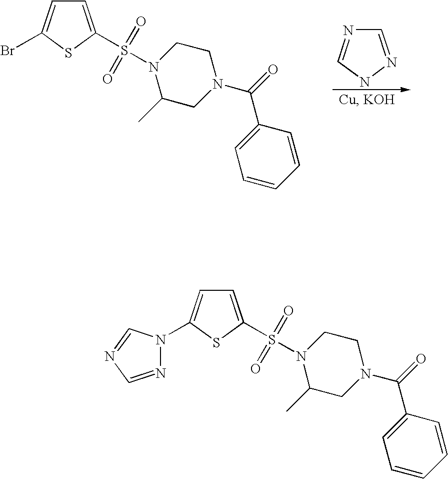

Mixture of [4-(5-bromo-thiophene-2-sulfonyl)-3-methyl-piperazin-1-yl]-phenyl-methanone (0.35 g, 0.8 mmol), Cu-powder (0.1 g, 1.6 mmol), 1,2,4-triazole (1.65 mL, 24.0 mmol), and powered KOH (0.09 g, 1.6 mmol) was heated to 155° C. and kept under stirring at this temperature for 1 hour (TLC monitoring, 1:1 EtOAc/n-hexane). The reaction mixture was then cooled to 50° C., co-evaporated with methanol (50 mL) and silica gel (15 mL), placed onto a silica gel column, eluted with 2% MeOH/CH₂Cl₂ and evaporated. The residue was purified by reverse phase preparative HPLC to afford [3-methyl-4-(5-[1,2,4]triazol-1-yl-thiophene-2-sulfonyl)-piperazin-1-yl]-phenyl-methanone (0.09 g, 27%). LCMS: Calc'd for C₁₈H₁₉N₅O₃S₂.H⁺: m/z=418.5. Found: m/z=418, 419. ¹H NMR (DMSO-d₆): 9.38 (s, 1H); 8.30 (s, 1H); 7.72 (d, J=4.2 Hz, 1H); 7.65 (d, J=4.2 Hz, 1H); 7.45-7.37 (m, 5H); 4.47-3.87 (br signal, 2H); 3.79-3.37 (br signal, 2H); 3.28-2.72 (br signal, 3H); 1.05 (br signal, 3H).

### [4-(5-Bromo-thiophene-2-sulfonyl)-3-methyl-piperazin-1-yl]-phenyl-methanone

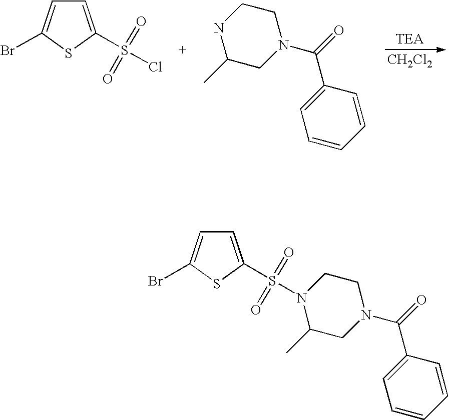

Triethylamine (0.50 mL, 3.6 mmol) was added to a solution of 5-bromo-thiophene-2-sulfonyl chloride (0.94 g, 3.6 mmol) and (3-methyl-piperazin-1-yl)-phenyl-methanone (0.72 g, 3.6 mmol) in DCM (20 mL). The reaction mixture was stirred at 40° C. for 2 hours (TLC monitoring, MeOH/CHCl₃ 5:95), washed with water, dried over sodium sulfate and filtered. Solvent was evaporated to afford [4-(5-bromo-thiophene-2-sulfonyl)-3-methyl-piperazin-1-yl]-phenyl-methanone (1.4 g, 92%). ¹H NMR (DMSO-d₆): 7.55 (d, J=4.2 Hz, 1H); 7.46-7.36 (m, 6H); 4.45-3.86 (br signal, 2H); 3.76-3.29 (br signal, 2H); 3.22 (m, 1H); 3.15-2.68 (br signal, 2H); 1.01 (br signal, 3H).

### Example 110

### {4-[5-(Furan-2-carbonyl)-thiophene-2-sulfonyl]-3-methyl-piperazin-1-yl}-phenylmethanone (General Procedure B)

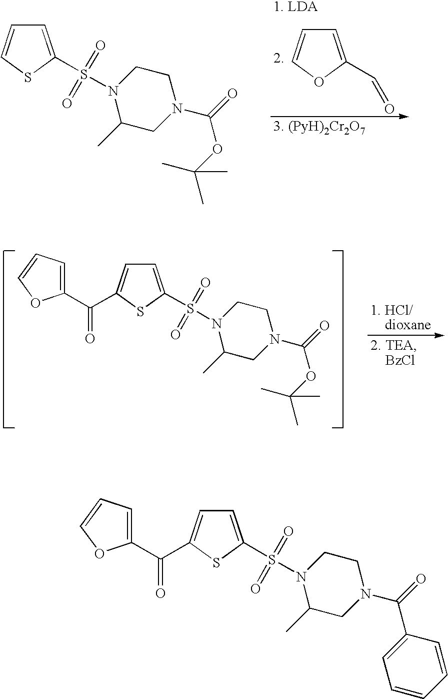

To a solution of diisopropylamine (0.17 mL, 1.2 mmol) in anhydrous THF (10 mL) was added by drops 15% solution of butyllithium in THF (0.8 mL, 1.28 mmol) at −70° C. under stirring and inert atmosphere. The reaction mixture was kept for 15 minutes, then a solution of tert-butyl 3-methyl-4-(thiophen-2-ylsulfonyl)piperazine-1-carboxylate (0.4 g, 1.16 mmol) in anhydrous THF (5 mL) was added. After 30 minutes of stirring at the same temperature a solution of furan-2-carbaldehyde (0.11 g, 1.16 mmol) in anhydrous THF (5 mL) was added. The reaction mixture was stirred at −70° C. for 30 minutes additionally, then heated to room temperature, quenched with water (30 mL), extracted with DCM, washed with water, dried with sodium sulfate and evaporated. To the crude residue, DMF (5 mL) and pyridinium dichromate (0.47 g, 1.25 mmol) were added. The reaction mixture was kept under stirring at room temperature overnight, then poured into water (30 mL), extracted with EtOAc washed with brine, dried with sodium sulfate and evaporated to give a compound tentatively identified as tert-butyl 4-(5-(furan-2-carbonyl)thiophen-2-ylsulfonyl)-3-methylpiperazine-1-carboxylate (0.16 g). LCMS: Calc'd for C₁₄H₁₆N₂O₄S₂.H⁺ (without tert-butoxycarbonyl group): m/z=340.5. Found: m/z=341.

A 4M solution of HCl in dioxane (20 mL) was added to the above material (0.16 g, 0.364 mmol), and the mixture was stirred at room temperature for 1 hour, and evaporated. To the residue a solution of benzoyl chloride (0.058 g, 0.414 mmol) in DCM (10 mL) and then TEA (0.12 mL, 0.83 mmol) were added. The reaction mixture was stirred for 1 hour, and evaporated. The residue was purified by a silica gel column chromatography using EtOAc/n-hexane (2:1) as eluent to afford {4-[5-(furan-2-carbonyl)-thiophene-2-sulfonyl]-3-methyl-piperazin-1-yl}-phenyl-methanone (0.13 g, total 25% yield from tert-butyl 3-methyl-4-(thiophen-2-ylsulfonyl)piperazine-1-carboxylate). LCMS: Calc'd for C₂₁H₂₀N₂O₅S₂.H⁺: m/z=445.5. Found: m/z=445, 446. ¹H NMR (DMSO-d₆): 8.19 (m, 2H); 7.82 (d, J=4.0 Hz, 1H); 7.69 (d, J=4.0 Hz, 1H); 7.43-7.37 (m, 5H); 6.87 (m, 1H); 4.55-3.87 (br signal, 2H); 3.83-3.36 (br signal, 2H); 3.29-2.80 (br signal, 3H); 1.04 (br signal, 3H).

### tert-butyl 3-methyl-4-(thiophen-2-ylsulfonyl)piperazine-1-carboxylate

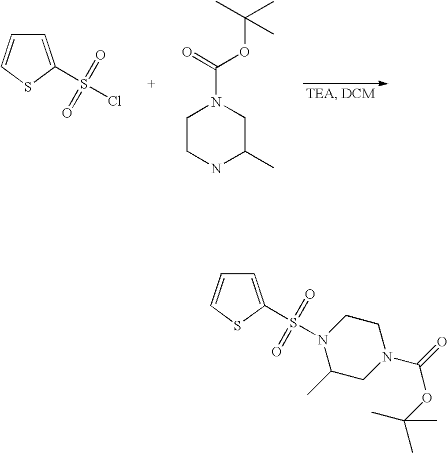

Triethylamine (2.8 mL, 19.5 mmol) was added to a mixture of 2-chlorosulfonyl-thiophene (3.56 g, 19.5 mmol) and 3-methyl-piperazine-1-carboxylic acid tert-butyl ester (3.90 g, 19.5 mmol) in DCM (50 mL). The reaction mixture was kept at room temperature for 1 hour under stirring (TLC monitoring, MeOH/CHCl₃ 5:95), washed with water, dried with sodium sulfate, and evaporated. The residue was purified by a silica gel column chromatography using EtOAc/n-hexane (2:1) as eluent to give tert-butyl 3-methyl-4-(thiophen-2-ylsulfonyl)piperazine-1-carboxylate (6.1 g, 90%). ¹H NMR (DMSO-d₆): 7.99 (dd, J=5.1 Hz, J=1.2 Hz, 1H); 7.67 (dd, J=3.9 Hz, J=1.2 Hz, 1H); 7.22 (dd, J=5.1 Hz, J=3.9 Hz, 1H); 4.03 (m, 1H); 3.86 (br signal, 1H); 3.67 (dm, J=13.0 Hz, 1H); 3.54 (dm, J=13.0 Hz, 1H); 3.07 (m, 1H); 2.90 (br signal, 1H); 2.77 (br signal, 1H); 1.37 (s, 9H); 0.93 (d, J=6.9 Hz, 3H).

### Example 111

### {4-[5-(3H-Imidazole-4-carbonyl)-thiophene-2-sulfonyl]-3-methyl-piperazin-1-yl}-phenyl-methanone

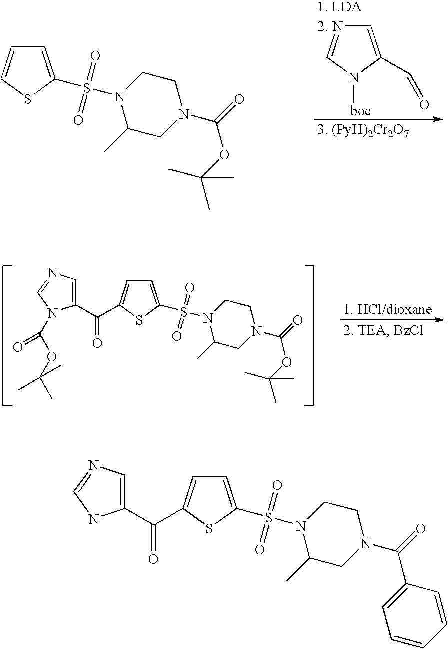

{4-[5-(3H-Imidazole-4-carbonyl)-thiophene-2-sulfonyl]-3-methyl-piperazin-1-yl}-phenyl-methanone (0.21 g, total 33% yield from tert-butyl 3-methyl-4-(thiophen-2-ylsulfonyl)piperazine-1-carboxylate) was prepared according to general procedure B using 5-formyl-imidazole-1-carboxylic acid tert-butyl ester instead of furan-2-carbaldehyde. LCMS: Calc'd for C₂₀H₂₀N₄O₄S₂.H⁺: m/z=445.5. Found: m/z=444, 446. ¹H NMR (DMSO-d₆): 13.00 (br, s, 1H); 8.36 (d, J=3.9 Hz, 1H); 8.14 (brs, 1H); 7.96 (s, 1H); 7.75 (d, J=3.9 Hz, 1H); 7.43-7.37 (m, 5H); 4.51-3.91 (br signal, 2H); 3.85-3.38 (br signal, 2H); 3.27-2.81 (br signal, 3H); 1.01 (br signal, 3H).

### Example 112

### {4-[5-(Isoxazole-3-carbonyl)-thiophene-2-sulfonyl]-3-methyl-piperazin-1-yl}-phenyl-methanone

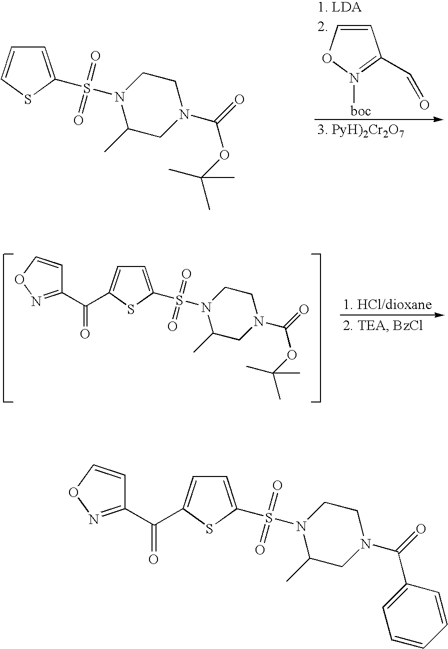

{4-[5-(Isoxazole-3-carbonyl)-thiophene-2-sulfonyl]-3-methyl-piperazin-1-yl}-phenyl-methanone (0.27 g, total 52% yield from tert-butyl 3-methyl-4-(thiophen-2-ylsulfonyl)piperazine-1-carboxylate) was prepared according to General Procedure B, using isoxazole-3-carbaldehyde instead of furan-2-carbaldehyde. LCMS: Calc'd for C₂₀H₁₉N₃O₅S₂.H⁺: m/z=446.5. Found: m/z=446, 447. ¹H NMR (DMSO-d₆): 9.26 (s, 1H); 8.33 (d, J=4.2 Hz, 1H); 7.84 (d, J=4.2 Hz, 1H); 7.43-7.37 (m, 5H); 7.14 (s, 1H); 4.51-3.95 (br signal, 2H); 3.91-3.30 (br signal, 3H); 3.21-2.74 (br signal, 2H); 1.04 (br signal, 3H).

### Examples 113

### [3-Methyl-4-(3-[1,2,3]triazol-1-yl-benzenesulfonyl)-piperazin-1-yl]-phenyl-methanone

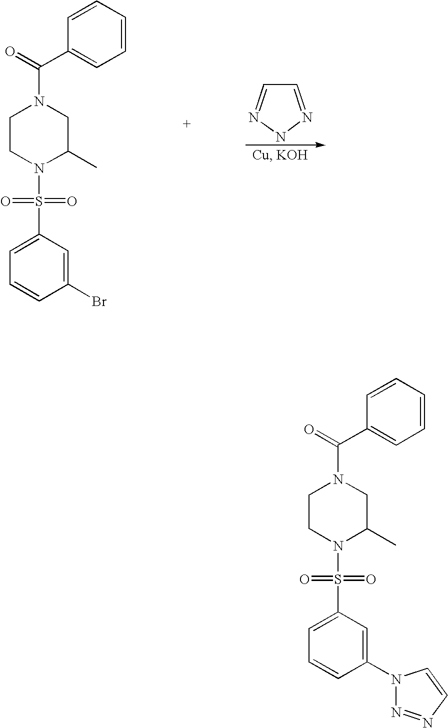

Mixture of [4-(3-bromo-benzenesulfonyl)-3-methyl-piperazin-1-yl]-phenyl-methanone (0.30 g, 0.71 mmol), Cu-powder (0.09 g, 1.42 mmole), 1,2,3-triazole (1.47 g, 1.23 mL, 21.3 mmole), and powered KOH (0.079 g, 1.42 mmole) was heated to 160° C. and kept under stirring at this temperature for 24 hours (TLC monitoring, 10% MeOH/CHCl₃). The reaction mixture was then cooled to room temperature, diluted with CH₂Cl₂ (˜2 mL), placed onto a silica gel column and eluted with 2% MeOH/CH₂Cl₂. Fractions containing the product (together with isomeric side product) were collected and evaporated. The residue was purified by reverse phase preparative HPLC to afford [3-methyl-4-(3-[1,2,3]triazol-1-yl-benzenesulfonyl)-piperazin-1-yl]-phenyl-methanone (0.035 g, 12%). LCMS: Calc'd for C₂₀H₂₁N₅O₃S H⁺: m/z=412.5. Found: m/z=412, 413. ¹H NMR (DMSO-d₆): 9.01 (s, 1H); 8.31 (s, 1H); 8.26 (d, J=8.1 Hz, 1H); 7.95 (s,1H); 7.90 (d, J=7.8 Hz, 1H); 7.86 (dd, J=7.8 Hz, J=8.1 Hz, 1H); 7.42-7.34 (m, 5H); 4.39-3.94 (br signal, 2H); 3.83-3.46 (br signal, 2H); 3.23 (m, 1H); 3.09-2.78 (br signal, 2H); 0.98 (br signal, 3H).

### [4-(3-Bromo-benzenesulfonyl)-3-methyl-piperazin-1-yl]-phenyl-methanone (General Procedure C)

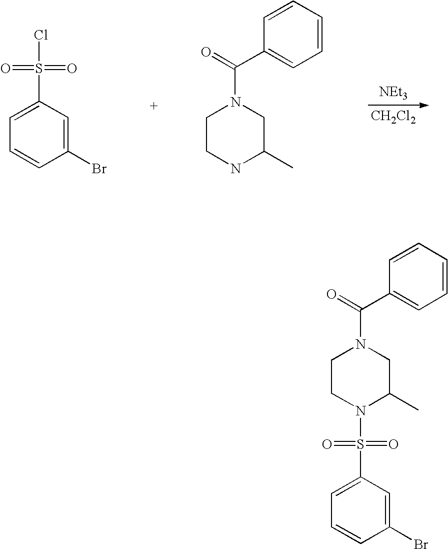

Triethylamine (2.11 g, 2.9 mL, 21 mmol) was added to a solution of 3-bromo-benzenesulfonyl chloride (5.00 g, 20 mmol) and (3-methyl-piperazin-1-yl)-phenyl-methanone (4.10 g, 20 mmol) in DCM (50 mL). The reaction mixture was stirred at 40° C. for 2 hours (TLC monitoring, MeOH/CHCl₃ 5:95), washed with water (3×20 mL), dried over sodium sulfate and filtered. Solvent was evaporated to give [4-(3-bromo-benzenesulfonyl)-3-methyl-piperazin-1-yl]-phenyl-methanone (7.12 g, 85%). ¹H NMR (DMSO-d₆): 7.95 (br s, 1H); 7.90 (d, J=7.8 Hz, 1H); 7.84 (d, J=8.0 Hz, 1H); 7.58 (m, 1H); 7.42-7.34 (m, 5H); 4.78-4.16 (br signal, 2H); 3.86-3.29 (br signal, 2H); 3.18 (m, 1H); 3.15-2.90 (br signal, 2H); 0.94 (br signal, 3H).

### Example 114

### [3-Methyl-4-(3-[1,2,4]triazol-1-yl-benzenesulfonyl)-piperazin-1-yl]-phenyl-methanone

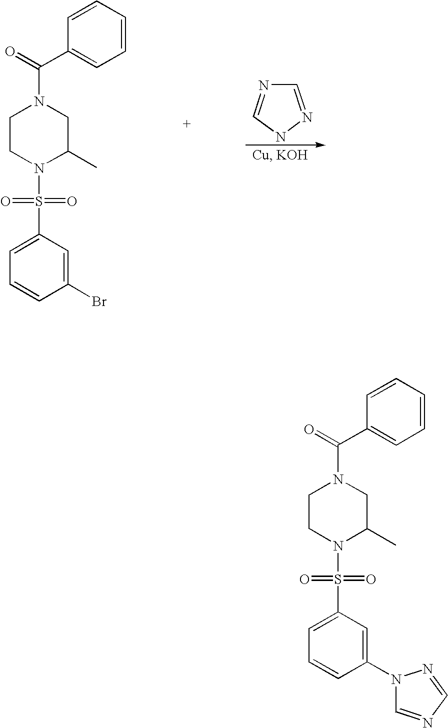

Mixture of [4-(3-bromo-benzenesulfonyl)-3-methyl-piperazin-1-yl]-phenyl-methanone (0.30 g, 0.71 mmol), Cu-powder (0.09 g, 1.42 mmole), 1,2,4-triazole (1.23 mL, 21.3 mmole), and powered KOH (0.079 g, 1.42 mmole) was heated to 160° C. and kept under stirring at this temperature for 24 hours (TLC monitoring, 10% MeOH/CHCl₃). The reaction mixture was then cooled to room temperature, diluted with CH₂Cl₂ (˜2 mL), placed onto a silica gel column and eluted with CH₂Cl₂, then 2% MeOH/CH₂Cl₂ and evaporated to afford [3-methyl-4-(3-[1,2,4]triazol-1-yl-benzenesulfonyl)-piperazin-1-yl]-phenyl-methanone (0.15 g, 52%). LCMS: Calc'd for C₂₀H₂₁N₅O₃S H⁺: m/z=412.5. Found: m/z=412, 413. ¹H NMR (DMSO-d₆): 9.46 (s, 1H); 8.29 (s, 1H); 8.25 (s, 1H); 7.95 (d, J=7.8 Hz, 1H); 7.83 (m, 2H); 7.42-7.34 (m, 5H); 4.39-3.94 (br signal, 2H); 3.83-3.46 (br signal, 2H); 3.23 (m, 1H); 3.09-2.78 (br signal, 2H); 0.98 (br signal, 3H).

### Example 115

### [3-Methyl-4-(3-pyrazol-1-yl-benzenesulfonyl)-piperazin-1-yl]-phenyl-methanone

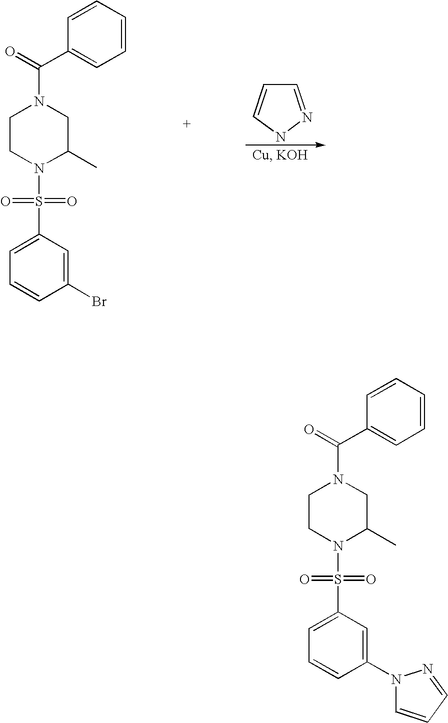

[3-Methyl-4-(3-pyrazol-1-yl-benzenesulfonyl)-piperazin-1-yl]-phenyl-methanone (0.17 g, 59%) was prepared according to Example 114 above, using pyrazole instead of 1,2,4-triazole. LCMS: Calc'd for C₂₁H₂₂N₄O₃SH⁺: m/z=411.5. Found: m/z=411, 412. ¹H NMR (DMSO-d₆): 8.68 (s, 1H); 8.24 (s, 1H); 8.16 (br s, 1H); 7.82 (s, 1H); 7.74 (m, 2H); 7.42-7.34 (m, 5H); 6.60 (br s, 1H); 4.42-3.90 (br signal, 2H); 3.85-3.46 (br signal, 2H); 3.23 (m, 1H); 3.10-2.75 (br signal, 2H); 0.97 (br signal, 3H).

### Examples 116 and 117

### [3-Methyl-4-(4-[1,2,3]triazol-1-yl-benzenesulfonyl)-piperazin-1-yl]-phenyl-methanone and [3-methyl-4-(4-[1,2,3]triazol-2-yl-benzenesulfonyl)-piperazin-1-yl]-phenyl-methanone

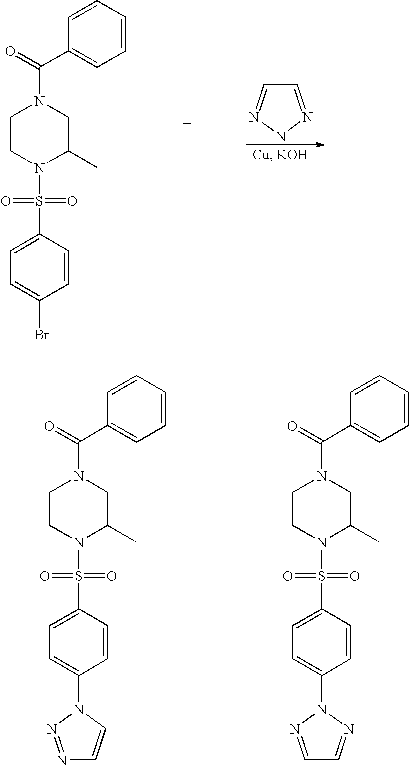

Mixture of [4-(4-bromo-benzenesulfonyl)-3-methyl-piperazin-1-yl]-phenyl-methanone (0.30 g, 0.71 mmol), Cu-powder (0.09 g, 1.42 mmole), 1,2,3-triazole (1.47 g, 1.23 mL, 21.3 mmole), and powered KOH (0.08 g, 1.42 mmole) was heated to 160° C. and kept under stirring at this temperature for 24 hours (TLC monitoring, 10% MeOH/CHCl₃). The reaction mixture was then cooled to room temperature, diluted with MeOH (˜5 mL), filtered and evaporated. The residue was purified by a silica gel column chromatography using ethylacetate as eluent to give [3-methyl-4-(4-[1,2,3]triazol-1-yl-benzenesulfonyl)-piperazin-1-yl]-phenyl-methanone (0.11 g, 38%) and [3-methyl-4-(4-[1,2,3]triazol-2-yl-benzenesulfonyl)-piperazin-1-yl]-phenyl-methanone (0.09 g, 31%).

[3-methyl-4-(4-[1,2,3]triazol-1-yl-benzenesulfonyl)-piperazin-1-yl]-phenyl-methanone LCMS: Calc'd for C₂₀H₂₁N₅O₃S H⁺: m/z=412.5. Found: m/z=412, 413. ¹H NMR (DMSO-d₆): 8.99 (d, J=1.0 Hz, 1H); 8.19 (d, J=8.8 Hz, 2H); 8.04 (d, J=8.8 Hz, 2H); 8.04 (d, J=1.0 Hz, 1H); 7.42 (m, 3H); 7.35 (m, 2H); 4.46-3.86 (br signal, 2H); 3.84-3.38 (br signal, 2H); 3:21 (m, 1H); 3.14-2.70 (br signal, 2H); 0.98 (br signal, 3H).

[3-methyl-4-(4-[1,2,3]triazol-2-yl-benzenesulfonyl)-piperazin-1-yl]-phenyl-methanone LCMS: Calc'd for C₂₀H₂₁N₅O₃S H⁺: m/z=412.5. Found: m/z=412, 413. ¹H NMR (DMSO-d₆): 8.24 (d, J=8.5 Hz, 2H); 8.23 (s, 2H); 8.01 (d, J=8.5 Hz, 2H); 7.41 (m, 3H); 7.35 (m, 2H); 4.39-3.84 (br signal, 2H); 3.82-3.37 (br signal, 2H); 3.20 (m, 1H); 3.09-2.70 (br signal, 2H); 0.97 (br signal, s 3H).

### [4-(4-Bromo-benzenesulfonyl)-3-methyl-piperazin-1-yl]-phenyl-methanone

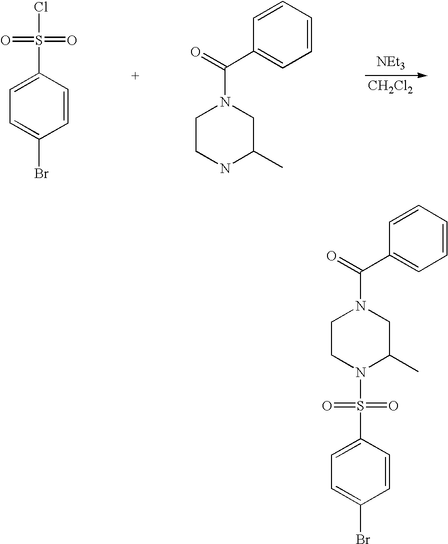

[4-(4-Bromo-benzenesulfonyl)-3-methyl-piperazin-1-yl]-phenyl-methanone (6.70 g, 80%) was prepared according to General procedure C using 4-bromo-benzenesulfonyl chloride in place of 3-bromo-benzenesulfonyl chloride. ¹H NMR (DMSO-d₆): 7.82 (d, J=8.6 Hz, 2H); 7.75 (d, J=8.6 Hz, 2H); 7.45-7.35 (m, 5H); 4.44-3.85 (br signal, 2H); 3.79-3.40 (br signal, 2H); 3.16 (m, 1H); 3.08-2.65 (br signal, 2H); 0.94 (br signal, 3H).

### Example 118

### [3-Methyl-4-(4-[1,2,4]triazol-1-yl-benzenesulfonyl)-piperazin-1-yl]-phenyl-methanone

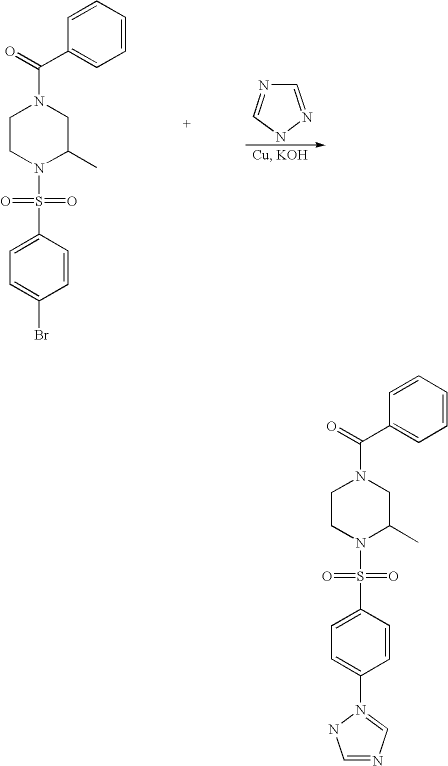

[3-Methyl-4-(4-[1,2,4]triazol-1-yl-benzenesulfonyl)-piperazin-1-yl]-phenyl-methanone (0.15 g, 51%) was prepared according to the procedure in Examples 116 and 117, using 1,2,4-triazole instead of 1,2,3-triazole. LCMS: Calc'd for C₂₀H₂₁N₅O₃S H⁺: m/z=412.5. Found: m/z=412, 413. ¹H NMR (DMSO-d₆): 9.47 (s, 1H); 8.32 (s, 1H); 8.12 (d, J=8.8 Hz, 2H); 8.00 (d, J=8.8 Hz, 2H); 7.42 (m, 3H); 7.35 (m, 2H); 4.40-3.86 (br signal, 2H); 3.82-3.38 (br signal, 2H); 3.20 (m, 1H); 3.11-2.60 (br signal, 2H); 0.98 (br signal, 3H).

### Example 119

### [3-Methyl-4-(4-pyrazol-1-yl-benzenesulfonyl)-piperazin-1-yl]-phenyl-methanone

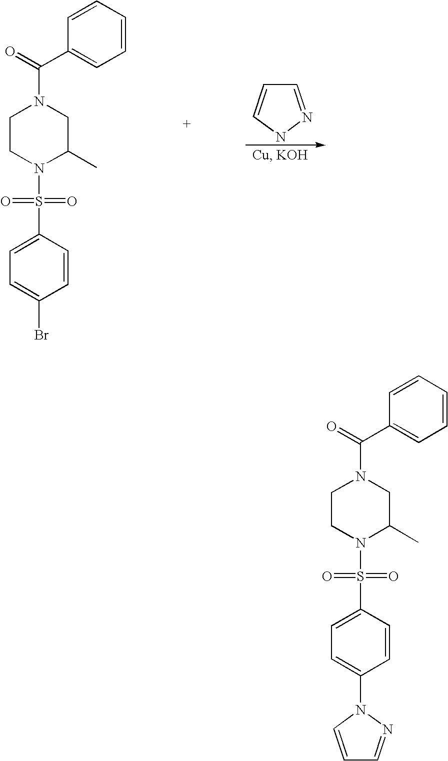

[3-Methyl-4-(4-pyrazol-1-yl-benzenesulfonyl)-piperazin-1-yl]-phenyl-methanone (0.09 g, 29%) was prepared according to the procedure in Examples 116 and 117, using pyrazole instead of 1,2,4-triazole. LCMS: Calc'd for C₂₁H₂₂N₄O₃S H⁺: m/z=411.5. Found: m/z=411, 412. ¹H NMR (DMSO-d₆): 8.67 (d, J=2.7 Hz, 1H); 8.09 (d, J=8.8 Hz, 2H); 8.00 (d, J=8.8 Hz, 2H); 7.84 (d, J=1.5 Hz, 1H); 7.42-7.34 (m, 5H); 6.63 (m, 1H); 4.43-3.96 (br signal, 2H); 3.78-3.41 (br signal, 2H); 3.18 (m, 1H); 3.08-2.67 (br signal, 2H); 0.96 (br signal, 3H).

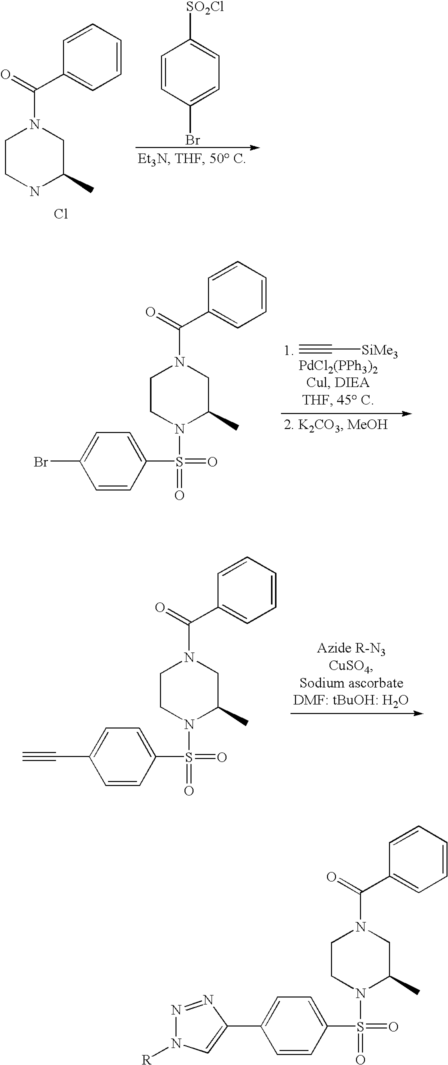

General procedure A for the 1,3-dipolar reactions of azides with [(R)-4-(4-ethynyl-benzenesulfonyl)-3-methyl-piperazin-1-yl]-phenyl-methanone

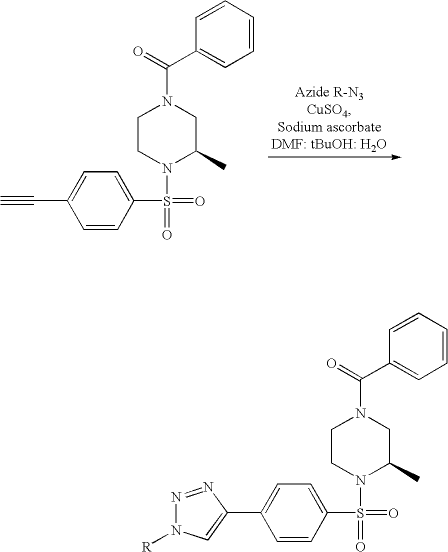

To a mixture of [(R)-4-(4-ethynyl-benzenesulfonyl)-3-methyl-piperazin-1-yl]-phenyl-methanone (40 mg, 0.13 mmol) and azide (0.13 mmol) in 1:1 ^{t}BuOH:H₂O (2 mL) containing 100 uL of DMF was added freshly prepared aqueous solutions (1 mM) of sodium ascorbate (11 uL) and copper sulfate (1 uL) and the resulting suspension was stirred at ambient temperatures (25° C. to 80° C.) until all the starting material is consumed (8 to 24 hours). If necessary, additional reagents were added. Solvents were evaporated under high vacuum and the products were purified by HPLC.

### Example 120

### {(R)-3-Methyl-4-[4-(1H-[1,2,3]triazol-4-yl)-benzenesulfonyl]-piperazin-1-yl)-phenyl-methanone

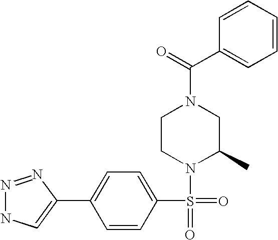

Prepared according to General Procedure A using trimethylsily azide. LCMS: m/e 412 (M+H). ¹H NMR (CDCl₃, 300 MHz): δ 8.00 (s, 1H), 7.97 (d, J=8.5 Hz, 2H), 7.85 (d, J=8.5 Hz, 2H), 7.45-7.33 (m, 5H), 4.20-3.01 (m, 7H), 4.2-3.01 (m, 7H), 1.03 (br s, 3H).

### [(R)-4-(4-bromo-benzenesulfonyl)-3-methyl-piperazin-1-yl]-Phenyl-methanone

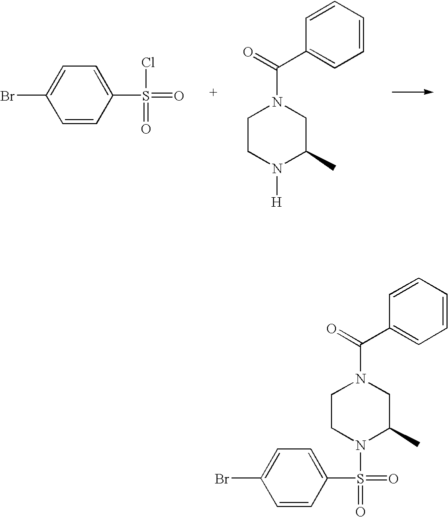

To a solution of ((R)-3-methyl-piperazine-1-yl)-phenyl-methanone hydrochloride (720 mg, 3 mmol) in 15% triethylamine/tetrahydrofuran (20 mL) was added 4-bromo-benzenesulfonyl chloride (712 mg, 3.3 mmol) and mixture was stirred at 45° C. for 3 hours. Solvent was evaporated and the residue was partitioned between ethyl acetate and water. Separate organic layer and the aqueous layer was extracted with ethyl acetate (2×20 mL). Combined extract was dried (MgSO₄), filtered and concentrated to afford [(R)-4-(4-bromo-benzenesulfonyl)-3-methyl-piperazin-1-yl]-phenyl-methanone as light brown solid (1.27 g, 100%) which was used in next reactions without further purification. LCMS: m/e 424 (M+H). ¹H NMR (CDCl₃, 300 MHz): δ 7.71-7.62 (m, 4H), 7.48-7.29 (m, 5H), 4.6-3.16 (m, 7H), 1.03 (br s, 3H).

### (R)-(3-methyl-4-(4-((trimethylsilyl)ethynyl)phenylsulfonyl)piperazin-1-yl)(phenyl)methanone

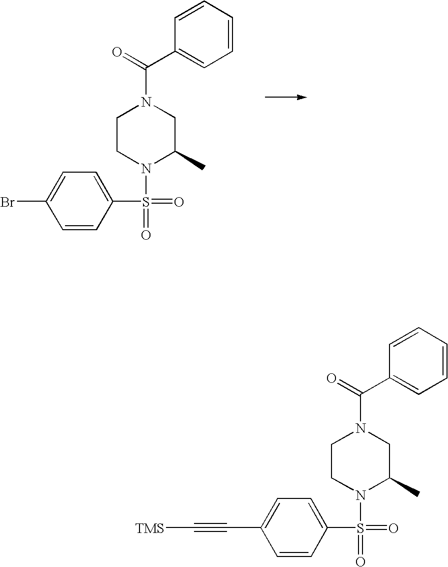

A heterogeneous mixture containing [(R)-4-(4-bromo-benzenesulfonyl)-3-methyl-piperazin-1-yl]-phenyl-methanone (800 mg, 1.9 mmol), trimethylsilylacetylene (222 mg, 2.2 mmol), palladium dichloride bis(triphenyl)phosphene (67 mg, 5 mol %), copper iodide (5 mol %) and diisopropyl ethylamine (575 mg, 5.7 mmol) in anhydrous tetrahydrofuran (20 mL) was heated at 50° C. for 12 hours. The reaction mixture was cooled and the diluted with ethyl acetate (20 mL) and filtered though a Celite bed. The filtrate was concentrated and the crude mixture was purified by a silica gel column chromatography using 20% ethyl aceate in hexanes to afford [(R)-3-methyl-4-(4-trimethylsilanylethynyl-benzenesulfonyl)-piperazin-1-yl]-phenyl-methanone as half white solid (607 mg, 73%). LCMS: m/e 441 (M⁺); ¹H NMR (CDCl₃, 300 MHz): δ 7.73 (d, J=9 Hz, 2H), 7.58 (d, J=9 Hz, 2H), 7.46-7.31 (m, 5H), 4.16-2.95 (m, 7H), 0.94 (s, 3H), 0.27 (s, 9H).

### (R)-(4-(4-ethynylphenylsulfonyl)-3-methylpiperazin-1-yl)(phenyl)methanone

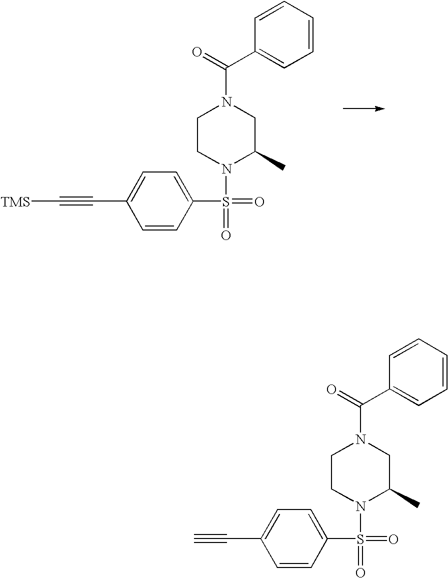

(R)-(3-methyl-4-(4-((trimethylsilyl)ethynyl)phenylsulfonyl)piperazin-1-yl)(phenyl)methanone was dissolved in methanol (10 mL) and was added K₂CO₃ (300 mg, 2.17 mmol). The mixture was stirred at room temperature for 1 h. Solvent was evaporated under vacuum and the residue was partitioned between ethyl acetate and water (20 mL). Aqueous layer was extracted with ethyl acetate (2×10 mL). Combined organic extract was dried (MgSO₄), filtered and concentrated to dryness to afford [(R)-4-(4-ethynyl-benzenesulfonyl)-3-methyl-piperazin-1-yl]-phenyl-methanone which was used in next step without further purification. LCMS: m/e 369 (M+H). ¹H NMR (CDCl₃, 300 MHz): δ 7.76 (d, J=9 Hz, 2H), 7.62 (d, J=9 Hz, 2H), 7.50-7.33 (m, 5H), 3.26 (s, 1H), 4.62-2.85 (m, 7H), 1.00 (br s, 3H).

### Example 121

### {(R)-4-[4-(1-Isobutyl-1H-[1,2,3]triazol-4-yl)-benzenesulfonyl]-3-methyl-piperazin-1-yl}-phenyl-methanone

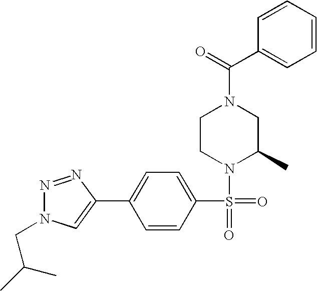

Prepared according to General Procedure A using isobutyl azide, which was prepared in situ using isobutyl bromide and sodium azide in DMF solvent. LCMS: m/e 468 (M+H). ¹H NMR (CDCl₃, 300 MHz): δ 7.99 (d, J=8.8 Hz, 2H), 7.85 (d, J=8.8 Hz, 2H), 7.66 (s, 1H), 7.41-7.37 (m, 5H), 4.24 (d, J=7.17 Hz, 2H), 4.65-2.65 (m, 7H), 2.28 (m, 1H), 1.03 (m, 9H).

### Example 122

### [(R)-4-[4-(1-Benzyl-1H-[1,2,3]triazol-4-yl)-benzenesulfonyl]-3-methyl-piperazine-1-yl}-phenyl-methanone

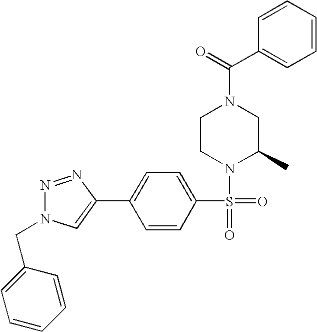

Prepared according to General Procedure A using benzyl azide. LCMS: m/e 502 (M+H). ¹H NMR (CDCl₃, 300 MHz): δ 7.99 (d, J=9 Hz, 2H), 7.82 (d, J=9 Hz, 2H), 7.77 (s, 1H), 7.41-7.31 (m, 10H), 5.60 (s, 2H), 4.59-3.14 (m, 7H), 1.01 (m, 3H).

### Example 123

### 1-(4-{4-[4((R)-4-Benzoyl-2-methyl-piperazine-1-sulfonyl)-phenyl]-[1,2,3]triazol-1-yl}-phenyl-ethanone

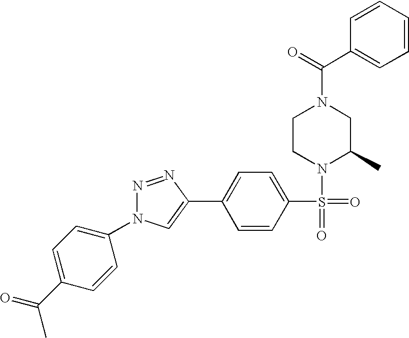

Prepared according to General Procedure A using 4-acetylphenylazide. LCMS: m/e 530 (M+H). ¹H NMR (CDCl₃, 300 MHz): δ 8.45 (s, 1H), 8.18 (d, J=8.5 Hz, 2H), 7.08 (d, J=8.5 Hz, 2H), 7.96-7.89 (m, 4H), 7.41-7.35 (m, 5H), 4.26-2.89 (m, 7H), 2.68 (s, 3H), 1.03 (m, 3H).

### Example 124

### {(R)-3-Methyl-4-[4-(3-phenyl-isoxazol-5-yl)-benzenesulfonyl]-piperazin-yl}-phenyl-methanone

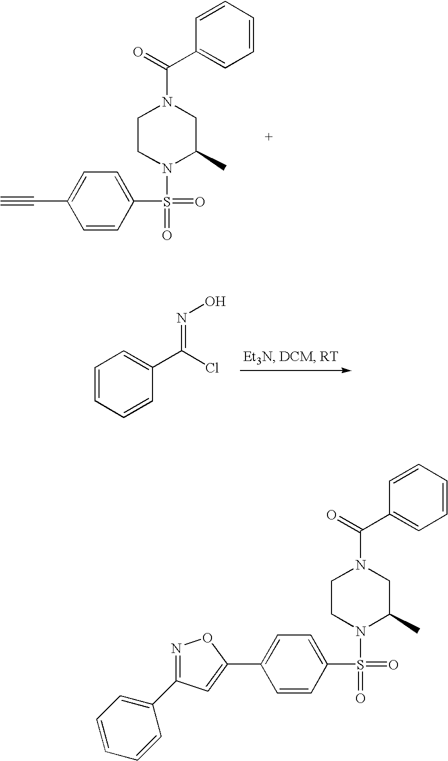

To solution of [(R)-4-(4-ethynyl-benzenesulfonyl)-3-methyl-piperazin-1-yl]-phenyl-methanone (40 mg, 0.1 mmol ) in anhydrous dichloromethane (2 mL) was added phenylcarbomoyl chloride (18.5 mg, 0.11 mmol) and triethylamine (48 mg, 0.48 mmol). Reaction was allowed stir at room temperature for 24 minutes. Solvent was evaporated and the product was purified by HPLC. LCMS: m/e 488 (M+H). ¹H NMR (CDCl₃, 300 MHz): δ 7.91-7.86 (m, 6H), 7.51-7.49 (m, 3H), 7.41-7.32 (m, 5H), 6.98 (s, 1H), 4.30-2.94 (m, 7H), 1.00 (br s, 3H).

### General Procedure D for the 1,3-dipolar Reactions of Azides with [(R)-4-(5-ethynyl-thiophene-2-sulfonyll)-3-methyl-piperazin-1-yl]-phenyl-methanone

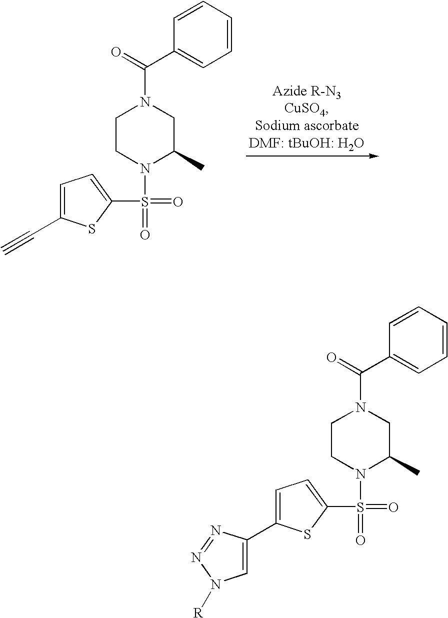

To a mixture of [(R)-4-(5-ethynyl-thiophene-2-sulfonyll)-3-methyl-piperazin-1-yl]-phenyl-methanone (40 mg, 0.1 mmol) and azide (0.1 mmol) in 1:1 ^{t}BuOH:H₂O (2 mL) containing 100 uL of DMF was added freshly prepared aqueous solutions (1 mM) of sodium ascorate (11 uL) and copper sulfate (1 uL) and the suspension was stirred at ambient temperatures (25° C. to 80° C.) until all the starting material is consumed (8 to 24 hours). If necessary, additional reagents were added. Solvents were evaporated under high vacuum and the products were purified by preparative TLC or HPLC.

### [(R)-4-(5-bromo-thiophene-2-sulfonyl)-3-methyl-piperazin-1-yl]-phenyl-methanone

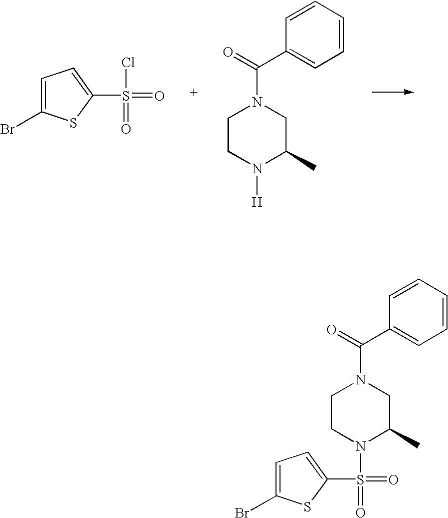

To a solution of ((R)-3-methyl-piperazine-1-yl)-phenyl-methanone hydrochloride (720 mg, 3 mmol) in 15% triethylamine/tetrahydrofuran (20 mL) was added 5-bromo-thiophenesulfonyl chloride (860 mg, 3.3 mmol) and the mixture was stirred at 45° C. for 3 hours. Solvent was evaporated and the residue was partitioned between ethyl acetate and water. Separate organic layer and the aqueous layer was extracted with ethyl acetate (2×20 mL).Combined extract was dried (MgSO₄), filtered and concentrated to afford [(R)-4-(5-bromo-thiophene-2-sulfonyl)-3-methyl-piperazin-1-yl]-phenyl-methanone as light brown solid (1.27 g, 100%) which was used in next reactions without further purification. LCMS: m/e 430 (M+H).

### (R)-(3-methyl-4-(5-((trimethylsilyl)ethynyl)thiophen-2-ylsulfonyl)piperazin-1-yl)(phenyl)methanone

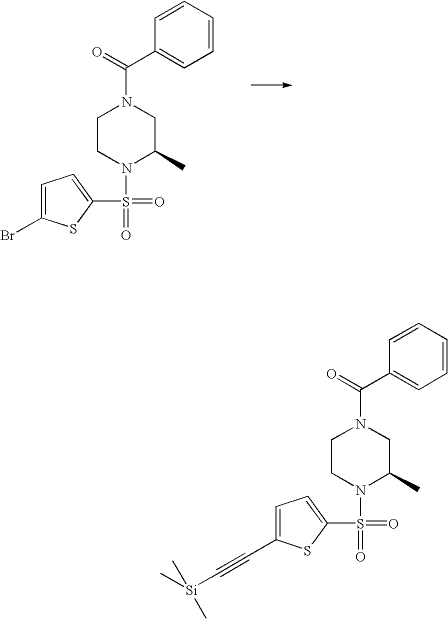

A heterogeneous mixture containing afford [(R)-4-(5-bromo-thiophene-2-sulfonyl)-3-methyl-piperazin-1-yl]-phenyl-methanone (1 g, 2.3 mmol), trimethyl-silylacetylene (270 mg, 2.76 mmol), palladium dichloride bis(triphenyl)phosphine (80 mg, 5 mol %), copper iodide (21.5 mg, 5 mol %) and diisopropyl ethylamine (696 mg, 6.9 mmol) in anhydrous tetrahydrofuran (20 mL) was heated at 45° C. until all the starting material has been consumed (24 to 36 hours). The reaction mixture was cooled and then diluted with ethyl acetate (20 mL), filtered though a Celite bed. The filtrate was concentrated and the crude product was purified by a silica gel column chromatography using 5% ethyl acetate in dichloromethane to afford [(R)-3-methyl-4-(4-trimethyl-silanylethynyl-benzenesulfonyl)-piperazin-1-yl]-phenyl-methanone as half white solid (678 mg, 45%). LCMS: m/e 447 (M+H); ¹H NMR (CDCl₃, 300 MHz): δ 7.43-7.36 (m, 6H), 7.14 (d, J=3 Hz, 1H), 4.16-3.15 (m, 7H), 1.09 (s, 3H), 0.25 (s, 9H).

### (R)-(4-(5-ethynylthiophen-2-ylsulfonyl)-3-methylpiperazin-1-yl)(phenyl)methanone

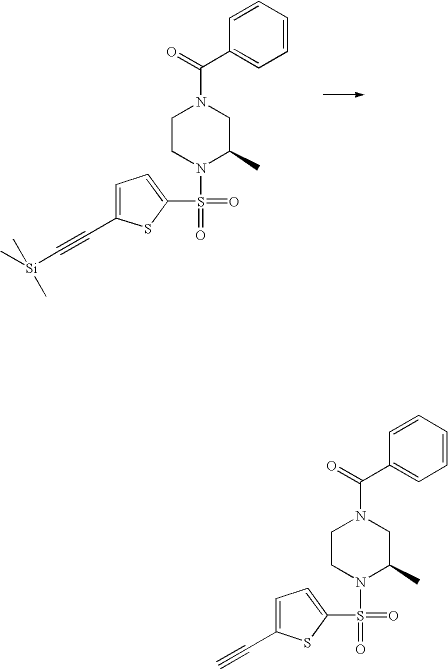

Above silyl derivative was dissolved in methanol (10 mL) and was added K₂CO₃ (800 mg, 5.7 mmol). The mixture was stirred at room temperature for 30 minutes. Solvent was evaporated under vacuum and the residue was partitioned between ethyl acetate and water (20 mL). Aqueous layer was extracted with ethyl acetate (2×10 mL). Combined organic extract was dried (MgSO₄), filtered and concentrated to dryness to afford [(R)-4-(5-ethynyl-thiophene-2-sulfonyll)-3-methyl-piperazin-1-yl]-phenyl-methanone. LCMS: m/e 375 (M+H). LCMS: m/e 447 (M+H); ¹H NMR (CDCl₃, 300 MHz): δ 7.43-7.36 (m, 6H), 7.13 (d, J=3 Hz, 1H), 3.53(s, 1H), 4.76-3.10 (m, 7H), 0.91 (s, 3H).

### Example 125

### {(R)-3-Methyl-4-[5-(1H-[1,2,3]triazol-4-yl)-thiophene-2-sulfonyl]-piperazin-1-yl}-phenyl-methanone

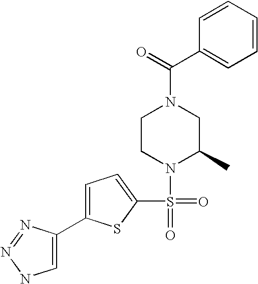

Prepared according to General Procedure D using trimethyl silylazide. LCMS: m/e 412 (M+H). ¹H NMR (CDCl₃, 300 MHz): δ 8.00 (s, 1H), 7.97 (d, J=8.5 Hz, 2H), 7.85 (d, J=8.5 Hz, 2H), 7.45-7.33 (m, 5H), 4.2-3.01 (m, 7H), 1.03 (br s, 3H), 3.01-4.2 (m, 7H), 7.33-7.45 (m, 5H).

### Example 126

### {(R)-4-[4-(1-Isobutyl-1H-[1,2,3]triazol-4-yl)-benzenesulfonyl]-3-methyl-piperazin-1-yl}-phenyl-methanone

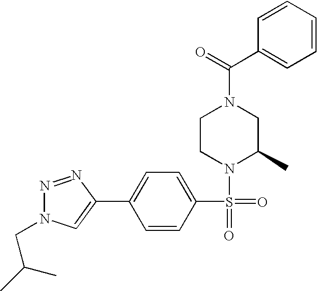

Prepared according to General Procedure D from isobutyl azide, which was prepared in situ using isobutyl bromide and sodium azide in DMF solvent. LCMS: m/e 468 (M+H). ¹H NMR (CDCl₃, 300 MHz): δ 7.99 (d, J=8.8 Hz, 2H), 7.85 (d, J=8.8 Hz, 2H), 7.66 (s, 1H), 7.41-7.37 (m, 5H), 4.24 (d, J=7.17 Hz, 2H), 4.65-2.65 (m, 7H), 2.28 (m, 1H), 1.03 (m, 9H).

### Example 127

### {(R)-4-[5-(1-Benzyl-1H-[1,2,3]triazol-4-yl)-thiophene-2-sulfonyl]-3-methyl-piperazin-1-yl}-phenyl-methanone

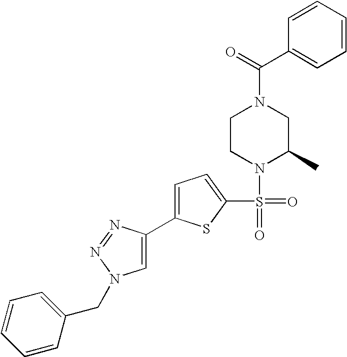

Prepared according to General Procedure D using benzyl azide. LCMS: m/e 508 (M+H); ¹H NMR (CDCl₃, 300 MHz): δ 7.65 (d, 1H), 7.50 (d, J=3.7 Hz, 1H), 7.42-7.24 (m, 11H), 5.58 (s, 2H), 4.49-3.06 (m, 7H), 1.09 (m, 3H).

### Example 128

### 1-(4-{4-[5-((R)-4-Benzoyl-2-methyl-piperazine-1-sulfonyl)-thiophen-2-yl]-[1,2,3]triazol-1-yl}-phenyl-ethanone

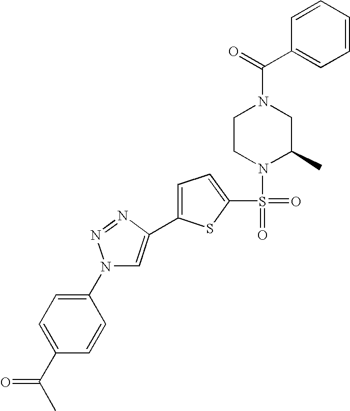

Prepared according to General Procedure D using 4-acetylphenylazide. LCMS: m/e 536 (M+H); ¹H NMR (DMSO-d₆, 300 MHz): δ 8.10 (dd, J₁=8.1 Hz, J₂=3.4 Hz, 4H), 7.77 (d, J=3.7 Hz, 1H), 7.61 (d, J=3.7 Hz, 1H), 7.44-7.42 (m, 6H), 4.21-2.89 (m, 7H), 2.66 (s, 3H), 1.03 (br s, 3H).

### Example 129

### ((R)-3-Methyl-4-{5-[1-(4-methyl-1H-imidazol-2-yl)-1H-[1,2,3]triazol-4-yl]-thiophene-2-sulfonyl}-piperazin-1-yl)-phenyl-methanone

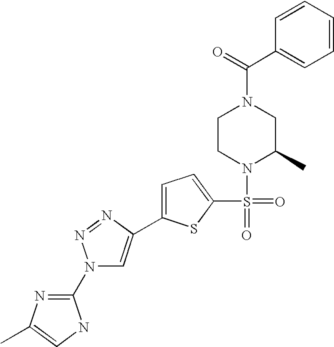

Prepared according to General Procedure D using 2-azido-4-methyl-1H-imidazole. LCMS: m/e 499 (M+H); ¹H NMR (CDCl₃, 300 MHz): δ 8.55 (s, 1H), 7.57 (d, J=3.8 Hz, 1H), 7.43-7.31 (m, 7H), 4.16-3.16 (m, 7H), 2.62 (s, 3H), 1.15 (m, 3H).

### Example 130

### 4-{4-[5-((R)-4-Benzoyl-2-methyl-piperazine-1-sulfonyl)-thiophen-2-yl]-[1,2,3]triazol-1-yl}-2-methylsulfanyl-thiazole-5-carboxylic acid ethyl ester

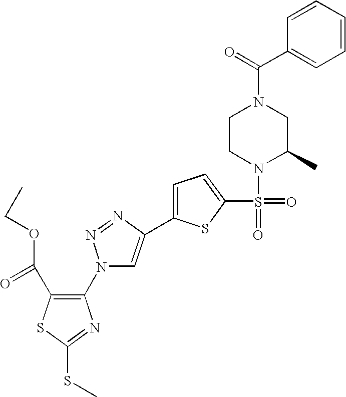

Prepared according to General Procedure D using 4-azido-2-methylsulfanyl-thiozole-5-carboxylic acid ethyl ester. LCMS: m/e 619 (M+H); ¹H NMR (CDCl₃, 300 MHz): δ 8.39 (s, 1H), 7.56 (d, J=4 Hz, 1H), 7.43-7.32 (m, 6H), 4.29 (q, J=7.17 Hz, 2H), 4.16-3.11 (m, 7H), 2.76 (s, 3H), 1.32 (t, J=7.1 Hz, 3H), 1.13 (s, 3H).

### Example 131

### {(R)-3-Methyl-4-[5-(3-phenyl-sioxazol-5-yl)-thiophene-2-sulfonyl]-piperazin-1-yl}-phenyl-methanone

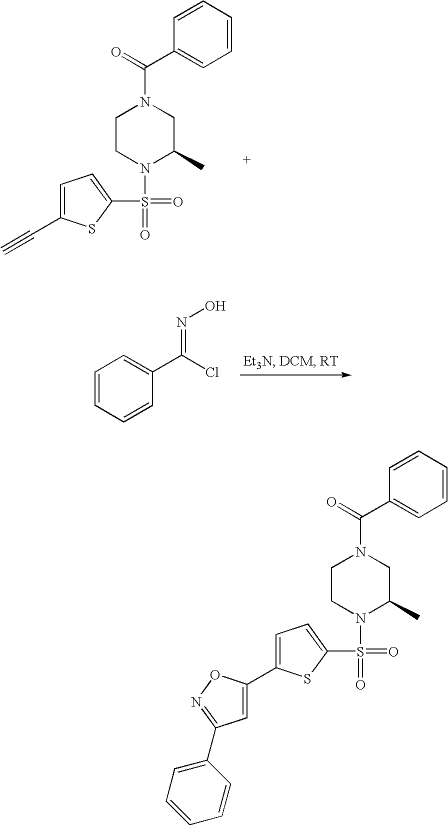

To a solution of [(R)-4-(5-ethynyl-thiphene-2-sulfonyl)-3-methyl-piperazin-1-yl]-phenyl-methanone (50 mg, 0.133 mmol ) in anhydrous dichloromethane (2 mL) was added phenylcarbomoyl chloride (22.7 mg, 0.14 mmol) and triethylamine (48 mg, 0.48 mmol). Reaction was allowed stir at room temperature for 24 minutes. Solvent was evaporated and the product was purified by HPLC. LCMS: m/e 494 (M+H). ¹H NMR (CDCl₃, 300 MHz): δ 7.83 (m, 2H), 7.58-7.35 (m, 10H), 6.82 (s, 1H), 4.70-3.22 (m, 7H), 1.13 (br s, 3H).

### Example 132

### ((R)-4-{5-[3-(4-Chloro-benzoyl)-isoxazol-5-yl]-thiophene-2-sulfonyl}-3-methyl-piperazin-1-yl)-phenyl-methanone

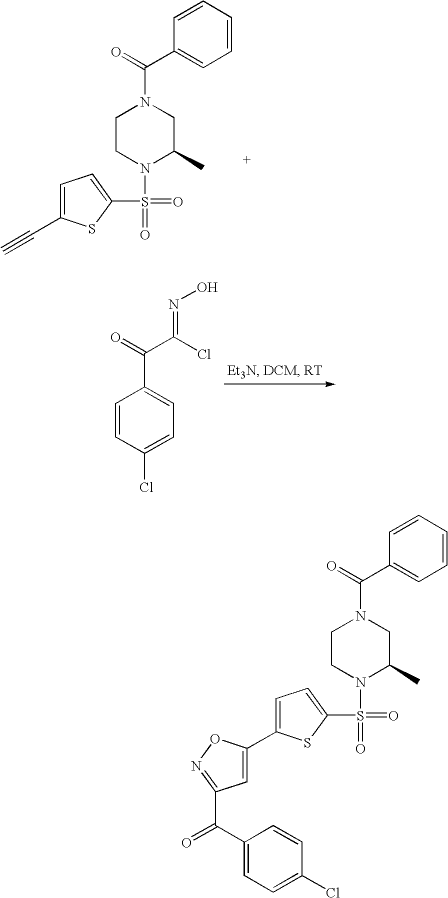

Prepared as described in the procedure of Example 131 above, and purified by HPLC. LCMS: m/e 556 (M+). ¹H NMR (CDCl₃, 300 MHz): δ 8.30 (d, J=8.5 Hz, 2H), 7.60-7.39 (m, 9H), 7.04 (s, 1H), 4.68-3.07 (m, 7H), 1.13 (brs, 3H).

### Example 133

### [(R)-3-methyl-4-(4-pyrazole-1-yl-benzenesulfonyl)-piperazin-1-yl]-phenyl-methanone

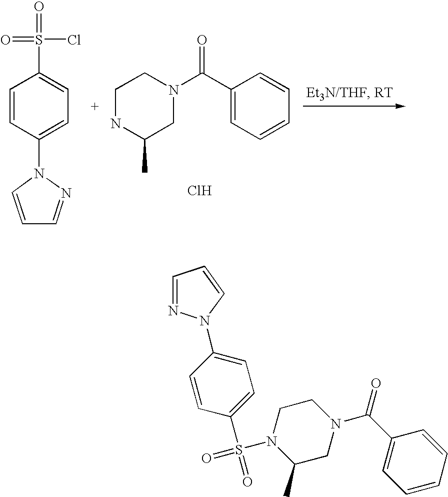

To a solution of phenyl-piperazine-1-yl-methanone hydrochloride (40 mg, 0.166 mmol) in 30% triethylamine/tetrahydrofuran (3 mL) was added 4-pyrazol-1-yl-benzenesulfonyl chloride (48 mg, 0.2 mmol) and mixture was stirred at 45° C. for 8 hours. Resulting white suspension was concentrated and the crude reaction mixture was purified by HPLC purification to afford [(R)-3-methyl-4-(4-pyrazole-1-yl-benzenesulfonyl)-piperazin-1-yl]-phenyl-methanone. LCMS: m/e 411 (M+H). ¹H NMR (CDCl₃, 300 MHz): δ7.91-7.85 (m, 4H), 7.75 (d, J=1.5 Hz, 1H), 7.43-7.32 (m, 5H), 7.13(d, J=2 Hz, 1H), 6.54 (t, 1.9 Hz, 1H), 3.6-3.16 (m, 7H), 1.03 (br s, 3H).

### Example 134

### [(R)-3-methyl-4-(4-[1,2,3]thiadiazol-4-yl-benzenesulfonyl)-piperazin-1-yl]-phenyl-methanone

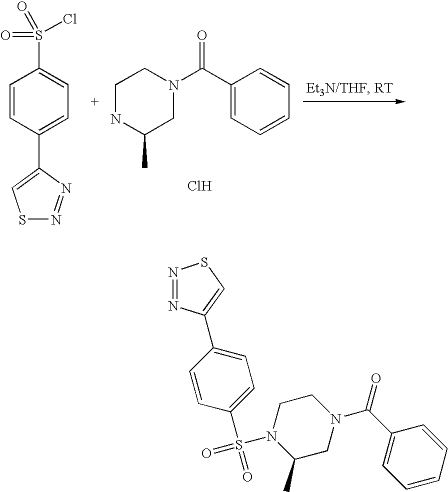

To a solution of phenyl-piperazine-1-yl-methanone hydrochloride (40 mg, 0.166 mmol) in 30% triethylamine/tetrahydrofuran (3 mL) was added 4-[1,2,3]thiadiazol-4-yl-benzenesulfonyl chloride(52 mg, 0.2 mmol) and mixture was stirred at 45° C. for 8 hours. Resulting white suspension was concentrated and the crude reaction mixture was purified by HPLC purification to afford [(R)-3-methyl-4-(4-[1,2,3]thiadiazol-4-yl-benzenesulfonyl)-piperazin-1-yl]-phenyl-methanone. LCMS: m/e 429 (M+H). ¹H NMR (CDCl₃, 300 MHz): d 8.80 (s, 1H), 8.22 (d, J=8.1 Hz, 2H), 7.95 (d, J=8.1 Hz, 2H), 7.43-7.32 (m, 5H), 4.6-2.69 (m, 7H), 1.05 (br s, 3H).

### General Procedure E for Coupling Reactions Between Aryl Halides and 5-(4-benzoyl-2-methylpiperazin-1-ylsulfonyl)thiophen-2-ylboronic acid

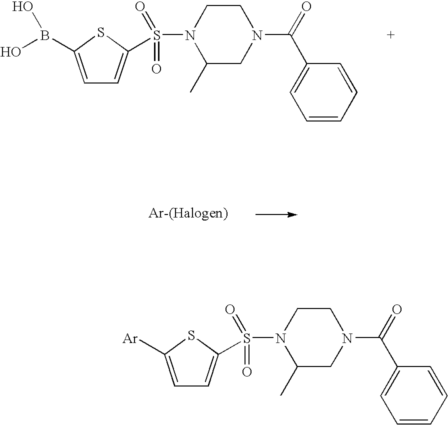

A solution of 100 mg of 5-(4-benzoyl-2-methylpiperazin-1-ylsulfonyl)thiophen-2-ylboronic acid, 63 mg of sodium bicarbonate, 7 mg of palladium acetate, 0.32 mmol of aryl halide and 25 mg of 2′-(dicyclohexylphosphino)-N,N-dimethylbiphenyl-2-amine in 2 mL of 4:1 ethylene glycol dimethyl ether/distilled water was heated to 40° C. under nitrogen. After stirring for 18 hours the mixture was diluted with 5 mL of ethyl acetate and 1 mL of water. The mixture was filtered and then the phases were separated. The solvent was evaporated from the organic phase at reduced pressure. The products geberated by General Procedure E, as listed in Table 1, were purified by reverse phase HPLC and, if necessary, further purified by silica gel chromatography. Typical yields of purified products were 5 to 15 mg.

TABLE 1

| Ex. | | | | Aryl Halide |
|---|---|---|---|---|
| No. | Structure | Name | Mass Spec | Used |
| 135 | 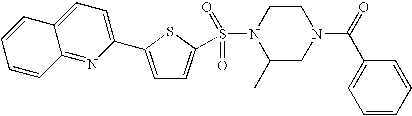 | (3-methyl-4-(5- (quinolin-2- yl)thiophen-2- ylsulfonyl)piperazin- 1- yl)(phenyl)methanone | Calc'd for C₂₅H₂₃N₃O₃S₂•H⁺: 478.1. Found: 478.4 | 2-chloro quinoline |
| 136 | 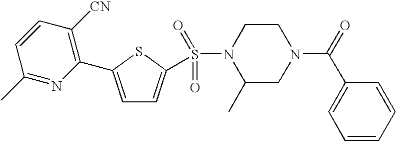 | 2-(5-(4-benzoyl-2- methylpiperazin-1- ylsulfonyl)thiophen- 2-yl)-6- methylnicotinonitrile | Calc'd for C₂₃H₂₂N₄O₃S₂•H⁺: 467.1. Found: 467.4 | 2-chloro-6- methylpyridine- 3-carbonitrile |
| 137 | 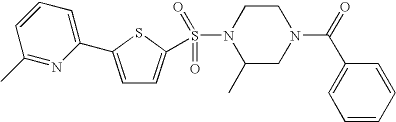 | (3-methyl-4-(5-(6- methylpyridin-2- yl)thiophen-2- ylsulfonyl)piperazin- 1- yl)(phenyl)methanone | Calc'd for C₂₂H₂₃N₃O₃S₂•H⁺: 442.1. Found: 442.0 | 2-bromo-6- methylpyridine |
| 138 | 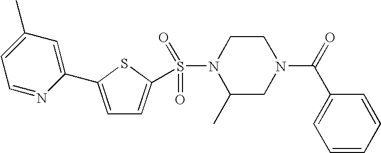 | (3-methyl-4-(5-(4- methylpyridin-2- yl)thiophen-2- ylsulfonyl)piperazin- 1- yl)(phenyl)methanone | Calc'd for C₂₂H₂₃N₃O₃S₂•H⁺: 442.1. Found: 442.3 | 2-bromo-4- methylpyridine |
| 139 | 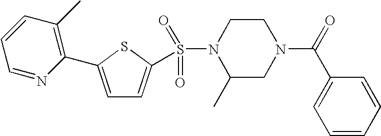 | (3-methyl-4-(5-(3- methylpyridin-2- yl)thiophen-2- ylsulfonyl)piperazin- 1- yl)(phenyl)methanone | Calc'd for C₂₂H₂₃N₃O₃S₂•H⁺: 442.1. Found: 442.4 | 2-bromo-3- methylpyridine |
| 140 | 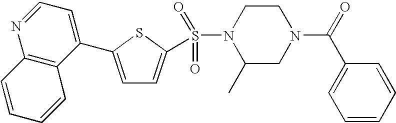 | (3-methyl-4-(5- (quinolin-4- yl)thiophen-2- ylsulfonyl)piperazin-1- yl)(phenyl)methanone | Calc'd for C₂₅H₂₃N₃O₃S₂•H⁺: 478.1. Found: 478.1 | 4-chloro quinoline |
| 141 | 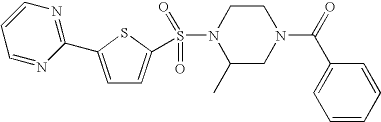 | (3-methyl-4-(5- (pyrimidin-2- yl)thiophen-2- ylsulfonyl)piperazin-1- yl)(phenyl)methanone | Calc'd for C₂₀H₂₀N₄O₃S₂•H⁺: 429.1. Found: 429.3 | 2-bromo pyrimidine |
| 142 | 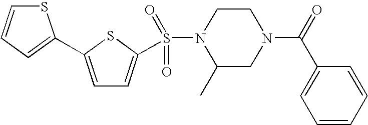 | (4-(2,2′-bithiophen-5- ylsulfonyl)-3- methylpiperazin-1- yl)(phenyl)methanone | Calc'd for C₂₀H₂₀N₂O₃S₃•H⁺: 433.1. Found: 433.3 | 2-bromo thiophene |
| 143 | 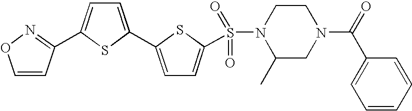 | (4-(5′-(isoxazol-3-yl)- 2,2′-bithiophen-5- ylsulfonyl)-3- methylpiperazin-1- yl)(phenyl)methanone | Calc'd for C₂₃H₂₁N₃O₄S₃•H⁺: 500.1. Found: 500.5 | 3-(5-bromo thiophen-2- yl)isoxazole |
| 144 | 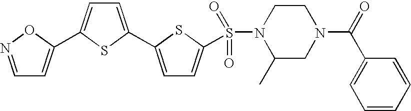 | (4-(5′-(isoxazol-5-yl)- 2,2′-bithiophen-5- ylsulfonyl)-3- methylpiperazin-1- yl)(phenyl)methanone | Calc'd for C₂₃H₂₁N₃O₄S₃•H⁺: 500.1. Found: 500.4 | 5-(5-bromo thiophen-2- yl)isoxazole |
| 145 | 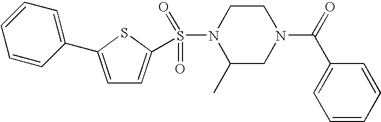 | (3-methyl-4-(5- phenylthiophen-2- ylsulfonyl)piperazin-1- yl)(phenyl)methanone | Calc'd for C₂₂H₂₂N₂O₃S₂•H⁺: 427.1. Found: 427.4 | Bromo benzene |
| 146 | 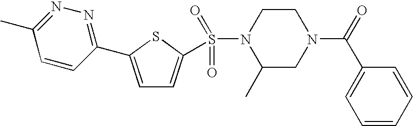 | (3-methyl-4-(5-(6- methylpyridazin-3- yl)thiophen-2- ylsulfonyl)piperazin-1- yl)(phenyl)methanone | Calc'd for C₂₁H₂₂N₄O₃S₂•H⁺: 443.1. Found: 443.4 | 3-chloro-6- methyl pyridazine |
| 147 | 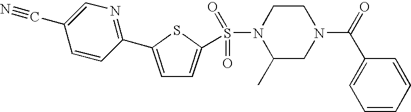 | (6-(5-(4-benzoyl-2- methylpiperazin-1- ylsulfonyl)thiophen-2- yl)nicotinonitrile | Calc'd for C₂₂H₂₃N₃O₃S₂•H⁺: 453.1. Found: 453.1 | 2-bromo-5- cyanopyridine |
| 148 | 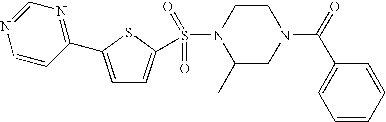 | (3-methyl-4-(5- (pyrimidin-4- yl)thiophen-2- ylsulfonyl)piperazin-1- yl)(phenyl)methanone | Calc'd for C₂₀H₂₀N₄O₃S₂•H⁺: 429.1. Found: 429.1 | 6-bromo pyrimidine |
| 149 | 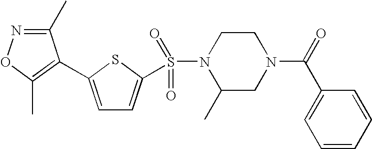 | (4-(5-(3,5- dimethyl isoxazol-4- yl)thiophen-2- ylsulfonyl)-3- methylpiperazin-1- yl)(phenyl)methanone | Calc'd for C₂₁H₂₃N₃O₄S₂•H⁺: 446.1. Found: 446.5 | 4-iodo-3,5- dimethyl isoxazole |
| 150 | 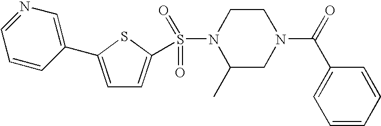 | (3-methyl-4-(5- (pyridin-3-yl)thiophen- 2-ylsulfonyl)piperazin- 1- yl)(phenyl)methanone | Calc'd for C₂₁H₂₁N₃O₃S₂•H⁺: 428.1. Found: 428.3. | 3-bromo pyridine |
| 151 | 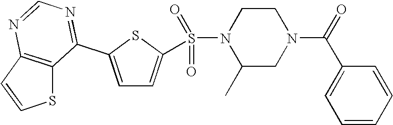 | (3-methyl-4-(5- (thieno[3,2- d]pyrimidin-4- yl)thiophen-2- ylsulfonyl)piperazin-1- yl)(phenyl)methanone | Calc'd for C₂₂H₂₀N₄O₃S₃•H⁺: 485.1 Found: 485.3 | 4-chloro thieno[3,2- d]pyrimidine |
| 152 | 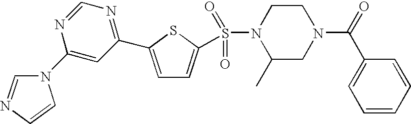 | (4-(5-(6-(1H-imidazol- 1-yl)pyrimidin-4- yl)thiophen-2- ylsulfonyl)-3- methylpiperazin-1- yl)(phenyl)methanone | Calc'd for C₂₃H₂₂N₆O₃S₂•H⁺: 495.1 Found: 495.4 | 4-chloro-6- (1H-imidazol- 1-yl)pyrimidine |
| 153 | 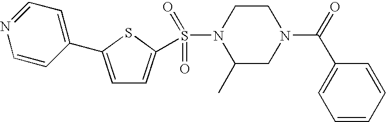 | (3-methyl-4-(5- (pyridin-4-yl)thiophen- 2-ylsulfonyl)piperazin- 1- yl)(phenyl)methanone | Calc'd for C₂₁H₂₁N₃O₃S₂•H⁺: 428.1. Found: 428.3. | 4-bromo pyridine hydrochloride |
| 154 | 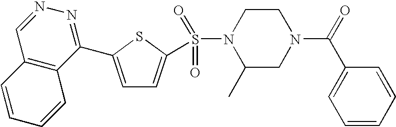 | (3-methyl-4-(5- (phthalazin-1- yl)thiophen-2- ylsulfonyl)piperazin-1- yl)(phenyl)methanone | Calc'd for C₂₄H₂₂N₄O₃S₂•H⁺: 479.1. Found: 479.4. | 2-chloro quinoxaline |
| 155 | 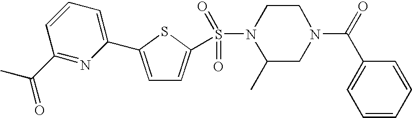 | 1-(6-(5-(4-benzoyl-2- methylpiperazin-1- ylsulfonyl)thiophen-2- yl)pyridin-2- yl)ethanone | Calc'd for C₂₃H₂₃N₃O₄S₂•H⁺: 470.1. Found: 470.5 | 2-acetyl-6- bromopyridine |
| 156 | 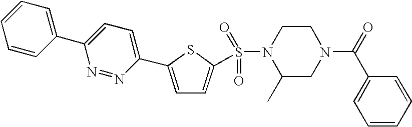 | (3-methyl-4-(5-(6- phenylpyridazin-3- yl)thiophen-2- ylsulfonyl)piperazin-1- yl)(phenyl)methanone | Calc'd for C₂₆H₂₄N₄O₃S₂•H⁺: 505.1 Found: 505.5. | 3-chloro-6- phenyl pyridazine |
| 157 | 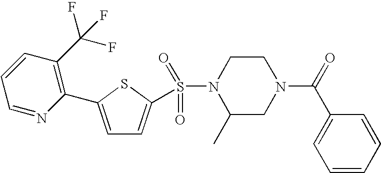 | (3-methyl-4-(5-(3- (trifluoro- methyl)pyridin-2- yl)thiophen-2- ylsulfonyl)piperazin-1- yl)(phenyl)methanone | Calc'd for C₂₂H₂₀F₃N₃O₃S₂•H⁺: 496.1 Found: 496.4 | 2-chloro-3- (trifluoromethyl)pyridine |
| 158 | 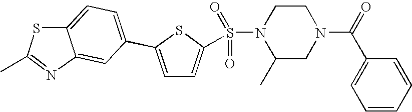 | (3-methyl-4-(5-(2- methylbenzo[d]thiazol- 5-yl)thiophen-2- ylsulfonyl)piperazin-1- yl)(phenyl)methanone | Calc'd for C₂₄H₂₃N₃O₃S₃•H⁺: 498.1 Found: 498.4 | 5-bromo-2- methyl benzothiazole |

### 5-(4-benzoyl-2-methylpiperazin-1-ylsulfonyl)thiophen-2-ylboronic acid

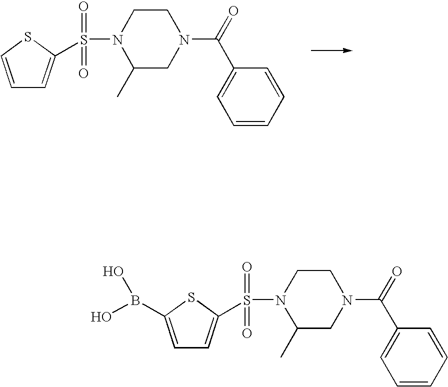

(3-Methyl-4-(thiophen-2-ylsulfonyl)piperazin-1-yl)(phenyl)methanone was prepared by the same procedure used in Example 133, and was used in this preparation without characterization. A −78° C. solution of 6.3 mL of diisopropylamine in 50 mL of THF was treated with 16 mL of 2.5 M butyllithium in hexanes over 5 minutes. The mixture was allowed to warm to room temperature, and 24 mL of the resulting solution was immediately added dropwise to a −78° C. solution of 4.0 grams of (3-methyl-4-(thiophen-2-ylsulfonyl)piperazin-1-yl)(phenyl)methanone and 8.0 mL of triisopropyl borate in 100 mL of THF. After stirring for 2 hours at −78° C., an addition 5 mL of the lithium diisopropylamine solution was added dropwise. The mixture was stirred 2 more hours. A solution prepared by mixture 25 mL of 3 N hydrochloric acid and 150 mL of methanol was then added to the cold solution. The mixture was warmed to 25° C. and then it was partitioned between ethyl acetate and dilute aqueous sodium chloride solution. The aqueous phase was washed with ethyl acetate three times and the solvent was evaporated from the combined organic extracts at reduced pressure. The procedure yielded 4.3 g of a white foam. MS: calc'd for C₁₆H₁₉BN₂O₅S₂ H⁺: m/z=395.1. Found: 395.4.

### General Procedure F for Coupling Reactions Between Acyl Chlorides and (3-methyl-4-(4-(trimethylstannyl)phenylsulfonyl)piperazin-1-yl)(phenyl)methanone

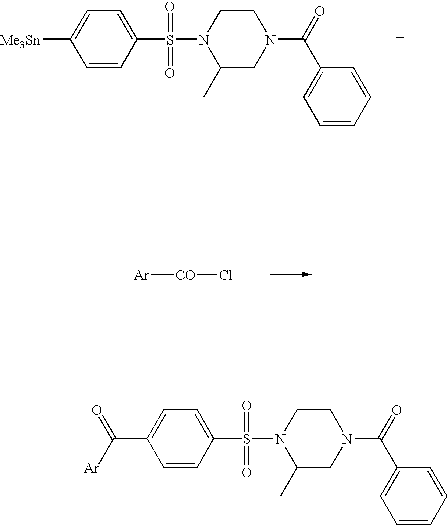

A mixture of 75 mg of (3-methyl-4-(4-(trimethylstannyl)phenylsulfonyl)piperazin-1-yl)(phenyl)methanone, 3.8 mg of (Ph₃P)₂PhCH₂Pd Cl, and 0.17 mmol of acid chloride in 1 mL of chloroform was heated to 85° C. in a sealed vial for 12 hours. The mixture was filtered. The solvent was evaporated and the residue was dissolved in 3 mL of THF. The resulting solution was treated with 0.2 mL of 3 N aqueous NaOH solution and stirred for 1 hour. The solvent was evaporated at reduced pressure and the residue was partitioned between water and ethyl acetate. The organic phase was dried over magnesium sulfate, and filtered. The solvent was evaporated at reduced pressure and the residue was purified by reverse-phase chromatography. Products generated by General Procedure F are listed in Table 2.

TABLE 2

| | | | | Acid |
|---|---|---|---|---|
| Example | | | | Chloride |
| No. | Structure | Name | Mass Spec | Used |
| 159 | 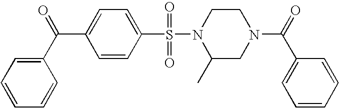 | (4-(4-benzoyl-2- methylpiperazin-1- ylsulfonyl)phenyl)(phenyl)methanone | Calc'd for C₂₅H₂₄N₂O₄S•H⁺: 449.1. Found: 449.4 | Benzoyl chloride |
| 160 | 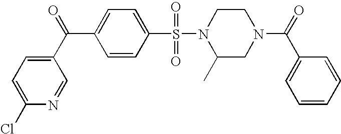 | (4-(4-benzoyl-2- methylpiperazin-1- ylsulfonyl)phenyl)(6- chloropyridin-3- yl)methanone | Calc'd for C₂₄₂H₂₃N₃O₄S•H⁺: 484.1. Found: 484.4 | 6- chloronicotinoyl chloride |
| 161 | 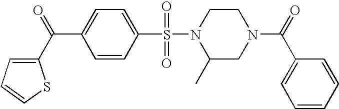 | (4-(4-benzoyl-2- methylpiperazin-1- ylsulfonyl)phenyl)(thiophen- 2-yl)methanone | Calc'd for C₂₃H₂₂N₂O₄S₂•H⁺: 455.1. Found: 455.1.0 | 2-thiophene- carbonyl chloride |
| 162 | 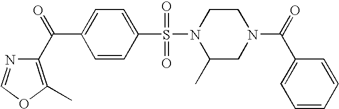 | (4-(4-benzoyl-2- methylpiperazin-1- ylsulfonyl)phenyl)(5- methyloxazol-4- yl)methanone | Calc'd for C₂₃H₂₃N₃O₅S•H⁺: 454.1. Found: 454.1 | 5-methyl-4- oxazolecarbonyl chloride |
| 163 | 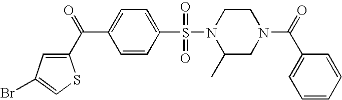 | (4-(4-benzoyl-2- methylpiperazin-1- ylsulfonyl)phenyl)(4- bromothiophen-2- yl)methanone | Calc'd for C₂₃H₂₁BrN₂O₄S₂•H⁺: 533.0 Found: 533.3 | 4-bromo-2- thiophene- carbonyl chloride |

### (R)-(4-(4-iodophenylsulfonyl)-3-methylpiperazin-1-yl)(phenyl)methanone

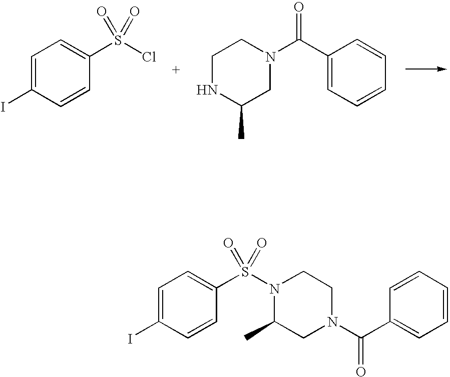

A 0° C. solution of 9.5 grams of (R)-1-benzoyl-2-methylpiperazine hydrochloride and 22 mL of Hunig's base in 300 mL of dichloromethane was treated with 12 grams of 4-iodobenzenesulfonyl chloride. The mixture was allowed to warm to 25° C. and stirred overnight. The mixture was washed with excess aqueous sodium carbonate and dried over magnesium sulfate. The solvent was evaporated and the residue was dissolved in a minimum volume of ethyl acetate. Several volumes of ethyl ether was added and the resulting precipitate was collected by filtration. ¹H NMR (CDCl₃, 300 MHz): δ 7.9 (d, J=7 Hz, 2H), 7.5 (d, J=7 Hz, 2H), 7.5-7.3 (m, 5H), 4.8-2.7 (broad m, 7H), 1.2-0.8 (broad multiplet, 3H).

### (3-methyl-4-(4-(trimethylstannyl)phenylsulfonyl)piperazin-1-yl)(phenyl)methanone

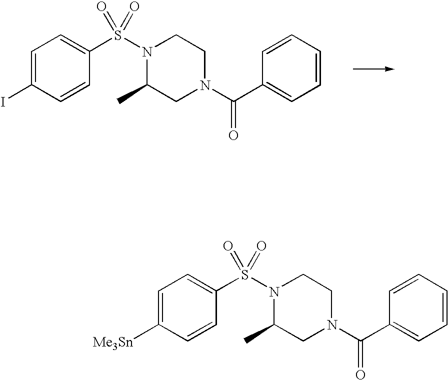

A mixture of 1.4 g (3.0 mmo)l of (R)-(4-(4-iodophenylsulfonyl)-3-methylpiperazin-1-yl)(phenyl)methanone, 2.6 grams (4.5 mmol) of hexamethylditin, and 0.22 grams of PhPdCl(Ph₃P)₂ in 15 mL of dioxane was heated at 60° C. for 4 days. The solvent was evaporated and the residue was chromatographed on silica gel eluting with a mixture of dichlormethane and hexanes. The product was obtained as a colorless oil. ¹H NMR (CDCl₃, 300 MHz): δ 7.7 (d, J=7 Hz, 2H), 7.6 (d, J=7 Hz, 2H), 7.5-7.3 (m, 5H), 4.8-2.7 (broad m, 7H), 1.2-0.8 (broad multiplet, 3H), 0.35 (s, 9H). Side peaks of about 10% intensity are visible on either side of the 0.35 ppm singlet due to proton-tin coupling with minor isotopes of tin.

### Example 164

### [4-(5-Isoxazol-3-yl-thiophene-2-sulfonyl)-3-methyl-piperazin-1-yl]-phenyl-methanone

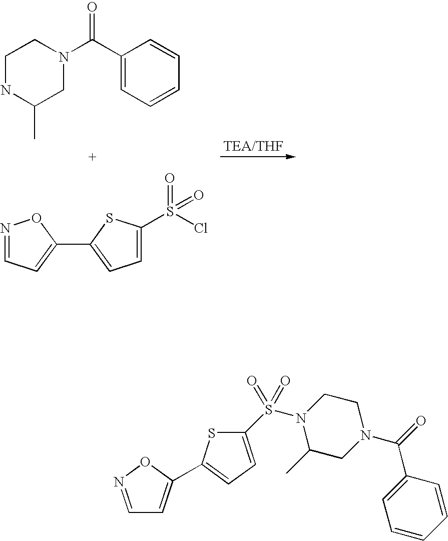

(3-methyl-piperazin-1-yl)-phenyl-methanone (20 mg, 0.1mmol) and 5-isoxazol-3-yl-thiophene-2-sulfonyl chloride (25 mg, 0.1 mmol) were each dissolved in 1 mL of a solvent consisting of 30% triethylamine in anhydrous THF. The two solutions were combined and the resulting mixture stirred overnight at room temperature. The volatiles were removed under vacuum and the remaining crude product was purified by reverse-phase HPLC. Yield: 5.1 mg (12%). The purified material was characterized by LC-MS (95% purity by ELSD). MS: Calculated for C₁₉H₁₉N₃O₄S₂.H⁺: 418.08. Found: 417.9.

### Example 165

### [(R)-3-Methyl-4-(5-[1,2,3]thiadiazol-4-yl-thiophene-2-sulfonyl)-piperazin-1-yl]-phenyl-methanone

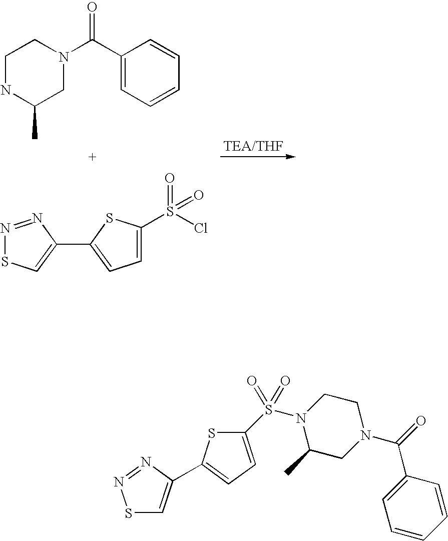

((R)-3-Methyl-piperazin-1-yl)-phenyl-methanone (75 mg, 0.375 mmol) was dissolved in 5 mL of 30% triethylamine in THF. 5-[1,2,3]thiadiazol-4-yl-thiophene-2-sulfonyl chloride (100 mg, 0.375 mmol) was then added directly with stirring. The mixture was stirred for 2 to 3 hours at room temperature. Precipitated salts were removed via vacuum filtration and the filtrate concentrated under vacuum. The remaining residue was partitioned between EtOAc and aqueous ammonium chloride. The organic phase was washed once additionally with ammonium chloride, once with brine, dried over MgSO₄, and then concentrated to afford 155 mg of a brown residue that was further purified by HPLC. Isolated yield: 27 mg (17%) The purified material was characterized by LC-MS (98.6% purity by ELSD). MS: Calculated for C₁₈H₁₈N₄O₃S₃.H⁺: 435.05. Found: 435.4. ¹HNMR (CDCl₃): 8.67 (s, 1H), 7.61 (d, J=3.8 Hz, 1H), 7.58 (d, J=3.9 Hz, 1H) 7.43-7.31 (m, 5H), 4.8-2.9 (br. m, 7H), 1.21 (br. s, 3H).

### Example 166

### [3-Methyl-4-(4-oxazol-5-yl-benzenesulfonyl)-piperazin-1-yl]-phenyl methanone

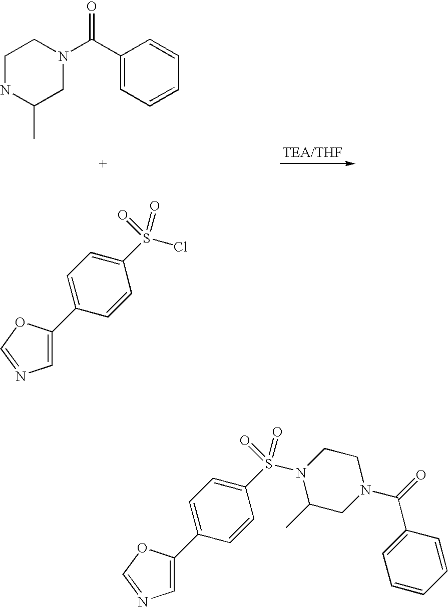

(3-methyl-piperazin-1-yl)-phenyl-methanone (20 mg, 0.1 mmol) and 4-oxazol-5-yl-benzenesulfonyl chloride (24 mg, 0.1 mmol) were each dissolved in 1 mL of a solvent consisting of 30% triethylamine in anhydrous THF. The two solutions were combined and the resulting mixture stirred overnight at room temperature. The volatiles were removed under vacuum and the remaining crude product was purified by reverse-phase HPLC. Yield: 9 mg (22%). The purified material was characterized by LC-MS (100% purity by ELSD). MS: Calculated for C₂₁H₂₁N₃O₄S.H⁺: 412.1. Found: 412.5.

### Example 167

### [(R)-3-Methyl-4-(5-[(5-trifluoromethyl)-isoxazol-3-yl]-thiophene-2-sulfonyl)-piperazin-1-yl]-phenyl-methanone

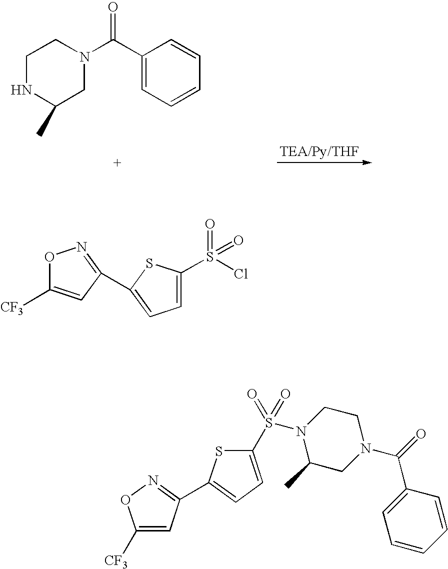

R-(3-methyl-piperazin-1-yl)-phenyl-methanone (51 mg, 0.25 mmol) was dissolved in 2 ml pyridine and 5-[(5-trifluoromethyl)-3-oxazol]-2-yl-thiophenesulfonyl chloride (79.47 mg, 0.25 mmol) was dissolved in 4 ml of THF. The two solutions were combined and the resulting mixture was added 2.5 mmol of triethylamine and then stirred overnight at room temperature. The volatiles were removed under vacuum and the remaining crude product was purified by reverse-phase HPLC. Yield: 18 mg (15%). The purified material was characterized by LC-MS MS: Calculated for C₂₁H₁₈N₃F₃O₄S₂: 485.07. Found: 485.00. purity via HPLC 99.2%.

### Example 168

### [(R)-3-Methyl-4-(5-pyridinyl-2-yl-thiophene-2-sulfonyl)-piperazin-1-yl]-phenyl-methanone

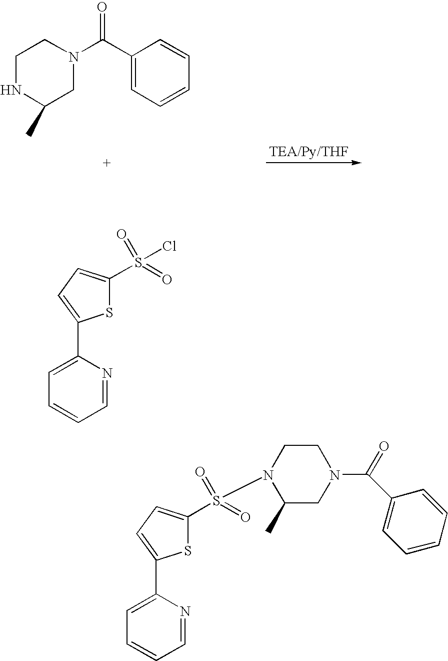

R-(3-methyl-piperazin-1-yl)-phenyl-methanone (51 mg, 0.25 mmol) was dissolved in 2 ml pyridine and 5-(pyridinyl-2-yl)-2-yl-thiophenesulfonyl chloride (65 mg, 0.25 mmol) was dissolved in 4 ml of THF. The two solutions were combined and the resulting mixture was added 2.5 mmol of triethylamine and then stirred overnight at room temperature. The volatiles were removed under vacuum and the remaining crude product was purified by reverse-phase HPLC. Yield: 21 mg (20%). The purified material was characterized by LC-MS MS: Calculated for C₂₁H₂₁N₃O₃S₂: 427.10. Found: 427.06. purity via HPLC 98.8%.

### Example 168

### 1-(4-Benzoyl-2-methyl-piperazin-1-yl)-2-[4-(2H-pyrazol-3-yl)-phenyl]-ethane-1,2-dione

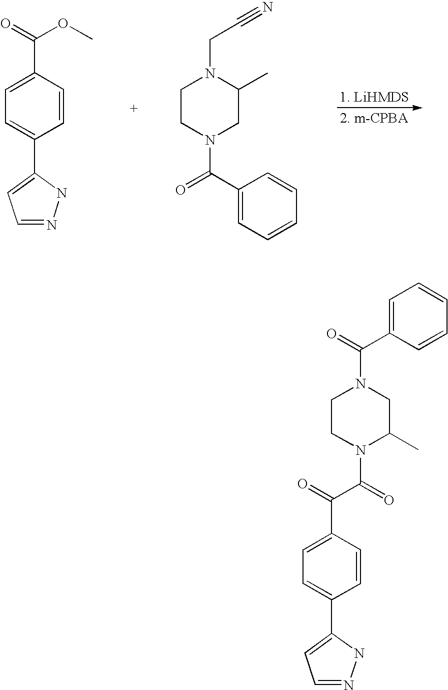

To a solution of 4-(2H-pyrazol-3-yl)-benzoic acid methyl ether (0.2 g, 1 mmol) and (4-benzoyl-2-methyl-piperazin-1-yl)-acetonitrile (0.24 g, 1 mmol) in anhydrous THF (2 mL) was added by drops 1.06 M solution LiHMDS in THF (4 mL, 4 mmol). The reaction mixture was stirred for 1 hour, and m-CPBA was added (0.67 g, 4 mmol). The mixture was stirred for 1 hour additionally, quenched with water, extracted with EtOAc, dried over sodium sulfate, filtered and evaporated. The residue was purified by SiO₂ chromatography using 10% MeOH/ethyl acetate as eluent to obtain 1-(4-benzoyl-2-methyl-piperazin-1-yl)-2-[4-(2H-pyrazol-3-yl)-phenyl]-ethane-1,2-dione (0.09 g, 22%). LCMS: Calc'd for C₂₃H₂₂N₄O₃.H⁺: m/z=403.5. Found: m/z=403, 404. ¹H NMR (DMSO-d₆): 13.59 and 13.16 (two br s, 1H, NH-pyrazole); 8.06 (m, 2H); 7.95-7.86 (m, 3H); 7.45-7.40 (m, 5H); 6.89 (br s, 1H); 4.68-4.30 (br signal, 2H); 3.29-3.43 (br signal, 2H); 3.16 (m, 1H); 3.08-2.85 (br signal, 2H); 1.20 (br signal, 3H).

### 4-(2H-Pyrazol-3-yl)-benzoic acid methyl ester

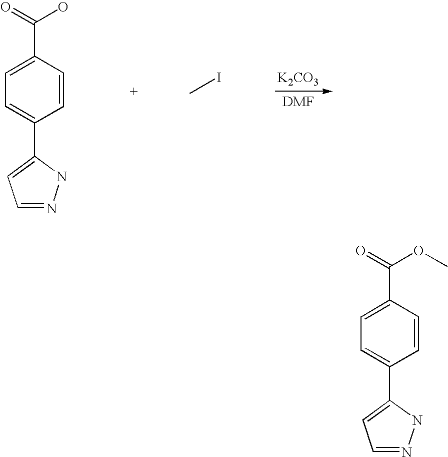

Mixture of 4-(2H-pyrazol-3-yl)-benzoic acid (0.30 g, 1.6 mmol), K₂CO₃ (0.66 g, 7.78 mmol) and methyl iodide (0.34 g, 2.4 mmole) in DMF (8 mL) was kept under stirring at RT temperature for 1 h (TLC monitoring, 10% MeOH/CHCl₃). The reaction mixture was diluted with water (30 mL), extracted with EtOAc (2×30 mL), organic layers combined, washed with water, brine, dried over sodium sulfate, filtered and evaporated to afford 4-(2H-pyrazol-3-yl)-benzoic acid methyl ether (0.31 g, 96%). ¹H NMR (DMSO-d₆): 13.10 (br s, 1H); 7.98 (m, 4H); 7.80 (br signal, 1H); 6.83 (d, J=2.2 Hz, 1H); 3.86 (s, 3H).

### 4-(1-Methyl-1H-pyrazol-3-yl)-benzoic acid methyl ester

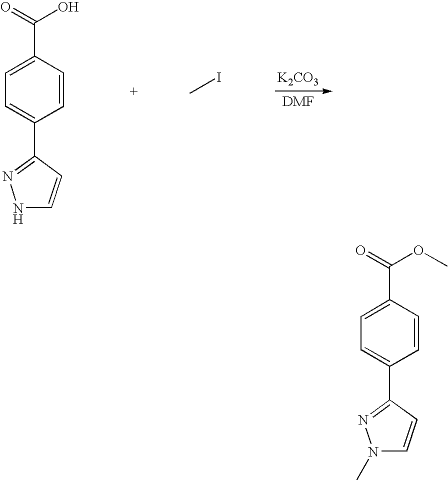

Mixture of 4-(2H-pyrazol-3-yl)-benzoic acid (0.30 g, 1.6 mmol), K₂CO3 (0.66 g, 7.78 mmol) and methyl iodide (0.68 g, 4.8 mmole) in DMF (8 mL) was kept under stirring at RT temperature for 24 h (LC/MS monitoring). The reaction mixture was diluted with water (30 mL), extracted with EtOAc (2×30 mL), organic layers combined, washed with water, brine, dried over sodium sulfate, filtered and evaporated to afford 4-(1-methyl-2H-pyrazol-3-yl)-benzoic acid methyl ether (0.28 g, 80%). LCMS: Calc'd for C₁₂H₁₂N₂O₂.H⁺: m/z=217.24. Found: m/z=217, 218.

### Example 170

### 1-(4-Benzoyl-2-methyl-piperazin-1-yl)-2-(4-[1,2,4]triazol-1-yl-phenyl)-ethane-1,2-dione

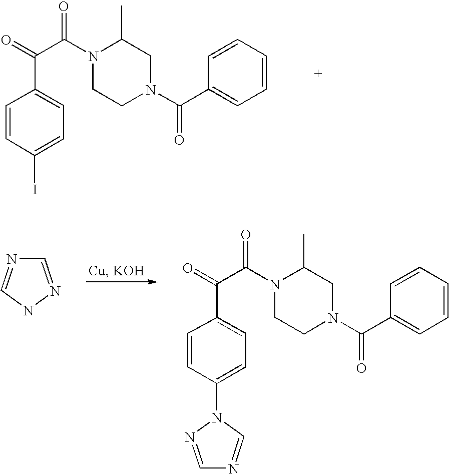

Mixture of 1-(4-benzoyl-2-methyl-piperazin-1-yl)-2-(4-iodo-phenyl)-ethane-1,2-dione (0.33 g, 0.71 mmol), Cu-powder (0.09 g, 1.42 mmole), 1,2,4-triazole (1.47 mL, 21.3 mmole), and powdered KOH (0.08 g, 1.42 mmole) was heated to 160° C. and kept under stirring at this temperature for 24 h (TLC monitoring, 10% MeOH/CHCl₃). The reaction mixture was then cooled to RT, diluted with EtOAc (˜2 mL), placed onto a silica gel column and eluted with EtOAc, and evaporated to afford 1-(4-benzoyl-2-methyl-piperazin-1-yl)-2-(4-[1,2,4]triazol-1-yl-phenyl)-ethane-1,2-dione (0.17 g, 59%). LCMS: Calc'd for C₂₂H₂₁N₅O₃.H⁺: m/z=404.5. Found: m/z=404, 405. ¹H NMR (DMSO-d₆): 9.50 (d, J=3.1 Hz, 1H); 8.34 (s, 1H); 8.14-7.98 (m, 4H); 7.45-7.42 (m, 5H); 4.90-4.10 (br signal, 2H); 3.95-3.35 (br signal, 3H); 3.23-2.80 (br signal, 2H); 1.20 (br signal, 3H).

### 1-(4-Benzoyl-2-methyl-piperazin-1-yl)-2-(4-iodo-phenyl)-ethane-1,2-dione

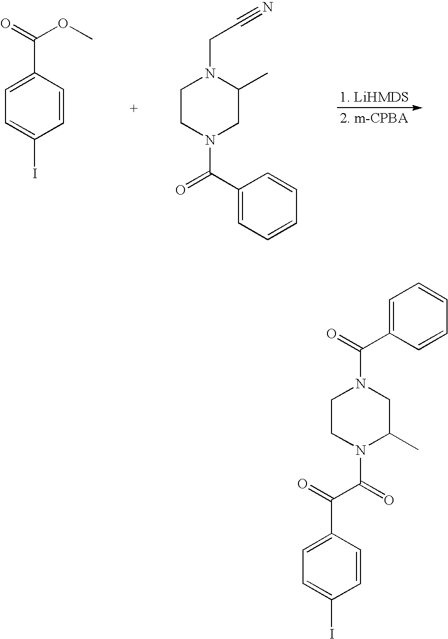

1-(4-Benzoyl-2-methyl-piperazin-1-yl)-2-(4-iodo-phenyl)-ethane-1,2-dione (35%) was prepared according to the procedure above, using 4-iodo-benzoic acid methyl ester. ¹H NMR (DMSO-d₆): 8.03 (m, 2H); 7.46 (m, 2H); 7.46-7.42 (m, 5H); 4.84-4.03 (br signal, 2H); 3.99-3.34 (br signal, 3H); 3.23-2.79 (broad signal, 2H); 1.21 (br signal, 3H).

### Example 171

### 1-(4-Benzoyl-2-methyl-piperazin-1-yl)-2-(4-furan-2-yl-phenyl)-ethane-1,2-dione

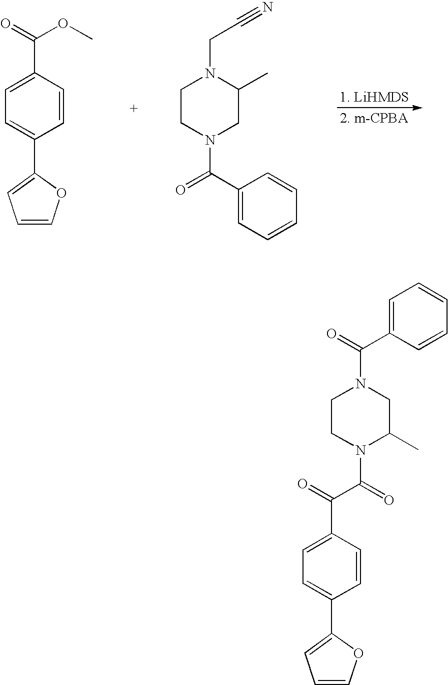

1-(4-Benzoyl-2-methyl-piperazin-1-yl)-2-(4-furan-2-yl-phenyl)-ethane-1,2-dione (0.02 g, 20%) was prepared according to the procedure above, using 4-furan-2-yl-benzoic acid methyl ester. The product was purified by reverse phase preparative HPLC. LCMS: Calc'd for C₂₄H₂₂N₂O₄.H⁺: m/z=403.5. Found: m/z=403, 404. ¹H NMR (DMSO-d₆): 7.95-7.89 (m, 5H); 7.45-7.40 (m, 5H); 7.25 (m, 1H); 6.69 (m, 1H); 4.82-4.18 (br signal, 2H); 4.15-3.40 (br signal, 3H); 3.19-2.86 (broad signal, 2H); 1.22 (br signal, 3H).

### Example 172

### 1-(4-Benzoyl-2-methyl-piperazin-1-yl)-2-[4-(1-methyl-1H-pyrazol-3-yl)-phenyl]-ethane-1,2-dione

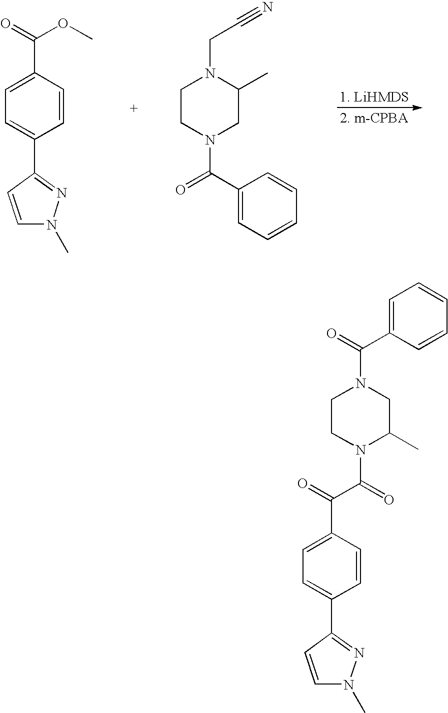

1-(4-Benzoyl-2-methyl-piperazin-1-yl)-2-[4-(1-methyl-1H-pyrazol-3-yl)-phenyl]-ethane-1,2-dione (37%) was prepared according to the procedure above, using 4-(1-methyl-1H-pyrazol-3-yl)-benzoic acid methyl ester. LCMS: Calc'd for C₂₄H₂₄N₄O₃.H⁺: m/z=417.5. Found: m/z=417, 418. ¹H NMR (DMSO-d₆): 8.02 (m, 2H); 7.94-7.88 (m, 2H); 7.81 (m, 1H); 7.45-7.40 (m, 5H); 6.87 (m, 1H); 4.84-4.18 (br signal, 2H); 3.92 (s, 3H); 3.90-2.85 (br signal, 5H); 1.20 (br signal, 3H).

### Example 173

### 1-(4-Benzoyl-2-methyl-piperazin-1-yl)-2-[4-(5-methyl-furan-2-yl)-phenyl]-ethane-1,2-dione

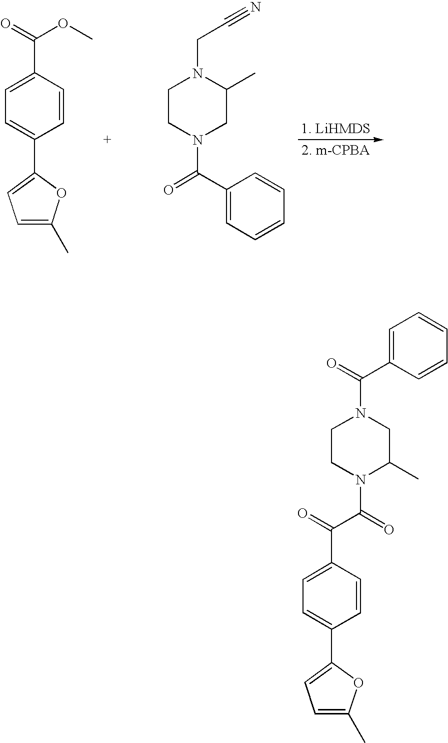

1-(4-Benzoyl-2-methyl-piperazin-1-yl)-2-[4-(5-methyl-furan-2-yl)-phenyl]-ethane-1,2-dione (19%) was prepared according to the procedure above, using 4-(5-methyl-furan-2-yl)-benzoic acid methyl ester. The product was purified by reverse phase preparative HPLC. LCMS: Calc'd for C₂₅H₂₄N₂O₄.H⁺: m/z=417.5. Found: m/z=417, 418. ¹H NMR (DMSO-d₆): 7.93-7.83 (m, 4H); 7.45-7.40 (m, 5H); 7.13 (m, 1H); 6.31 (m, 1H); 4.80-4.18 (br signal, 2H); 4.15-3.39 (br signal, 3H); 3.19-2.97 (broad signal, 2H); 2.88 (s, 3H); 1.22 (br signal, 3H).

### 1-((R)-4-Benzoyl-2-methyl-piperazin-1-yl)-2-(4-bromo-phenyl)-ethane-1,2-dione

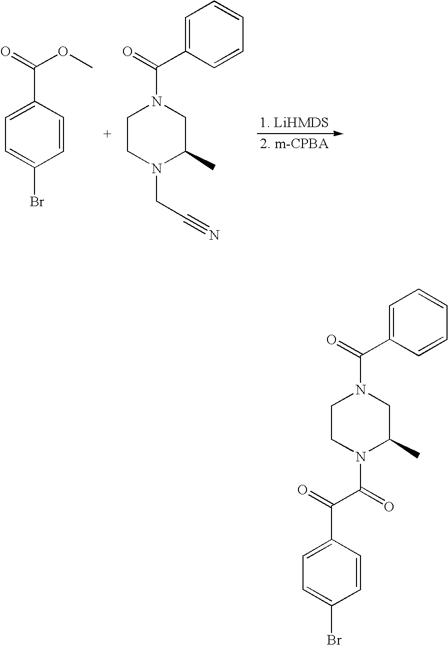

1-((R)-4-Benzoyl-2-methyl-piperazin-1-yl)-2-(4-bromo-phenyl)-ethane-1,2-dione (31%) was prepared according to the procedure above, using 4-bromo-benzoic acid methyl ester and ((R)-4-benzoyl-2-methyl-piperazin-1-yl)-acetonitrile. ¹H NMR (DMSO-d₆): 7.85 (m, 4H); 7.46-7.42 (m, 5H), 4.88-4.09 (br signal, 2H); 3.97-3.35 (br signal, 3H); 3.20-2.77 (broad signal, 2H); 1.18 (br signal, 3H).

### 1-((R)-4-Benzoyl-2-methyl-piperazin-1-yl)-2-(4-bromo-2-fluoro-phenyl)-ethane-1,2-dione

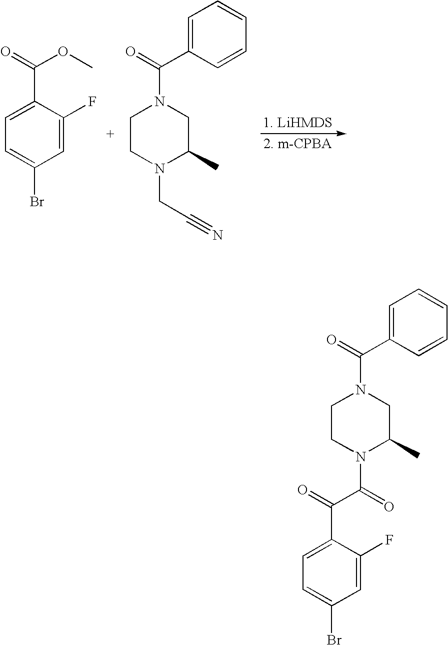

1-((R)-4-Benzoyl-2-methyl-piperazin-1-yl)-2-(4-bromo-2-fluoro-phenyl)-ethane-1,2-dione (25%) was prepared according to the procedure above, using 4-bromo-2-fluoro-benzoic acid methyl ester and ((R)-4-benzoyl-2-methyl-piperazin-1-yl)-acetonitrile. ¹H NMR (DMSO-d₆): 7.86-7.81 (m, 2H); 7.68 (m, 1H); 7.46-7.42 (m, 5H); 4.74-3.41 (br signal, 5H); 3.19-2.80 (broad signal, 2H); 1.18 (br signal, 3H).

### Example 174

### 1-(4-Benzoyl-2-methyl-piperazin-1-yl)-2-(4-[1,2,3]triazol-2-yl-phenyl)-ethane-1,2-dione

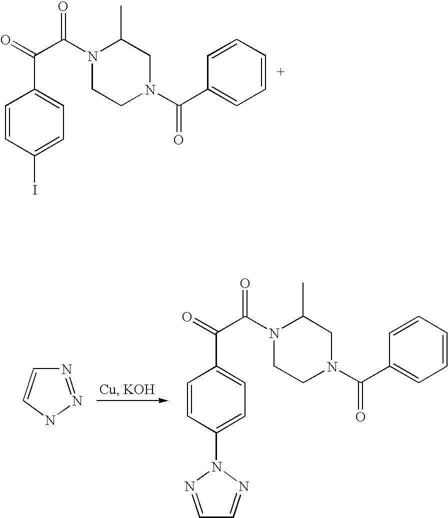

1-(4-Benzoyl-2-methyl-piperazin-1-yl)-2-(4-[1,2,3]triazol-2-yl-phenyl)-ethane-1,2-dione (0.17 g, 59%) was prepared according to the procedure above, using 1,2,3-triazole instead of 1,2,4-triazole and 2% MeOH/CH₂Cl₂ as eluent. LCMS: Calc'd for C₂₂H₂₁N₅O₃.H⁺: m/z=404.5. Found: m/z=404, 405. ¹H NMR (DMSO-d₆): 8.25 (m+s, 2H+2H); 8.10 (m, 2H); 7.45 (m, 5H); 4.80-4.10 (br signal, 2H); 3.90-3.30 (br signal, 3H); 3.23-2.82 (br signal, 2H); 1.19 (br signal, 3H).

### Example 181

### 1-((R)-4-Benzoyl-2-methyl-piperazin-1-yl)-2-[2-methyl-4-(1H-pyrazol-3-yl)-phenyl]-ethane-1,2-dione

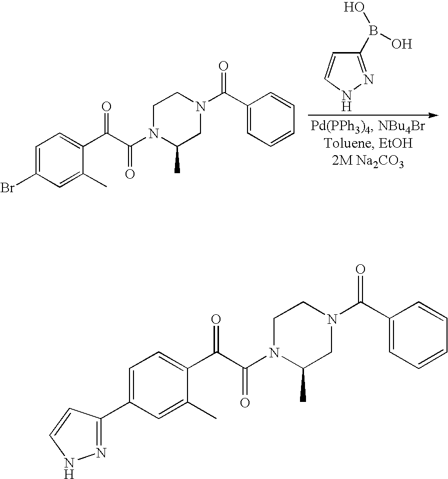

Toluene (10 mL), EtOH (10 mL) and 2M Na₂CO₃ (1.15 mL) were added to a mixture of 1-((R)-4-benzoyl-2-methyl-piperazin-1-yl)-2-(4-bromo-2-methyl-phenyl)-ethane-1,2-dione (0.3 g, 0.7 mmol), pyrazoleboronic acid (of ˜88% purity from stock, 0.117 g, 1.05 mmol) and NBu₄Br (0.02 g). Argon was bubbled within reaction mixture for ˜20 min. Then Pd(PPh₃)₄ was added (0.042 g, 0.036 mmol). The reaction mixture was stirring under reflux (20 min; TLC-monitoring, CHCl₃/MeOH 9:1), then cooled to RT and diluted with water (20 mL). The product was extracted with ethylacetate (2×50 mL). Organic solution was dried with Na₂SO₄, evaporated, and the residue was purified by chromatography (silica gel, 10% MeOH/CHCl₃). Solvent was evaporated to give product 1-((R)-4-benzoyl-2-methyl-piperazin-1-yl)-2-[2-methyl-4-(1H-pyrazol-3-yl)-phenyl]-ethane-1,2-dione (0.20 g, 68%). LCMS: Calc'd for C₂₄H₂₄N₄O₃.H⁺: m/z=417.5. Found: m/z=417, 418. ¹H NMR (DMSO-d₆): 13.58, 13.12 (two broad signals, 1H, NH-pyrazole); 7.87 (m, 3H); 7.78-7.69 (m, 1H); 7.45 (m, 5H); 6.88 (m, 1H); 4.88-4.10 (br signal, 2H); 4.06-3.34 (br signal, 3H); 3.19-2.82 (br signal, 2H); 2.64 (m, 3H); 1.22 (br signal, 3H).

### Example 184

### 1-((R)-4-Benzoyl-2-methyl-piperazin-1-yl)-2-[3-methyl-4-(1H-pyrazol-3-yl)-phenyl]-ethane-1,2-dione

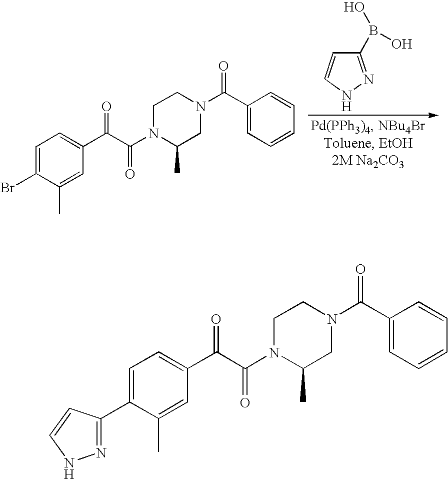

1-((R)-4-Benzoyl-2-methyl-piperazin-1-yl)-2-[3-methyl-4-(1H-pyrazol-3-yl)-phenyl]-ethane-1,2-dione (55%) was prepared according to the procedure above (2 h 30 min; TLC-monitoring, CHCl₃/MeOH 9:1), using 1-((R)-4-benzoyl-2-methyl-piperazin-1-yl)-2-(4-bromo-3-methyl-phenyl)-ethane-1,2-dione. LCMS: Calc'd for C₂₄H₂₄N₄O₃.H⁺: m/z=417.5. Found: m/z=417, 418. ¹H NMR (DMSO-d₆): 13.26, 13.16 (two broad signals, 1H, NH-pyrazole); 7.87-7.72 (m, 4H); 7.45 (m, 5H); 6.70, 6.61 (two m, 1H); 4.88-4.07 (br signal, 2H); 4.03-3.34 (br signal, 3H); 3.21-2.82 (br signal, 2H); 2.59 (s, 3H); 1.21 (br signal, 3H).

### 1-((R)-4-Benzoyl-2-methyl-piperazin-1-yl)-2-(4-bromo-3-methyl-phenyl)-ethane-1,2-dione

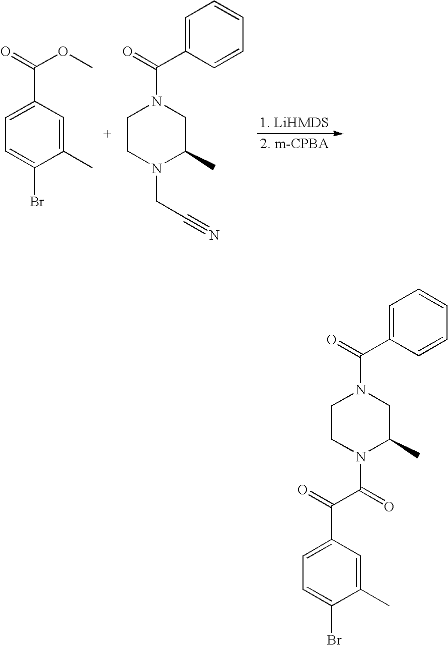

1-((R)-4-Benzoyl-2-methyl-piperazin-1-yl)-2-(4-bromo-3-methyl-phenyl)-ethane-1,2-dione (49%) was prepared according to the procedure above, using 4-bromo-3-methyl-benzoic acid methyl ester and ((R)-4-benzoyl-2-methyl-piperazin-1-yl)-acetonitrile. ¹H NMR (DMSO-d₆): 7.86-7.82 (m, 2H); 7.66-7.58 (m, 1H); 7.45 (m, 5H); 4.86-4.01 (br signal, 2H); 3.98-3.36 (br signal, 3H); 3.21-2.78 (broad signal, 2H); 2.45 (m, 3H); 1.20 (br signal, 3H).

### Example 186

### 1-((R)-4-Benzoyl-2-methyl-piperazin-1-yl)-2-[6-(1H-pyrazol-3-yl)-pyridin-3-yl]-ethane-1,2-dione

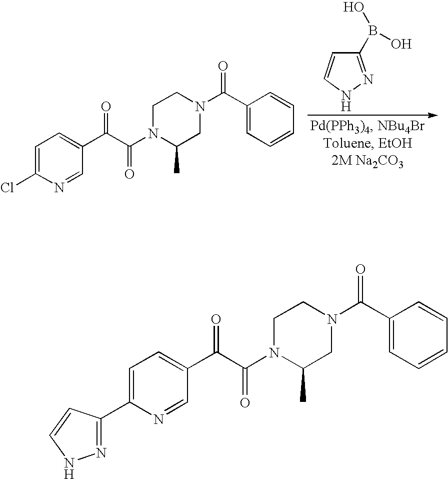

1-((R)-4-Benzoyl-2-methyl-piperazin-1-yl)-2-[6-(1H-pyrazol-3-yl)-pyridin-3-yl]-ethane-1,2-dione (20%) -was prepared according to the procedure above (3 h; LC/MS-monitoring), using 1-((R)-4-benzoyl-2-methyl-piperazin-1-yl)-2-(6-chloro-pyridin-3-yl)-ethane-1,2-dione and CHCl₃/MeOH 20:1 (1^{st} column), then EtOAc (2^{nd} column) as eluents. LCMS: Calc'd for C₂₂H₂₁N₅O₃.H⁺: m/z=404.5. Found: m/z=404, 405. ¹H NMR (DMSO-d₆): 13.83, 13.34 (two broad signals, 1H, NH-pyrazole); 9.03 (m, 1H); 8.30 (m, 1H); 8.20 (m, 1H); 7.90 (s, 1H); 7.45 (m, 5H); 7.07, 7.97 (two m, 1H); 4.86-4.09 (br signal, 2H); 4.03-3.37 (br signal, 3H); 3.22-2.86 (br signal, 2H); 1.22 (br signal, 3H).

### Example 193

### 1-((R)-4-Benzoyl-2-methyl-piperazin-1-yl)-2-(4-pyrazol-1-yl-phenyl)-ethane-1,2-dione

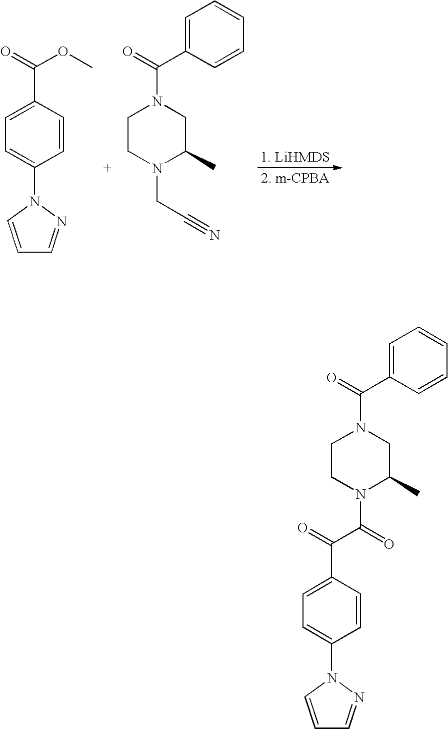

1-((R)-4-Benzoyl-2-methyl-piperazin-1-yl)-2-(4-pyrazol-1-yl-phenyl)-ethane-1,2-dione (43%) was prepared according to the procedure above, using 4-pyrazol-1-yl-benzoic acid methyl ester and ((R)-4-benzoyl-2-methyl-piperazin-1-yl)-acetonitrile. LCMS: Calc'd for C₂₃H₂₂N₄O₃.H⁺: m/z=403.5. Found: m/z=403, 404. ¹H NMR (DMSO-d₆): 8.69 (m, 1H); 8.11-7.98 (m, 4H); 7.87 (m, 1H); 7.45-7.40 (m, 5H); 6.65 (m, 1H); 4.86-4.10 (br signal, 2H); 3.96-3.41 (br signal, 3H); 3.18 (m, 1H); 3.08-2.85 (br signal, 1H); 1.20 (br signal, 3H).

### Example 195

### 1-((R)-4-Benzoyl-2-methyl-piperazin-1-yl)-2-(2-methyl-4-pyrazol-1-yl-phenyl)-ethane-1,2-dione

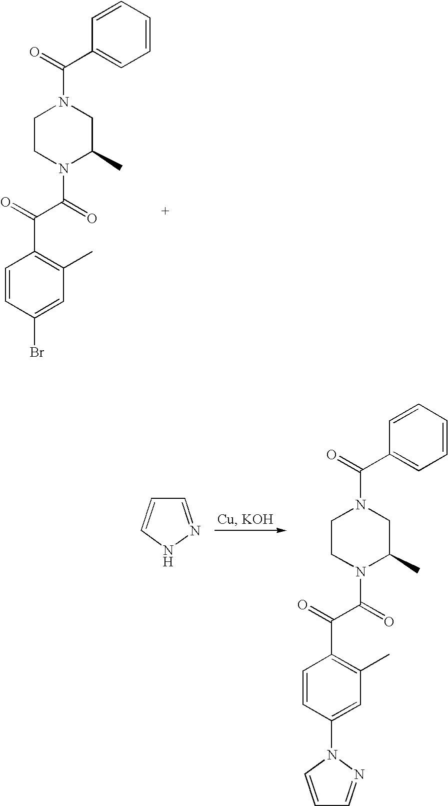

1-((R)-4-Benzoyl-2-methyl-piperazin-1-yl)-2-(2-methyl-4-pyrazol-1-yl-phenyl)-ethane-1,2-dione (22%) was prepared according to the procedure above (reaction time 2 h), using 1-((R)-4-benzoyl-2-methyl-piperazin-1-yl)-2-(4-bromo-2-methyl-phenyl)-ethane-1,2-dione and pyrazole, and CHCl₃/MeOH 50:1 (1^{st} column), then EtOAc/n-hexane 2:1 (2^{nd} column) as eluents. LCMS: Calc'd for C₂₄H₂₄N₄O₃.H⁺: m/z=417.5. Found: m/z=417, 418. ¹H NMR (DMSO-d₆): 8.66 (m, 1H); 7.95-7.78 (m, 4H); 7.45 (m, 5H); 6.63 (m, 1H); 4.83-4.03 (br signal, 2H); 3.93-3.33 (br signal, 3H); 3.19-2.90 (br signal, 2H); 2.67 (m, 3H); 1.24 (br signal, 3H).

### 1-((R)-4-Benzoyl-2-methyl-piperazin-1-yl)-2-(4-bromo-2-methyl-phenyl)-ethane-1,2-dione

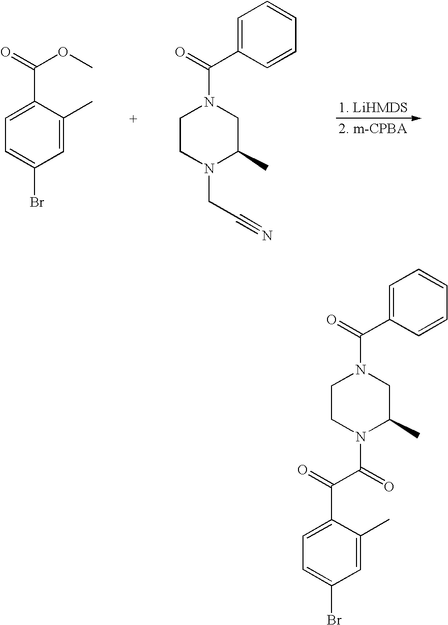

1-((R)-4-Benzoyl-2-methyl-piperazin-1-yl)-2-(4-bromo-2-methyl-phenyl)-ethane-1,2-dione (57%) was prepared according to the procedure above, using 4-bromo-2-methyl-benzoic acid methyl ester and ((R)-4-benzoyl-2-methyl-piperazin-1-yl)-acetonitrile. ¹H NMR (DMSO-d₆): 7.71-7.59 (m, 3H); 7.45 (m, 5H); 4.83-4.09 (br signal, 2H); 3.93-3.35 (br signal, 3H); 3.19-2.78 (broad signal, 2H); 2.56 (m, 3H); 1.20 (br signal, 3H).

### Example 213

### 1-((R)-4-Benzoyl-2-methyl-piperazin-1-yl)-2-(3-methyl-4-pyrazol-1-yl-phenyl)-ethane-1,2-dione

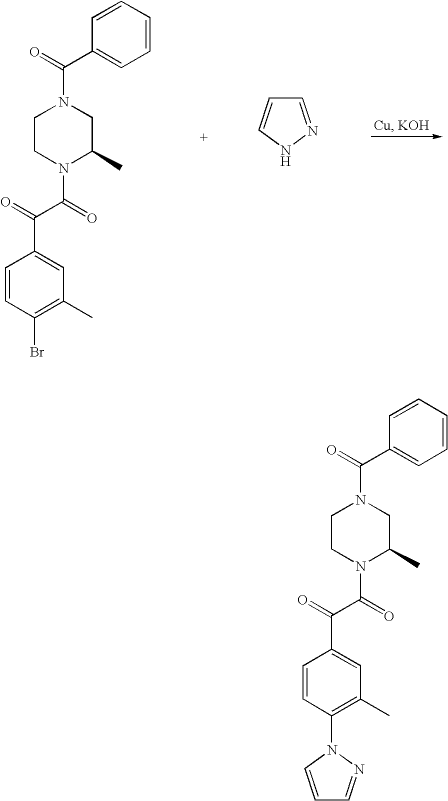

1-((R)-4-Benzoyl-2-methyl-piperazin-1-yl)-2-(3-methyl-4-pyrazol-1-yl-phenyl)-ethane-1,2-dione (31%) was prepared according to the procedure above (reaction time 2 h), using 1-((R)-4-benzoyl-3-methyl-piperazin-1-yl)-2-(4-bromo-2-methyl-phenyl)-ethane-1,2-dione and pyrazole, and CHCl₃/MeOH 20:1 (1^{st} column), then EtOAc/n-hexane 2:1 (2^{nd} column) as eluents. LCMS: Calc'd for C₂₄H₂₄N₄O₃.H⁺: m/z=417.5. Found: m/z=417, 418. ¹H NMR (DMSO-d₆): 8.21 (m, 1H); 7.94-7.81 (m, 3H); 7.64 (m, 1H); 7.45 (m, 5H); 6.58 (m, 1H); 4.84-4.08 (br signal, 2H); 4.03-3.39 (br signal, 3H); 3.22-2.80 (br signal, 2H); 2.40 (m, 3H); 1.22 (br signal, 3H).

### Example 214

### 1-((R)-4-Benzoyl-2-methyl-piperazin-1-yl)-2-(6-pyrazol-1-yl-pyridin-3-yl)-ethane-1,2-dione

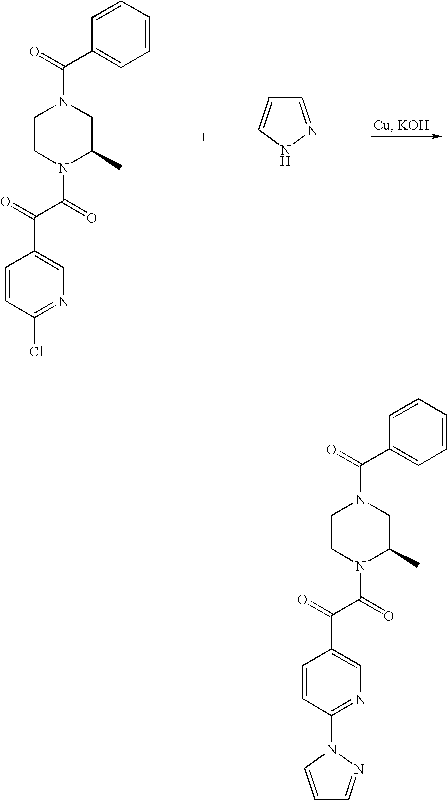

1-((R)-4-Benzoyl-2-methyl-piperazin-1-yl)-2-(6-pyrazol-1-yl-pyridin-3-yl)-ethane-1,2-dione (19%) was prepared according to the procedure above (reaction time 30 min), using 1-((R)-4-benzoyl-2-methyl-piperazin-1-yl)-2-(6-chloro-pyridin-3-yl)-ethane-1,2-dione and pyrazole, and Et₂O/MeOH 9:1 as eluent. LCMS: Calc'd for C₂₂H₂₁N₅O₃.H⁺: m/z=404.5. Found: m/z=404, 405. ¹H NMR (DMSO-d₆): 8.94 (m, 1H); 8.72 (m, 1H); 8.45 (m, 1H); 8.12 (d, J=8.8 Hz, 1H); 7.96 (s, 1H); 7.45 (m, 5H); 6.68 (m, 1H); 4.84-4.12 (br signal, 2H); 4.09-3.41 (br signal, 3H); 3.20-2.82 (br signal, 2H); 1.19 (br signal, 3H).

### 1-((R)-4-Benzoyl-2-methyl-piperazin-1-yl)-2-(6-chloro-pyridin-3-yl)-ethane-1,2-dione

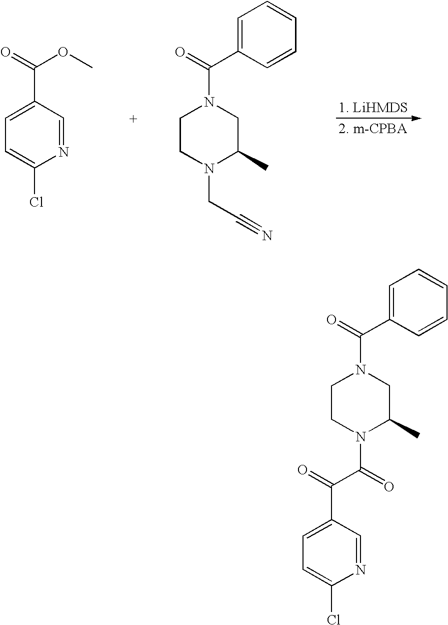

1-((R)-4-Benzoyl-2-methyl-piperazin-1-yl)-2-(6-chloro-pyridin-3-yl)-ethane-1,2-dione (28%) was prepared according to the procedure above, using 6-chloro-nicotinic acid methyl ester and ((R)-4-benzoyl-2-methyl-piperazin-1-yl)-acetonitrile. LCMS: Calc'd for C₁₉H₁₈ClN₃O₃.H⁺: m/z=372.8. Found: m/z=373, 374.

### Example 215

### 1-((R)-4-Benzoyl-2-methyl-piperazin-1-yl)-2-(4-imidazol-1-yl-2-methyl-phenyl)-ethane-1,2-dione

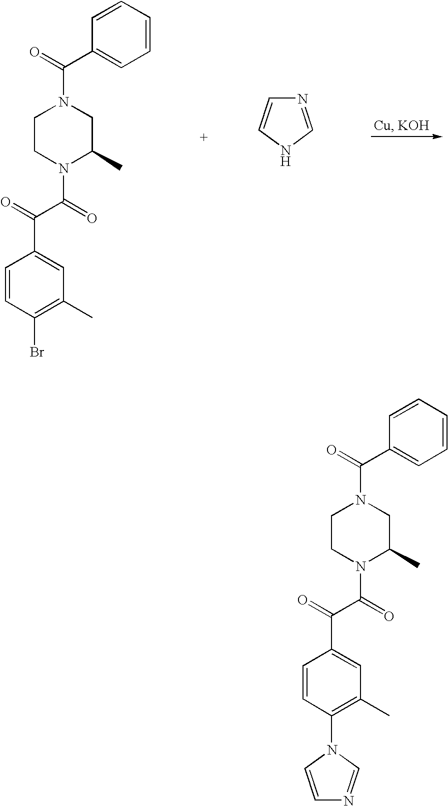

1-((R)-4-Benzoyl-2-methyl-piperazin-1-yl)-2-(4-imidazol-1-yl-2-methyl-phenyl)-ethane-1,2-dione (14%) was prepared according to the procedure above (reaction time 1 h 30 min), using 1-((R)-4-benzoyl-3-methyl-piperazin-1-yl)-2-(4-bromo-2-methyl-phenyl)-ethane-1,2-dione and imidazole, and CHCl₃/MeOH 20:1 as eluent. The product was additionally purified by HPLC. LCMS: Calc'd for C₂₄H₂₄N₄O₃.H⁺: m/z=417.5. Found: m/z=417, 418. ¹H NMR (DMSO-d₆): 7.96-7.82 (m, 3H); 7.57-7.43 (m, 7H); 7.15 (s, 1H); 4.89-4.05 (br signal, 2H); 4.01-3.37 (br signal, 3H); 3.22-2.81 (br signal, 2H); 2.30 (m, 3H); 1.21 (br signal, 3H).

### Example 211

### 1-((R)-4-Benzoyl-2-methyl-piperazin-1-yl)-2-(4-pyrrolidin-1-yl-phenyl)-ethane-1,2-dione

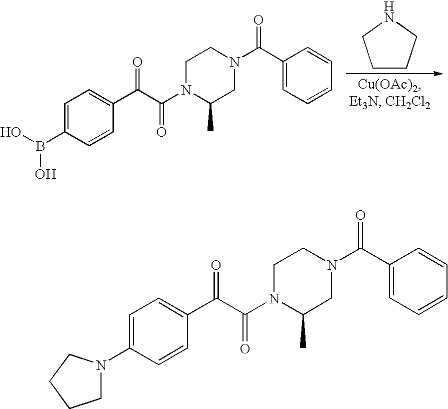

Mixture of boronic acid (0.20 g, 0.53 mmol), pyrrolidine (0.04 g, 0.56 mmol), Et₃N (0.16 mL, 1.11 mmol) and anhydrous Cu(OAc)₂ (0.10 g, 0.55 mmol) in CH₂Cl₂ (5 mL) was stirring for 1 h under RT. Then H₂O (10 mL) and CH₂Cl₂ (20 mL) were added. The organic layer was separated, washed with brine, dried over sodium sulfate, filtered and concentrated. The crude residue was purified by SiO₂ chromatography using ethyl acetate as eluent to obtain 1-((R)-4-benzoyl-2-methyl-piperazin-1-yl)-2-(4-pyrrolidin-1-yl-phenyl)-ethane-1,2-dione (0.12 g, 56%). LCMS: Calc'd for C₂₄H₂₇N₃O₃.H⁺: m/z=406.5. Found: m/z=406, 407. ¹H NMR (DMSO-d₆): 7.65 (m, 2H); 7.45 (m, 5H); 6.63 (m, 2H); 4.81-4.03 (br signal, 2H); 3.99-3.49 (br signal, 2H); 3.36 (m, 4H); 3.21-2.77 (br signal, 3H); 1.98 (m, 4H); 1.18 (br signal, 3H).

### 4-[2-((R)-4-Benzoyl-2-methyl-piperazin-1-yl)-2-oxo-acetyl]-boronic acid

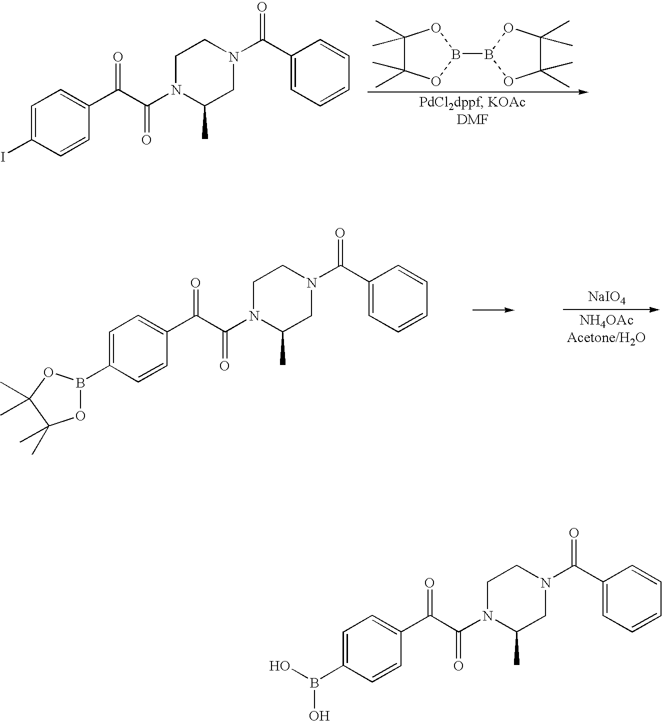

Anhydrous DMF (100 mL) was added to a mixture of 1-(4-benzoyl-2-methyl-piperazin-1-yl)-2-(4-iodo-phenyl)-ethane-1,2-dione (3.75 g, 8.1 mmol), 4,4,5,5,4′,4′,5′,5′-octamethyl-[2,2′]bi[[1,3,2]dioxaborolanyl] (2.68 g, 10 mmol) and anhydrous KOAc (2.38 g, 24 mmol). Argon was bubbled within reaction mixture for ˜20 min. Then PdCl₂dppf (0.18 g, 0.24 mmol) was added. The reaction mixture was stirring under 75° C. for 1.5 h (LCMS-monitoring,), then cooled to RT, diluted with benzene (300 mL) and washed with water (3×200 mL). Organic layer was evaporated, the residue was extracted with Et₂O and filtered. Mother solution was evaporated to give crude 1-((R)-4-benzoyl-2-methyl-piperazin-1-yl)-2-[4-(4,4,5,5-tetramethyl-[1,3,2]dioxaborolan-2-yl)-phenyl]-ethane-1,2-dione which was used for the next step without additional purification. LCMS: Calc'd for C₂₆H₃₂BN₂O₅.H⁺: m/z=463.4. Found: m/z=463,464.

NaIO₄ (6.5 g, 30 mmol) and NH₄OAc (2.42 g, 31 mmol) were added to acetone-water (1:1, 200 mL) solution of crude 1-((R)4-benzoyl-2-methyl-piperazin-1-yl)-2-[4-(4,4,5,5-tetramethyl-[1,3,2]dioxaborolan-2-yl)-phenyl]-ethane-1,2-dione from previous step. The reaction mixture was kept under stirring for 24 h at RT, diluted with water (200 mL), and precipitate was filtered off. Solution was extracted with EtOAc (2×200 mL), organic phase was dried with Na₂SO₄, and evaporated to afford 4-[2-((R)-4-benzoyl-2-methyl-piperazin-1-yl)-2-oxo-acetyl]-boronic acid (2.3 g, 74% per two steps). LCMS: Calc'd for C₂₀H₂₁BN₂O₅.H⁺: m/z=381.2. Found: m/z=381, 382.

### Example 212

### 1-((R)-4-Benzoyl-2-methyl-piperazin-1-yl)-2-[4-(2-oxo-oxazolidin-3-yl)-phenyl]-ethane-1,2-dione

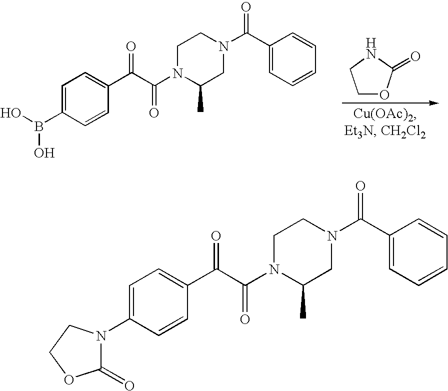

1-((R)-4-Benzoyl-2-methyl-piperazin-1-yl)-2-[4-(2-oxo-oxazolidin-3-yl)-phenyl]-ethane-1,2-dione (36%) was prepared according to the procedure above, using oxazolidin-2-one. The product was additionally purified by HPLC. LCMS: Calc'd for C₂₃H₂₃N₃O₅.H⁺: m/z=422.5. Found: m/z=422, 423. ¹H NMR (DMSO-d₆): 7.96-7.90 (m, 2H); 7.80 (m, 2H); 7.45 (m, 5H); 4.87-4.53 (br signal, 1H); 4.50 (m, 2H); 4.43-4.19 (br signal, 1H); 4.14 (m, 2H); 3.90-3.34 (br signal, 3H); 3.19-2.77 (br signal, 2H); 1.20 (br signal, 3H).

### Example 216

### 1-((R)-4-Benzoyl-2-methyl-piperazin-1-yl)-2-[2-dimethylamino-4-(1H-pyrazol-3-yl)-phenyl]-ethane-1,2-dione

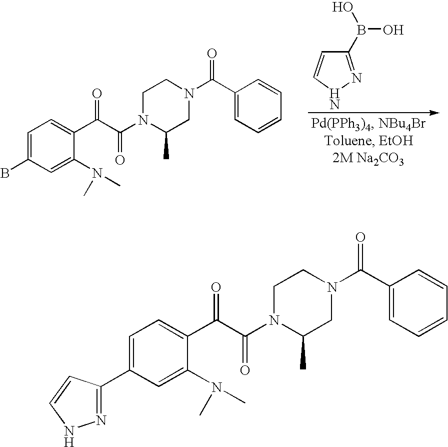

1-((R)-4-Benzoyl-2-methyl-piperazin-1-yl)-2-[2-dimethylamino-4-(1H-pyrazol-3-yl)-phenyl]-ethane-1,2-dione (86%) was prepared according to the procedure above (30 min; TLC-monitoring, CHCl₃/MeOH 20:1), using 1-((R)-4-benzoyl-2-methyl-piperazin-1-yl)-2-(4-bromo-2-dimethylamino-phenyl)-ethane-1,2-dione, and CHCl₃/MeOH 20:1 (1^{st} column), then CHCl₃/MeOH 9:1 (2^{nd} column) as eluents. LCMS: Calc'd for C₂₅H₂₇N₅O₃.H⁺: m/z=446.5. Found: m/z=446, 447. ¹H NMR (DMSO-d₆): 13.51, 13.07 (two broad signals, 1H, NH-pyrazole); 7.83-7.46 (m, 9H); 6.86 (m, 1H); 4.81-4.11 (br signal, 2H); 4.07-3.35 (br signal, 3H); 3.15-2.91 (br signal, 2H); 2.84 (s, 3H); 2.78 (s, 3H); 1.19 (br signal, 3H).

### 1-((R)-4-Benzoyl-2-methyl-piperazin-1-yl)-2-(4-bromo-2-dimethylamino-phenyl)-ethane-1,2-dione

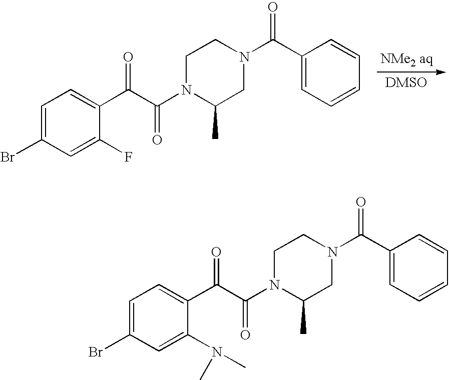

To a solution of 1-((R)-4-benzoyl-2-methyl-piperazin-1-yl)-2-(4-bromo-2-fluoro-phenyl)-ethane-1,2-dione (0.5 g, 1.15 mmol) in DMSO (5 mL), 40% aqueos solution of dimethylamine (3 mL) was added. The reaction mixture was heated to 80° C. and kept under stirring at this temperature for 1 h (TLC monitoring, EtOAc/n-hexane 3:1), cooled to RT, poured into ice water (50 mL), and extracted with EtOAc (˜2 mL). Organic phase was washed with water (3×30 mL), dried over sodium sulfate, and then concentrated. The crude product was purified by silica-gel chromatography using EtOAc/n-hexane 3:1 as eluent to afford 1-((R)-4-benzoyl-2-methyl-piperazin-1-yl)-2-(4-bromo-2-dimethylamino-phenyl)-ethane-1,2-dione (0.35 g, 66%). LCMS: Calc'd for C₂₂H₂₄BrN₃O₃.H⁺: m/z=459. Found: m/z=459, 460.

### Example 219

### 1-((R)-4-Benzoyl-2-methyl-piperazin-1-yl)-2-[2-amino-4-(1H-pyrazol-3-yl)-phenyl]-ethane-1,2-dione

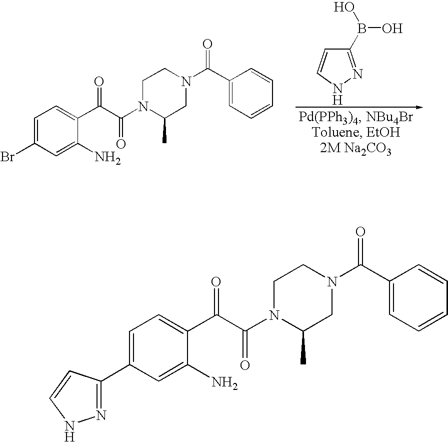

1-((R)-4-Benzoyl-2-methyl-piperazin-1-yl)-2-[2-dimethylamino-4-(1H-pyrazol-3-yl)-phenyl]-ethane-1,2-dione (52%) was prepared according to the procedure above (40 min; TLC-monitoring, CHCl₃/MeOH 20:1), using 1-((R)-4-benzoyl-2-methyl-piperazin-1-yl)-2-(4-bromo-2-amino-phenyl)-ethane-1,2-dione, and EtOAc as eluent. LCMS: Calc'd for C₂₃H₂₃N₅O₃.H⁺: m/z=418.5. Found: m/z=418, 419. ¹H NMR (DMSO-d₆): 13.14 (broad signal, 1H, NH-pyrazole); 7.78 (m, 1H); 7.45-7.30 (m, 9H); 7.05 (m, 1H); 6.68 (m, 1H); 4.85-3.36 (br signal, 5H); 3.15-2.84 (br signal, 2H); 1.19 (br signal, 3H).

### 1-(2-Amino-4-bromo-phenyl)-2-((R)-4-benzoyl-2-methyl-piperazin-1-yl)-ethane-1,2-dione

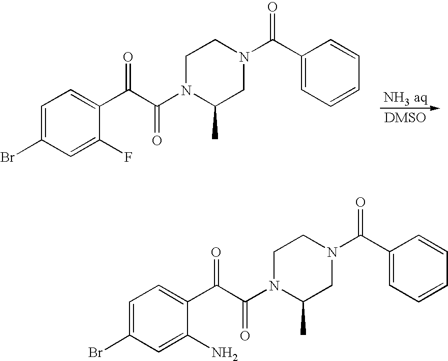

1-(2-Amino-4-bromo-phenyl)-2-((R)-4-benzoyl-2-methyl-piperazin-1-yl)-ethane-1,2-dione was prepared according to the procedure above, using aqueos solution of NH₃. LCMS: Calc'd for C₂₀H₂₀BrN₃O₃.H⁺: m/z=431. Found: m/z=432, 433, 434.

### Example 4

In this example, illustrated are compounds of Formula (I) according to some embodiments of the present invention. Measurements for antiviral activity, performed according to the methods described in Example 1 herein, are noted by reference to a range in Table 3, with “A” denoting antiviral activity represented by an IC₅₀ less than 5 μm; and “B” denoting antiviral activity represented by an IC₅₀ greater than 5 μm. Where the stereochemistry is depicted, the activity of the compound was assayed using an enantiomerically purified compound.

TABLE 3

| Example | | |
|---|---|---|
| Number | Structure | Activity |
| 101 | 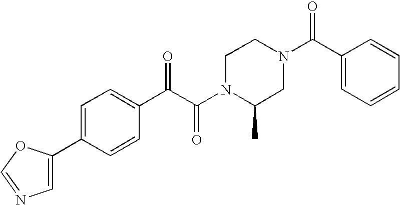 | A |
| 102 | 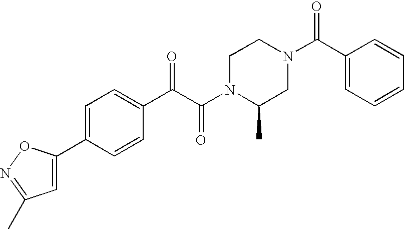 | A |
| 103 | 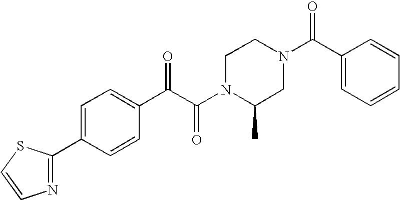 | A |
| 104 | 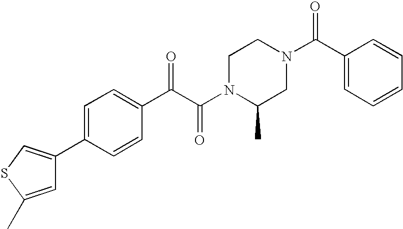 | B |
| 105 | 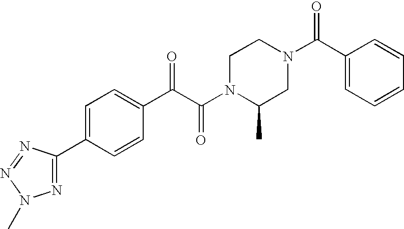 | A |
| 106 | 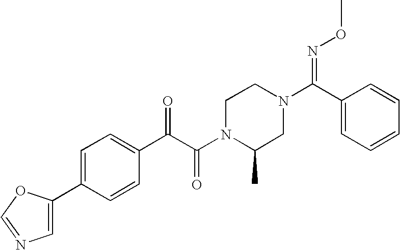 | A |
| 107 | 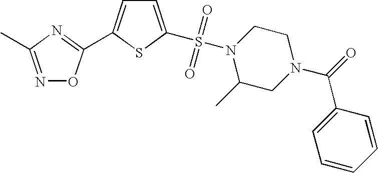 | B |
| 108 | 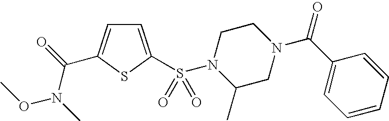 | A |
| 109 | 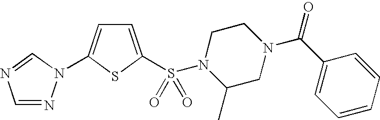 | B |
| 110 | 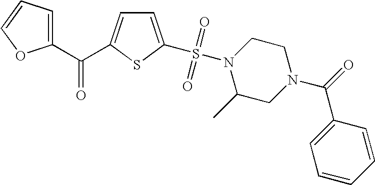 | B |
| 111 | 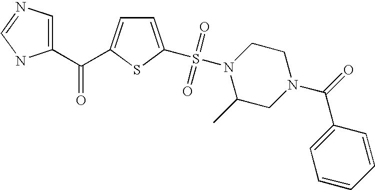 | B |
| 112 | 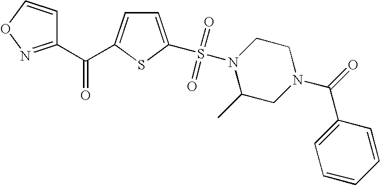 | B |
| 113 | 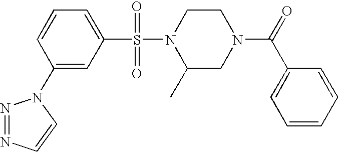 | B |
| 114 | 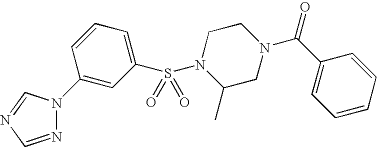 | B |
| 115 | 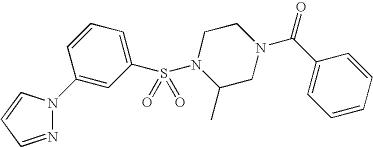 | B |
| 116 | 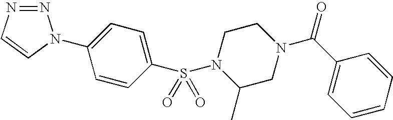 | B |
| 117 | 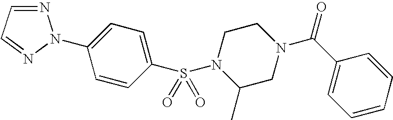 | B |
| 118 | 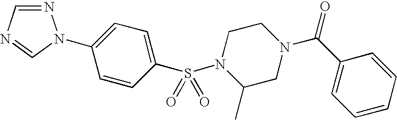 | B |
| 119 | 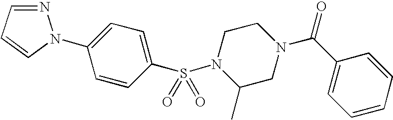 | B |
| 120 | 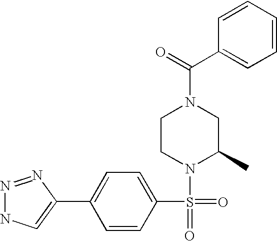 | A |
| 121 | 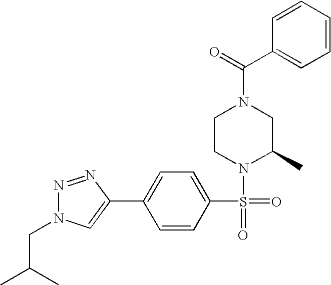 | B |
| 122 | 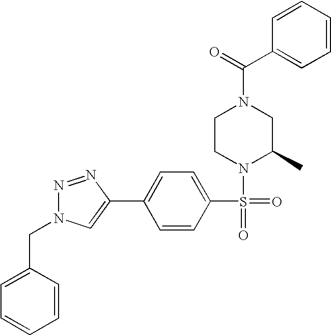 | B |
| 123 | 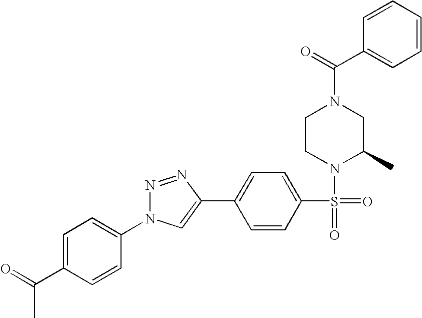 | B |
| 124 | 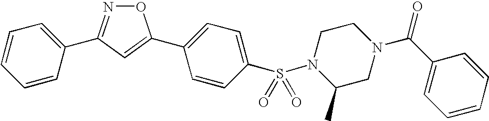 | B |
| 125 | 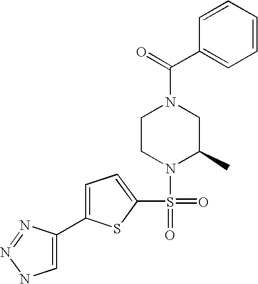 | B |
| 126 | 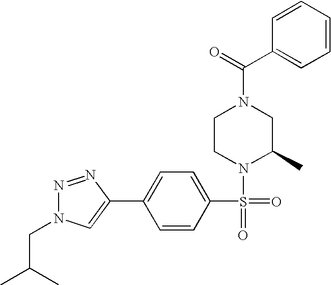 | B |
| 127 | 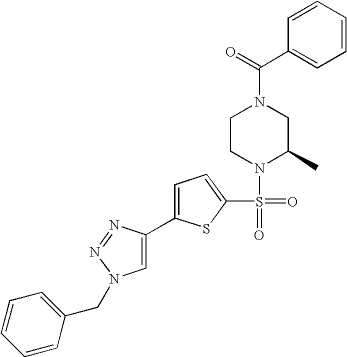 | B |
| 128 | 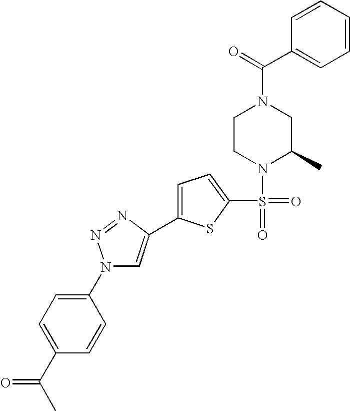 | B |
| 129 | 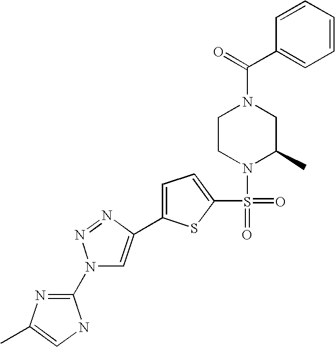 | B |
| 130 | 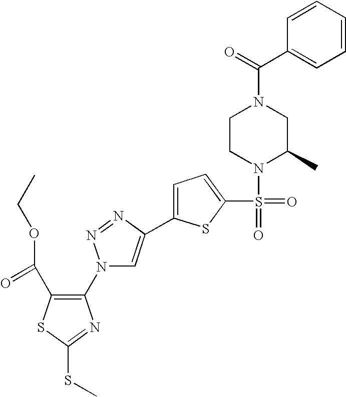 | B |
| 131 | 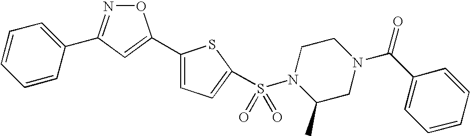 | B |
| 132 | 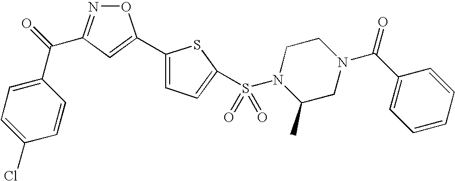 | B |
| 133 | 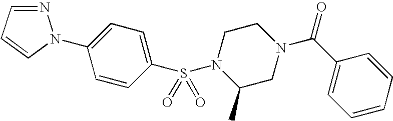 | B |
| 134 | 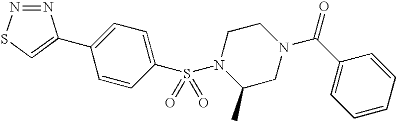 | B |
| 135 | 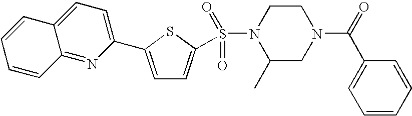 | B |
| 136 | 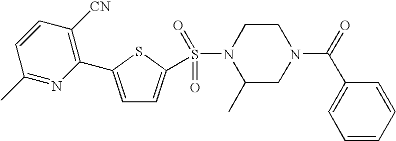 | B |
| 137 | 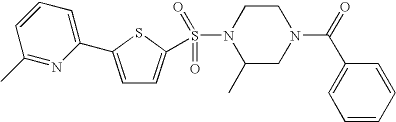 | B |
| 138 | 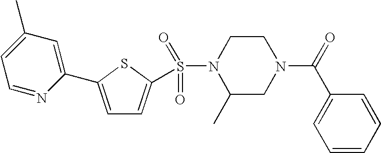 | B |
| 139 | 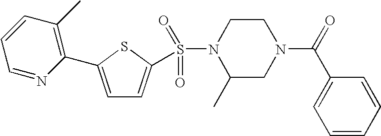 | B |
| 140 | 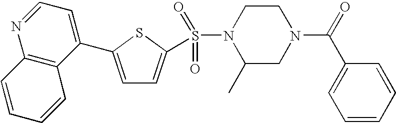 | |
| 141 | 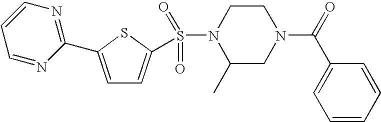 | A |
| 142 | 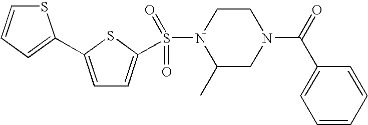 | A |
| 143 | 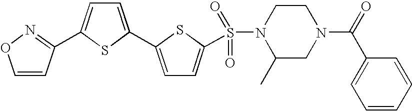 | B |
| 144 | 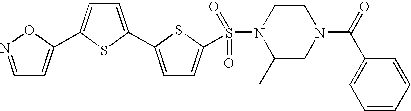 | A |
| 145 | 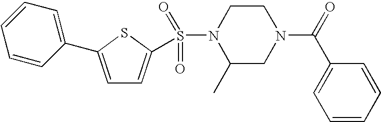 | B |
| 146 | 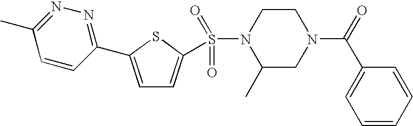 | B |
| 147 | 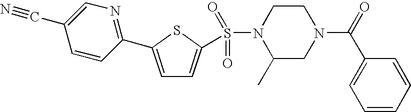 | A |
| 148 | 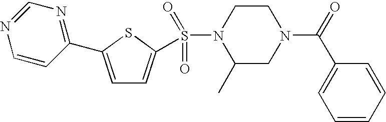 | B |
| 149 | 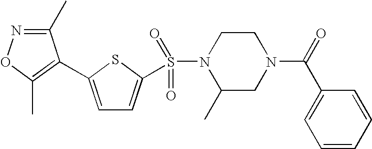 | Not available |
| 150 | 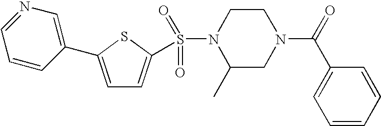 | B |
| 151 | 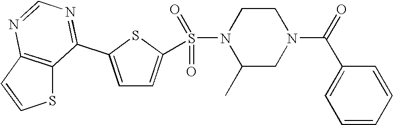 | B |
| 152 | 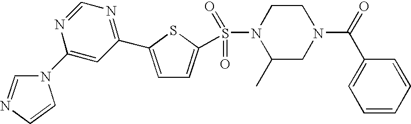 | B |
| 153 | 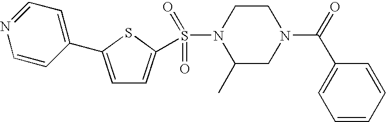 | B |
| 154 | 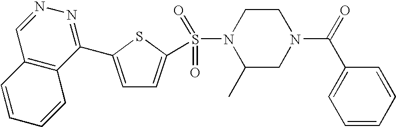 | B |
| 155 | 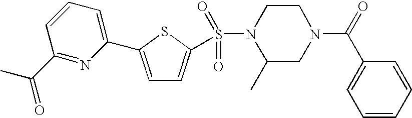 | B |
| 156 | 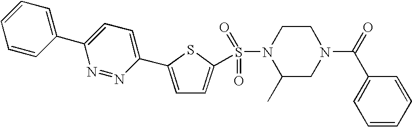 | B |
| 157 | 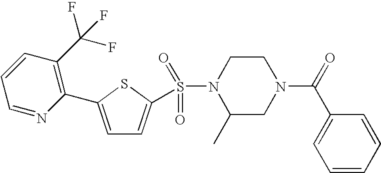 | B |
| 158 | 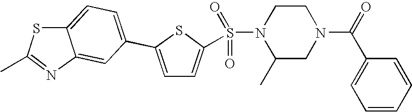 | B |
| 159 | 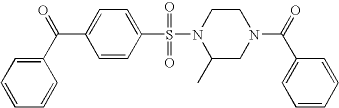 | Not available |
| 160 | 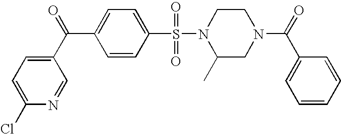 | Not available |
| 162 | 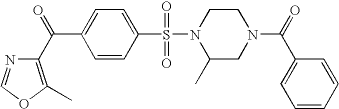 | Not available |
| 163 | 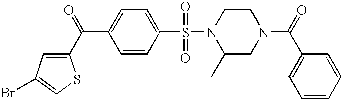 | Not available |
| 164 | 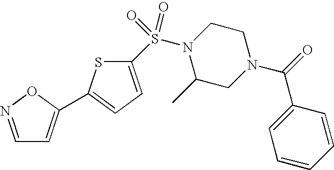 | A |
| 165 | 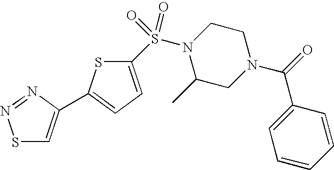 | B |
| 166 | 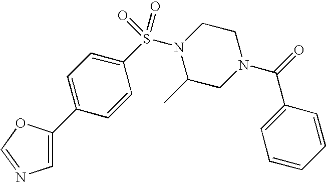 | B |
| 167 | 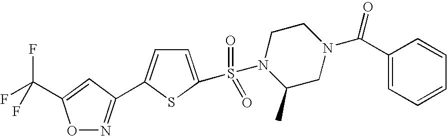 | B |
| 168 | 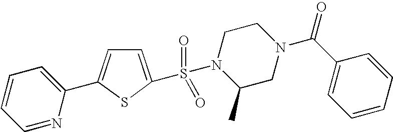 | B |
| 169 | 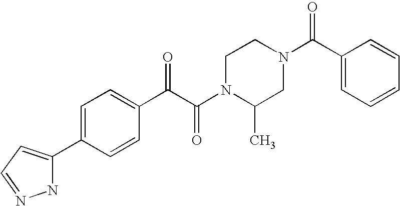 | A |
| 170 | 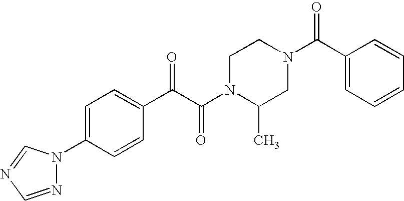 | A |
| 171 | 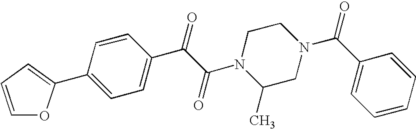 | A |
| 172 | 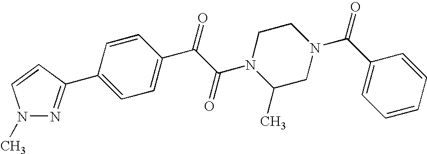 | A |
| 173 | 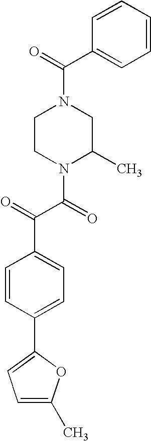 | A |
| 174 | 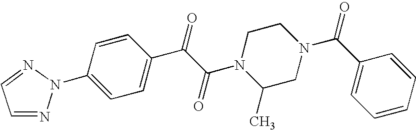 | A |
| 175 | 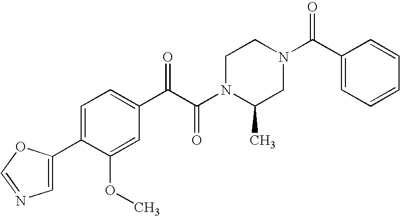 | A |
| 176 | 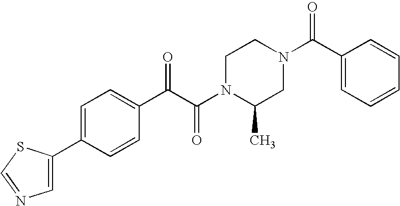 | A |
| 177 | 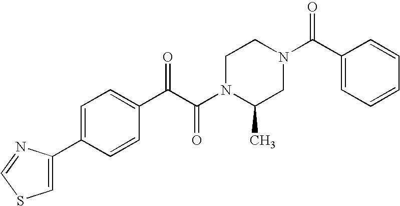 | A |
| 178 | 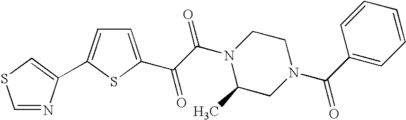 | A |
| 179 | 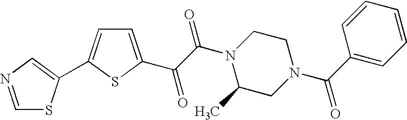 | A |
| 180 | 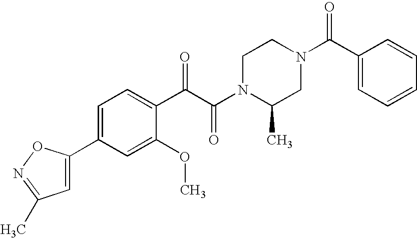 | A |
| 181 | 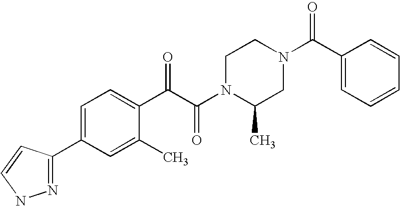 | A |
| 182 | 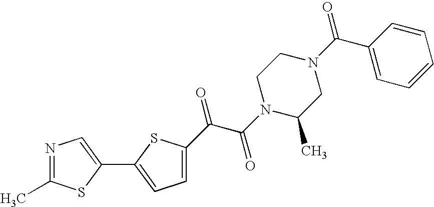 | B |
| 183 | 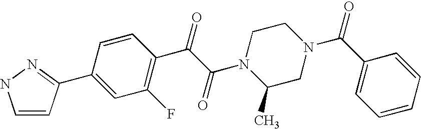 | A |
| 184 | 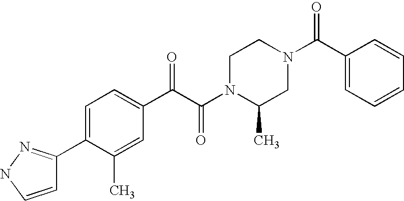 | A |
| 185 | 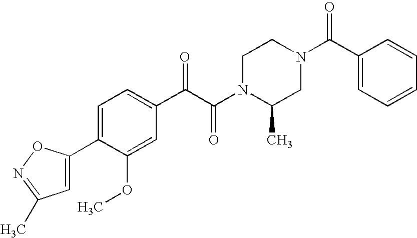 | A |
| 186 | 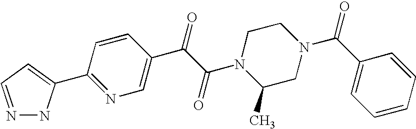 | A |
| 187 | 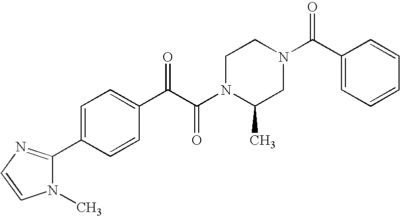 | B |
| 188 | 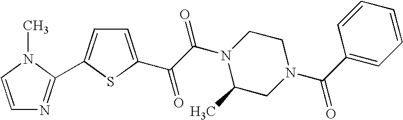 | B |
| 189 | 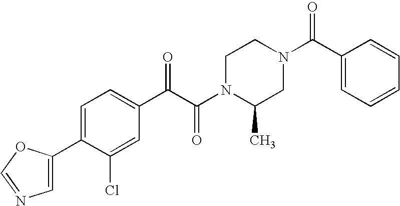 | A |
| 190 | 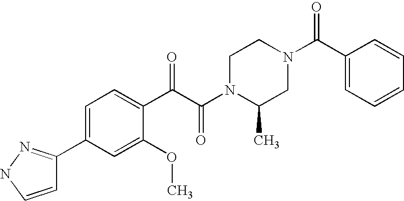 | A |
| 191 | 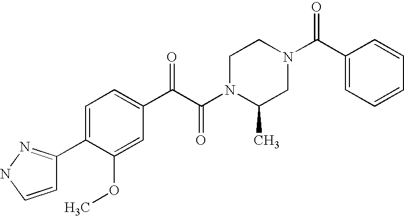 | A |
| 192 | 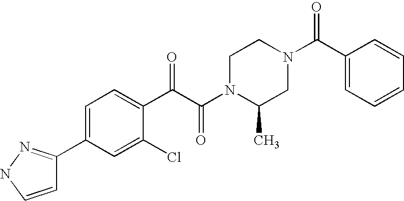 | A |
| 193 | 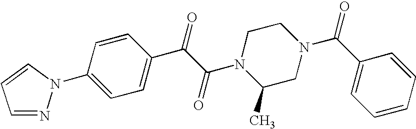 | A |
| 194 | 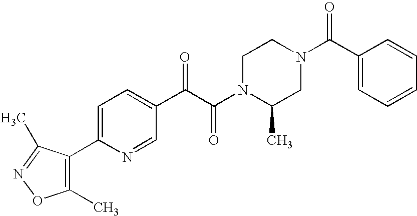 | B |
| 195 | 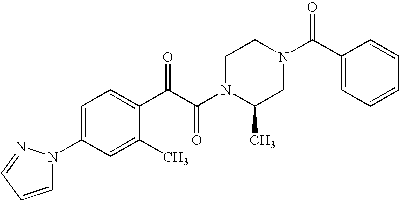 | B |
| 196 | 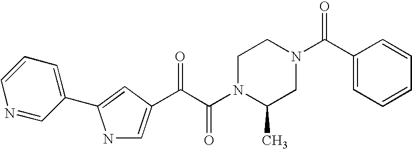 | B |
| 197 | 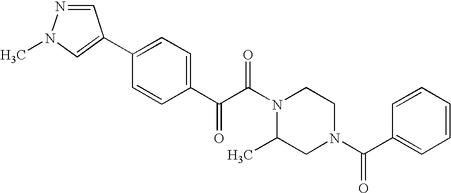 | A |
| 198 | 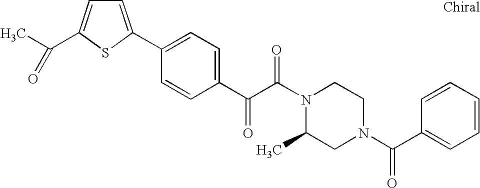 | A |
| 199 | 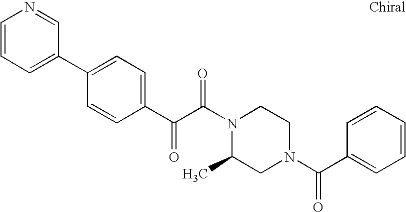 | A |
| 200 | 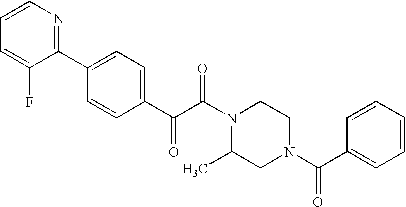 | A |
| 201 | 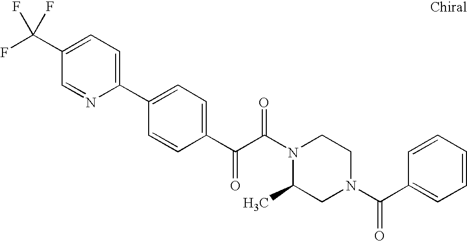 | A |
| 202 | 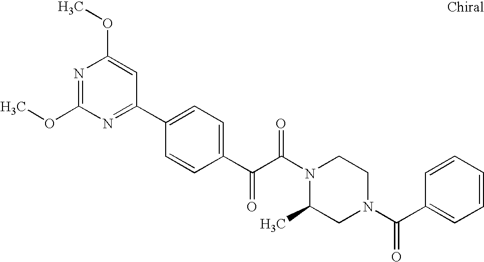 | A |
| 203 | 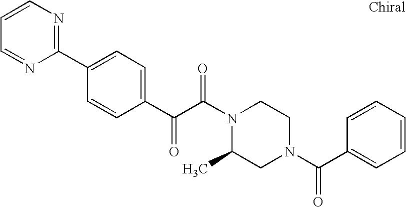 | A |
| 204 | 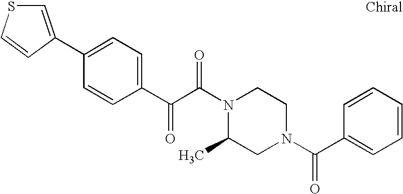 | A |
| 205 | 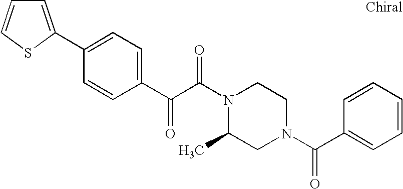 | A |
| 206 | 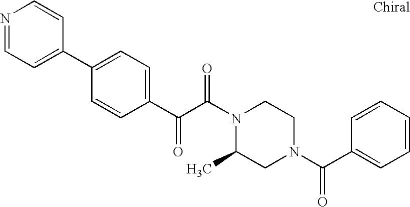 | A |
| 207 | 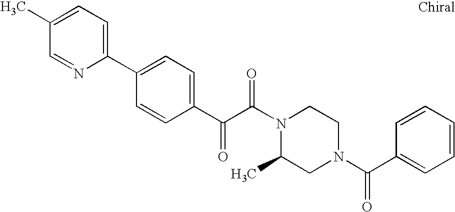 | A |
| 208 | 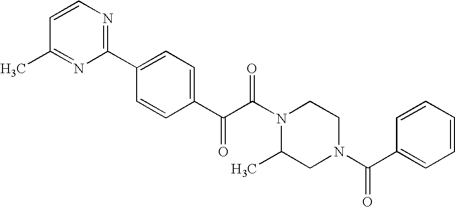 | A |
| 209 | 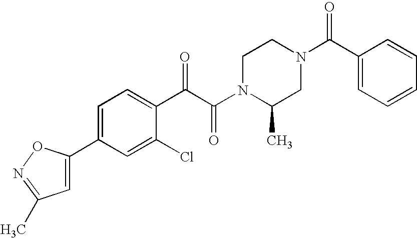 | A |
| 210 | 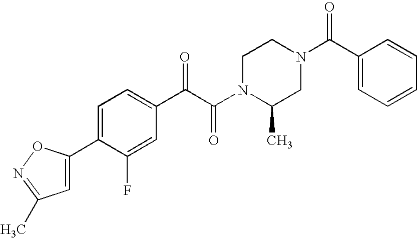 | A |
| 211 | 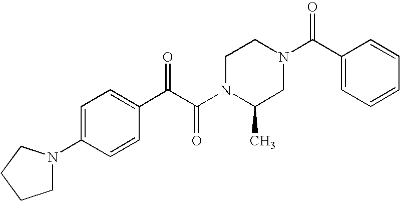 | B |
| 212 | 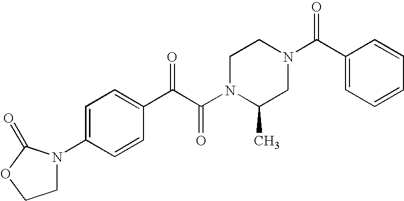 | B |
| 213 | 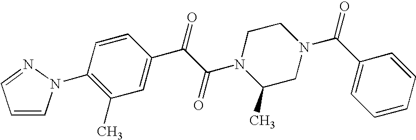 | B |
| 214 | 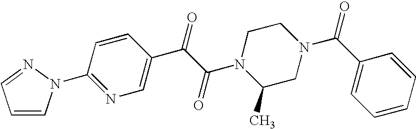 | A |
| 215 | 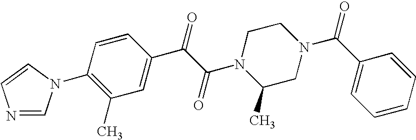 | A |
| 216 | 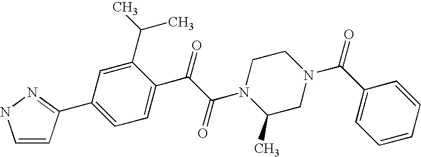 | B |
| 217 | 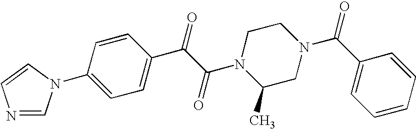 | A |
| 218 | 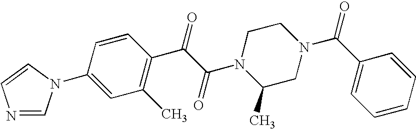 | A |
| 219 | 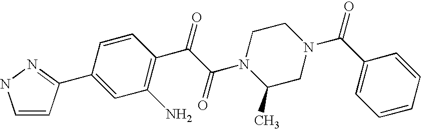 | A |
| 220 | 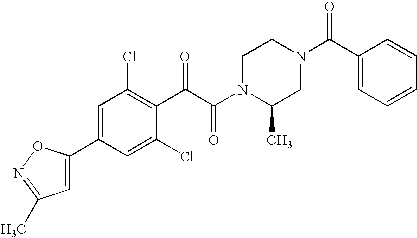 | B |
| 221 | 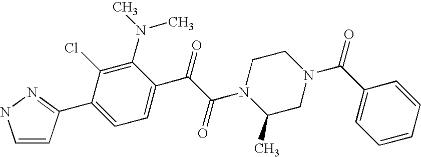 | A |
| 222 | 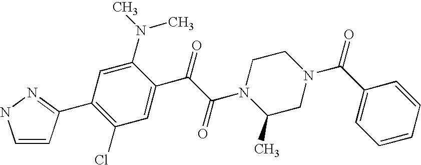 | B |
| 223 | 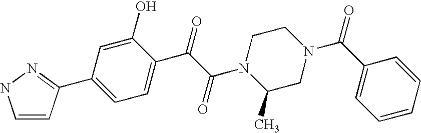 | A |
| 224 | 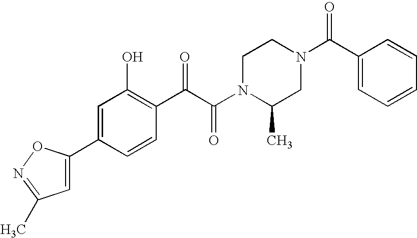 | B |
| 225 | 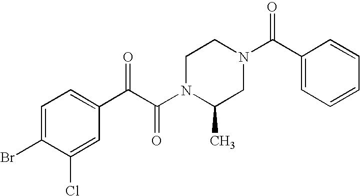 | A |
| 226 | 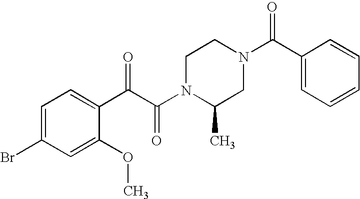 | A |
| 227 | 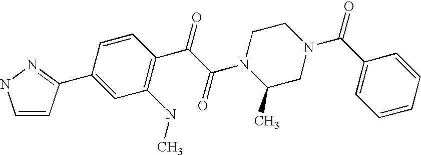 | B |
| 228 | 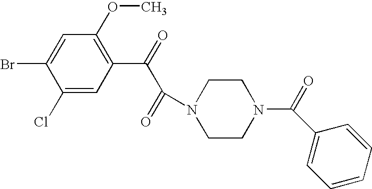 | A |
| 229 | 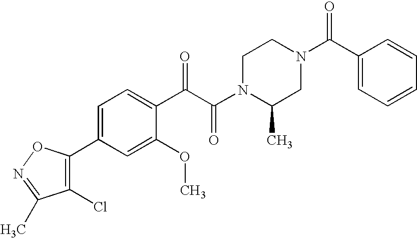 | A |
| 230 | 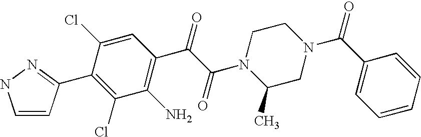 | B |
| 231 | 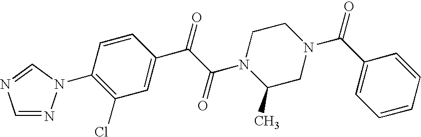 | B |
| 232 | 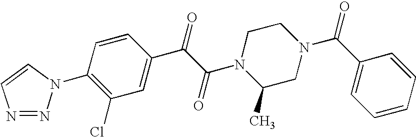 | B |
| 233 | 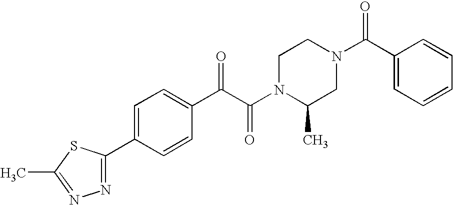 | A |
| 234 | 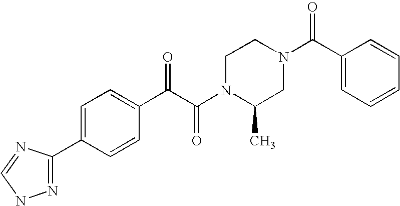 | A |
| 235 | 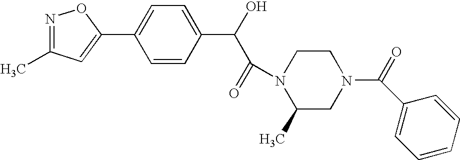 | B |
| 236 | 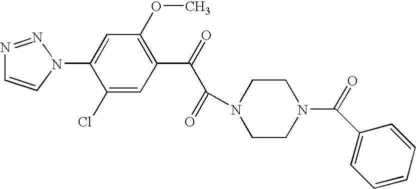 | A |
| 237 | 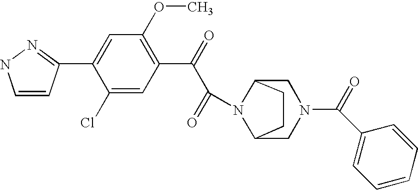 | B |
| 238 | 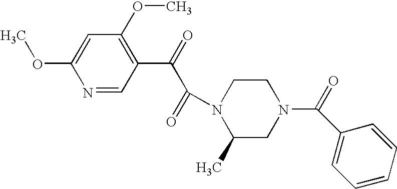 | B |
| 239 | 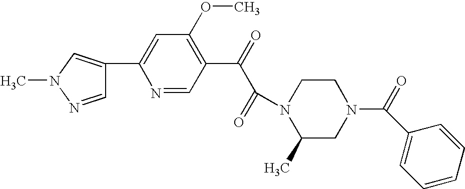 | A |
| 240 | 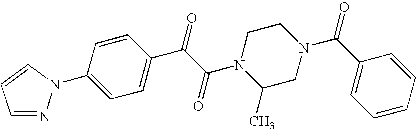 | A |
| 241 | 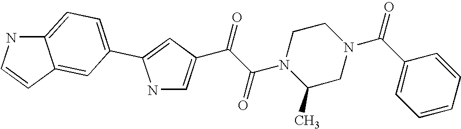 | B |
| 242 | 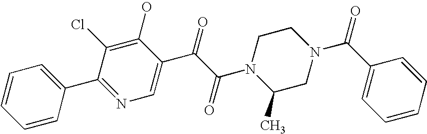 | B |
| 243 | 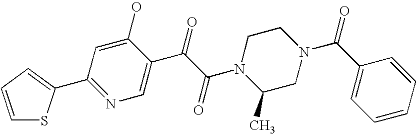 | B |
| 244 | 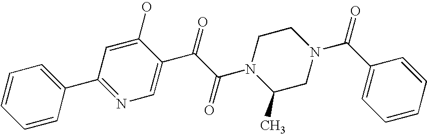 | B |
| 245 | 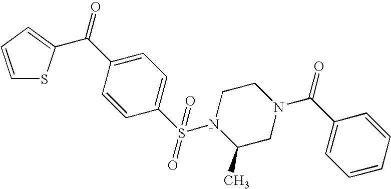 | B |
| 246 | 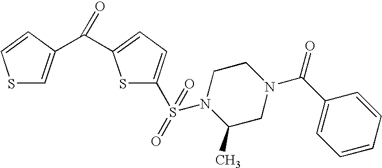 | B |
| 247 | 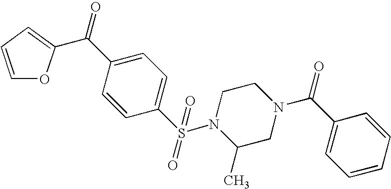 | B |
| 248 | 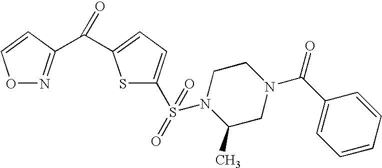 | B |
| 249 | 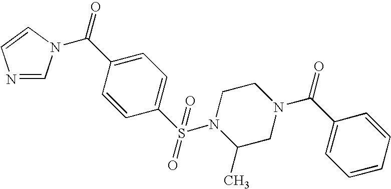 | B |
| 250 | 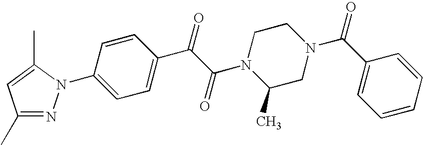 | B |
| 251 | 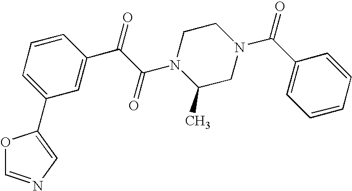 | B |
| 252 | 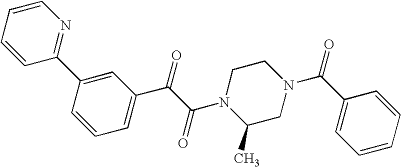 | B |
| 253 | 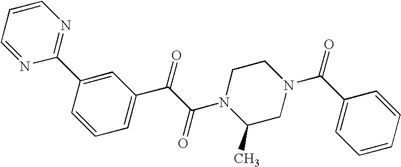 | B |
| 254 | 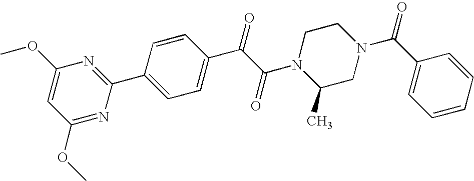 | B |

What is claimed is:

1. A compound of Formula (I) 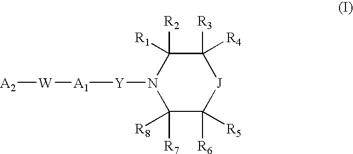
or a pharmaceutically acceptable prodrug, salt, polymorph, solvate, enantiomer, diastereomer, racemate, mixture of stereoisomers thereof, or derivative thereof, wherein:
W is selected from the group consisting of null, oxy, amino, thio, sulfinyl, sulfonyl, carbonyl, amide, alkylene and cycloalkylidene,
wherein at least one carbon atom of the alkylene or cycloalkylidene is optionally substituted with an oxy, amino, thio, sulfinyl, sulfonyl, carbonyl or amide group, and wherein the alkylene or cycloalkylidene is optionally substituted with at least one halogen atom;
A₁ is a monocyclic ring selected from the group consisting of monocyclic cycloalkylidene, monocyclic heterocycloalkylidene, monocyclic arylene and monocyclic heteroarylene, wherein the monocyclic ring is optionally substituted with at least one functional group selected from the group consisting of alkyl, alkoxy, fluoroalkyl, cycloalkyl, hydroxy, halo, fluoroalkoxy, alkenyl, alkenoxy, phosphoramide, phosphoramidralkyl, phosphonate, phosphonatealkyl and —R₉Q, wherein R₉ is null or alkylene and Q is selected from the group consisting of —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈, and —OR₂₉;
A₂ is selected from the group consisting of null, cycloalkyl, heterocycloalkyl, aryl and heteroaryl, wherein the cycloalkyl, heterocycloalkyl, heteroarylalkyl; or wherein R₃₂ and R₃₃ or R₃₆ and R₃₇, taken together with the nitrogen to which they are attached, are part of a heterocycloalkyl or heteroaryl; and
wherein the cycloalkyl, heterocycloalkyl, aryl and heteroaryl are optionally substituted with at least one functional group selected from the group consisting of halo, alkoxy, —CF₃, —OCF₃ and —CN;
J is 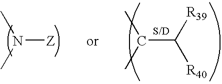
Z is selected from the group consisting of —COR₄₁, —C(═NR₄₃)R₄₁ and R₄₂;
R₄, is selected from the group consisting of cycloalkyl, heterocycloalkyl, aryl and heteroaryl; each optionally substituted with one or more functional groups selected from the group consisting of alkyl, cycloalkyl, alkoxy, fluoroalkoxy, fluoroalkyl, fluorocycloalkyl, halo, —CN, —CF₃, alkylthio, hydroxy, alkenyl, alkenoxy, acetyl and —R₉Q, wherein R₉ and Q are defined above;
R₄₂ is selected from the group consisting of aryl and heteroaryl, optionally substituted at least one functional group selected from the group consisting of halo, alkoxy, —CF₃, —OCF₃, —CN, alkyl, -cycloalkyl, -fluoroalkoxy, fluoroalkyl, fluorocycloalkyl, alkylthio, hydroxy, acetyl, alkenyl, alkenoxy and —R₉Q, wherein R₉ and Q are defined above;
R₄₃ is selected from the group consisting of hydrogen, alkoxy, cyano, fluoroalkoxy, alkyl, fluoroalkyl, cycloalkyl, fluorocycloalkyl, aryl, heteroaryl or heterocycloalkyl;
wherein the cycloalkyl, heterocycloalkyl, aryl and heteroaryl are optionally substituted with at least one functional group selected from the group consisting of halo, alkyl, alkoxy, —CF₃, —OCF₃, —CN, cycloalkyl, aryl or heteroaryl is optionally substituted with at least one functional group selected from the group consisting of alkyl, alkoxy, fluoroalkyl, cycloalkyl, hydroxy, halo, fluoroalkoxy, alkenyl, alkenoxy and —R₉Q, wherein R₉ and Q are as defined above;
R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are each independently selected from the group consisting of hydrogen, alkyl, aryl, heteroaryl, allyl, alkoxy, cycloalkyl, heterocycloalkyl, haloalkyl, fluorocycloalkyl, arylalkyl, and heteroarylalkyl; or wherein R₁₀ and R₁₁, R₁₆ and R₁₇, R₂₁ and R₂₂ or R₂₃ and R₂₄, taken together with the nitrogen to which they are attached, are part of a heterocycloalkyl or heteroaryl;
Y is selected from the group consisting of —CO—CO—, —SO₂—, —C═NRₓ—CO—, and —CO—C═NRₓ—, —O—CO—, and —NR₃₀CO—; wherein Rₓ is selected from the group consisting of hydrogen, cyano, alkyl, fluoroalkyl, alkoxyalkyl, cycloalkyl, heterocycloalkyl, aryl and heteroaryl, optionally substituted with at least one functional group selected from the group consisting of halo, alkyl, alkoxy, —CF₃, —OCF₃, and —CN;
R₁, R₂, R₃, R₄, R₅, R₆, R₇ and R₈ are each independently hydrogen, halo, or alkyl; at least one of R₁, R₂, R₃, R₄ is taken together with at least one of R₅, R₆, R₇ and R₈ to form an alkylene bridge,
wherein the alkyl or alkylene bridge is optionally substituted with at least one functional group selected from the group consisting of halogen, amino, hydroxyl, —CN, —NO₂, alkoxy, —CF₃, —OCF₃, alkyl, allyl, fluoroalkyl, cycloalkyl, fluorocycloalkyl, heterocycloalkyl, aryl, heteroaryl, arylalkyl, heteroarylalkyl, polyether and R₃₁-Q′ wherein R₃₁ is null or alkylene and Q′ is selected from the group consisting of —SO₂NR₃₂R₃₃, —NR₃₄COR₃₅, —CONR₃₆R₃₇ and —COOR₃₈;
R₃₀, R₃₂, R₃₃, R₃₄, R₃₅, R₃₆, R₃₇ and R₃₈ are each independently selected from the group consisting of hydrogen, alkyl, allyl, fluoroalkyl, cycloalkyl, heterocycloalkyl, fluorocycloalkyl, alkoxy, aryl, heteroaryl, arylalkyl, fluoroalkoxy, fluoroalkyl, fluorocycloalkyl, alkylthio, hydroxy, alkenyl, alkenoxy and —R₉Q, wherein R₉ and Q are defined above;
R₃₉ is selected from the group consisting of cycloalkyl, heterocycloalkyl, aryl and heteroaryl, each optionally substituted with at least one functional group selected from the group consisting of halogen, alkyl, alkoxy, —CF₃, —OCF₃, —CN, cycloalkyl, fluoroalkoxy, fluoroalkyl, fluorocycloalkyl, S-alkyl, hydroxy, alkenyl, alkenoxy, acetyl and —R₉Q, wherein R₉ and Q are defined above; and
R₄₀ is selected from the group consisting of hydrogen, —CN, alkyl, halo, —CF₃, cycloalkyl, fluoroalkyl, fluorocycloalkyl, aryl, heteroaryl, arylalkyl, heteroarylalkyl and heterocycloalkyl,
wherein the cycloalkyl, heterocycloalkyl, aryl, heteroaryl, arylalkyl and heteroarylalkyl are optionally substituted with at least one functional group selected from the group consisting of halo, alkyl, alkoxy, —CF₃, —OCF₃, —CN, cycloalkyl, fluoroalkoxy, fluoroalkyl, fluorocycloalkyl, alkylthio, hydroxy, alkenyl, alkenoxy and —R₉Q, wherein R₉ and Q are defined above.

2. The compound of claim 1, wherein
W is selected from the group consisting of null, C₀-C₆ alkylene, (C₀-C₃ alkylene)-O—(C₀-C₃ alkylene), (C₀-C₃ alkylene)-NR′—(C₀-C₃ alkylene), (C₀-C₃ alkylene)-S—(C₀-C₃ alkylene), (C₀-C₃ alkylene)-S(═O)—(C₀-C₃ alkylene), (C₀-C₃ alkylene)-SO₂—(C₀-C₃ alkylene), (C₀-C₃ alkylene)-C(═O)—(C₀-C₃ alkylene), (C₀-C₃ alkylene)-C(═O)NR′—(C₀-C₃ alkylene) and (C₀-C₆ cycloalkylidene), wherein the alkylene and cycloalkylidene groups are optionally substituted at least one halogen atom;
A₁ is phenylene or monocyclic heteroarylene, wherein the phenylene and monocyclic heteroarylene are optionally substituted with at least one functional group selected from the group consisting of C₁-C₆ alkyl, C₁-C₆ alkoxy, C₁-C₆ fluoroalkyl, C₃-C₇ cycloalkyl, hydroxy, halo, C₁-C₆ fluoroalkoxy, C₂-C₆ alkenyl, C₂-C₆ alkenoxy and —R₉Q, wherein R₉ is null or C₁-C₂ alkylene and Q is selected from the group consisting of —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈, and —OR₂₉;
A₂ is phenyl or heteroaryl, wherein the phenyl and heteroaryl are optionally substituted with at least one functional group selected from the group consisting of C₁-C₆ alkyl, C₁-C₆ alkoxy, C₁-C₆ fluoroalkyl, C₃-C₇ cycloalkyl, hydroxy, halogen, C₁-C₆ fluoroalkoxy, C₂-C₆ alkenyl, C₂-C₆ alkenoxy and —R₉Q, wherein R₉ is null or C₁-C₂ alkylene and Q is defined above;
R′, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are each independently selected from the group consisting of hydrogen, C₁-C₆ alkyl, allyl, C₁-C₆ fluoroalkyl, C₃-C₇ cycloalkyl, C₃-C₇ fluorocycloalkyl, C₁-C₆ alkoxy, phenyl, phenylmethyl, phenylethyl, heteroaryl, heteroarylmethyl, heteroarylethyl, heterocycloalkyl, heterocycloalkylmethyl and heterocycloalkylethyl; or wherein R₁₀ and R₁₁, R₁₆ and R₁₇, R₂₁ and R₂₂, or R₂₃ and R₂₄, taken together with the nitrogen to which they are attached, are part of a ring selected from the group consisting of azetidine, azetidin-2-one, pyrrolidine, pyrrolidin-2-one, pyrrolidin-3-one, piperidine, piperidin-2-one, piperidin-3-one, piperidin-4-one, morpholine, morpholin-2-one, morpholin-3-one and N-alkylpiperazine;
wherein the heterocycloalkyl comprises
0 to 4 nitrogen atoms;
0 to 2 nitrogen atoms and 0 to 1 oxygen atom;
0 to 2 nitrogen atoms and 0 to 1 sulfur atom; or
0 to 2 nitrogen atoms, 0 to 1 oxygen atom and 0 to 1 sulphur atom; and
wherein the heteroaryl is selected from the group consisting of imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenzimidazolyl, indazolyl, quinazolinyl, phthalazinyl, benzoxazolyl and quinoxalinyl; and
wherein the phenyl, heteroaryl or heterocycloalkyl is optionally substituted with 1 to 5 functional groups selected from the group consisting of hydrogen, halo, C₁-C₆ alkoxy, —CF₃, —OCF₃ and —CN;
Rₓ is selected from the group consisting of alkyl, fluoroalkyl, alkoxyalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl and quinoxalinyl; wherein each heteroaryl ring is optionally substituted with at least one functional group selected from the group consisting of halo, C₁-C₆ alkyl, C₁-C₆ alkoxy, —CF₃, —OCF₃ and —CN;
R₁, R₂, R₃, R₄, R₅, R₆, R₇, and R₈ are each independently hydrogen or C₁-C₆ alkyl,
wherein the C₁-C₆ alkyl is optionally substituted with at least one functional group selected from the group consisting of hydrogen, halo, amino, hydroxyl, —CN, —NO₂, C₁-C₆ alkoxy, —CF₃, —OCF₃, C₁-C₆ alkyl, allyl, C₁-C₆ fluoroalkyl, C₃-C₇ cycloalkyl, C₃-C₇ fluorocycloalkyl, phenyl, phenylmethyl, phenylethyl, heteroaryl, heteroarylmethyl, heteroarylethyl, heterocycloalkyl, heterocycloalkylmethyl, heterocycloalkylethyl, (CRₐR_{b})_{U}-T-(CR_{c}R_{d})_{U′}Rₑ and R₃₁Q′ wherein R₃₁ is null or C₁-C₂ alkylene and Q′ is selected from the group consisting of —SO₂NR₃₂R₃₃, —NR₃₄COR₃₅, —CONR₃₆R₃₇ and —COOR₃₈;
R₃₀, R₃₂, R₃₃, R₃₄, R₃₅, R₃₆, R₃₇, R₃₈, Rₐ, R_{b}, R_{c}, R_{d} and Rₑ are each independently selected from the group consisting of hydrogen, C₁-C₆ alkyl, allyl, C₁-C₆ fluoroalkyl, C₃-C₇ cycloalkyl, C₃-C₇ fluorocycloalkyl, C₁-C₆ alkoxy, phenyl-(C₀-C₂ alkyl), heteroaryl-(C₀-C₂ alkyl) and heterocycloalkyl-(C₀-C₂ alkyl);
wherein the heterocycloalkyl comprises
0 to 4 nitrogen atoms;
0 to 2 nitrogen atoms and 0 to 1 oxygen atom;
0 to 2 nitrogen atoms and 0 to 1 sulfur atom; or
0 to 2 nitrogen atoms, 0 to 1 oxygen atom and 0 to 1 sulphur atom; and
wherein the heteroaryl group is selected from the group consisting of imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenzimidazolyl, indazolyl, quinazolinyl, phthalazinyl, benzoxazolyl, and quinoxalinyl;
wherein the phenyl, heteroaryl or heterocycloalkyl is optionally substituted with 1 to 5 functional groups selected from group consisting of halo, C₁-C₆ alkoxy, —CF₃, —OCF₃ and —CN; or wherein R₃₂ and R₃₃ or R₃₆ and R₃₇, taken together with the nitrogen to which they are attached, are part of a heterocycloalkyl selected from the group consisting of aziridine, azetidine, pyrrolidine, pyrrolidin-2-one, piperidine, morpholine and N-alkylpiperazine;
U and U′ are each independently 0, 1 or 2;
T is null or oxy;
R₄₁ is selected from the group consisting of phenyl, pyridinyl, pyrazinyl, pyridazinyl, pyrimidinyl, thienyl, furyl, thiazolyl, isothiazolyl, oxazolyl, isoxazolyl, pyrazoyl, imidazolyl, triazolyl, oxadiazolyl, thiadiazolyl, and tetrazolyl; each of which is optionally substituted with one or more C₁-C₃ alkyl, C₃-C₆ cycloalkyl, C₁-C₃ alkoxy, C₁-C₆ fluoroalkoxy, C₁-C₆ fluoroalkyl, C₃-C₇ fluorocycloalkyl, —CN, —F, —Cl, —Br, —CF₃, C₀-C₃ alkylthio, hydroxy, C₂-C₆ alkenyl, C₂-C₆ alkenoxy, acetyl and —R₉Q, wherein R₉ is null or C₁-C₂ alkylene and Q is defined above;
R₄₂ is selected from the group consisting of phenyl, heteroaryl, quinolinyl, isoquinolinyl, benzimidazolyl, azabenimidazolyl, benzothienyl, benzofuryl, benzoindazolyl, quinazolinyl, phthalazinyl, benzoxazolyl, quinoxalinyl, thienopyridine, thienopyrimidine, thienopyridazine, thienopyrazine, furopyridine, furoopyrimidine, furopyridazine, furopyrazine, oxazolopyridine, oxazolopyrimidine, oxazolopyridazine, oxazolopyrazine, thiazolopyridine, thiazolopyrimidine, thiazolopyridazine, thiazolopyrazine, napthyridine, pyridopyrimidine, pyridopyridazine and pyridopyrazine;
each optionally substituted with at least one functional group selected from the group consisting of halo, C₁-C₆ alkoxy, —CF₃, —OCF₃ or —CN, C₁-C₃ alkyl, C₃-C₆ cycloalkyl, C₁-C₆ fluoroalkoxy, C₁-C₆ fluoroalkyl, C₃-C₇ fluorocycloalkyl, C₀-C₃ alkylthio, hydroxy, C₂-C₆ alkenyl, C₂-C₆ alkenoxy, acetyl and —R₉Q, wherein R₉ is null or C₁-C₂ alkylene and Q is defined above;
R₄₃ is selected from the group consisting of hydrogen, —CN, C₁-C₆ alkoxy, C₁-C₆ fluoroalkoxy, C₁-C₆ alkyl, C₁-C₆ fluoroalkyl, C₃-C₇ cycloalkyl or C₃-C₇ fluorocycloalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl, and quinoxalinyl;
wherein the aryl or heteroaryl are optionally substituted with at least one functional group selected from the group consisting of halo, C₁-C₆ alkyl, C₁-C₆ alkoxy, —CF₃, —OCF₃ or —CN, C₁-C₃ alkyl, C₃-C₆ cycloalkyl, C₁-C₆ fluoroalkoxy, C₁-C₆ fluoroalkyl, C₃-C₇ fluorocycloalkyl, C₀-C₃ alkylthio, hydroxy, C₂-C₆ alkenyl, C₂-C₆ alkenoxy, acetyl and —R₉Q, wherein R₉ is null or C₁-C₂ alkylene and Q is defined above;
R₃₉ is selected from the group consisting of phenyl, imidazolyl, pyrazolyl, triazolyl, tetrazolyl, thienyl, furyl, thiazolyl, isothiazolyl, oxazolyl,isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl, and quinoxalinyl;
each optionally substituted with at least one functional group selected from the group consisting of halo, C₁-C₆ alkyl, C₁-C₆ alkoxy, —CF₃, —OCF₃, —CN, C₁-C₃ alkyl, C₃-C₆ cycloalkyl, C₁-C₆ fluoroalkoxy, C₁-C₆ fluoroalkyl, C₃-C₇ fluorocycloalkyl, S—(C₀-C₃ alkyl), hydroxy, C₂-C₆ alkenyl, C₂-C₆ alkenoxy, acetyl and —R₉Q, wherein R₉ is null or C₁-C₂ alkylene and Q is defined above; and
R₄₀ is selected from the group consisting of hydrogen, —CN, C₁-C₆ alkyl, halo, —CF₃, C₃-C₆ cycloalkyl, C₁-C₆ fluoroalkyl, C₃-C₇ fluorocycloalkyl, and heterocycloalkyl, heterocycloalkylmethyl, heterocycloalkylethyl, R₄₁, R₄₁methyl and R₄₁ethyl;
wherein the heterocycloalkyl comprises
0 to 4 nitrogen atoms;
0 to 2 nitrogen atoms and 0 to 1 oxygen atom;
0 to 2 nitrogen atoms and 0 to 1 sulfur atom; or
0 to 2 nitrogen atoms, 0 to 1 oxygen atom and 0 to 1 sulphur atom; and
wherein R₄₁ is selected from the group consisting of phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, azabenimidazolyl, indazolyl, quinazolinyl, phthalazinyl, benzoxazolyl, and quinoxalinyl; and is optionally substituted with at least one functional group selected from the group consisting of halogen, C₁-C₆ alkyl, C₁-C₆ alkoxy, —CF₃, —OCF₃, —CN, hydrogen, C₁-C₃ alkyl, C₃-C₆ cycloalkyl, O—(C₁-C₆ fluoroalkyl), C₁-C₆ fluoroalkyl, C₃-C₇ fluorocycloalkyl, S—(C₀-C₃ alkyl), hydroxy, C₂-C₆ alkenyl, C₂-C₆ alkenoxy, acetyl and —R₉Q, wherein R₉ is null or C₁-C₂ alkylene and Q is defined above.

3. A compound of Formula (II) 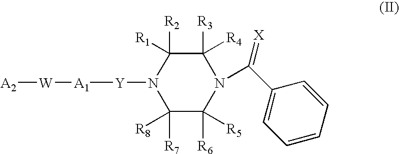
or a pharmaceutically acceptable prodrug, salt, polymorph, solvate, enantiomer, diastereomer, racemate, mixture of stereoisomers thereof, or derivative thereof, wherein:
W is selected from the group consisting of null, oxy, amino, thio, sulfinyl, sulfonyl, carbonyl, amide, alkylene and cycloalkylidene,
wherein at least one carbon atom of the alkylene or cycloalkylidene is optionally substituted with an oxy, amino, thio, sulfinyl, sulfonyl, carbonyl or amide group, and wherein the alkylene or cycloalkylidene is optionally substituted with 1 to 3 halogen atoms;
A₁ is a monocyclic ring selected from the group consisting of monocyclic cycloalkylidene, monocyclic heterocycloalkylidene, monocyclic arylene and monocyclic heteroarylene, wherein the monocyclic ring is optionally substituted with 1 to 5 functional groups, each independently selected from the group consisting of alkyl, alkoxy, fluoroalkyl, cycloalkyl, hydroxy, halo, fluoroalkoxy, alkenyl, alkenoxy and —R₉Q, wherein Q is selected from the group consisting of —NR₁₀R₁₁, —CN, —CO₂R₁₂, —SR₁₃, —SOR₁₄, —SO₂R₁₅, —SO₂NR₁₆R₁₇, —NR₁₈COR₁₉, —NR₂₀CONR₂₁R₂₂, —CONR₂₃R₂₄, —NR₂₅SOR₂₆, —R₂₇COR₂₈ and —OR₂₉;
A₂ is selected from the group consisting of null, cycloalkyl, heterocycloalkyl, aryl and heteroaryl, wherein the cycloalkyl, heterocycloalkyl, aryl or heteroaryl is optionally substituted with 1 to 5 functional groups, each independently selected from the group consisting of alkyl, alkoxy, fluoroalkyl, cycloalkyl, hydroxy, halo, fluoroalkoxy, alkenyl, alkenoxy and —R₉Q, wherein R₉ and Q are as defined above;
R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈ and R₂₉ are each independently selected from the group consisting of hydrogen, alkyl, aryl, heteroaryl, allyl, alkoxy, cycloalkyl, heterocycloalkyl, fluoroalkyl, fluorocycloalkyl, arylalkyl, and heteroarylalkyl; or wherein R₁₀ and R₁₁, R₁₆ and R₁₇, R₂₁ and R₂₂, or R₂₃ or R₂₄, taken together with the nitrogen to which they are attached, are part of a heterocycloalkyl or heteroaryl;
Y is selected from the group consisting of —CO—CO—, —SO₂—, —C═NRₓ—CO—, and —CO—C═NRₓ—, —O—CO—, and —NR₃₀CO—; wherein Rₓ is selected from the group consisting alkyl, fluoroalkyl, alkoxyalkyl, cycloalkyl, heterocycloalkyl, aryl and heteroaryl, optionally substituted with 1 to 5 functional groups selected from the group consisting of halogen, alkyl, alkoxy, —CF₃, —OCF₃ and —CN;
R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge,
wherein the alkyl or alkylene bridge is optionally substituted with 1 to 3 functional groups, each independently selected from the group consisting of halogen, amino, hydroxyl, —CN, —NO₂, alkoxy, —CF₃, —OCF₃, alkyl, allyl, fluoroalkyl, cycloalkyl, fluorocycloalkyl, heterocycloalkyl, aryl, heteroaryl, arylalkyl, heteroarylalkyl, polyether and R₃₁-Q′ wherein R₃₁ is null or alkylene and Q′ is selected from the group consisting of —SO₂NR₃₂R₃₃, —NR₃₄COR₃₅, —CONR₃₆R₃₇ and —COOR₃₈;
R₃₀, R₃₂, R₃₃, R₃₄, R₃₅, R₃₆, R₃₇ and R₃₈ are each independently selected from the group consisting of hydrogen, alkyl, allyl, fluoroalkyl, cycloalkyl, heterocycloalkyl, fluorocycloalkyl, alkoxy, aryl, heteroaryl, arylalkyl, heteroarylalkyl; or wherein R₃₂ and R₃₃ or R₃₆ and R₃₇, taken together with the nitrogen to which they are attached, are part of a heterocycloalkyl or heteroaryl; and
wherein the cycloalkyl, heterocycloalkyl, aryl and heteroaryl are each independently optionally substituted with 1 to 5 functional groups selected from the group consisting of halo, alkoxy, —CF₃, —OCF₃ and —CN; and
X is O, S or NR₃₉, wherein R₃₉ is selected from the group consisting of hydrogen, —CN, alkoxy, fluoroalkoxy, alkyl, fluoroalkyl, cycloalkyl, fluorocycloalkyl, phenyl, imidazolyl, pyrazolyl, triazolyl, tetrazolyl, thienyl, furyl, thiazolyl, isothiazolyl, oxazolyl, isoxazolyl, thiadiazolyl, oxadiazolyl, pyridinyl, pyrimidinyl, pyridazinyl, pyrazinyl, quinolinyl, isoquinolinyl, benzimidazolyl, indazolyl, quinazolinyl, phthalazinyl, benzoxazolyl and quinoxalinyl;
optionally substituted with 1 to 5 functional groups selected from the group consisting of halo, alkyl, alkoxy, —CF₃, —OCF₃ or —CN, cycloalkyl, fluoroalkoxy, fluoroalkyl, fluorocycloalkyl, alkylthio, hydroxy, alkenyl, alkenoxy, acetyl and —R₉Q, wherein R₉ and Q are defined above.

4. The compound of claim 3, wherein
W is —(CH₂)ₓ(CO)_{y}(CH₂)_{z}—, wherein x, y and z are each independently 0, 1, 2 or 3;
A₁ is a monocyclic ring selected from the group consisting of a cycloalkylidene, heterocycloalkylidene, arylene and heteroarylene, each optionally substituted with 1 to 3 functional groups selected from the group consisting of halo, alkyl, alkoxy, fluoroalkyl, fluoroalkoxy, hydroxy, amino, alkylamino, dialkylamino and thiol;
A₂ is a monocyclic or bicyclic ring selected from the group consisting of monocyclic or bicyclic cycloalkyl, monocyclic or bicyclic heterocycloalkyl, monocyclic or bicyclic aryl and monocyclic or bicyclic heteroaryl,
each optionally substituted with 1 to 3 functional groups selected from the group consisting of halo, —CN, alkyl, alkoxy, acetyl, oxo, fluoroalkyl, fluoroalkoxy, hydroxy, amino, methylamino, dimethylamino, —SH, cycloalkyl, heterocycloalkyl, aryl, heteroaryl, arylalkyl and arylcarbonyl;
wherein the cycloalkyl, heterocycloalkyl, aryl or heteroaryl substituted onto the monocyclic or bicyclic ring is optionally substituted with a halo, alkyl, acetyl or alkoxycarbonyl;
Y is —(CH₂)ₘ(C═O)ₙ— or —SO₂—, wherein m and n are each independently 0, 1, 2 or 3;
R₁, R₂, R₃, R₄, R₅, R₆, R₇ and R₈ are each independently hydrogen or alkyl; and/or at least one of R₁, R₂, R₃, R₄ is taken together with at least one of R₅, R₆, R₇ and R₈ to form an alkylene bridge; and
X is O, —CN or N—O-alkyl.

5. The compound of claim 1, wherein the compound is selected from the group consisting of:

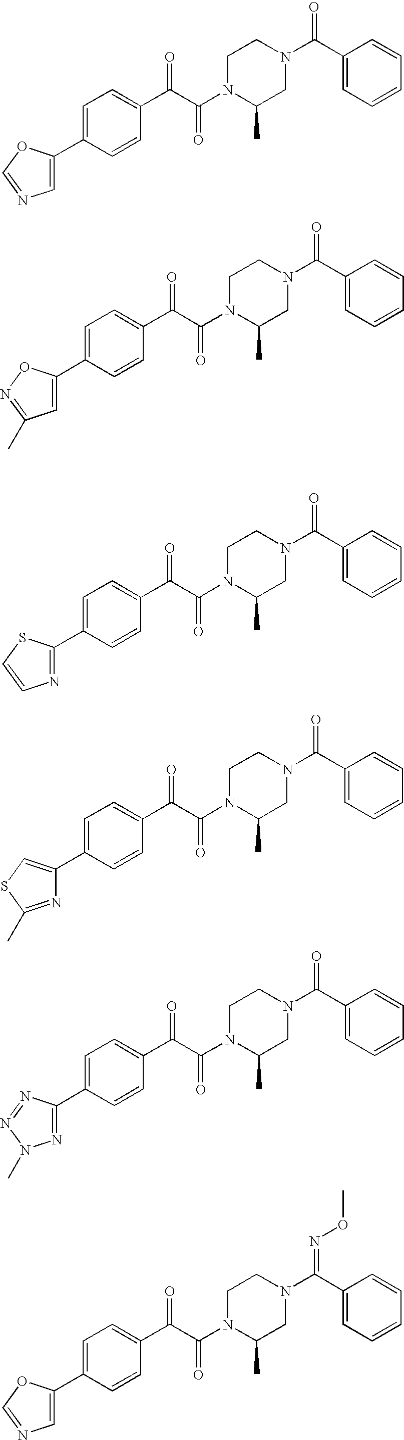

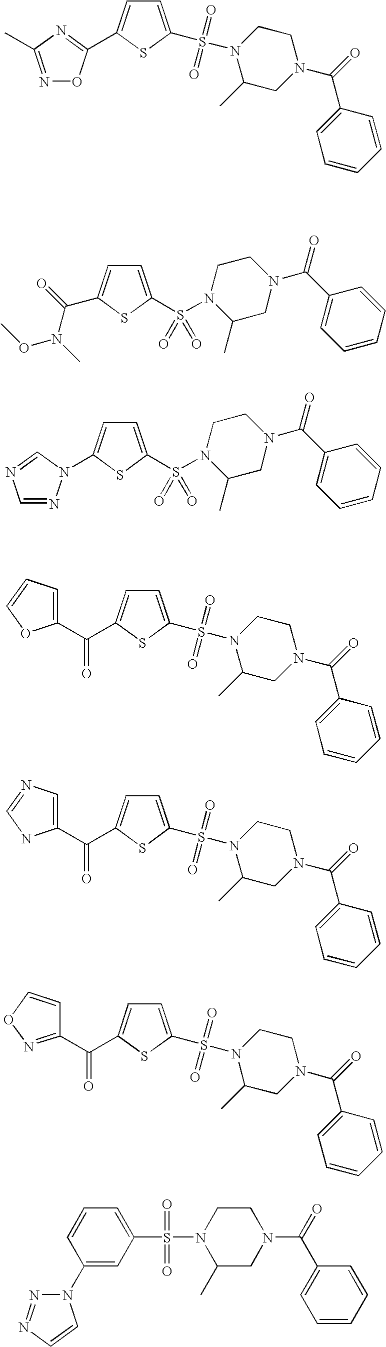

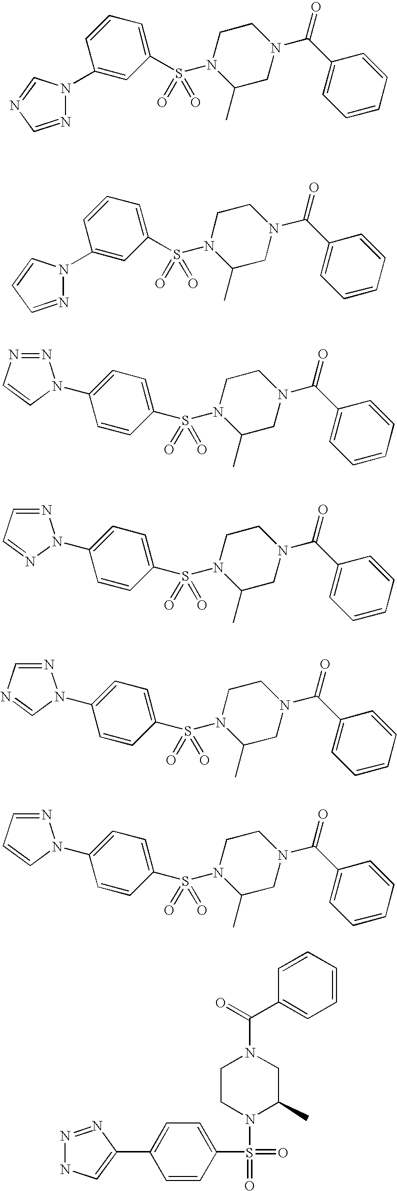

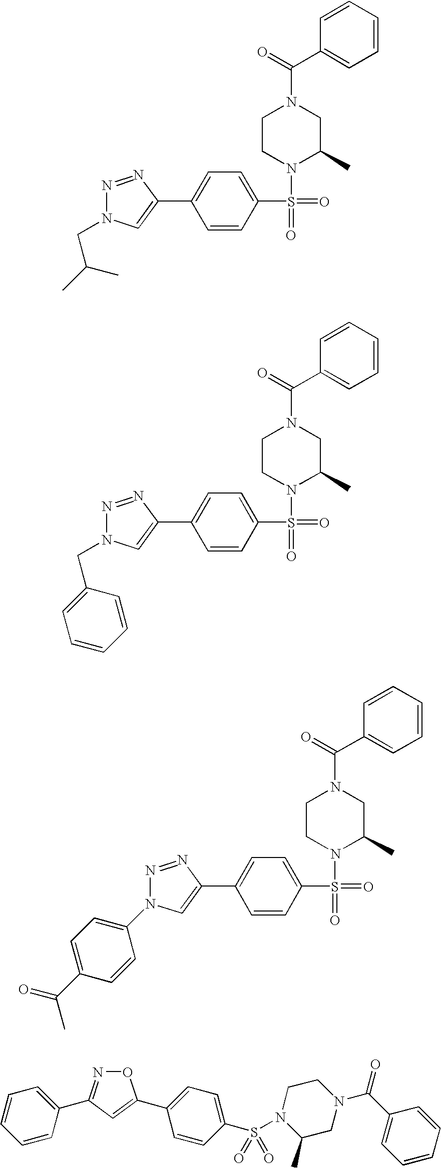

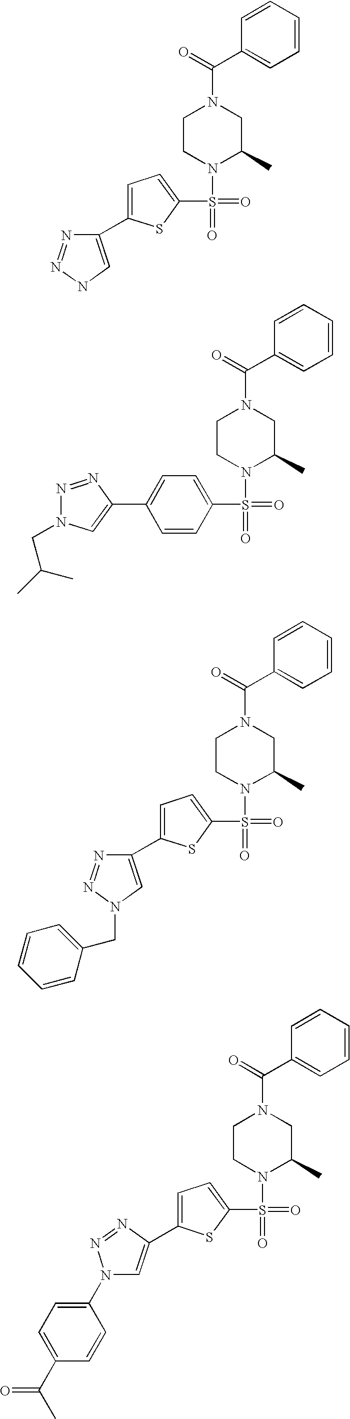

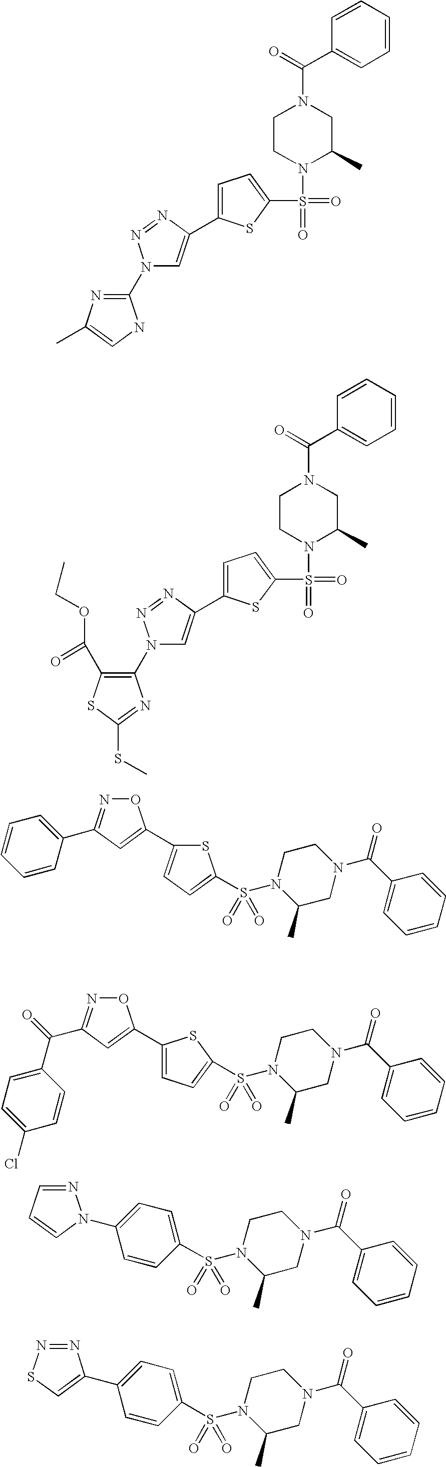

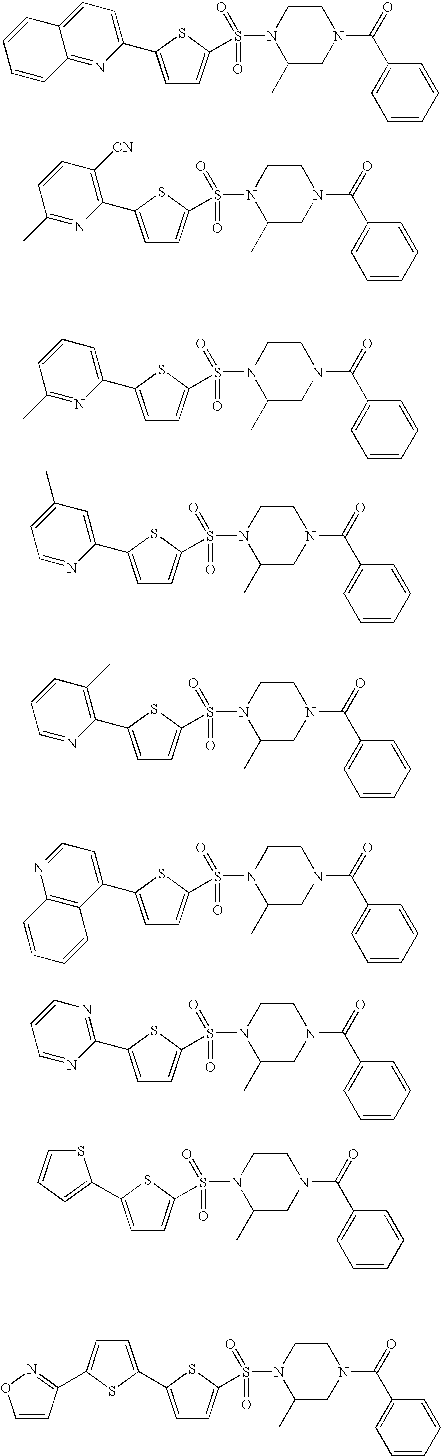

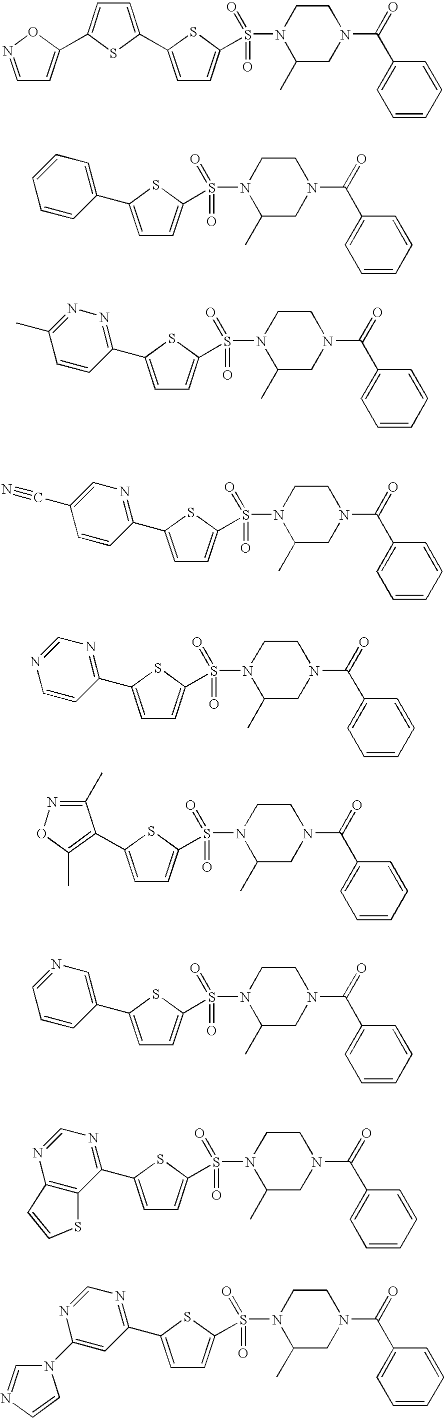

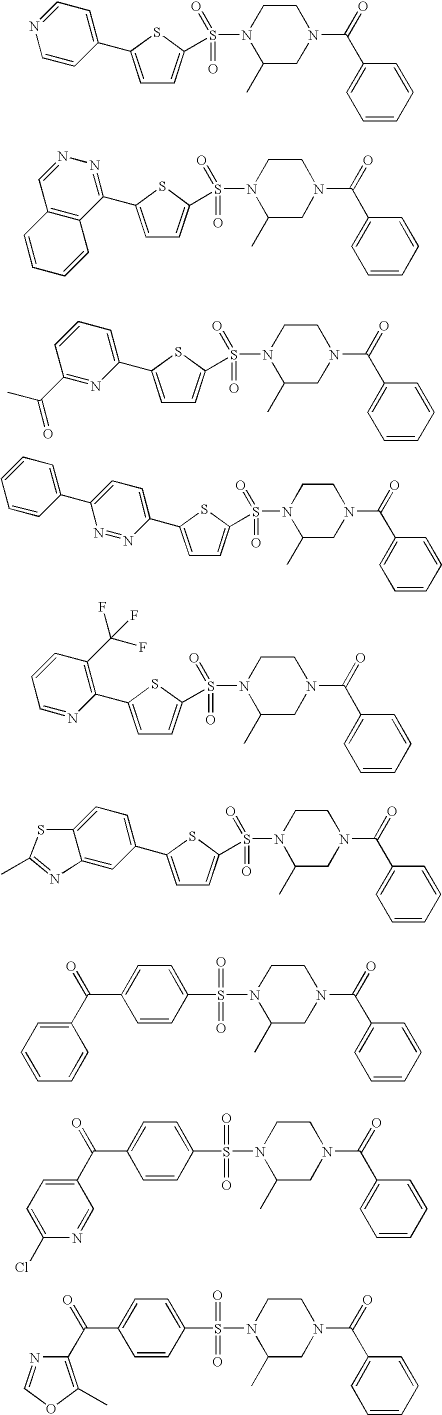

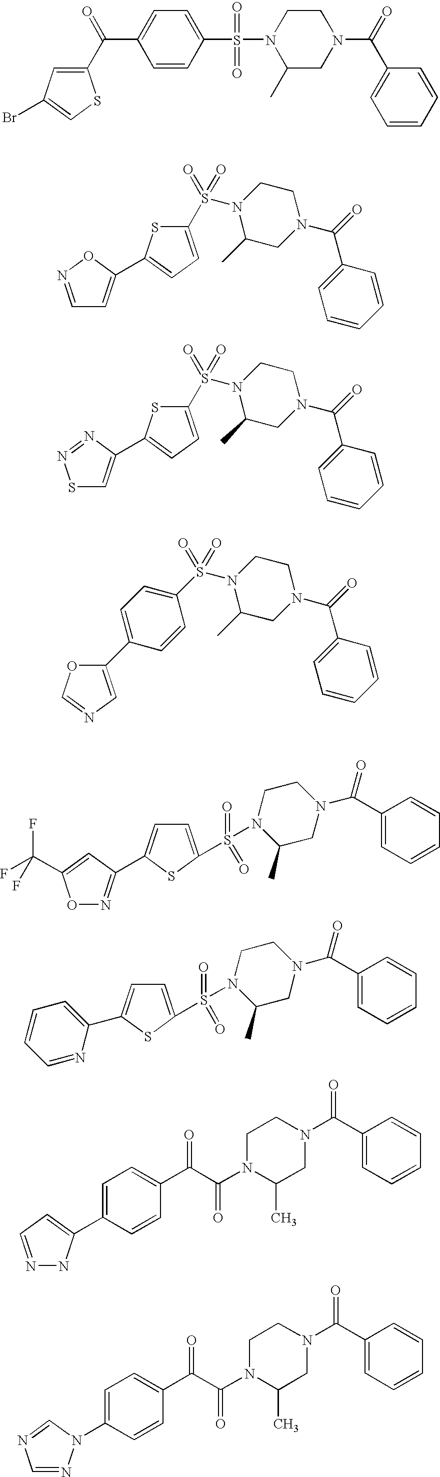

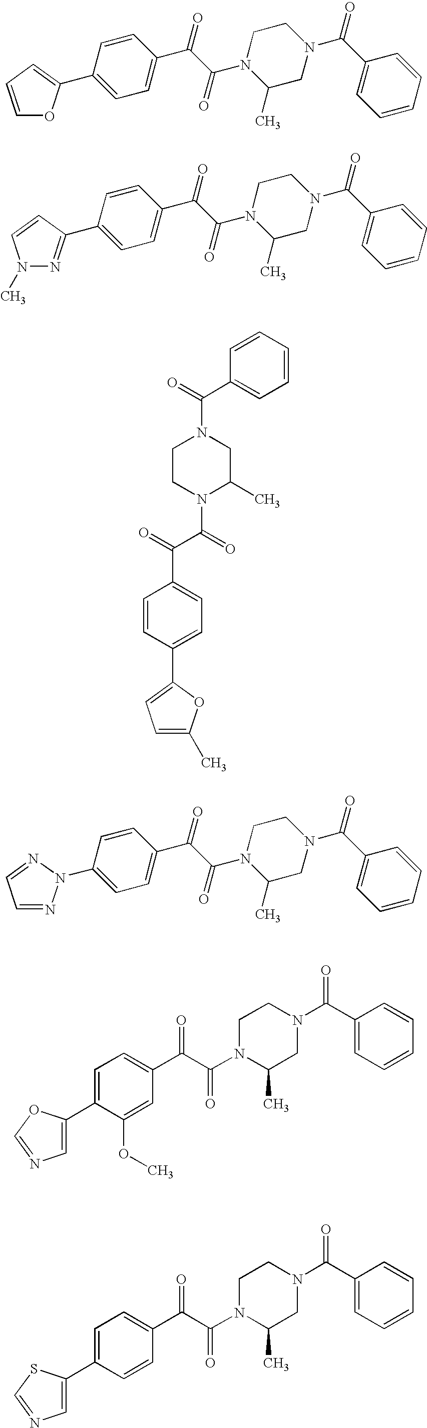

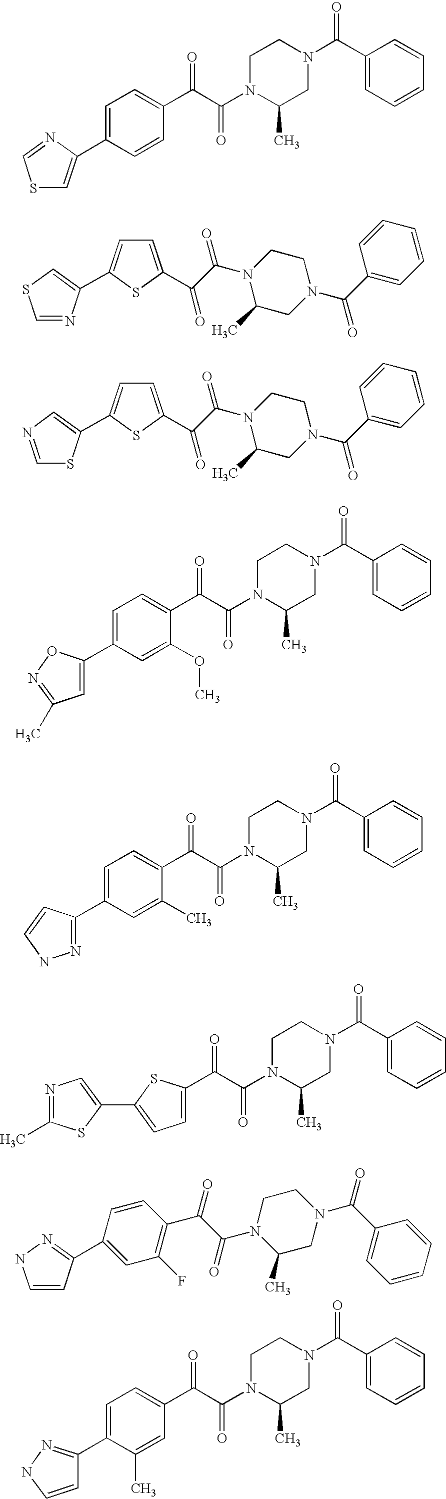

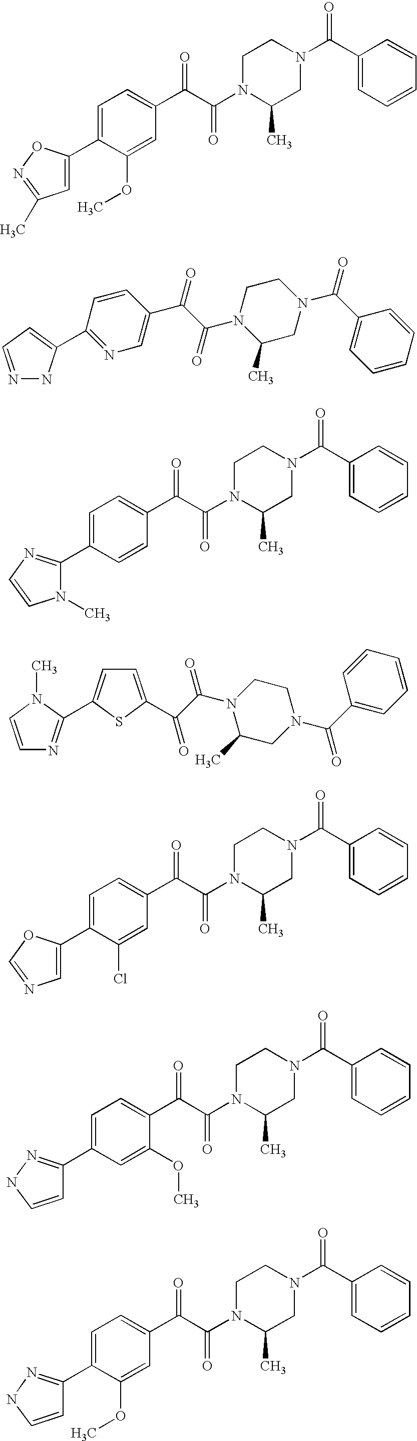

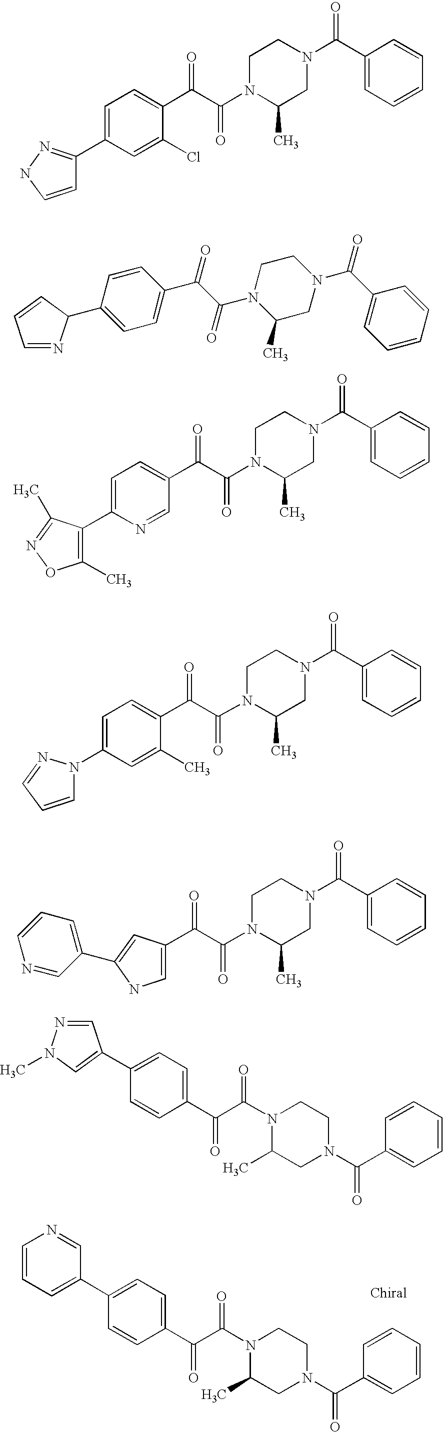

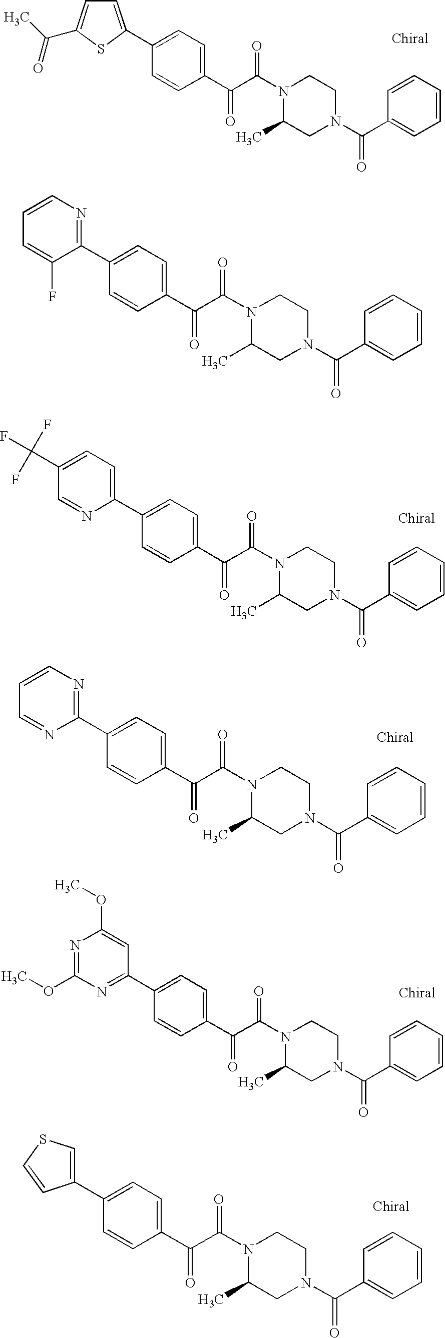

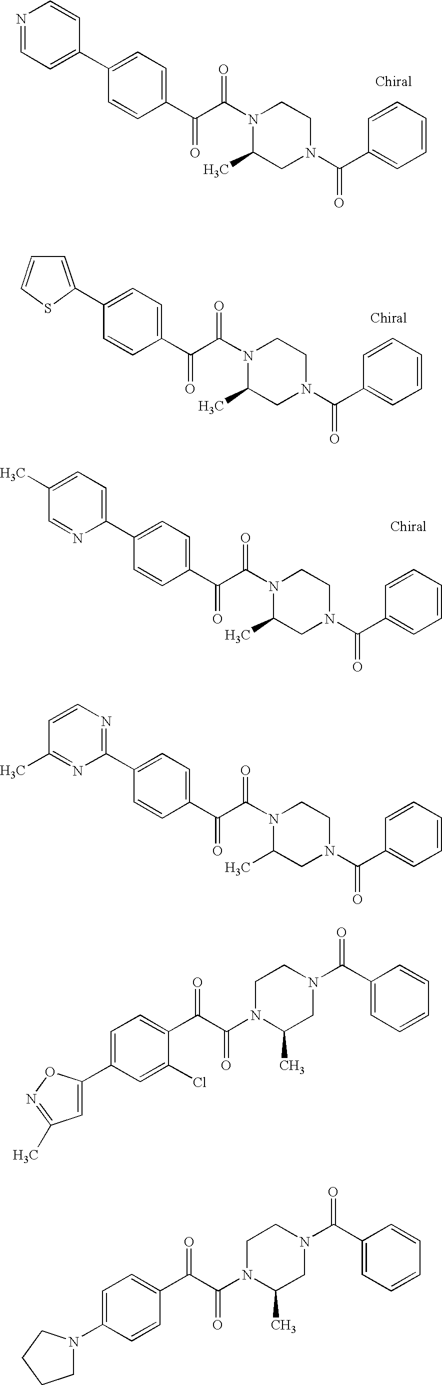

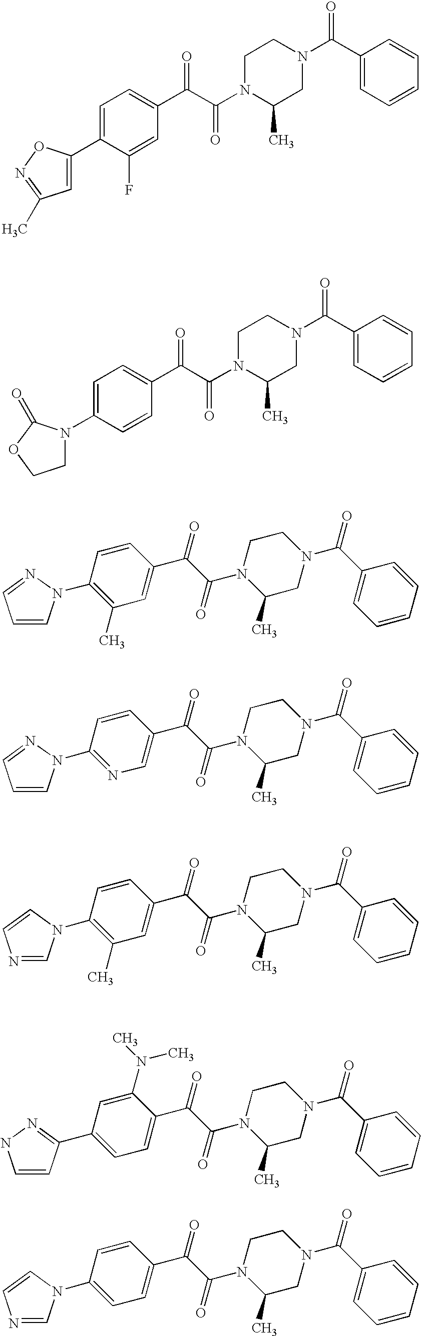

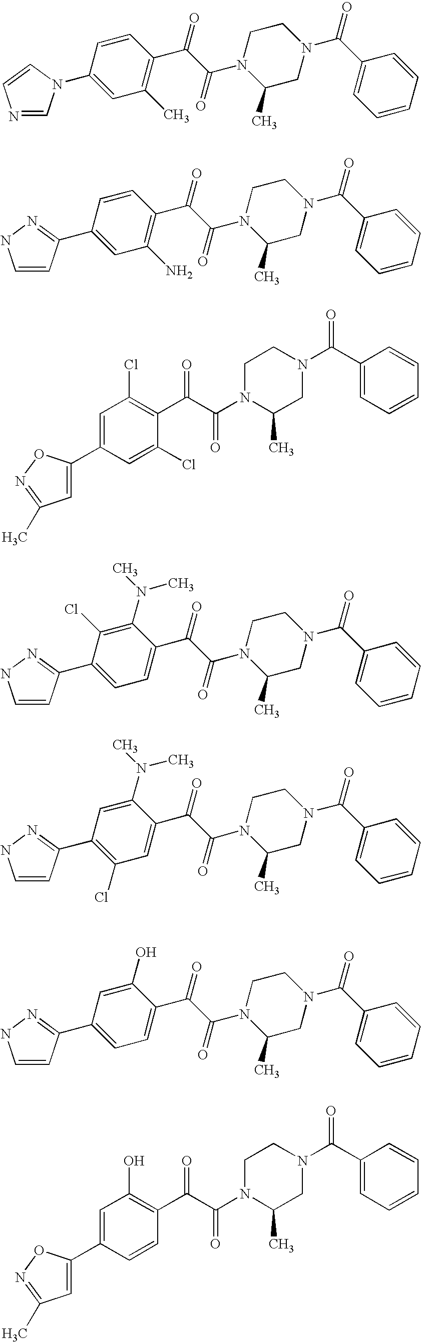

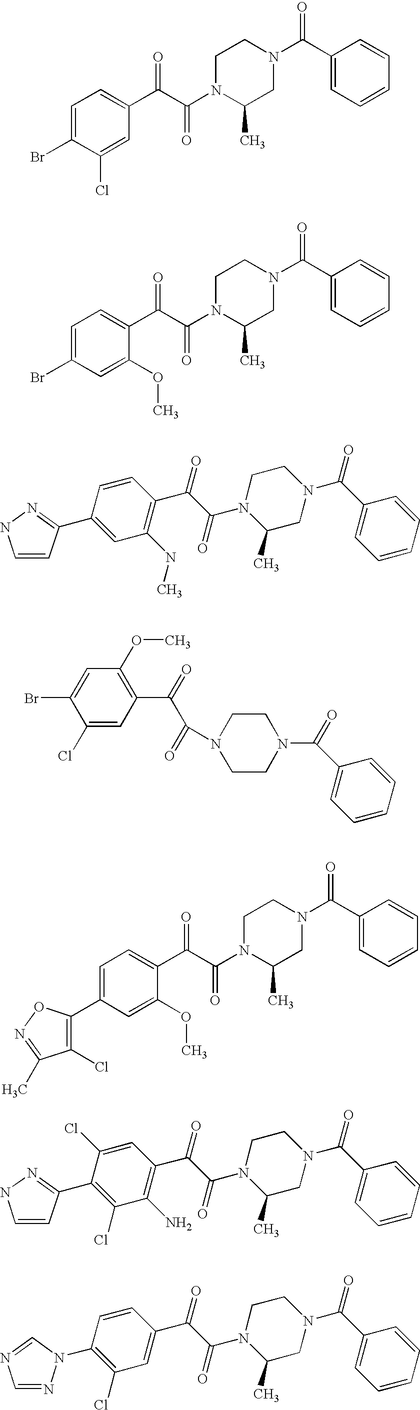

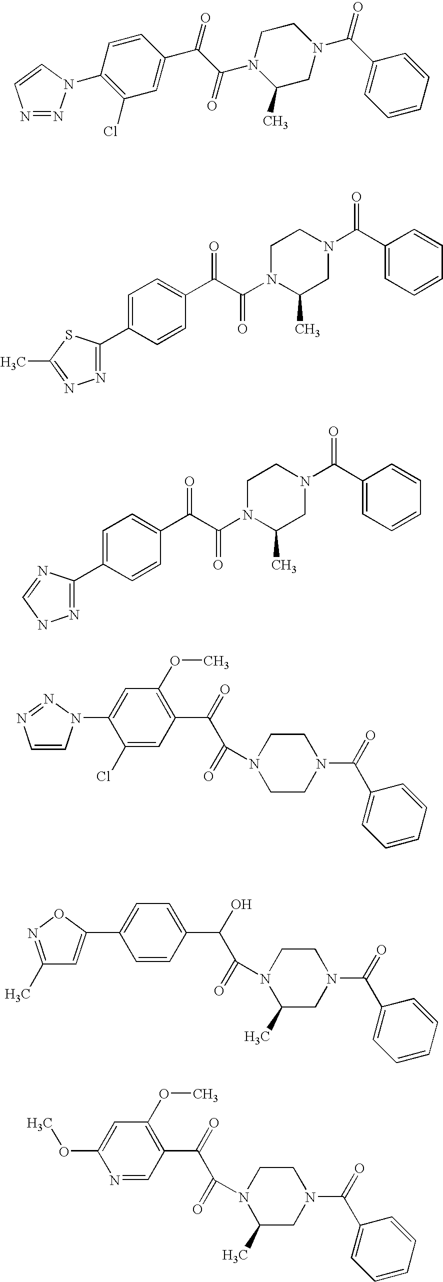

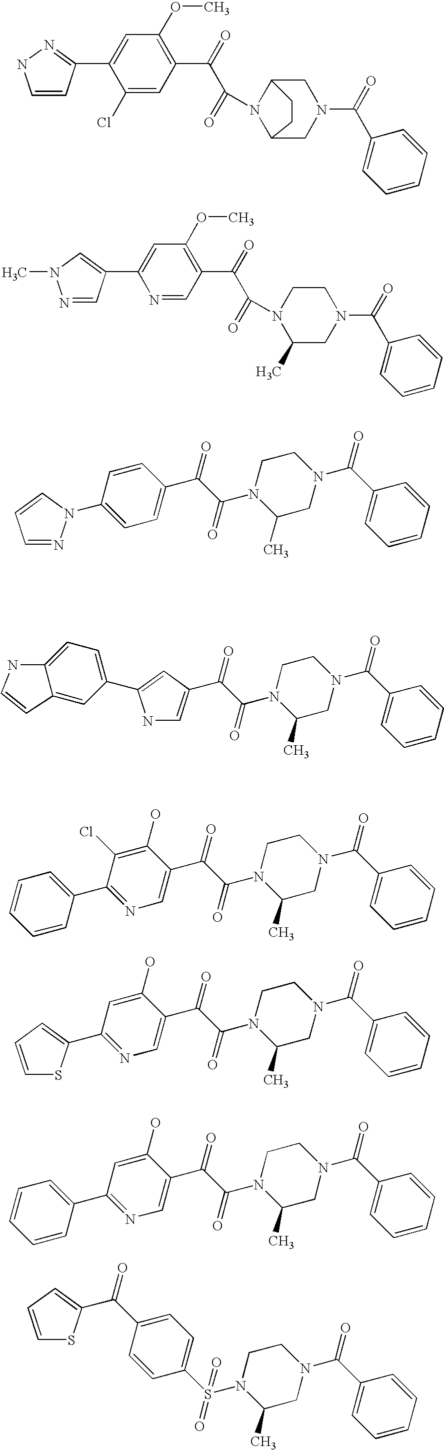

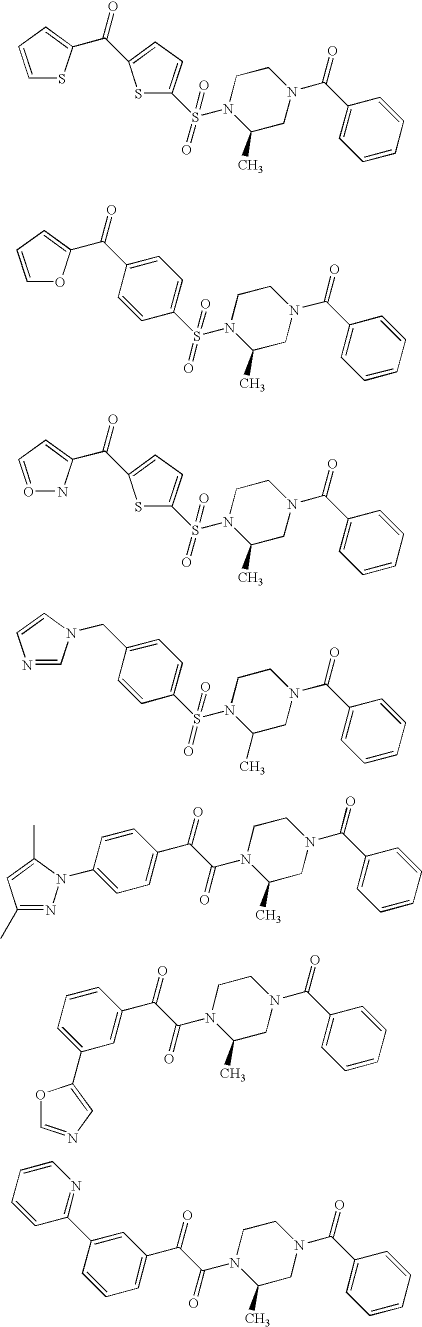

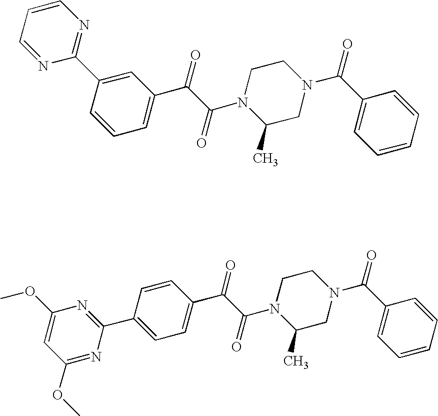

6. The compound of claim 1, selected from the group consisting of the compounds 101, 102, 103, 105, 106, 108, 120, 141, 142, 144, 147, 164, 169, 170, 171, 172, 173, 174, 175, 176, 177, 178, 179, 180, 181, 183, 184, 185, 186, 189, 190, 191, 192, 193, 197, 198, 199,

7. The compound of claim 1, wherein the compound is present as a racemic mixture.

8. The compound of claim 1, wherein the compound is present substantially as the (R) enantiomer.

9. A pharmaceutical composition comprising:
a compound of Formula I of claim 1, or a pharmaceutically acceptable prodrug, salt, polymorph, solvate, enantiomer, diastereomer, racemate, mixture of stereoisomers thereof, or derivative thereof; and
a pharmaceutically acceptable carrier, excipient or diluent.

10. A pharmaceutical composition comprising:
a compound of Formula II of claim 3, or a pharmaceutically acceptable prodrug, salt, polymorph, solvate, enantiomer, diastereomer, racemate, mixture of stereoisomers thereof, or derivative thereof; and
a pharmaceutically acceptable carrier, excipient or diluent.

11. A method for the inhibition of transmission of an HIV virus to a cell, comprising contacting the cell with an effective concentration of the compound of claim 1 under conditions sufficient wherein fusion of the virus is inhibited.

12. A method of treating HIV infection in a subject, comprising administering to the subject an effective amount of the compound of claim 1 or a pharmaceutically acceptable prodrug, salt, polymorph, solvate, enantiomer, diastereomer, racemate, mixture of stereoisomers thereof, or derivative thereof.

13. The method of claim 12, wherein the method further comprises administering an effective amount of at least one other therapeutic agent.

14. The method of claim 13, wherein the therapeutic agent is a reverse transcriptase inhibitor, a viral protease inhibitor, a cytokine, a cytokine inhibitor, a glycosylation inhibitor or a viral mRNA processing inhibitor.

15. The method of claim 13, wherein the therapeutic agent is a nucleoside analogue.

16. The method of claim 15, wherein the nucleoside analogue is azidothymidine (AZT), ddI, ddC, ddA, d4T or 3TC.

17. The method of claim 13 wherein the therapeutic agent is interferon-α, interferon-β or interferon-γ.

18. The method of claim 14, wherein the protease inhibitor is an inhibitor of HIV-1 protease.

19. The method of claim 18, wherein the inhibitor of HIV-1 protease is indinavir.

20. The method of claim 13, wherein the administration is sequential.

21. The method of claim 20, wherein the sequential administration is a cycling therapy.

22. The method of claim 21, wherein the sequential administration of each agent comprising the cycling therapy is repeated one or more times in fixed order.

23. The method of claim 21, wherein the cycling therapy comprises administration of the compound of claim 1 or a pharmaceutically acceptable prodrug, salt, polymorph, solvate, enantiomer, diastereomer, racemate, mixture of stereoisomers thereof, or derivative thereof, in alternation with at least one therapeutic agent selected from the group consisting of a reverse transcriptase inhibitor, a viral protease inhibitor, a cytokine, a cytokine inhibitor, a glycosylation inhibitor or a viral mRNA processing inhibitor.

24. The method of claim 13, wherein the administration is simultaneous.

25. The method of claim 13, wherein the compound of claim 1 or or a pharmaceutically acceptable prodrug, salt, polymorph, solvate, enantiomer, diastereomer, racemate, mixture of stereoisomers thereof, or derivative thereof is administered before the other therapeutic agent.

26. The method of claim 13, wherein the compound of claim 1 or or a pharmaceutically acceptable prodrug, salt, polymorph, solvate, enantiomer, diastereomer, racemate, mixture of stereoisomers thereof, or derivative thereof is administered after the therapeutic agent.

27. The method of claim 13, wherein the administration of at least one therapeutic agent is oral.

28. The method of claim 13, wherein the administration is parenteral.

29. The method of claim 28, wherein the parenteral administration is subcutaneous.

30. A method of treating HIV infection in a subject comprising administering an effective amount of a compound of claim 3, or a pharmaceutically acceptable prodrug, salt, polymorph, solvate, enantiomer, diastereomer, racemate, mixture of stereoisomers thereof, or a derivative thereof.

31. The method of claim 30, wherein the method further comprises administering an effective amount of at least one other therapeutic agent.

32. A method of inhibiting HIV replication comprising administering to a subject an effective amount of the compound of claim 1, or a pharmaceutically acceptable prodrug, salt, polymorph, solvate, enantiomer, diastereomier, racemate, mixture of stereoisomers thereof.

33. The method of claim 32, wherein the method further comprises administering an effective amount of at least one other therapeutic agent.

34. A method for the inhibition of transmission of an HIV retrovirus to a cell, comprising contacting the cell with an effective amount of a compound of claim 1, or a pharmaceutically acceptable prodrug, salt, polymorph, solvate, enantiomer, diastereomer, racemate, mixture of stereoisomers thereof, or a derivative therof.

35. The method of claim 34, wherein the method further comprises administering an effective amount of at least one other therapeutic agent.

36. A kit comprising the compound of formula (I) of claim 1, or a pharmaceutically acceptable prodrug, salt, polymorph, solvate, enantiomer, diastereomer, racemate, mixture of stereoisomers thereof, or a derivative therof.

37. The kit of claim 36, wherein the kit further includes instructions for administration for the treatment of HIV infection and AIDS.

38. A compound selected from the group consisting of:
((R)-4-{Methoxyimino]-phenyl-methyl}-2-methyl-piperazin-1-yl)-acetonitrile;
((R)-3-Methyl-piperazin-1-yl)-phenyl-methanone O-methyl-oxime;
(3R)-3-methyl-1-(phenylcarbonothioyl)piperazine;
5-(4-Benzoyl-2-methyl-piperazine-1-sulfonyl)-thiophene-2-carboxylic acid ethyl ester;
[4-(5-Bromo-thiophene-2-sulfonyl)-3-methyl-piperazin-1-yl]-phenyl-methanone;
[4-(5-Bromo-thiophene-2-sulfonyl)-3-methyl-piperazin-1-yl]-phenyl-methanone;
tert-butyl 3-methyl-4-(thiophen-2-ylsulfonyl)piperazine-1-carboxylate;
[4-(4-Bromo-benzenesulfonyl)-3-methyl-piperazin-1-yl]-phenyl-methanone;
(R)-(4-(4-ethynylphenylsulfonyl)-3-methylpiperazin-1-yl)(phenyl)methanone;
4-(1-Methyl-1H-pyrazol-3-yl)-benzoic acid methyl ester;
1-(4-Benzoyl-2-methyl-piperazin-1-yl)-2-(4-iodo-phenyl)-ethane-1,2-dione;
1-((R)-4-Benzoyl-2-methyl-piperazin-1-yl)-2-(4-bromo-phenyl)-ethane-1,2-dione;
1-((R)-4-Benzoyl-2-methyl-piperazin-1-yl)-2-(4-bromo-2-fluoro-phenyl)-ethane-1,2-dione;
1-((R)-4-Benzoyl-2-methyl-piperazin-1-yl)-2-(4-bromo-3-methyl-phenyl)-ethane-1,2-dione;
1-((R)-4-Benzoyl-2-methyl-piperazin-1-yl)-2-(4-bromo-2-methyl-phenyl)-ethane-1,2-dione;
1-((R)-4-Benzoyl-2-methyl-piperazin-1-yl)-2-(6-chloro-pyridin-3-yl)-ethane-1,2-dione;
4-[2-((R)-4-Benzoyl-2-methyl-piperazin-1-yl)-2-oxo-acetyl]-boronic acid;
1-((R)-4-Benzoyl-2-methyl-piperazin-1-yl)-2-(4-bromo-2-dimethylamino-phenyl)-ethane-1,2-dione; and
1-(2-Amino-4-bromo-phenyl)-2-((R)-4-benzoyl-2-methyl-piperazin-1-yl)-ethane-1,2-dione.
